(19) United States
(12) Patent Application Publication
(10) Pub. No.: US 2025/0135010 A1
(43) Pub. Date: May 1, 2025

(54) CONCATEMERIC PEPTIDE EPITOPE RNAS

(71) Applicant: ModernaTX, INC., Cambridge, MA (US)

(72) Inventors: Giuseppe CIARAMELLA, Sudbury, MA (US); Eric YI-CHUN HUANG, Boston, MA (US); Nicholas VALIANTE, Cambridge, MA (US); Tal ZAKS, Newton, MA (US)

(73) Assignee: ModernaTX, INC., Cambridge, MA (US)

(21) Appl. No.: 18/946,487

(22) Filed: Nov. 13, 2024

Related U.S. Application Data
(63) Continuation of application No. 15/748,773, filed on Jan. 30, 2018, now Pat. No. 12,150,980.
(60) Provisional application No. 62/199,204, filed on Jul. 30, 2015.
(60) Provisional application No. 62/245,145, filed on Oct. 22, 2015.
(60) Provisional application No. 62/247,367, filed on Oct. 28, 2015.

(51) Int. Cl. A61K 47/54; A61K 9/1271; A61K 39/00; A61K 45/06; A61K 47/26; A61K 47/68; A61P 35/00; C12N 15/62

(52) U.S. Cl. CPC A61K 47/543; A61K 9/1271; A61K 39/0011; A61K 39/001114; A61K 45/06; A61K 47/26; A61K 47/6807; A61P 35/00; C12N 15/62; A61K 2039/51; A61K 2039/53; A61K 2039/542; A61K 2039/645; A61K 2039/70

(57) ABSTRACT

The invention relates to concatemeric peptide epitope RNAs, as well as methods and compositions thereof. mRNA vaccines are also provided according to the invention, including cancer vaccines.

### RELATED APPLICATIONS

This application is a continuation of U.S. application Ser. No. 15/748,773, filed Jan. 30, 2018, which was the U.S. national stage under 35 U.S.C. § 371 of international application PCT/US2016/044918, filed Jul. 29, 2016, which claims priority to U.S. Provisional Application No. 62/199,204, filed Jul. 30, 2015, U.S. Provisional Application No. 62/247,367, filed Oct. 28, 2015 and U.S. Provisional Application No. 62/245,145, filed Oct. 22, 2015, the contents of each of which are incorporated herein by reference in their entireties.

### SEQUENCE LISTING

The instant application contains a sequence listing which has been submitted in XML file format that is incorporated by reference herein. Said XML file was created on Nov. 7, 2024, is named 131986-6482_SL.xml and is 1,475,661 bytes in size.

### BACKGROUND OF INVENTION

Cancer vaccines include preventive or prophylactic vaccines, which are intended to prevent cancer from developing in healthy people; and therapeutic vaccines, which are intended to treat an existing cancer by strengthening the body's natural defenses against the cancer. Cancer preventive vaccines may, for instance, target infectious agents that cause or contribute to the development of cancer in order to prevent infectious diseases from causing cancer. Gardasil® and Cervarix®, are two examples of commercially available prophylactic vaccines. Each vaccine protects against HPV infection. Other preventive cancer vaccines may target host proteins or fragments that are predicted to increase the likelihood of an individual developing cancer in the future.

Most commercial or developing vaccines are based on whole microorganisms, protein antigens, peptides, polysaccharides or deoxyribonucleic acid (DNA) vaccines and their combinations. DNA vaccination is one technique used to stimulate humoral and cellular immune responses to antigens. The direct injection of genetically engineered DNA (e.g., naked plasmid DNA) into a living host results in a small number of its cells directly producing an antigen, resulting in a protective immunological response. With this technique, however, comes potential problems of DNA integration into the vaccine's genome, including the possibility of insertional mutagenesis, which could lead to the activation of oncogenes or the inhibition of tumor suppressor genes.

### SUMMARY OF INVENTION

The methods of the invention enable the production of highly antigenic RNA cancer vaccines, including mRNAs encoding concatemeric cancer peptide epitopes. The peptide epitopes are designed to be processed intracellulary and presented to the immune system in an efficient manner. The mRNA cancer vaccines described herein are useful for generating a desired immune response by selecting appropriate T or B cell epitopes which are able to be presented more effectively on MHC-I or MHC-II molecules (depending on whether they are T or B-cell epitope, respectively). In some embodiments T cell epitopes are preferred.

The concatemeric vaccines may be personalized cancer vaccines. For instance, the vaccines may include RNA encoding for one or more cancer antigens specific for each subject.

An mRNA cancer vaccine is provided in some aspects of the invention. The vaccine includes an mRNA having an open reading frame encoding a concatemeric cancer antigen comprised of 2-100 peptide epitopes, a pharmaceutically acceptable carrier or excipient and wherein the mRNA cancer vaccine further comprises one or more of:
a) the of 2-100 peptide epitopes are interspersed by cleavage sensitive sites; b) the mRNA encoding each peptide epitope is linked directly to one another without a linker;
c) the mRNA encoding each peptide epitope is linked to one another with a single nucleotide linker;
d) each peptide epitope comprises a 25-35 amino acids and includes a centrally located SNP mutation;
e) at least 30% of the peptide epitopes have a highest affinity for class I MHC molecules from the subject;
f) at least 30% of the peptide epitopes have a highest affinity for class II MHC molecules from the subject;
g) at least 50% of the peptide epitopes have a predicted binding affinity of IC>500 nM for HLA-A, HLA-B and/or DRB1;
h) the mRNA encodes 20 peptide epitopes;
i) 50% of the peptide epitopes have a binding affinity for class I MHC and 50% of the peptide epitopes have a binding affinity for class II MHC; and/or
j) the mRNA encoding the peptide epitopes is arranged such that the peptide epitopes are ordered to minimize pseudo-epitopes.

In some embodiments each peptide epitope comprises 31 amino acids and includes a centrally located SNP mutation with 15 flanking amino acids on each side of the SNP mutation.

In some aspects the invention is an mRNA cancer vaccine having a mRNA with an open reading frame encoding a concatemeric cancer antigen comprised of peptide epitopes interspersed by cleavage sensitive sites and a pharmaceutically acceptable carrier or excipient. In some embodiments, the concatemeric cancer antigen comprises between 2-100 peptide epitopes interspersed by cleavage sensitive sites.

In some aspects the mRNA cancer vaccine is a mRNA having an open reading frame encoding the following peptide (Z₁)ₘ—(Y₁)ₚ— (X₁—(Y₁)ₚ—X₂—(Y₁)ₚ—X_{3 . . .} —(Y₁)ₚ—Xₙ)—(Y₁)ₚ—(Z₂)ₒ wherein Z₁ and Z₂ are targeting sequences, independent of one another, wherein X is a cancer peptide epitope, each cancer peptide epitope independent of one another, wherein Y is a cleavage sensitive site, wherein m is 0-1, wherein n is 4-100, wherein p is 0-5 and wherein o is 0-1.

An mRNA cancer vaccine comprising an mRNA having an open reading frame encoding a concatemeric antigen and a recall antigen is provided in other aspects of the invention. In some embodiments the recall antigen is an infectious disease antigen. In other embodiments the recall antigen is an mRNA having an open reading frame encoding the recall antigen. In other embodiments the recall antigen is a peptide epitope in the concatemeric antigen. In yet other embodiments the recall antigen is an influenza antigen.

The vaccine in some embodiments is a personalized cancer vaccine and the concatemeric antigen comprises subject specific cancer peptide epitopes. The subject specific cancer peptide epitopes may be representative of an exome of a tumor sample of the subject or a transcriptome of a tumor sample of the subject.

In some embodiments the concatemeric antigen encodes 5-10 cancer peptide epitopes. In yet other embodiments the concatemeric antigen encodes 25-100 cancer peptide epitopes. In yet other embodiments the concatemeric antigen encodes 10-1,000 cancer peptide epitopes.

The concatemeric antigen encodes peptide epitopes of 10-50 amino acids in length in some embodiments. In other embodiments the concatemeric antigen encodes peptide epitopes of 15-20 amino acids in length. In other embodiments the concatemeric antigen encodes peptide epitopes of 20-50, 25-100, 100-200, 200-300, 300-400, or 400-500 amino acids in length.

In some embodiments, the peptide epitopes comprise at least one MHC class I epitope and at least one MHC class II epitope. In some embodiments, at least 10% of the epitopes are MHC class I epitopes. In some embodiments, at least 20% of the epitopes are MHC class I epitopes. In some embodiments, at least 30% of the epitopes are MHC class I epitopes. In some embodiments, at least 40% of the epitopes are MHC class I epitopes. In some embodiments, at least 50%, 60%, 70%, 80%, 90% or 100% of the epitopes are MHC class I epitopes. In some embodiments, at least 10% of the epitopes are MHC class II epitopes. In some embodiments, at least 20% of the epitopes are MHC class II epitopes. In some embodiments, at least 30% of the epitopes are MHC class II epitopes. In some embodiments, at least 40% of the epitopes are MHC class II epitopes. In some embodiments, at least 50%, 60%, 70%, 80%, 90% or 100% of the epitopes are MHC class II epitopes. In some embodiments, the ratio of MHC class I epitopes to MHC class II epitopes is a ratio selected from about 10%:about 90%; about 20%:about 80%; about 30%:about 70%; about 40%:about 60%; about 50%:about 50%; about 60%:about 40%; about 70%:about 30%; about 80%:about 20%; about 90%:about 10% MHC class 1:MHC class II epitopes. In some embodiments, the ratio of MHC class II epitopes to MHC class I epitopes is a ratio selected from about 10%:about 90%; about 20%:about 80%; about 30%:about 70%; about 40%:about 60%; about 50%:about 50%; about 60%:about 40%; about 70%:about 30%; about 80%:about 20%; about 90%:about 10% MHC class II:MHC class I epitopes. In some embodiments, at least one of the peptide epitopes of the cancer vaccine is a B cell epitope. In some embodiments, the T cell epitope of the cancer vaccine comprises between 8-11 amino acids. In some embodiments, the B cell epitope of the cancer vaccine comprises between 13-17 amino acids.

The vaccines described herein may be formulated in a lipid nanoparticle having a mean diameter of 50-200 nm.

In some embodiments, the mRNA cancer vaccine further comprises one or more additional mRNAs having an open reading frame encoding an antigen.

In some embodiments, the peptide epitopes of the mRNA cancer vaccine are T cell epitopes and/or B cell epitopes. In other embodiments, the peptide epitopes of the mRNA cancer vaccine comprise a combination of T cell epitopes and B cell epitopes. In yet another embodiment, at least 1 of the peptide epitopes of the mRNA cancer vaccine is a T cell epitope. In other embodiments, at least 1 of the peptide epitopes of the mRNA cancer vaccine is a B cell epitope.

In some embodiments, the T cell epitope of the mRNA cancer vaccine comprises between 8-11 amino acids. In other embodiments, the B cell epitope of the mRNA cancer vaccine comprises between 13-17 amino acids.

In some embodiments, the mRNA of the mRNA cancer vaccine further comprises one or more ubiquitination signals. In some embodiments, the ubiquitination signal of the mRNA cancer vaccine is located at the C-terminus of the mRNA.

In some embodiments, the cleavage sensitive sites of the mRNA cancer vaccine are protease cleavage sites. In other embodiments, the protease cleavage site of the mRNA cancer vaccine is a cleavage site for a serine protease, a threonine protease, a cysteine protease, an aspartate protease, a glutamic acid protease, or a metalloprotease.

In some embodiments, the protease cleavage site of the mRNA cancer vaccine is for a cysteine protease. In some embodiments, the cysteine protease is cathepsin B.

In some embodiments, the protease cleavage site comprises the amino acid sequence GFLG (SEQ ID NO: 1), KVSR (SEQ ID NO: 2), TVGLR (SEQ ID NO: 3), PMGLP (SEQ ID NO: 4), or PMGAP (SEQ ID NO: 5).

In some embodiments, mRNA of the mRNA cancer vaccine further comprises a 5′ terminal cap. In some embodiments, the 5′ terminal cap of the mRNA cancer vaccine is 7mG(5′)ppp(5′)NlmpNp.

In some embodiments, the mRNA of the mRNA cancer vaccine further comprises an endosomal targeting sequence. In some embodiments, the endosomal targeting sequence of the mRNA cancer vaccine comprises at least a portion of the transmembrane domain of lysosome associated membrane protein (LAMP-1). In other embodiments, the endosomal targeting sequence of the mRNA cancer vaccine comprises at least a portion of the transmembrane domain of invariant chain (Ii).

In some embodiments, the mRNA cancer vaccine does not comprise a stabilization agent.

In some embodiments, the mRNA of the mRNA cancer vaccine is formulated in a lipid nanoparticle carrier. In further embodiments, the lipid nanoparticle carrier comprises a molar ratio of about 20-60% cationic lipid:5-25% non-cationic lipid:25-55% sterol; and 0.5-15% PEG-modified lipid. In other embodiments, the cationic lipid is selected from the group consisting of for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319). In some embodiments the lipid nanoparticle has a polydispersity value of less than 0.4. In other embodiments the lipid nanoparticle has a net neutral charge at a neutral pH value.

In some embodiments, the mRNA of the mRNA cancer vaccine includes at least one chemical modification. In some embodiments, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, and 2′-O-methyl uridine.

In some embodiments, the mRNA of the mRNA cancer vaccine further includes an open reading frame encoding one or more traditional cancer antigens. In some embodiments, the mRNA of the mRNA cancer vaccine further includes an mRNA having an open reading frame encoding one or more traditional cancer antigens.

In other aspects the invention is an mRNA vaccine, comprising: a mRNA having an open reading frame encoding a concatemeric antigen comprised of 2-100 peptide epitopes interspersed by linkers and a pharmaceutically acceptable carrier or excipient, wherein each peptide epitope comprises an antigenic region and a MHC stabilizing region. In some embodiments the peptide epitopes are cancer peptide epitopes. In other embodiments the MHC stabilizing region is 5-10 amino acids in length. In yet other embodiments the antigenic region is 5-100 amino acids in length.

A method for vaccinating a subject by administering to a subject having cancer a mRNA vaccine having an open reading frame encoding a concatemeric antigen comprised of 2-100 cancer peptide epitopes interspersed by linkers and a pharmaceutically acceptable carrier or excipient, wherein each peptide epitope comprises an antigenic region and a MHC stabilizing region in order to vaccinate the subject is provided in other aspects of the invention. In some embodiments the MHC stabilizing region is designed based on the subject's MHC.

In some embodiments, the mRNA vaccine further encodes one or more traditional cancer antigens. In some embodiments, the one or more traditional cancer antigens are encoded by the same mRNA that encode the set of neoepitopes. In some embodiments, the one or more traditional cancer antigens are encoded by a different mRNA than the mRNA which encodes the set of neoepitopes.

In some embodiments, the mRNA vaccine is administered in combination with a cancer therapeutic agent. In some embodiments, the cancer therapeutic agent is a traditional cancer vaccine.

In other aspects the invention is a method for preparing a mRNA cancer vaccine, by isolating a sample from a subject, identifying a plurality of cancer antigens in the sample, determining T-cell epitopes from the plurality of cancer antigens, preparing a mRNA cancer vaccine having an open reading frame encoding a concatemeric antigen, wherein the concatemeric antigen is comprised of 2-100 of the T-cell epitopes interspersed by linkers. In some embodiments the method further involves determining binding strength of the T-cell epitopes to a MHC of a subject. In other embodiments the method further involves determining a T-cell receptor face (TCR face) for each epitope and selecting epitopes having a TCR face with low similarity to endogenous proteins.

A method for vaccinating a subject by administering to a subject a mRNA cancer vaccine having an open reading frame encoding a concatemeric antigen comprised of 2-100 T-cell epitopes from the plurality of cancer antigens of a subject, interspersed by linkers, wherein the T-cell epitopes have been optimized for binding strength to a MHC of the subject is provided in aspects of the invention.

In other aspects an mRNA vaccine, comprising: a mRNA having an open reading frame encoding a concatemeric antigen comprised of 2-100 T-cell epitopes from the plurality of cancer antigens of a subject, interspersed by linkers, wherein the T-cell epitopes have been optimized for binding strength to a MHC of the subject is provided. In some embodiments a TCR face for each epitope has a low similarity to endogenous proteins.

Another aspect of the present disclosure includes a method for administering any one of the mRNA cancer vaccines as described above, in an effective amount to a subject in need thereof. In some embodiments, the administration is orally, intranasally, intravenously, intradermally, intramuscularly, or intraperitoneally. In some embodiments, the administration is a single administration. In some embodiments, the subject is administered the vaccine more than once.

A method of eliciting an immune response in a subject against a cancer antigen is provided in aspects of the invention. The method involves administering to the subject a RNA vaccine comprising at least one RNA polynucleotide having an open reading frame encoding at least one antigenic polypeptide or an immunogenic fragment thereof, thereby inducing in the subject an immune response specific to the antigenic polypeptide or an immunogenic fragment thereof, wherein the anti-antigenic polypeptide antibody titer in the subject is increased following vaccination relative to anti-antigenic polypeptide antibody titer in a subject vaccinated with a prophylactically effective dose of a traditional vaccine against the cancer. An “anti-antigenic polypeptide antibody” is a serum antibody the binds specifically to the antigenic polypeptide.

A prophylactically effective dose is a therapeutically effective dose that prevents advancement of cancer at a clinically acceptable level. In some embodiments the therapeutically effective dose is a dose listed in a package insert for the vaccine. A traditional vaccine, as used herein, refers to a vaccine other than the mRNA vaccines of the invention. For instance, a traditional vaccine includes but is not limited to live microorganism vaccines, killed microorganism vaccines, subunit vaccines, protein antigen vaccines, DNA vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA.) In some embodiments the anti-antigenic polypeptide antibody titer in the subject is increased 1 log to 10 log following vaccination relative to anti-antigenic polypeptide antibody titer in a subject vaccinated with a prophylactically effective dose of a traditional vaccine against the cancer.

In some embodiments the anti-antigenic polypeptide antibody titer in the subject is increased 1 log following vaccination relative to anti-antigenic polypeptide antibody titer in a subject vaccinated with a prophylactically effective dose of a traditional vaccine against the cancer.

In some embodiments the anti-antigenic polypeptide antibody titer in the subject is increased 2 log following vaccination relative to anti-antigenic polypeptide antibody titer in a subject vaccinated with a prophylactically effective dose of a traditional vaccine against the cancer.

In some embodiments the anti-antigenic polypeptide antibody titer in the subject is increased 3 log following vaccination relative to anti-antigenic polypeptide antibody titer in a subject vaccinated with a prophylactically effective dose of a traditional vaccine against the cancer.

In some embodiments the anti-antigenic polypeptide antibody titer in the subject is increased 5 log following vaccination relative to anti-antigenic polypeptide antibody titer in a subject vaccinated with a prophylactically effective dose of a traditional vaccine against the or cancer.

In some embodiments the anti-antigenic polypeptide antibody titer in the subject is increased 10 log following vaccination relative to anti-antigenic polypeptide antibody titer in a subject vaccinated with a prophylactically effective dose of a traditional vaccine against the or cancer.

A method of eliciting an immune response in a subject against a cancer antigen is provided in other aspects of the invention. The method involves administering to the subject a RNA vaccine comprising at least one RNA polynucleotide having an open reading frame encoding at least one antigenic polypeptide or an immunogenic fragment thereof, thereby inducing in the subject an immune response specific to antigenic polypeptide or an immunogenic fragment thereof, wherein the immune response in the subject is equivalent to an immune response in a subject vaccinated with a traditional vaccine against the cancer antigen at 2 times to 100 times the dosage level relative to the RNA vaccine.

In some embodiments the immune response in the subject is equivalent to an immune response in a subject vaccinated with a traditional vaccine at twice the dosage level relative to the RNA vaccine.

In some embodiments the immune response in the subject is equivalent to an immune response in a subject vaccinated with a traditional vaccine at three times the dosage level relative to the RNA vaccine.

In some embodiments the immune response in the subject is equivalent to an immune response in a subject vaccinated with a traditional vaccine at 4 times the dosage level relative to the RNA vaccine.

In some embodiments the immune response in the subject is equivalent to an immune response in a subject vaccinated with a traditional vaccine at 5 times the dosage level relative to the RNA vaccine.

In some embodiments the immune response in the subject is equivalent to an immune response in a subject vaccinated with a traditional vaccine at 10 times the dosage level relative to the RNA vaccine.

In some embodiments the immune response in the subject is equivalent to an immune response in a subject vaccinated with a traditional vaccine at 50 times the dosage level relative to the RNA vaccine.

In some embodiments the immune response in the subject is equivalent to an immune response in a subject vaccinated with a traditional vaccine at 100 times the dosage level relative to the RNA vaccine.

In some embodiments the immune response in the subject is equivalent to an immune response in a subject vaccinated with a traditional vaccine at 10 times to 1000 times the dosage level relative to the RNA vaccine.

In some embodiments the immune response in the subject is equivalent to an immune response in a subject vaccinated with a traditional vaccine at 100 times to 1000 times the dosage level relative to the RNA vaccine.

In other embodiments the immune response is assessed by determining antibody titer in the subject.

In other aspects the invention is a method of eliciting an immune response in a subject against a by administering to the subject a RNA vaccine comprising at least one RNA polynucleotide having an open reading frame encoding at least one cancer antigenic polypeptide or an immunogenic fragment thereof, thereby inducing in the subject an immune response specific to the antigenic polypeptide or an immunogenic fragment thereof, wherein the immune response in the subject is induced 2 days to 10 weeks earlier relative to an immune response induced in a subject vaccinated with a prophylactically effective dose of a traditional vaccine against the cancer antign. In some embodiments the immune response in the subject is induced in a subject vaccinated with a prophylactically effective dose of a traditional vaccine at 2 times to 100 times the dosage level relative to the RNA vaccine.

In some embodiments the immune response in the subject is induced 2 days earlier relative to an immune response induced in a subject vaccinated with a prophylactically effective dose of a traditional vaccine.

In some embodiments the immune response in the subject is induced 3 days earlier relative to an immune response induced in a subject vaccinated a prophylactically effective dose of a traditional vaccine.

In some embodiments the immune response in the subject is induced 1 week earlier relative to an immune response induced in a subject vaccinated with a prophylactically effective dose of a traditional vaccine.

In some embodiments the immune response in the subject is induced 2 weeks earlier relative to an immune response induced in a subject vaccinated with a prophylactically effective dose of a traditional vaccine.

In some embodiments the immune response in the subject is induced 3 weeks earlier relative to an immune response induced in a subject vaccinated with a prophylactically effective dose of a traditional vaccine.

In some embodiments the immune response in the subject is induced 5 weeks earlier relative to an immune response induced in a subject vaccinated with a prophylactically effective dose of a traditional vaccine.

In some embodiments the immune response in the subject is induced 10 weeks earlier relative to an immune response induced in a subject vaccinated with a prophylactically effective dose of a traditional vaccine.

A method of eliciting an immune response in a subject against an cancer by administering to the subject a cancer RNA vaccine having an open reading frame encoding a first antigenic polypeptide, wherein the RNA polynucleotide does not include a stabilization element, and wherein an adjuvant is not coformulated or co-administered with the vaccine.

In some aspects the invention is a method for vaccinating a subject, by administering to a subject having cancer an mRNA vaccine comprising one or more polynucleotides having an open reading frame encoding cancer peptide epitopes, wherein the mRNA vaccine encodes 5-100 peptide epitopes as single antigens or as a concatemer or a combination thereof in order to vaccinate the subject and further comprising administering to the subject having cancer an immune checkpoint modulator. In some embodiments the immune checkpoint modulator is an inhibitory checkpoint polypeptide. The inhibitory checkpoint polypeptide is an anti-CTLA4 or anti-PD1 antibody in some embodiments. In other embodiments the inhibitory checkpoint polypeptide is an antibody or fragment thereof that specifically binds to a molecule selected from the group consisting of PD-1, TIM-3, VISTA, A2AR, B7-H3, B7-H4, BTLA, CTLA-4, IDO, KIR and LAG3.

In other aspects the invention is an mRNA cancer vaccine, having an mRNA having an open reading frame encoding a concatemeric cancer antigen comprised of 2-100 peptide epitopes and a pharmaceutically acceptable carrier or excipient, wherein the mRNA is preparable by ligating 2-100 individual mRNAs encoding each of the 2-100 peptide epitopes.

In yet other aspects the invention is a method of producing an mRNA encoding a concatemeric cancer antigen comprising between 1000 and 3000 nucleotides, the method by
(a) binding a first polynucleotide comprising an open reading frame encoding the concatemeric cancer antigen and a second polynucleotide comprising a 5′-UTR to a polynucleotide conjugated to a solid support;
(b) ligating the 3′-terminus of the second polynucleotide to the 5′-terminus of the first polynucleotide under suitable conditions, wherein the suitable conditions comprise a DNA Ligase, thereby producing a first ligation product;
(c) ligating the 5′ terminus of a third polynucleotide comprising a 3′-UTR to the 3′-terminus of the first ligation product under suitable conditions, wherein the suitable conditions comprise an RNA Ligase, thereby producing a second ligation product; and
(d) releasing the second ligation product from the solid support,
thereby producing an mRNA encoding the concatemeric cancer antigen comprising between 1000 and 3000 nucleotides.

A kit for preparing an mRNA cancer vaccine is provided in other aspects of the invention. The kit has one or more containers housing one or more polynucleotides comprising a 5′-ORF, one or more polynucleotides comprising a 3′-ORF, one or more polynucleotides comprising a poly(A) tail, a ligase enzyme, and instructions for ligating one or more polynucleotides comprising an ORF encoding a patient specific epitope to the one or more polynucleotides comprising the a 5′-ORF, 3′-ORF, and poly(A) tail.

A method for treating a subject with a personalized mRNA cancer vaccine, by isolating a sample from a subject, identifying a set of neoepitopes by analyzing a patient transcriptome and/or a patient exome from the sample to produce a patient specific mutanome, selecting a set of neoepitopes for the vaccine from the mutanome based on MHC binding strength, MHC binding diversity, predicted degree of immunogenicity, low self reactivity, and/or T cell reactivity, preparing the mRNA vaccine to encode the set of neoepitopes and administering the mRNA vaccine to the subject within two months of isolating the sample from the subject is provided in other aspects of the invention. In some embodiments the mRNA vaccine is administered to the subject within one month of isolating the sample from the subject.

In other aspects the invention is a method of identifying a set of neoepitopes for use in a personalized mRNA cancer vaccine having one or more polynucleotides that encode the set of neoepitopes by
a. identifying a patient specific mutanome by analyzing a patient transcriptome and a patient exome,
b. selecting a subset of 15-500 neoepitopes from the mutanome using a weighted value for the neoepitopes based on at least three of: an assessment of gene or transcript-level expression in patient RNA-seq; variant call confidence score; RNA-seq allele-specific expression; conservative vs. non-conservative amino acid substitution; position of point mutation (Centering Score for increased TCR engagement); position of point mutation (Anchoring Score for differential HLA binding); Selfness: <100% core epitope homology with patient WES data; HLA-A and -B IC50 for 8mers-llmers; HLA-DRB1 IC50 for 15mers-20mers; promiscuity Score (i.e. number of patient HLAs predicted to bind); HLA-C IC50 for 8mers-llmers; HLA-DRB3-5 IC50 for 15mers-20mers; HLA-DQB1/A1 IC50 for 15mers-20mers; HLA-DPB1/A1 IC50 for 15mers-20mers; Class I vs Class II proportion; Diversity of patient HLA-A, —B and DRB1 allotypes covered; proportion of point mutation vs complex epitopes (e.g. frameshifts); and/or pseudo-epitope HLA binding scores, and
c. selecting the set of neoepitopes for use in a personalized mRNA cancer vaccine from the subset based on the highest weighted value, wherein the set of neoepitopes comprise 15-40 neoepitopes.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1D show the results of an assay to demonstrate a mRNA vaccine antigen specific CD8 response.

FIG. 2 shows the results of an assay to demonstrate a mRNA vaccine induced antigen specific effector/memory CD8 T cell response.

FIG. 3 is a schematic depicting a multi-factorial consideration of antigen design of mRNA-based neoepitopes. The sequences, from top to bottom, correspond to SEQ ID NOs: 1130 and 1131.

FIG. 4 is a table depicting a multi-factorial consideration of antigen design of mRNA-based neoepitopes.

FIG. 5 depicts the results of a validation of FACS-based assay of mRNA encoded epitopes in MCF7 (HLA*201). Specific MHC1/mut.MART1peptide presentation by anti-mut.MART1TCRmer was detected on MCF7 cells. The sequences, from top to bottom, correspond to SEQ ID NOs: 1123-1127.

FIGS. 6A and 6B are schematics of an exemplary peptide epitopes. The polypeptide of FIG. 6A includes two or more epitopes. The epitopes can be of the same sequence or different sequence and can be all T-cell epitopes, all B-cell epitopes or a combination of both. The schematic of FIG. 6B shows the peptide epitope with various end units for enhancing MHC processing of the peptides.

FIG. 7 depicts exemplary T cell response elicited with mRNA encoding concatamers of 20 epitopes. mRNA concatamers induced both class I and class II T cell responses.

FIG. 8 depicts exemplary T cell response elicited with mRNA encoding concatamers with and without RP.

FIG. 9A depicts exemplary T cell response elicited with mRNA encoding concatamers with epitopes in differing positions. CA80 and CA81 encode the same 20 epitopes known to elicit T cell responses. They include 5 class II epitopes, 10 murine class I epitopes, a murine positive control (SIINFEKL (SEQ ID NO: 6), derived from ovalbumin), and 4 human (HLA-A2) epitopes (not shown). CA80 and CA81 differ only in the relative positions of the different epitopes. FIG. 9B depicts exemplary correlation between interferon-gamma spot forming units (SFUs) and CD8+INFN-g+ responses.

FIG. 10 is a schematic of an exemplary mRNA component of mRNA-4379.

FIG. 11 is a schematic of an exemplary general molecular sequence of mRNA-4379, in which the patient specific coding region is depicted by reference as (N). The sequences shown are ggggaaauaagagagaaaagaagaguaagaagaaauauaagagccaccaug (SEQ ID NO: 1128), and ugauaauaggcuggagccucgguggccaugcuucuugccccuugggccuccccccagccccuccuccccuuccugcaccc guacccccguggucuuugaauaaagucugagugggcggcaaaaaaaaaaaaaaaaaaaaaaaaaaaaaaaaaaaaaaaaaa aaaaaaaaaaaaaaaaaaaaaaaaaaaaaaaaaaaaaaaaaaaaaaaaaaaaaaaaaaaaucuag (SEQ ID NO: 1129).

FIG. 12 is a block diagram of an exemplary computer system on which some embodiments may be implemented.

### DETAILED DESCRIPTION

The generation of antigens that elicit a desired immune response (e.g. T-cell responses) against targeted polypeptide sequences in vaccine development remains a challenging task. The invention involves technology that overcome hurdles associated with vaccine development. Through the use of the technology of the invention, it is possible to tailor the desired immune response by selecting appropriate T or B cell epitopes which, by virtue of the fact that they are processed intra-cellularly, are able to be presented more effectively on MHC-1 or MHC-2 molecules (depending on whether they are T or B-cell epitope, respectively). In particular, the invention involves the generation of concatemers of epitopes (particularly T cell epitopes) preferably interspersed with cleavage sites by proteases that are abundant in Antigen Presenting Cells (APCs). These methods mimic antigen processing and may lead to a more effective antigen presentation than can be achieved with peptide antigens.

The fact that the peptide epitopes of the invention are expressed from RNA as intracellular peptides provides advantages over prior art peptides that are delivered as exogenous peptides or as DNA. The RNA is delivered intra-cellularly and expresses the epitopes in proximity to the appropriate cellular machinery for processing the epitopes such that they will be recognized by the appropriate immune cells. Additionally, a targeting sequence will allow more specificity in the delivery of the peptide epitopes. For instance, the invention may involve the addition of a C-terminus Ubiquin Ligase targeting protein (FBox Protein) to target the polypeptide processing to the proteasome and more closely mimic the MHC processing. The constructs of the invention also may include linkers such as proteolytic cleavage sites optimized for APCs. These proteolytic sites provide an advantage because they enhance the processing of the peptides in APCs.

Thus, the invention relates to mRNA vaccines. mRNA vaccines are described in International Patent Application No. PCT/US2015/027400, filed on Apr. 23, 2015, herein incorporated by reference in its entirety.

The mRNA cancer vaccines provide unique therapeutic alternatives to peptide based or DNA vaccines. When the mRNA cancer vaccine is delivered to a cell, the mRNA will be processed into a polypeptide by the intracellular machinery which can then process the polypeptide into immunosensitive fragments capable of stimulating an immune response against the tumor.

The concatemeric cancer vaccines may be personalized cancer vaccines. For instance, the vaccines may include RNA encoding for one or more cancer antigens specific for each subject, referred to as neoepitopes. Antigens that are expressed in or by tumor cells are referred to as “tumor associated antigens”. A particular tumor associated antigen may or may not also be expressed in non-cancerous cells. Many tumor mutations are well known in the art. Tumor associated antigens that are not expressed or rarely expressed in non-cancerous cells, or whose expression in non-cancerous cells is sufficiently reduced in comparison to that in cancerous cells and that induce an immune response induced upon vaccination, are referred to as neoepitopes. Neoepitopes are completely foreign to the body and thus would not produce an immune response against healthy tissue or be masked by the protective components of the immune system. In some embodiments personalized vaccines based on neoepitopes are desirable because such vaccine formulations will maximize specificity against a patients specific tumor. Mutation-derived neoepitopes can arise from point mutations, non-synonymous mutations leading to different amino acids in the protein; read-through mutations in which a stop codon is modified or deleted, leading to translation of a longer protein with a novel tumor-specific sequence at the C-terminus; splice site mutations that lead to the inclusion of an intron in the mature mRNA and thus a unique tumor-specific protein sequence; chromosomal rearrangements that give rise to a chimeric protein with tumor-specific sequences at the junction of 2 proteins (i.e., gene fusion); frameshift mutations or deletions that lead to a new open reading frame with a novel tumor-specific protein sequence; and translocations.

Methods for generating personalized cancer vaccines generally involve identification of mutations, e.g., using deep nucleic acid or protein sequencing techniques, identification of neoepitopes, e.g., using application of validated peptide-MHC binding prediction algorithms or other analytical techniques to generate a set of candidate T cell epitopes that may bind to patient HLA alleles and are based on mutations present in tumors, optional demonstration of antigen-specific T cells against selected neoepitopes or demonstration that a candidate neoepitope is bound to HLA proteins on the tumor surface and development of the vaccine. The concatemeric vaccines of the invention may include multiple copies of a single neoepitope, multiple different neoepitopes based on a single type of mutation, i.e. point mutation, multiple different neoepitopes based on a variety of mutation types, neoepitopes and other antigens, such as tumor associated antigens or recall antigens.

Examples of techniques for identifying mutations include but are not limited to dynamic allele-specific hybridization (DASH), microplate array diagonal gel electrophoresis (MADGE), pyrosequencing, oligonucleotide-specific ligation, the TaqMan system as well as various DNA “chip” technologies i.e. Affymetrix SNP chips, and methods based on the generation of small signal molecules by invasive cleavage followed by mass spectrometry or immobilized padlock probes and rolling-circle amplification.

The deep nucleic acid or protein sequencing techniques are known in the art. Any type of sequence analysis method can be used. For instance nucleic acid sequencing may be performed on whole tumor genomes, tumor exomes (protein-encoding DNA) or tumor transcriptomes. Real-time single molecule sequencing-by-synthesis technologies rely on the detection of fluorescent nucleotides as they are incorporated into a nascent strand of DNA that is complementary to the template being sequenced. Other rapid high throughput sequencing methods also exist. Protein sequencing may be performed on tumor proteomes. Additionally, protein mass spectrometry may be used to identify or validate the presence of mutated peptides bound to MHC proteins on tumor cells. Peptides can be acid-eluted from tumor cells or from HLA molecules that are immunoprecipitated from tumor, and then identified using mass spectrometry. The results of the sequencing may be compared with known control sets or with sequencing analysis performed on normal tissue of the patient.

Accordingly, the present invention relates to methods for identifying and/or detecting neoepitopes of an antigen, such as T-cell epitopes. Specifically, the invention provides methods of identifying and/or detecting tumor specific neoepitopes that are useful in inducing a tumor specific immune response in a subject. Optionally, these neoepitopes bind to class I HLA proteins with a greater affinity than the wild-type peptide and/or are capable of activating anti-tumor CD8 T-cells. Identical mutations in any particular gene are rarely found across tumors.

Proteins of MHC class I are present on the surface of almost all cells of the body, including most tumor cells. The proteins of MHC class I are loaded with antigens that usually originate from endogenous proteins or from pathogens present inside cells, and are then presented to cytotoxic T-lymphocytes (CTLs). T-Cell receptors are capable of recognizing and binding peptides complexed with the molecules of MHC class I. Each cytotoxic T-lymphocyte expresses a unique T-cell receptor which is capable of binding specific MHC/peptide complexes.

Using computer algorithms, it is possible to predict potential neoepitopes such as T-cell epitopes, i.e. peptide sequences, which are bound by the MHC molecules of class I or class II in the form of a peptide-presenting complex and then, in this form, recognized by the T-cell receptors of T-lymphocytes. Examples of programs useful for identifying peptides which will bind to MHC include for instance: Lonza Epibase, SYFPEITHI (Rammensee et al., Immunogenetics, 50 (1999), 213-219) and HLA_BIND (Parker et al., J. Immunol., 152 (1994), 163-175).

Once putative neoepitopes are selected, they can be further tested using in vitro and/or in vivo assays. Conventional in vitro lab assays, such as Elispot assays may be used with an isolate from each patient, to refine the list of neoepitopes selected based on the algorithm's predictions.

In some embodiments the mRNA cancer vaccines and vaccination methods include epitopes or antigens based on specific mutations (neoepitopes) and those expressed by cancer-germline genes (antigens common to tumors found in multiple patients, referred to herein as “traditional cancer antigens” or “shared cancer antigens”). In some embodiments, a traditional antigen is one that is known to be found in cancers or tumors generally or in a specific type of cancer or tumor. In some embodiments, a traditional cancer antigen is a non-mutated tumor antigen. In some embodiments, a traditional cancer antigen is a mutated tumor antigen.

In some embodiments, the vaccines may further include RNA encoding for one or more non-mutated tumor antigens. In some embodiments, the vaccines may further include RNA encoding for one or more mutated tumor antigens.

Antigens that are expressed in or by tumor cells are referred to as “tumor associated antigens”. A particular tumor associated antigen may or may not also be expressed in non-cancerous cells.

Many tumor antigens are known in the art. In some embodiments, the cancer or tumor antigen is one of the following antigens: CD2, CD19, CD20, CD22, CD27, CD33, CD37, CD38, CD40, CD44, CD47, CD52, CD56, CD70, CD79, CD137, 4-IBB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGF1R, Integrin av, Integrin avo, LAG-3, Lewis Y, Mesothelin, c-MET, MN Carbonic anhydrase IX, MUC1, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, and variants thereof.

In some embodiments, the mRNA cancer vaccines and vaccination methods include an mRNA encoding a concatemeric cancer antigen comprised of one or more neoepitopes and one or more traditional, cancer antigens. In some embodiments, the mRNA encodes 1, 2, 3, 4, 5, 6, 7, 8, 9, 10, 11, 12, 13, 14, 15, 16, 17, 18, 19, 20, or more traditional, cancer antigens in addition to the encoded neoepitopes.

The mRNA cancer vaccines of the invention are compositions, including pharmaceutical compositions. The invention also encompasses methods for the selection, design, preparation, manufacture, formulation, and/or use of mRNA cancer vaccines. Also provided are systems, processes, devices and kits for the selection, design and/or utilization of the mRNA cancer vaccines described herein.

In some embodiments the mRNA cancer vaccine of the invention is a poly-epitopic RNA. Poly-epitopes consist of strings of epitopes on the same mRNA. The RNA sequences that code for the peptide epitopes may be interspersed by sequences that code for amino acid sequences recognized by proteolytic enzymes.

Thus, in some embodiments an mRNA cancer vaccine is an mRNA having an open reading frame encoding a propeptide. A propeptide, as used herein, refers to a peptide sequence which includes multiple peptide epitopes linked together either directly or through a linker such as a cleavage sensitive site. An exemplary propeptide has the following peptide sequence: (Z₁)ₘ—(Y₁)ₚ— (X₁—(Y₁)ₚ—X₂—(Y₁)ₚ—X_{3 . . .} —(Y₁)ₚ—Xₙ)— (Y₁)ₚ—(Z₂)ₒ

Z₁ and Z₂ are each targeting sequences, where m=0-1. The mRNA may include a polynucleotide sequence encoding the targeting sequence at either the N or C terminus or both ends of the central peptide epitope region. If a polypeptide has two or more targeting sequences, those targeting sequences may be the same or different targeting sequences. Alternatively the polypeptide encoded by the RNA may not include a targeting sequence.

X refers to a peptide epitope. Each peptide epitope designated by an X may represent a unique peptide epitope in the peptide or it may refer to a copy of an epitope. Thus, the peptide encoded by the mRNA may be composed of multiple peptide epitopes each having a unique sequence. Alternatively the peptide may include 1 or more copies of the same peptide epitope. In some embodiments a peptide may have 3 or more, 4 or more, 5 or more, 6 or more, 7 or more, 8 or more, 9 or more, or 10 or more copies of the same peptide epitope. In other embodiments the peptide may include peptide epitopes that all have the same amino acid sequence.

Y is a linker, optionally a cleavage sensitive site, wherein p is 0-5. Each peptide epitope may optionally have one or more linkers, optionally cleavage sensitive sites adjacent to the N and/or C terminal end. In a concatemeric peptide, two or more of the peptide epitopes may have a cleavage sensitive site between them. Alternatively two or more of the peptide epitopes may be connected directly to one another or through a linker that is not a cleavage sensitive site. The targeting sequence may also be connected to the peptide epitope through a cleavage sensitive site or it may be connected directly to the peptide epitope through a linker that is not a cleavage sensitive site.

A concatemeric peptide as used herein is series of at least two peptide epitopes linked together to form the propeptide. In some embodiments a concatemeric peptide is composed of 3 or more, 4 or more, 5 or more 6 or more 7 or more, 8 or more, 9 or more peptide epitopes. In other embodiments the concatemeric peptide is composed of 1000 or less, 900 or less, 500 or less, 100 or less, 75 or less, 50 or less, 40 or less, 30 or less, 20 or less or 100 or less peptide epitopes. In yet other embodiments, a concatemeric peptide has 3-100, 5-100, 10-100, 15-100, 20-100, 25-100, 30-100, 35-100, 40-100, 45-100, 50-100, 55-100, 60-100, 65-100, 70-100, 75-100, 80-100, 90-100, 5-50, 10-50, 15-50, 20-50, 25-50, 30-50, 35-50, 40-50, 45-50, 100-150, 100-200, 100-300, 100-400, 100-500, 50-500, 50-800, 50-1,000, or 100-1,000 peptide epitopes.

In some embodiments the concatemeric antigen encodes 5-10 cancer peptide epitopes. In yet other embodiments the concatemeric antigen encodes 25-100 cancer peptide epitopes. In some embodiments the mRNA cancer vaccines and vaccination methods include epitopes or antigens based on specific mutations (neoepitopes) and those expressed by cancer-germline genes (antigens common to tumors found in multiple patients). In some embodiments, the mRNA cancer vaccines and vaccination methods include one or more traditional epitopes or antigens, e.g., one or more epitopes or antigens found in a traditional cancer vaccine.

An epitope, also known as an antigenic determinant, as used herein is a portion of an antigen that is recognized by the immune system in the appropriate context, specifically by antibodies, B cells, or T cells. Epitopes include B cell epitopes and T cell epitopes. B-cell epitopes are peptide sequences which are required for recognition by specific antibody producing B-cells. B cell epitopes refer to a specific region of the antigen that is recognized by an antibody. The portion of an antibody that binds to the epitope is called a paratope. An epitope may be a conformational epitope or a linear epitope, based on the structure and interaction with the paratope. A linear, or continuous, epitope is defined by the primary amino acid sequence of a particular region of a protein. The sequences that interact with the antibody are situated next to each other sequentially on the protein, and the epitope can usually be mimicked by a single peptide. Conformational epitopes are epitopes that are defined by the conformational structure of the native protein. These epitopes may be continuous or discontinuous, i.e. components of the epitope can be situated on disparate parts of the protein, which are brought close to each other in the folded native protein structure.

T-cell epitopes are peptide sequences which, in association with proteins on APC, are required for recognition by specific T-cells. T cell epitopes are processed intracellularly and presented on the surface of APCs, where they are bound to MHC molecules including MHC class II and MHC class I.

The present disclosure, in some aspects, relates to a process of developing T or B cell concatemeric epitopes or concatemeric epitopes composed of both B and T cell epitopes. Several tools exist for identifying various peptide epitopes. For instance, epitopes can be identified using a free or commercial database (Lonza Epibase, antitope for example). Such tools are useful for predicting the most immunogenic epitopes within a target antigen protein. The selected peptides may then be synthesized and screened in human HLA panels, and the most immunogenic sequences are used to construct the mRNAs encoding the concatemeric antigens. One strategy for mapping epitopes of Cytotoxic T-Cells based on generating equimolar mixtures of the four C-terminal peptides for each nominal 11-mer across a protein. This strategy would produce a library antigen containing all the possible active CTL epitopes

The peptide epitope may be any length that is reasonable for an epitope. In some embodiments the peptide epitope is 9-30 amino acids. In other embodiments the length is 9-22, 9-29, 9-28, 9-27, 9-26, 9-25, 9-24, 9-23, 9-21, 9-20, 9-19, 9-18, 10-22, 10-21, 10-20, 11-22, 22-21, 11-20, 12-22, 12-21, 12-20,13-22, 13-21, 13-20, 14-19, 15-18, or 16-17 amino acids.

In some embodiments, the optimal length of a peptide epitope may be obtained through the following procedure: synthesizing a V5 tag concatemer-test protease site, introducing it into DC cells (for example, using an RNA Squeeze procedure, lysing the cells, and then running an anti-V5 Western blot to assess the cleavage at protease sites.

The RNA Squeeze technique is an intracellular delivery method by which a variety of materials can be delivered to a broad range of live cells. Cells are subjected to microfluidic construction, which causes rapid mechanical deformation. The deformation results in temporary membrane disruption and the newly-formed transient pores. Material is then passively diffused into the cell cytosol via the transient pores. The technique can be used in a variety of cell types, including primary fibroblasts, embryonic stem cells, and a host of immune cells, and has been shown to have relatively high viability in most applications and does not damage sensitive materials, such as quantum dots or proteins, through its actions. Sharei et al., PNAS (2013); 110(6):2082-7.

In some embodiments the concatemeric antigen may include a recall antigen, also sometimes referred to as a memory antigen. A recall antigen is an antigen that has previously been encountered by an individual and for which there are pre-existent memory lymphocytes. In some embodiments the recall antigen may be an infectious disease antigen that the individual has likely encountered such as an influenza antigen. The recall antigen helps promote a more robust immune response.

The neoepitopes may be designed to optimally bind to MHC in order to promote a robust immune response. In some embodiments each peptide epitope comprises an antigenic region and a MHC stabilizing region. An MHC stabilizing region is a sequence which stabilizes the peptide in the MHC. The MHC stabilizing region may be 5-10, 5-15, 8-10, 1-5, 3-7, or 3-8 amino acids in length. In yet other embodiments the antigenic region is 5-100 amino acids in length. The peptides interact with the molecules of MHC class I by competitive affinity binding within the endoplasmic reticulum, before they are presented on the cell surface. The affinity of an individual peptide is directly linked to its amino acid sequence and the presence of specific binding motifs in defined positions within the amino acid sequence. The peptide being presented in the MHC is held by the floor of the peptide-binding groove, in the central region of the al/a2 heterodimer (a molecule composed of two nonidentical subunits). The sequence of residues, of the peptide-binding groove's floor determines which particular peptide residues it binds.

Optimal binding regions may be identified by a computer assisted comparison of the affinity of a binding site (MHC pocket) for a particular amino acid at each amino acid in the binding site for each of the target epitopes to identify an ideal binder for all of the examined antigens. The MHC stabilization regions of the epitopes may be identified using amino acid prediction matrices of data points for a binding site. An amino acid prediction matrix is a table having a first and a second axis defining data points. Prediction matrices can be generated as shown in Singh, H. and Raghava, G. P. S. (2001), “ProPred: prediction of HLA-DR binding sites.” Bioinformatics, 17(12), 1236-37).

In some embodiments the MHC stabilizing region is designed based on the subject's particular MHC. In that way the MHC stabilizing region can be optimized for each patient.

In some instances each epitope of a concatemeric antigen may include a MHC stabilizing region. All of the MHC stabilizing regions within the epitopes may be the same or they may be different. The MHC stabilizing regions may be at the N terminal portion of the peptide or the C terminal portion of the peptide. Alternatively the MHC stabilizing regions may be in the central region of the peptide. The neoepitopes in some embodiments are 13 residues or less in length and usually consist of between about 8 and about 11 residues, particularly 9 or 10 residues. In other embodiments the neoepitopes may be designed to be longer. For instance, the neoepitopes may have extensions of 2-5 amino acids toward the N- and C-terminus of each corresponding gene product. The use of a longer peptide may allow endogenous processing by patient cells and may lead to more effective antigen presentation and induction of T cell responses.

The neoepitopes selected for inclusion in the concatemeric antigen typically will be high affinity binding peptides. In some aspect the neoepitopes binds an HLA protein with greater affinity than a wild-type peptide. The neoepitope has an IC50 of at least less than 5000 nM, at least less than 500 nM, at least less than 250 nM, at least less than 200 nM, at least less than 150 nM, at least less than 100 nM, at least less than 50 nM or less in some embodiments. The neoepitopes in the concatemeric construct may be the same or different, e.g., they vary by length, amino acid sequence or both. Typically, peptides with predicted IC50<50 nM, are generally considered medium to high affinity binding peptides and will be selected for testing their affinity empirically using biochemical assays of HLA-binding. Finally, it will be determined whether the human immune system can mount effective immune responses against these mutated tumor antigens and thus effectively kill tumor but not normal cells.

Neoepitopes having the desired activity may be modified as necessary to provide certain desired attributes, e.g. improved pharmacological characteristics, while increasing or at least retaining substantially all of the biological activity of the unmodified peptide to bind the desired MHC molecule and activate the appropriate T cell or B cell. For instance, the neoepitopes may be subject to various changes, such as substitutions, either conservative or non-conservative, where such changes might provide for certain advantages in their use, such as improved MHC binding. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications may be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

The neoepitopes can also be modified by extending or decreasing the compound's amino acid sequence, e.g., by the addition or deletion of amino acids. The peptides, polypeptides or analogs can also be modified by altering the order or composition of certain residues, it being readily appreciated that certain amino acid residues essential for biological activity, e.g., those at critical contact sites or conserved residues, may generally not be altered without an adverse effect on biological activity.

Typically, a series of peptides with single amino acid substitutions are employed to determine the effect of electrostatic charge, hydrophobicity, etc. on binding. For instance, a series of positively charged (e.g., Lys or Arg) or negatively charged (e.g., Glu) amino acid substitutions are made along the length of the peptide revealing different patterns of sensitivity towards various MHC molecules and T cell or B cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an MHC molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding.

The neoepitopes may also comprise isosteres of two or more residues in the neoepitopes. An isostere as defined here is a sequence of two or more residues that can be substituted for a second sequence because the steric conformation of the first sequence fits a binding site specific for the second sequence. The term specifically includes peptide backbone modifications well known to those skilled in the art. Such modifications include modifications of the amide nitrogen, the α-carbon, amide carbonyl, complete replacement of the amide bond, extensions, deletions or backbone crosslinks. See, generally, Spatola, Chemistry and Biochemistry of Amino Acids, Peptides and Proteins, Vol. VII (Weinstein ed., 1983).

In other aspects the invention is a method for preparing a mRNA cancer vaccine, by isolating a sample from a subject, identifying a plurality of cancer antigens in the sample, determining T-cell epitopes from the plurality of cancer antigens, preparing a mRNA cancer vaccine having an open reading frame encoding a concatemeric antigen, wherein the concatemeric antigen is comprised of 2-100 of the T-cell epitopes interspersed by linkers. In some embodiments the method further involves determining binding strength of the T-cell epitopes to a MHC of a subject. In other embodiments the method further involves determining a T-cell receptor face (TCR face) for each epitope and selecting epitopes having a TCR face with low similarity to endogenous proteins. The T-cell epitopes may have been optimized for binding strength to a MHC of the subject is provided. In some embodiments a TCR face for each epitope has a low similarity to endogenous proteins.

For instance a technology referred to as JanusMatrix (Epivax), which examines cross-reactive T cell epitopes from both HLA binding and TCR-facing sides to allow comparison across large genome sequence databases can be used to identify epitopes having a desirable TCR face and binding strength to MHC. A suite of algorithms can be used alone or together with the JanusMatrix to optimize epitope selection. For example EpiMatrix takes overlapping 9-mer frames derived from the conserved target protein sequences and scores them for potential binding affinity against a panel of Class I or Class II HLA alleles; each frame-by-allele assessment that scores highly and is predicted to bind is a putative T cell epitope. ClustiMer takes EpiMatrix output and identifies clusters of 9-mers that contain large numbers of putative T cell epitopes. BlastiMer automates the process of submitting the previously identified sequences to BLAST to determine if any share similarities with the human genome; any such similar sequences would be likely to be tolerated or to elicit an unwanted autoimmune response. EpiAssembler takes the conserved, immunogenic sequences identified by Conservatrix and EpiMatrix and knits them together to form highly immunogenic consensus sequences. JanusMatrix can be used to screen out sequences which could potentially elicit an undesired autoimmune or regulatory T cell response due to homology with sequences encoded by the human genome. VaccineCAD can be used to link candidate epitopes into a string-of-beads design while minimizing nonspecific junctional epitopes that may be created in the linking process.

In addition to peptide epitopes, the concatemeric antigen may have one or more targeting sequences. A targeting sequence, as used herein, refers to a peptide sequence that facilitates uptake of the peptide into intracellular compartments such as endosomes for processing and/or presentation within MHC class I or II determinants.

The targeting sequence may be present at the N-terminus and/or C-terminus of an epitope of the concatemeric antigen, either directly adjacent thereto or separated by a linker of a cleavage sensitive site. Targeting sequences have a variety of lengths, for instance 4-50 amino acids in length.

The targeting sequence may be, for instance, an endosomal targeting sequence. An endosomal targeting sequence is a sequence derived from an endosomal or lysosomal protein known to reside in MHC class II Ag processing compartments, such as invariant chain, lysosome-associated membrane proteins (LAMP1,4 LAMP2), and dendritic cell (DC)-LAMP or a sequence having at least 80% sequence identity thereto. Additionally, an exemplary nucleic acid encoding a MHC class I signal peptide fragment (78 bp, secretion signal (sec)) and the transmembrane and cytosolic domains including the stop-codon (MHC class I trafficking signal (MITD), 168 bp) both amplified from activated PBMC, may be used (sec sense, 5′-aag ctt age ggc cgc acc atg cgg gtc acg gcg ccc cga acc-3′ (SEQ ID NO: 7); sec antisense, 5′-ctg cag gga gcc ggc cca ggt ctc ggt cag-3′ (SEQ ID NO: 8); MITD sense, 5′-gga tcc atc gtg ggc att gtt get ggc ctg get-3′ (SEQ ID NO: 9); and MITD antisense, 5′-gaa ttc agt ctc gag tca age tgt gag aga cac ate aga gcc-3′ (SEQ ID NO: 10).

MHC Class I presentation is typically an inefficient process (only 1 peptide of 10,000 degraded molecules is actually presented). Priming of CD8 T cells with APCs provides insufficient densities of surface peptide/MHC I complexes results in weak responders exhibiting impaired cytokine secretion and a decreased memory pool. The methods of the invention are capable of increasing the efficiency of MHC Class I presentation. MHC class I targeting sequences include MHC Class I trafficking signal (MITD) and PEST sequences (increase antigen-specific CD8 T cell responses presumably by targeting proteins for rapid degradation).

In some embodiments the RNA vaccines can be combined with agents for promoting the production of antigen presenting cells (APCs), for instance, by converting non-APCs into Pseudo-APCs. Antigen presentation is a key step in the initiation, amplification and duration of an immune response. In this process fragments of antigens are presented through the Major Histocompatibility Complex (MHC) or Human Leukocyte Antigens (HLA) to T cells driving an antigen-specific immune response. For immune prophylaxis and therapy, enhancing this response is important for improved efficacy. The RNA vaccines of the invention may be designed or enhanced to drive efficient antigen presentation. One method for enhancing APC processing and presentation, is to provide better targeting of the RNA vaccines to antigen presenting cells (APC). Another approach involves activating the APC cells with immune-stimulatory formulations and/or components. Alternatively, methods for reprograming non-APC into becoming APC may be used with the RNA vaccines of the invention. Importantly, most cells that take up mRNA formulations and are targets of their therapeutic actions are not APC. Therefore, designing a way to convert these cells into APC would be beneficial for efficacy. Methods and approaches for delivering RNA vaccines, e.g., mRNA vaccines to cells while also promoting the shift of a non-APC to an APC are provided herein. In some embodiments a mRNA encoding an APC reprograming molecule is included in the RNA vaccine or coadministered with the RNA vaccine.

An APC reprograming molecule, as used herein, is a molecule that promotes a transition in a non APC cell to an APC-like phenotype. An APC-like phenotype is property that enables MHC class II processing. Thus, an APC cell having an APC-like phenotype is a cell having one or more exogenous molecules (APC reprograming molecule) which has enhanced MHC class II processing capabilities in comparison to the same cell not having the one or more exogenous molecules. In some embodiments an APC reprograming molecule is a CIITA (a central regulator of MHC Class II expression); a chaperone protein such as CLIP, HLA-DO, HLA-DM etc. (enhancers of loading of antigen fragments into MHC Class II) and/or a costimulatory molecule like CD40, CD80, CD86 etc. (enhancers of T cell antigen recognition and T cell activation).

A CIITA protein is a transactivator that enhances activation of transcription of MHC Class II genes (Steimle et al., 1993, Cell 75:135-146) by interacting with a conserved set of DNA binding proteins that associate with the class II promoter region. The transcriptional activation function of CIITA has been mapped to an amino terminal acidic domain (amino acids 26-137). A nucleic acid molecule encoding a protein that interacts with CIITA, termed CIITA-interacting protein 104 (also referred to herein as CIP104). Both CITTA and CIP104 have been shown to enhance transcription from MHC class II promoters and thus are useful as APC reprograming molecule of the invention. In some embodiments the APC reprograming molecule are full length CIITA, CIP104 or other related molecules or active fragments thereof, such as amino acids 26-137 of CIITA, or amino acids having at least 80% sequence identity thereto and maintaining the ability to enhance activation of transcription of MHC Class II genes.

In preferred embodiments the APC reprograming molecule is delivered to a subject in the form of an mRNA encoding the APC reprograming molecule. As such the RNA vaccines of the invention may include an mRNA encoding an APC reprograming molecule. In some embodiments the mRNA in monocistronic. In other embodiments it is polycistronic. In some embodiments the mRNA encoding the one or more antigens is in a separate formulation from the mRNA encoding the APC reprograming molecule. In other embodiments the mRNA encoding the one or more antigens is in the same formulation as the mRNA encoding the APC reprograming molecule. In some embodiments the mRNA encoding the one or more antigens is administered to a subject at the same time as the mRNA encoding the APC reprograming molecule. In other embodiments the mRNA encoding the one or more antigens is administered to a subject at a different time than the mRNA encoding the APC reprograming molecule. For instance, the mRNA encoding the APC reprograming molecule may be administered prior to the mRNA encoding the one or more antigens. The mRNA encoding the APC reprograming molecule may be administered immediately prior to, at least 1 hour prior to, at least 1 day prior to, at least one week prior to, or at least one month prior to the mRNA encoding the antigens. Alternatively, the mRNA encoding the APC reprograming molecule may be administered after the mRNA encoding the one or more antigens. The mRNA encoding the APC reprograming molecule may be administered immediately after, at least 1 hour after, at least 1 day after, at least one week after, or at least one month after the mRNA encoding the antigens.

In other embodiments, the targeting sequence is a ubiquitination signal that is attached at either or both ends of the encoded peptide. In other embodiments, the targeting sequence is a ubiquitination signal that is attached at an internal site of the encoded peptide and/or to either end. Thus, the RNA may include a nucleic acid sequence encoding a ubiquitination signal at either or both ends of the nucleotides encoding the concatemeric peptide. Ubiquitination, a post-translational modification, is the process of attaching ubiquitin to a substrate target protein. A ubiquitination signal is a peptide sequence which enables the targeting and processing of a peptide to one or more proteasomes. By targeting and processing the peptide through the use of a ubiquitination signal the intracellular processing of the peptide can more closely recapitulate antigen processing in Antigen Presenting Cells (APCs).

Ubiquitin is an 8.5 kDa regulatory protein that is found in nearly all tissues of eukaryotic organisms. In the human genome, there are four genes that produce ubiquitin: UBB, UBC, UBA52, and RPS27A. UBA52 and RPS27A code for a single copy of ubiquitin fused to the ribosomal proteins L40 and S27a, respectively. The UBB and UBC genes code for polyubiquitin precursor proteins. There are three steps to ubiquitination, performed by three enzymes. Ubiquitin-activating enzymes, also called E1 enzymes, modify the ubiquitin so that it is in a reactive state. The E1 binds to both ATP and ubiquitin, catalyzing the acyl-adenylation of ubiquitin's C-terminal. Then, the ubiquitin is transferred to an active site cysteine residue, releasing AMP. Ultimately, a thioester linkage is formed between the ubiquitin's C-terminal carboxyl group and the E1 cysteine sulfhydryl group. In the human genome, UBA1 and UBA6 are the two genes that code for the E1 enzymes.

The activated ubiquitin is then subjected to E2 ubiquitin-conjugating enzymes, which transfer the ubiquitin from E1 to the active site cysteine of the E2 via a trans(thio)esterification reaction. The E2 binds to both the activated ubiquitin and the E1 enzyme. Humans have 35 different E2 enzymes, characterized by their highly conserved structure, which is known as the ubiquitin-conjugating catalytic (UBC) fold. The E3 ubiquitin ligases facilitate the final step of the ubiquitination cascade. Generally, they create an isopeptide bond between a lysine of the target protein and the C-terminal glycine of ubiquitin. There are hundreds of E3 ligases; some also activate the E2 enzymes. E3 enzymes function as the substrate recognition modules of the system and interact with both the E2 and the substrate. The enzymes possess one of two domains: the homologous to the E6-AP carboxyl terminus (HECT) domain or the really interesting new gene (RING) domain (or the closely related, U-box domain). HECT domain E3 enzymes transiently bind ubiquitin when an obligate thioester intermediate is formed with the active-site cysteine of the E3, whereas RING domain E3 enzymes catalyze the direct transfer from the E2 enzyme to the substrate.

The number of ubiquitins added to the antigen can enhance the efficacy of the processing step. For instance, in polyubiquitination, additional ubiquitin molecules are added after the first has been attached to the peptide. The resulting ubiquitin chain is created by the linking of the glycine residue of the ubiquitin molecule to a lysine of the ubiquitin bound to the peptide. Each ubiquitin contains seven lysine residues and an N-terminal that can serve as sites for ubiquitination. When four or more ubiquitin molecules are attached to a lysine residue on the peptide antigen, the 26S proteasome recognizes the complex, internalizes it, and degrades the protein into small peptides.

Ubiquitin wild type has the following sequence (Homo sapiens):

(SEQ ID NO: 11)
MQIFVKTLTGKTITLEVEPSDTIENVKAKIQDKEGIPPDQQRLIFAGKQL
EDGRTLSDYNIQKESTLHLVLRLRGG

The epitopes are connected in some embodiments by a cleavage sensitive site. A cleavage sensitive site is a peptide which is susceptible to cleavage by an enzyme or protease. These sites are also called protease cleavage sites. Preferably the protease is an intracellular enzyme. In some preferred embodiments the protease is a protease found in an Antigen Presenting Cell (APC). Thus, protease cleavage sites correspond to high abundance (highly expressed) proteases in APCs. A cleavage sensitive site that is sensitive to an APC enzyme is referred to as an APC cleavage sensitive site. Proteases expressed in APCs include but are not limited to Cysteine proteases, such as: Cathepsin B, Cathepsin H, Cathepsin L, Cathepsin S, Cathepsin F, Cathepsin Z, Cathepsin V, Cathepsin O, Cathepsin C, and Cathepsin K, and Aspartic proteases such as Cathepsin D, Cathepsin E, and Asparaginyl endopeptidase.

The following are exemplary APC cleavage sensitive sites:

Cathepsin B: cleavage on the caboxyl side of Arg-Arg bonds
Cathepsin D has the following preferential cleavage sequences:

| | | | | | | | | | | |
|---|---|---|---|---|---|---|---|---|---|---|
| P6 | P5 | P4 | P3 | P2 | P1 | ↓ | P1′ | P2′ | P3′ | P4′ |
| Xaa | Xaa | Xaa | Xaa | hydro | hydro | ↓ | hydro | Xaa | Xaa | Xaa |
| Xaa | Xaa | Xaa | Xaa | Glu | hydro | ↓ | hydro | Xaa | Xaa | Xaa, |

where Xaa = any amino acid residue, hydro = Ala, Val, Leu, Ile, Phe, Trp, or Tyr, and ↓ =
cleavage site
Cathepsin H: Arg-↓-NHMec; Bz-Arg-↓-NhNap; Bz-Arg-↓-NHMec; Bz-Phe-Cal-Arg-↓-
NHMec; Pro-Gly-↓-Phe
Cathepsin S and F: Xaa-Xaa-Val-Val-Arg-Xaa-Xaa
where Xaa = any amino acid residue
Cathepsin V: Z-Phe-Arg-NHMec; Z-Leu-Arg-NHMec; Z-Val-Arg-NHMec
Cathepsin O: Z-Phe-Arg-NHMec and Z-Arg-Arg-NHMec
Cathepsin C has the following preferential cleavage sequences:

| | | | | | | |
|---|---|---|---|---|---|---|
| P2 | P1 | ↓ | P1′ | P2′ | P3′ | P4′ |
| not Arg | not Pro | ↓ | not Pro | Xaa | Xaa | Xaa |
| not Lys | not Pro | ↓ | not Pro | Xaa | Xaa | Xaa, |

where Xaa = any amino acid residue and ↓ = cleavage site
Cathepsin E: Arg-X, Glu-X, and Arg-Arg
Asparaginyl endopeptidase: after asparagine residues
Cathepsin L has the following preferential cleavage sequences:

| | | | | | | | | | | |
|---|---|---|---|---|---|---|---|---|---|---|
| P6 | P5 | P4 | P3 | P2 | P1 | ↓ | P1′ | P2′ | P3′ | P4′ |
| Xaa | Xaa | Xaa | hydrophobic | Phe | Arg | ↓ | Xaa | Xaa | Xaa | Xaa |
| Xaa | Xaa | Xaa | aromatic | Phe | Arg | ↓ | Xaa | Xaa | Xaa | Xaa |
| Xaa | Xaa | Xaa | hydrophobic | Arg | Arg | ↓ | Xaa | Xaa | Xaa | Xaa |
| Xaa | Xaa | Xaa | aromatic | Arg | Arg | ↓ | Xaa | Xaa | Xaa | Xaa, |

where Xaa = any amino acid residue, hydrophobic = Ala, Val, Leu, Ile, Phe, Trp, or Tyr,
aromatic = Phe, Trp, His, or Tyr, and ↓ = cleavage site

In some preferred embodiments the cleavage sensitive site is a cathepsin B or S sensitive sites. Exemplary cathepsin B sensitive sites include but are not limited to:

(SEQ ID NO: 12)
EGAMVAATQGAAAAAGSGAGTGGGTASGGTEGGSAESEGAK,
(SEQ ID NO: 13)
AMVAATQGAAAAAGSGAGTGGGTASGGTEGGSAESEGAK,
(SEQ ID NO: 14)
GGGGGGGGAGAAGGGGGGENYDDPHK,
(SEQ ID NO: 15)
MVAATQGAAAAAGSGAGTGGGTASGGTEGGSAESEGAK,
(SEQ ID NO: 16)
QLLCGAAIGTHEDDKYR,
(SEQ ID NO: 17)
FSHHFEDADNIYIFLELCSRKS,
(SEQ ID NO: 18)
YXLVGAGAIGCELLK,
(SEQ ID NO: 19)
IPESCSFGYHAGGWGKPPVDETGKPL,
(SEQ ID NO: 20)
VAATQGAAAAAGSGAGTGGGTASGGTEGGSASEGAK,
(SEQ ID NO: 21)
SEADIEGPLPAKDIHLDLPSNN,
(SEQ ID NO: 22)
HFNALGGWGELQNSVK,
(SEQ ID NO: 23)
FAQALGLTEAVK,
(SEQ ID NO: 24)
TSVLAAANPIESQWNPK,
(SEQ ID NO: 25)
QLLQANPILESFGNAK,
(SEQ ID NO: 26)
TSILAAANPISGHYDR,
(SEQ ID NO: 27)
IXXANPLLEAFGNAK,
(SEQ ID NO: 28)
LYGAQFHPEVGLTENGK,
(SEQ ID NO: 29)
PQGQAPPLSQAQGHPGIQTPQR,
(SEQ ID NO: 30)
AAASAAAASAASGSPGPGEGSAGGEKR,
(SEQ ID NO: 31)
IXXXFLGASLKDEVLK,
(SEQ ID NO: 32)
LTISPDYAYGATGHPGIIPPH,
(SEQ ID NO: 33)
LTISPDYAYGATGHPGIIPPHA,
(SEQ ID NO: 34)
ILISLATGHREEGGENLDQ,
(SEQ ID NO: 35)
LSELTQQLAQATGKPPQYIAVHVVPDQ,
(SEQ ID NO: 36)
LSELTQQLAQATGKPPQYIAVHVVPDQL,
(SEQ ID NO: 37)
DATNVGDEGGFAPNILENK,
(SEQ ID NO: 38)
ILAQATSDLVNAIK,
(SEQ ID NO: 39)
VXXVXQHAVGIVVNK,
(SEQ ID NO: 40)
GSLAEAVGSPPPAATPTPTPPTR,
(SEQ ID NO: 41)
SXGLPVGAVINCADNTGAK,
(SEQ ID NO: 42)
YCFSEMAPVCAVVGGILAQEIVK,
(SEQ ID NO: 43)
HVYGYSMAYGPAQHAISTEK,
(SEQ ID NO: 44)
LWQLSKPRPGCSVLGPLPLL,
(SEQ ID NO: 45)
MILIQDGSQNTNVDKPLR,
(SEQ ID NO: 46)
TYSMVVVPLYDTLGPGAIRYII,
(SEQ ID NO: 47)
HFAMMHGGTGFAGIDSSSPEVK,
(SEQ ID NO: 48)
GXLKPGMVVTFAPVNVTTEVK,
(SEQ ID NO: 49)
FNALFAQGNYSEAAK,
(SEQ ID NO: 50)
GPIHIGGPPGFASSSGKPGPTVIK,
(SEQ ID NO: 51)
GFGFVTFDDHDPVDK,
(SEQ ID NO: 52)
DQGSCGSCWAFGAVEAISDR,
(SEQ ID NO: 53)
GXNFGFGDSRGGGGNFGPGPG,
(SEQ ID NO: 54)
HDLFDSGFGGGAGVETGGK,
(SEQ ID NO: 55)
CYLFGGLANDSEDPK,
(SEQ ID NO: 56)
TTEDSVMLNGFGTVVNALGK,
(SEQ ID NO: 57)
LTEGLHGFHVHEFGDNTAGC,
(SEQ ID NO: 58)
GYAFIEYEHER,
(SEQ ID NO: 59)
MFIGGLSWDTSKK,
(SEQ ID NO: 60)
MFIGGLSWDTTKK,
(SEQ ID NO: 61)
SMGFIGHYLDQK,
(SEQ ID NO: 62)
SMGFIGHYLDQK,
(SEQ ID NO: 63)
ALXGGIGFIHHNCTPEFQANE,
(SEQ ID NO: 64)
NLQSTFSGFGFINSENVFK,
(SEQ ID NO: 65)
GFCFITYTDEEPVKK,
(SEQ ID NO: 66)
MPMFIVNTNVPR,
(SEQ ID NO: 67)
VSEIFVELQGFLAAEQDIR,
(SEQ ID NO: 68)
GFCFLEYEDHK,
(SEQ ID NO: 69)
QAVSMFLGAVEEAKK,
(SEQ ID NO: 70)
KPXKPMQFLGDEETVRK,
(SEQ ID NO: 71)
GAAEPHTIAAFLGGAAAQEVIK,
(SEQ ID NO: 72)
MIPCDFLIPVQTQHPIR,
(SEQ ID NO: 73)
QGAPTSFLPPEASQLKPDR,
(SEQ ID NO: 74)
STGGAPTFNVTVTK,
(SEQ ID NO: 75)
MVYMFQYDSTHGK,
(SEQ ID NO: 76)
HFPMTHGNTGFSGIESSSPEVK,
(SEQ ID NO: 77)
AVAFSPVTELKK,
(SEQ ID NO: 78)
GFGFVTFSSMAEVDAAMAARPH,
(SEQ ID NO: 79)
TCGFDFTGAVEDISK,
(SEQ ID NO: 80)
EYSGLSDGYGFTTDLFGR
(SEQ ID NO: 81)
GQHVXGSPFQFTVGPLGEGGAHK,
(SEQ ID NO: 82)
GFGFVDFNSEEDAK,
(SEQ ID NO: 83)
FXFVEFEDPR,
(SEQ ID NO: 84)
FXFVEFEDPR,
(SEQ ID NO: 85)
IELFVGGELIDPADDRK,
(SEQ ID NO: 86)
MFVGGLSWDTSKK,
(SEQ ID NO: 87)
AFSAFVGQMHQQGILK,
(SEQ ID NO: 88)
GILFVGSGVSGGEEGAR,
(SEQ ID NO: 89)
IIAFVGSPVEDNEKDLVK,
(SEQ ID NO: 90)
DYAFVHFEDR,
(SEQ ID NO: 91)
GYAFVHFETQEAADK,
(SEQ ID NO: 92)
GYGFVHFETQEAAER,
(SEQ ID NO: 93)
NYGFVHIEDK,
(SEQ ID NO: 94)
ITLPVDFVTADKFDENAK,
(SEQ ID NO: 95)
GFGFVTFDDHDPVDK,
(SEQ ID NO: 96)
LPNFGFVVFDDSEPVQK,
(SEQ ID NO: 97)
GFGFVYFQNHDAADK,
(SEQ ID NO: 98)
YQFWDTQPVPK,
(SEQ ID NO: 99)
QLLCGAAIGTHEDDKYR,
(SEQ ID NO: 100)
QLLCGAAIGTHEDDKYR,
(SEQ ID NO: 101)
PPAGGGGGAGGAGGGPPPGPPGAGDR,
(SEQ ID NO: 102)
FGGSFAGSFGGAGGHAPGVAR,
(SEQ ID NO: 103)
CNPIISGLYQGAGGPGPGGFGAQGPK,
(SEQ ID NO: 104)
PGLNLPPPIGGAGPPLGLPKPK,
(SEQ ID NO: 105)
QPXVDGFLVGGASLKPEFVDIINAK,
(SEQ ID NO: 106)
VTGDHIPTPQDLPQR,
(SEQ ID NO: 107)
YGGELVPHFPAR,
(SEQ ID NO: 108)
YQGAGGPGPGGFGAQGPK,
(SEQ ID NO: 109)
EYFGGFGEVESIELPMDNK,
(SEQ ID NO: 110)
ALVLGGFAHMDTETK,
(SEQ ID NO: 111)
VSHVSTGGGASLELLEGK,
(SEQ ID NO: 112)
AEGGGGGGRPGAPAAGDGK,
(SEQ ID NO: 113)
RGGGGGGSGGIGYPYPR,
(SEQ ID NO: 114)
NMGGPYGGGNYGPGGSGGSGGYG,
(SEQ ID NO: 115)
GTGGVDTAATGGVFDISNLDR,
(SEQ ID NO: 116)
HFNALGGWGELQNSVK,
(SEQ ID NO: 117)
PESCSFGYHAGGWGKPPVDETGKPL,
(SEQ ID NO: 118)
SSLPNFCGIFNHLER,
(SEQ ID NO: 119)
AMALXGGIGFIHHNCTPEF,
(SEQ ID NO: 120)
AMALXGGIGFIHHNCTPEFQANE,
(SEQ ID NO: 121)
EWIKPIMFSGGIGSMEADHISK,
(SEQ ID NO: 122)
GDGPVQGIINFEQK,
(SEQ ID NO: 123)
EMAPVCAVVGGILAQEIVK,
(SEQ ID NO: 124)
LAFHGILLHGLEDR,
(SEQ ID NO: 125)
MGVVAGILVQNVLK
(SEQ ID NO: 126)
FTASAGIQVVGDDLTVTNPK,
(SEQ ID NO: 127)
TPYQIACGISQGLADNTVIAK,
(SEQ ID NO: 128)
YPIEHGIVTNWDDMEK,
(SEQ ID NO: 129)
VASGIPAGWXGLDCGPESSKK,
(SEQ ID NO: 130)
LFVGGLDWSTTQETLR,
(SEQ ID NO: 131)
HGGSLGLGLAAMGTAR,
(SEQ ID NO: 132)
IFVGGLSANTVVEDVK,
(SEQ ID NO: 133)
LFIGGLSFETTDDSLR,
(SEQ ID NO: 134)
LFIGGLSFETTDESLR,
(SEQ ID NO: 135)
LFIGGLSFETTEESLR,
(SEQ ID NO: 136)
MFXGGLSWDTSKK,
(SEQ ID NO: 137)
MFXGGLSWDTSKK,
(SEQ ID NO: 138)
MFXGGLSWDTSKK,
(SEQ ID NO: 139)
DAVSGMGVIVHIIEK,
(SEQ ID NO: 140)
GGNFGFGDSR,
(SEQ ID NO: 141)
GTTGSGAGSGGPGGLTSAAPAGGDKK,
(SEQ ID NO: 142)
IISGLYQGAGGPGPGGFGAQGPK,
(SEQ ID NO: 143)
IISGLYQGAGGPGPGGFGAQGPK,
(SEQ ID NO: 144)
GGGLLIGGQAWDWANQGEDERV,
(SEQ ID NO: 145)
GNFGGSFAGSFGGAGGHAPGVAR,
(SEQ ID NO: 146)
NFGGSFAGSFGGAGGHAPGVAR,
(SEQ ID NO: 147)
NFGGSFAGSFGGAGGHAPGVAR,
(SEQ ID NO: 148)
NFGGSFAGSFGGAGGHAPGVAR,
(SEQ ID NO: 149)
SAADTKPGTTGSGAGSGGPGGLTSAAPAGGDKK,
(SEQ ID NO: 150)
AATQGAAAAAGSGAGTGGGTASGGTEGGSAESEGAK,
(SEQ ID NO: 151)
GSSGGSGAKPSDAASEAAR,
(SEQ ID NO: 152)
IQFHFHWGSLDGQGSEHTVDK,
(SEQ ID NO: 153)
MILIQDGSQNTNVDKPLR,
(SEQ ID NO: 154)
KGTFTDDLHK,
(SEQ ID NO: 155)
QQSHFAMMHGGTGFAGIDSSSPEVK,
(SEQ ID NO: 156)
VAVLISGTGSNLQALIDSTR,
(SEQ ID NO: 157)
FLAAGTHLGGTNLDFQ,
(SEQ ID NO: 158)
LVLGTHTSDEQNHL,
(SEQ ID NO: 159)
TGGVDTAATGGVFDISNLDR,
(SEQ ID NO: 160)
TGGVDTAAVGGVFDVSNADR,
(SEQ ID NO: 161)
AVXIVAAGVGEFEAGISK,
(SEQ ID NO: 162)
EILTLLQGVHQGAGFQDIPK,
(SEQ ID NO: 163)
MKPLMGVIYVPLTDKEK,
(SEQ ID NO: 164)
ECISXHVGQAGVQIGNACWE,
(SEQ ID NO: 165)
HFNALGGWGELQNSVK,
(SEQ ID NO: 166)
ESCSFGYHAGGWGKPPVDETGKPL,
(SEQ ID NO: 167)
AGYVTHLMK,
(SEQ ID NO: 168)
TMFSSEVQFGHAGACANQASETAVAK,
(SEQ ID NO: 169)
MPFPVNHGASSEDTLLK,
(SEQ ID NO: 170)
FFLHHLIAEIHTAEIRAT,
(SEQ ID NO: 171)
NXSAXQVLIEHIGNLDR,
(SEQ ID NO: 172)
GGYVLHIGTIYGDLK,
(SEQ ID NO: 173)
DXHLGGEDFDNR,
(SEQ ID NO: 174)
GILGPPPPSFHLGGPAVGPR,
(SEQ ID NO: 175)
PTPPPTLHLVPEPAAPPPP,
(SEQ ID NO: 176)
YGPQYGHPPPPPPPPEYGPHADSPV,
(SEQ ID NO: 177)
KHSGPNSADSANDGFVR,
(SEQ ID NO: 178)
RPELLTHSTTEVTQPR,
(SEQ ID NO: 179)
LXGHVGFDSLPDQLVNK,
(SEQ ID NO: 180)
AASATQTIAAAQHAASTPK,
(SEQ ID NO: 181)
CLTQSGIAGGYKPF,
(SEQ ID NO: 182)
ELAQIAGRPTEDEDEKEK,
(SEQ ID NO: 183)
AITIAGVPQSVTECVK,
(SEQ ID NO: 184)
GLCAIAQAESLR,
(SEQ ID NO: 185)
KPTALIGVAAIGGAFSEQILK,
(SEQ ID NO: 186)
DYMNVQCHACIGGTNVGEDIR,
(SEQ ID NO: 187)
NTQNFQSLHNIGSVVQHSEGKPL,
(SEQ ID NO: 188)
LKPPTLIHGQAPSAGLPSQKPK,
(SEQ ID NO: 189)
VLIIGGGDGGVLR,
(SEQ ID NO: 190)
GCITIIGGGDTATCCAK,
(SEQ ID NO: 191)
GRPSETGIIGIIDPECR,
(SEQ ID NO: 192)
EAFGWHAIIVDGHSVEELCK,
(SEQ ID NO: 193)
LAAAILGGVDQIHIKPG,
(SEQ ID NO: 194)
LYSILGTTLKDEGK,
(SEQ ID NO: 195)
MILIQDGSQNTNVDKPLR,
(SEQ ID NO: 196)
LAMQEFMILPVGAANFR,
(SEQ ID NO: 197)
VPYLIAGIQHSCQDIGAK,
(SEQ ID NO: 198)
TVAGGVHISGLHTESAPR,
(SEQ ID NO: 199)
VAVLISGTGSNLQALIDSTR,
(SEQ ID NO: 200)
GITAIGGTSTISSEGTQHSYSEEEK,
(SEQ ID NO: 201)
AGVSISVVHGNLSEEAAK,
(SEQ ID NO: 202)
HVTQAHVQTGITAAPPPHPGAPHPPQ,
(SEQ ID NO: 203)
AGLFLPGSVGITDPCESGNFR,
(SEQ ID NO: 204)
AFAHITGGGLLENIPR,
(SEQ ID NO: 205)
ILAQITGTEHLK,
(SEQ ID NO: 206)
TFXNITPAEVGVLVGK,
(SEQ ID NO: 207)
HSSGIVADLSEQSLK,
(SEQ ID NO: 208)
EDGNEEDKENQGDETQGQQPPQR,
(SEQ ID NO: 209)
PGPSGITIPGKPGAQGVPGPPG,
(SEQ ID NO: 210)
GLTKPAALAAAPAKPGGAGGSK,
(SEQ ID NO: 211)
LGAQLADLHLDNK,
(SEQ ID NO: 212)
SLVASLAEPDFVVTDFAK,
(SEQ ID NO: 213)
MSLPLLAGGVADDINTNKK,
(SEQ ID NO: 214)
QPYAVSELAGHQTSAESWGTGR,
(SEQ ID NO: 215)
VTVAGLAGKDPVQC,
(SEQ ID NO: 216)
IITLAGPTNAIFK,
(SEQ ID NO: 217)
STHGLAILGPENPK,
(SEQ ID NO: 218)
ASAELALGENSEVLK,
(SEQ ID NO: 219)
ILISLATGHREEGGENLDQ,
(SEQ ID NO: 220)
AMSRPFGVALLFGGVDEK,
(SEQ ID NO: 221)
LQATAHAQAQLGCPVIIHPGR,
(SEQ ID NO: 222)
ILAGLGFDPEMQNRPT,
(SEQ ID NO: 223)
PERPQQLPHGLGGIGMGLGPGGQPIDANHLNK,
(SEQ ID NO: 224)
QLMQLIGPAGLGGLGGLGALTGPG,
(SEQ ID NO: 225)
HFNALGGWGELQNSVK,
(SEQ ID NO: 226)
MGAGLGHGMDR,
(SEQ ID NO: 227)
THMTAIVGMALGHRPIPNQPPT,
(SEQ ID NO: 228)
PHGLGGIGMGLGPGGQPIDANHLNK,
(SEQ ID NO: 229)
ASQGDSISSQLGPIHPPPR,
(SEQ ID NO: 230)
VWQLGSSSPNFTLEGHEK,
(SEQ ID NO: 231)
YVATLGVEVHPL,
(SEQ ID NO: 232)
KLIADYSPDDIFN,
(SEQ ID NO: 233)
TXGLIFVVDSNDR,
(SEQ ID NO: 234)
VPEFQFLIGDEAATHLK,
(SEQ ID NO: 235)
CNINLLPLPDPIPSGLME,
(SEQ ID NO: 236
LITEMVALNPDFKPPADYKPPA,
(SEQ ID NO: 237)
NQVALNPQNTVFDAK,
(SEQ ID NO: 238)
GLLKPGLNVVLEGPK,
(SEQ ID NO: 239)
GVNLPGAAVDLPAVSEK,
(SEQ ID NO: 240)
ISXGLPVGAVINCADNTGAK,
(SEQ ID NO: 241)
GQVCLPVISAENWK,
(SEQ ID NO: 242)
EILTLLQGVHQGAGFQDIPK,
(SEQ ID NO: 243)
NNQFQALLQYADPVSAQHAK,
(SEQ ID NO: 244)
LFIGGLSFETTDDSLR,
(SEQ ID NO: 245)
AIQLSGAEQLEALK,
(SEQ ID NO: 246)
DVSIEDSVISLSGDHCIIGR,
(SEQ ID NO: 247)
EYLLSGDISEAEHCLK,
(SEQ ID NO: 248)
VVISSDGQFALSGSWDGTLR,
(SEQ ID NO: 249)
VHEQLAALSQGPISKPK,
(SEQ ID NO: 250)
LVXLXXETALLSSGFSLEDPQTH,
(SEQ ID NO: 251)
GPDGLTAFEATDNQAIK,
(SEQ ID NO: 252)
ALYWLSGLTCTEQNFISK,
(SEQ ID NO: 253)
IITLTGPTNAIFK,
(SEQ ID NO: 254)
LATQLTGPVMPVR,
(SEQ ID NO: 255)
FPSLLTHNENMVAK,
(SEQ ID NO: 256)
LEXLXTINXGLTSIANLPK,
(SEQ ID NO: 257)
ALLLLLVGGVDQSPR,
(SEQ ID NO: 258)
GKPVGLVGVTELSDAQKK,
(SEQ ID NO: 259)
VNVAGLVLAGSADFK,
(SEQ ID NO: 260)
QGYIGAALVLGGVDVTGPH,
(SEQ ID NO: 261)
LYTLVLTDPDAPSR,
(SEQ ID NO: 262)
AQIHDLVLVGGSTR,
(SEQ ID NO: 263)
LNHVAAGLVSPSLKSDTSSK,
(SEQ ID NO: 264)
IEVGLVVGNSQVAFEK,
(SEQ ID NO: 265)
GYHQSASEHGLVVIAPDTSPR,
(SEQ ID NO: 266)
GYHQSASEHGLVVIAPDTSPR,
(SEQ ID NO: 267)
QDHPWLLSQNLVVKPDQLIK,
(SEQ ID NO: 268)
MGLAMGGGGGASFDR,
(SEQ ID NO: 269)
QLPHGLGGIGMGLGPGGQPIDANHLNK,
(SEQ ID NO: 270)
VVVLMGSTSDLGHCEK,
(SEQ ID NO: 271)
MALIQMGSVEEAVQA,
(SEQ ID NO: 272)
TTGFGMIYDSLDYAK,
(SEQ ID NO: 273)
WLLAEMLGDLSDSQLK,
(SEQ ID NO: 274)
QAQYLGMSCDGPFKPDH,
(SEQ ID NO: 275)
AHSSMVGVNLPQK,
(SEQ ID NO: 276)
SGPVVAMVWEGLNVVK,
(SEQ ID NO: 277)
VNTQNFQSLHNIGSVVQHSEGKPL,
(SEQ ID NO: 278)
LYVSNLGIGHTR,
(SEQ ID NO: 279)
VYVGNLGNNGNKTELER,
(SEQ ID NO: 280)
IVDLLQMLEMNMAIAFPA,
(SEQ ID NO: 281)
VLAQNSGFDLQETLVK,
(SEQ ID NO: 282)
QQSHFPMTHGNTGFSGIESSSPEVK,
(SEQ ID NO: 283)
ILIANTGMDTDKIK,
(SEQ ID NO: 284)
NNTVTPGGKPNK,
(SEQ ID NO: 285)
VVNVANVGAVPSGQDNIHR,
(SEQ ID NO: 286)
MPFPVNHGASSEDTLLK,
(SEQ ID NO: 287)
RPKDPGHPY,
(SEQ ID NO: 288)
ELDIMEPKVPDDIYK,
(SEQ ID NO: 289)
AETSQQEASEGGDPASPALSLS,
(SEQ ID NO: 290)
LLAAQNPLSQADRPHQ,
(SEQ ID NO: 291)
PDNFXFGQSGAGNNWAK,
(SEQ ID NO: 292)
MIAGQVLDINLAAEPK,
(SEQ ID NO: 293)
IILNSHSPAGSAAISQQDFHPK,
(SEQ ID NO: 294)
GAVAVSAAPGSAAPAAGSAPAAAEEK,
(SEQ ID NO: 295)
SAAGAAGSAGGSSGAAGAAGGGAGAGTRPGDGGTASAGAAGPGAATK,
(SEQ ID NO: 296)
FTASAGIQVVGDDLTVTNPK,
(SEQ ID NO: 297)
FGIVTSSAGTGTTEDTEAKK,
(SEQ ID NO: 298)
SLYQSAGVAPESFEYIEAHGTGTK,
(SEQ ID NO: 299)
VSEIDEMFEARKM,
(SEQ ID NO: 300)
FGGSFAGSFGGAGGHAPG,
(SEQ ID NO: 301)
FGGSFAGSFGGAGGHAPGVAR,
(SEQ ID NO: 302)
FGGSFAGSFGGAGGHAPGVAR,
(SEQ ID NO: 303)
FGGSFAGSFGGAGGHAPGVAR,
(SEQ ID NO: 304)
AADTKPGTTGSGAGSGGPGGLTSAAPAGGDKK,
(SEQ ID NO: 305)
ATQGAAAAAGSGAGTGGGTASGGTEGGSAESEGAK,
(SEQ ID NO: 306)
EIELIGSGGFGQVFK,
(SEQ ID NO: 307)
KPGTTGSGAGSGGPGGLTSAAPAGGDKK,
(SEQ ID NO: 308)
LYANXVXSGGTTMYPGIADR,
(SEQ ID NO: 309)
RSGKYDLDFK,
(SEQ ID NO: 310)
HDGYGSHGPLLPLPSR,
(SEQ ID NO: 311)
SLFSSIGEVESAK,
(SEQ ID NO: 312)
LQSIGTENTEENR,
(SEQ ID NO: 313)
SLVASLAEPDFVVTDFAK,
(SEQ ID NO: 314)
AEPMGEKPVGSLAGIGEVLGK,
(SEQ ID NO: 315)
VQEAINSLGGSVFPK,
(SEQ ID NO: 316)
SAAAASAASGSPGPGEGSAGGEKR,
(SEQ ID NO: 317)
QTIDNSQGAYQEAFDISKK,
(SEQ ID NO: 318)
FGIVTSSAGTGTTEDTEAK,
(SEQ ID NO: 319)
FGIVTSSAGTGTTEDTEAKK,
(SEQ ID NO: 320)
XSSFDLDYDFQR,
(SEQ ID NO: 321)
FQAGTSKPLHSSGINVNAAPF,
(SEQ ID NO: 322)
HIGGPPGFASSSGKPGPTVIK,
(SEQ ID NO: 323)
XSSGPGASSGTSGDHGELVVR,
(SEQ ID NO: 324)
ELVSSSSSGSDSDSEVDKK,
(SEQ ID NO: 325)
MDSTEPPYSQKR,
(SEQ ID NO: 326)
VVVLMGSTSDLGHCEK,
(SEQ ID NO: 327)
ILDSVGIEADDDRLNK,
(SEQ ID NO: 328)
STQPISSVGKPASVIK,
(SEQ ID NO: 329)
ALQSVGQIVGEVLK,
(SEQ ID NO: 330)
VSSLAEGSVTSVGSVNPAENFR,
(SEQ ID NO: 331)
TGSISSSVSVPAKPER,
(SEQ ID NO: 332)
YXXXXXXYSQSYGGYENQK,
(SEQ ID NO: 333)
IYWGTATTGKPHV,
(SEQ ID NO: 334)
MVQTAVVPVKK,
(SEQ ID NO: 335)
MMLGTEGGEGFVVK,
(SEQ ID NO: 336)
XTFIAIKPDGVQR,
(SEQ ID NO: 337)
VSHVSTGGGASLELLEGK,
(SEQ ID NO: 338)
AAAAAGSGAGTGGGTASGGTEGGSAESEGAK,
(SEQ ID NO: 339)
TIGNSCGTIGLIHAVANNQDK,
(SEQ ID NO: 340)
TGEEIFGTIGMRPNAK,
(SEQ ID NO: 341)
TTQFSCTLGEKFEETTADGR,
(SEQ ID NO: 342)
GCTATLGNFAK,
(SEQ ID NO: 343)
YVATLGVEVHPL,
(SEQ ID NO: 344)
LAATNALLNSLEFTK,
(SEQ ID NO: 345)
GPGASSGTSGDHGELVVR,
(SEQ ID NO: 346)
STTTGHLIYK,
(SEQ ID NO: 347)
ALSAADTKPGTTGSGAGSGGPGGLTSAAPAGGDKK,
(SEQ ID NO: 348)
STTTGHLIYK,
(SEQ ID NO: 349)
VTIIGPATVGGIKPGCFK,
(SEQ ID NO: 350)
VVFSHPPIGTVGLTEDEAIHK,
(SEQ ID NO: 351)
GSPTSLGTWGSWIGPDHDK,
(SEQ ID NO: 352)
GSPTSLGTWGSWIGPDHDKF,
(SEQ ID NO: 353)
IHFPLATYAPVISAEK,
(SEQ ID NO: 354)
ANPQVGVAFPHIK,
(SEQ ID NO: 355)
TCTTVAFTQVNSEDK,
(SEQ ID NO: 356)
VLTGVAGEDAECHAAK,
(SEQ ID NO: 357)
NIPPYFVALVPQEEELDDQK,
(SEQ ID NO: 358)
GQETAVAPSLVAPALNKPK,
(SEQ ID NO: 359)
QGQETAVAPSLVAPALNKPK,
(SEQ ID NO: 360)
GFVTFSSMAEVDAAMAARPH,
(SEQ ID NO: 361)
VDYYTTTPALVFGKPVR,
(SEQ ID NO: 362)
VDYYTTTPALVFGKPVR,
(SEQ ID NO: 363)
ASQPXVDGFLVGGASLKPEFVDIINAK,
(SEQ ID NO: 364)
TIIGPATVGGIKPGCFK,
(SEQ ID NO: 365)
GGVDTAAVGGVFDVSNADR,
(SEQ ID NO: 366)
TTVHAITATQK,
(SEQ ID NO: 367)
EEVRPQDTVSVIGGVAGGSK,
(SEQ ID NO: 368)
QVIGTGSFFPK,
(SEQ ID NO: 369)
ASGNYATVISHNPETK,
(SEQ ID NO: 370)
MKPLMGVIYVPLTDKEK,
(SEQ ID NO: 371)
FSVCVLGDQQHCDEAK,
(SEQ ID NO: 372)
ENAFCNLAAIVPDSVGRHSPA,
(SEQ ID NO: 373)
AYVGNLPFNTVQGDIDAIFK,
(SEQ ID NO: 374)
TLTTVQGIADDYDKK,
(SEQ ID NO: 375)
CISXHVGQAGVQIGNACWE,
(SEQ ID NO: 376)
THALQWPSLTVQWLPEVTKPEGK,
(SEQ ID NO: 377)
ASVPAGGAVAVSAAPGSAAPAAGSAPAAAEEK,
(SEQ ID NO: 378)
YEEVSVSGFEEFHR,
(SEQ ID NO: 379)
CMTTVSWDGDKLQCVQK,
(SEQ ID NO: 380)
MHGGGPTVTAGLPLPK,
(SEQ ID NO: 381)
LALVTGGEIASTFDHPELVK,
(SEQ ID NO: 382)
LEGTLLKPNMVTPGHACTQK,
(SEQ ID NO: 383)
XVVESAYEVIK,
(SEQ ID NO: 384)
ILAQVVGDVDTSLPR,
(SEQ ID NO: 385)
CFSEMAPVCAVVGGILAQEIVK,
(SEQ ID NO: 386)
ETEDTFXADLVVGLCTGQIK,
(SEQ ID NO: 387)
EGPAVVGQFIQDVK,
(SEQ ID NO: 388)
MLISGYALNCVVGSQGMPK,
(SEQ ID NO: 389)
HWPFMVVNDAGRPK
(SEQ ID NO: 390)
SGPVVAMVWEGLNVVK,
(SEQ ID NO: 391)
ALQDEWDAVMLHSFTLRQ,
(SEQ ID NO: 392)
EYFSWEGAFQHVGK,
(SEQ ID NO: 393)
ATVASGIPAGWMGLDCGPESSK,
(SEQ ID NO: 394)
ATVASGIPAGWMGLDCGPESSKK,
(SEQ ID NO: 395)
DCAFYDPTHAWSGGLDHQLK,
(SEQ ID NO: 396)
QFQALLQYADPVSAQHAK,
(SEQ ID NO: 397)
EQPQHPLHVTYAGAAVDELGK,
(SEQ ID NO: 398)
TFSYAGFEMQPK,
(SEQ ID NO: 399)
GYIWNYGAIPQTWEDPGHNDK,
(SEQ ID NO: 400)
DYTGYNNYYGYGDYSNQQSGYGK,
(SEQ ID NO: 401)
QSGYGGQTKPIFR,
(SEQ ID NO: 402)
VPLIESGTAGYLGQVTTIKK,
(SEQ ID NO: 403)
GILGYTEHQVVSSDFNSDTH,
(SEQ ID NO: 404)
GILGYTEHQVVSSDFNSDTHSS,
(SEQ ID NO: 405)
QTCVXHYTGMLEDGKK,
(SEQ ID NO: 406)
QTCVXHYTGMLEDGKKFDS,
and
(SEQ ID NO: 407)
AXYVTHLMK.

Exemplary cathepsin S sensitive sites include but are not limited to:

(SEQ ID NO: 408)
KVSVR,
(SEQ ID NO: 3)
TVGLR,
(SEQ ID NO: 4)
PMGLP,
(SEQ ID NO: 5)
PMGAP,
(SEQ ID NO: 409)
MDLAAAAEPGAGSQHLEVR,
(SEQ ID NO: 410)
EGAMVAATQGAAAAAGSGAGTGGGTASGGTEGGSAESEGAK,
(SEQ ID NO: 411)
GTSFDAAATSGGSASSEK,
(SEQ ID NO: 412)
AMVAATQGAAAAAGSGAGTGGGTASGGTEGGSAESEGAK,
(SEQ ID NO: 413)
GILAADESTGSIAK,
(SEQ ID NO: 414)
PAAPALSAADTKPGTTGSGAGSGGPGGLT,
(SEQ ID NO: 415)
MVAATQGAAAAAGSGAGTGGGTASGGTEGGSAESEGAK,
(SEQ ID NO: 416)
SSIQATTAAGSGHPTSCC,
(SEQ ID NO: 417)
NEAIQAAHDAVAQEGQCR,
(SEQ ID NO: 418)
QFGLPAEAVEAANKGDVEAFAK,
(SEQ ID NO: 419)
LVIPNTLAVNAAQDSTDLVAK,
(SEQ ID NO: 420)
EALAAMNAAQVKPLGK,
(SEQ ID NO: 421)
APRPPVSAASGRPQDDTDSSR,
(SEQ ID NO: 422)
GDPQEAKPQEAAVAPEKPPASDETK,
(SEQ ID NO: 423)
EGDMIVCAAYAHELPK,
(SEQ ID NO: 424)
GILAADESTGSIAK,
(SEQ ID NO: 425)
PEEACSFILSADFPALVVK,
(SEQ ID NO: 426)
GWNAYIDNLMADGTCQDAAIVGYK,
(SEQ ID NO: 427)
YLAADKDGNVTCER,
(SEQ ID NO: 428)
LPVDFVTADKFDENAK,
(SEQ ID NO: 429)
TXEAEAAHGTVTR,
(SEQ ID NO: 430)
TVFAEHISDECK,
(SEQ ID NO: 431)
TVFAEHISDECKR,
(SEQ ID NO: 432)
DLEAEHVEVEDTTLNR,
(SEQ ID NO: 433)
CAEIAHNVSSK,
(SEQ ID NO: 434)
EAAAAGGGVGAGAGGGCGPGGADSSKPR,
(SEQ ID NO: 435)
YXLVGAGAIGCELLK,
(SEQ ID NO: 436)
LIYAGKILNDDTALK,
(SEQ ID NO: 437)
FGDNTAGCTSAGPHFNPLSR,
(SEQ ID NO: 438)
IITLAGPTNAIFK,
(SEQ ID NO: 439)
EIVHXQAGQCGNQIGAK,
(SEQ ID NO: 440)
AICAGPTALLAHEIGFGSK,
(SEQ ID NO: 441)
SHEHSPSDLEAHFVPLVK,
(SEQ ID NO: 442)
ELQAHGADELLK,
(SEQ ID NO: 443)
SWADLVNAHVVPGSGVVK,
(SEQ ID NO: 444)
TFIAIKPDGVQR
(SEQ ID NO: 445)
GYIWNYGAIPQTWEDPGHNDK,
(SEQ ID NO: 446)
ATATXXAKPQITNPK,
(SEQ ID NO: 447)
TTETAQHAQGAKPQVQPQK,
(SEQ ID NO: 448)
VASYLLAALGGNSSPSAK,
(SEQ ID NO: 449)
IALPAPRGSGTASD,
(SEQ ID NO: 450)
QIGNVAALPGIVHR,
(SEQ ID NO: 451)
DGTVLCELINALYPEGQAPVK,
(SEQ ID NO: 452)
DGTVLCELINALYPEGQAPVKK,
(SEQ ID NO: 453)
DFTVSAMHGDMDQK,
(SEQ ID NO: 454)
LVTDCVAAMNPDAVLR,
(SEQ ID NO: 455)
ELQANCYEEVKDR,
(SEQ ID NO: 456)
CSLQAAAILDANDAHQTETSSSQVK,
(SEQ ID NO: 457)
SGLGRPQLQGAPAAEPMAVP,
(SEQ ID NO: 458)
QETAVAPSLVAPALNKPK,
(SEQ ID NO: 459)
AQXAAPASVPAQAPK,
(SEQ ID NO: 460)
GETIFVTAPHEATAGIIGVNR,
(SEQ ID NO: 461)
EYSSELNAPSQESDSHPR,
(SEQ ID NO: 462)
DQVTAQEIFQDNHEDGPTAK,
(SEQ ID NO: 463)
LHEEEIQELQAQIQEQHVQ,
(SEQ ID NO: 464)
QQQRPLEAQPSAPGHSVK,
(SEQ ID NO: 465)
AAHTANFLLNASGSTSTPAPSR,
(SEQ ID NO: 466)
DOCXFLGASLKDEVLK,
(SEQ ID NO: 467)
NVEEADAAMAASPHAVDGNTVELK,
(SEQ ID NO: 468)
ALLVTASQCQQPAENK,
(SEQ ID NO: 469)
QSSWGMMGMLASQQNQSGPSGNNQNQGNMQ,
(SEQ ID NO: 470)
LPPGFSASSTVEKPSK,
(SEQ ID NO: 471)
AAVPSGASTGIYEALE,
(SEQ ID NO: 472)
LNCQVIGASVDSHFCH,
(SEQ ID NO: 473)
GANQYTFHLEATENPGALIK,
(SEQ ID NO: 474)
LSELTQQLAQATGKPPQYIAVH,
(SEQ ID NO: 475)
AGEQEGAMVAATQGAAAAAGSGAGTGGGTA
SGGTEGGSAESEGAK,
(SEQ ID NO: 476)
ISSIQATTAAGSGHPTSCC,
(SEQ ID NO: 477)
GLGATTHPTAAVK,
(SEQ ID NO: 478)
IEPPPLDAVIEAEHTLR,
(SEQ ID NO: 479)
SXGLPVGAVINCADNTGAK,
(SEQ ID NO: 480)
VNVANVGAVPSGQDNIHR,
(SEQ ID NO: 481)
QFLECAQNQGDIK,
(SEQ ID NO: 482)
GGLTDEAALSCCSDADPSTK,
(SEQ ID NO: 483)
ILSCGEVIHVK,
(SEQ ID NO: 484)
AIVDCGFEHPSEVQ,
(SEQ ID NO: 485)
HYYEVSCHDQGLCR,
(SEQ ID NO: 486)
MLVQCMQDQEHPSIR,
(SEQ ID NO: 487)
DVVICPDASLEDAKK,
(SEQ ID NO: 488)
SGYAFVDCPDEHWAMK,
(SEQ ID NO: 489)
FVLCPECENPETDLHVNPK,
(SEQ ID NO: 490)
IAILTCPFEPPKPK,
(SEQ ID NO: 491)
IAILTCPFEPPKPK,
(SEQ ID NO: 492)
AATEQYHQVLCPGPSQDDPLHPLNK,
(SEQ ID NO: 493)
AIVICPTDEDLKDR,
(SEQ ID NO: 494)
ATAHAQAQLGCPVIIHPGR,
(SEQ ID NO: 495)
IIPGXMCQGGDFTR,
(SEQ ID NO: 496)
IIPGXMCQGGDFTR,
(SEQ ID NO: 497)
PALYWLSGLTCTEQNFISK,
(SEQ ID NO: 498)
MTVGCVAGDEESYEVFK,
(SEQ ID NO: 499)
ATVAFCDAQSTQEIHEK,
(SEQ ID NO: 500)
NYGILADATEQVGQHK,
(SEQ ID NO: 501)
LQDCEGLIVR,
(SEQ ID NO: 502)
QISAGYXPVXDCHTAHIACK,
(SEQ ID NO: 503)
QISAGYXPVXDCHTAHIACK,
(SEQ ID NO: 504)
TGEPCCDWVGDEGAGHFVK,
(SEQ ID NO: 505)
DATNVGDEGGFAPNILENK,
(SEQ ID NO: 506)
NILDFPQHVSPSK,
(SEQ ID NO: 507)
DHVVSDFSEHGSLK,
(SEQ ID NO: 508)
ADNELSPECLDGAQHFLK,
(SEQ ID NO: 509)
IQTLGYFPVGDGDFPHQK,
(SEQ ID NO: 510)
DXQEXXXFLLDGLHEDLNR,
(SEQ ID NO: 511)
MILIQDGSQNTNVDKPLR,
(SEQ ID NO: 512)
FTISDHPQPIDPLLK,
(SEQ ID NO: 513)
VNPTXFFDIAVDGEPLGR,
(SEQ ID NO: 514)
YEDICPSTHNMDVPNIK,
(SEQ ID NO: 515)
NGSIYNPEVLDITEETLHSR,
(SEQ ID NO: 516)
QHIVNDMNPGNLH,
(SEQ ID NO: 517)
MLLDSEQHPCQLK,
(SEQ ID NO: 518)
EGLMLDSHEELYK,
(SEQ ID NO: 519)
TAGDTHLGGEDFDNR,
(SEQ ID NO: 520)
PGGLLLGDVAPNFEANTTVGR,
(SEQ ID NO: 521)
NDGATILSMMDVDHQIAK,
(SEQ ID NO: 522)
GEEGLTLNLEDVQPHDLGK,
(SEQ ID NO: 523)
TIDNSQGAYQEAFDISKK,
(SEQ ID NO: 524)
IVGFFDDSFSEAHSEFLK,
(SEQ ID NO: 525)
QEEASGVALGEAPDHSYESLR,
(SEQ ID NO: 526)
DPVQEAWAEDVDLR,
(SEQ ID NO: 527)
PMIYICGECHTENEIK,
(SEQ ID NO: 528)
HMSEFMECNLNELVK,
(SEQ ID NO: 529)
MGYAEEAPYDAIHVG,
(SEQ ID NO: 530)
MADQLTEEQIAEFK,
(SEQ ID NO: 531)
YLAEFATGNDRK,
(SEQ ID NO: 532)
LAELEEFINGPNNAHIQ,
(SEQ ID NO: 533)
XEFTDHLVK,
(SEQ ID NO: 534)
FCVGFLEGGKDSCQGDSGGPVVC,
(SEQ ID NO: 535)
LLAEGHPDPDAELQR,
(SEQ ID NO: 536)
GLTEGLHGFH,
(SEQ ID NO: 537)
GLTEGLHGFH,
(SEQ ID NO: 538)
FVHWYVGEGMEEGEFSEAR,
(SEQ ID NO: 539)
MLLHEGQHPAQLR,
(SEQ ID NO: 540)
YHGYTFANLGEHEFVEEK,
(SEQ ID NO: 541)
TVFAEHISDECK,
(SEQ ID NO: 542)
VILEEHSTCENEVSK,
(SEQ ID NO: 543)
LLTEIHGGAGGPSGR,
(SEQ ID NO: 544)
AHLMEIQVNGGTVAEK,
(SEQ ID NO: 545)
ISWLDANTLAEKDEFEHK,
(SEQ ID NO: 546)
ISWLDANTLAEKDEFEHK,
(SEQ ID NO: 547)
XEKFEDENFILK,
(SEQ ID NO: 548)
SAVEAGSEVSEKPGQEAPVLPK,
(SEQ ID NO: 549)
SAVEAGSEVSEKPGQEAPVLPK,
(SEQ ID NO: 550)
ILNEKPTTDEPEK,
(SEQ ID NO: 551)
YLAEKYEWDVAEAR,
(SEQ ID NO: 552)
CLELFXELAEDKENY,
(SEQ ID NO: 553)
CLELFXELAEDKENYK,
(SEQ ID NO: 554)
MEELHNQEVQK,
(SEQ ID NO: 555)
GVNVAGVSLQELNPEMGTDNDSENWK,
(SEQ ID NO: 556)
ASDIAMTELPPTHPIR,
(SEQ ID NO: 557)
VVVAENFDEIVNNENK,
(SEQ ID NO: 558)
IXEGCEEPATHNALAK,
(SEQ ID NO: 559)
VTEQGAELSNEER,
(SEQ ID NO: 560)
AVTEQGHELSNEER,
(SEQ ID NO: 561)
QVDQEEPHVEEQQQQTPAENK,
(SEQ ID NO: 562)
QVDQEEPHVEEQQQQTPAENK,
(SEQ ID NO: 563)
VVFEQTKVIADNVK,
(SEQ ID NO: 564)
NIFVGENILEESENLHNADQPLR,
(SEQ ID NO: 565)
LFIHESIHDEVVNR,
(SEQ ID NO: 566)
VTNGIEEPLEESSHEPEPEPESETK,
(SEQ ID NO: 567)
GIVEESVTGVHR,
(SEQ ID NO: 568)
QCPSVVSLLSESYNPHVR,
(SEQ ID NO: 569)
ASLQETHFDSTQTK,
(SEQ ID NO: 570)
TFGETHPFTK,
(SEQ ID NO: 571)
VMLGETNPADSKPGTIR,
(SEQ ID NO: 572)
VMLGETNPADSKPGTIR,
(SEQ ID NO: 573)
VMLGETNPADSKPGTIR,
(SEQ ID NO: 574)
GADFLVTEVENGGSLGSK,
(SEQ ID NO: 575)
LPTEAYISVEEVHDDGTPTSK,
(SEQ ID NO: 576)
LPTEAYISVEEVHDDGTPTSK,
(SEQ ID NO: 577)
MEEVPHDCPGADSAQAGR,
(SEQ ID NO: 578)
VDENCVGFDHTVKPV,
(SEQ ID NO: 579)
VHVVPDQLMAFGGSSEPCALC,
(SEQ ID NO: 580)
IWCFGPDGTGPNILT,
(SEQ ID NO: 581)
YVXFGPHAGK,
(SEQ ID NO: 582)
EFAGFQCQIQFGPHNEQK,
(SEQ ID NO: 583)
KPXKPMQFLGDEETVRK,
(SEQ ID NO: 584)
MVYMFQYDSTHGK,
(SEQ ID NO: 585)
EELGFRPEYSASQLK,
(SEQ ID NO: 586)
HLEFSHDQYR,
(SEQ ID NO: 587)
TCGFDFTGAVEDISK,
(SEQ ID NO: 588)
GFGFVDFNSEEDAK,
(SEQ ID NO: 589)
GFGFVDFNSEEDAK,
(SEQ ID NO: 590)
NYGFVHIEDK,
(SEQ ID NO: 591)
GFGFVTFDDHDPVDK,
(SEQ ID NO: 592)
LPNFGFVVFDDSEPVQK,
(SEQ ID NO: 593)
QLLCGAAIGTHEDDK,
(SEQ ID NO: 594)
QLLCGAAIGTHEDDKYR,
(SEQ ID NO: 595)
MTNGFSGADLTEICQR,
(SEQ ID NO: 596)
VQGEVMEGADNQGAGEQGRPVR,
(SEQ ID NO: 597)
MGGHGYGGAGDASSGFHGGHF,
(SEQ ID NO: 598)
LGNVLGGLISGAGGGGGGGGGGGGGGGGGGGGTAMR,
(SEQ ID NO: 599)
FGGSFAGSFGGAGGHAPGVAR,
(SEQ ID NO: 600)
FGGSFAGSFGGAGGHAPGVAR,
(SEQ ID NO: 601)
VLVVGAGGIGCELLK,
(SEQ ID NO: 602)
VTADHGPAVSGAHNTIICAR,
(SEQ ID NO: 603)
CEALAGAPLDNAPK,
(SEQ ID NO: 604)
CEALAGAPLDNAPK,
(SEQ ID NO: 605)
STGGAPTFNVTVTK,
(SEQ ID NO: 606)
KGCDVVVIPAGVPR,
(SEQ ID NO: 607)
FSPAGVEGCPALPHK,
(SEQ ID NO: 608)
HSSLAGCQIINYR,
(SEQ ID NO: 609)
SSEVGYDAMAGDFVNMVEK,
(SEQ ID NO: 610)
SIEDSVISLSGDHCIIGR,
(SEQ ID NO: 611)
SIEDSVISLSGDHCIIGR,
(SEQ ID NO: 612)
VTGDHIPTPQDLPQR,
(SEQ ID NO: 613)
VTGDHIPTPQDLPQR,
(SEQ ID NO: 614)
NGDTFLGGEDFDQALLR,
(SEQ ID NO: 615)
IVYICCGEDHTAALTK,
(SEQ ID NO: 616)
MVDGNVSGEFTDLVPEK,
(SEQ ID NO: 617)
MAAQGEPQVQFK,
(SEQ ID NO: 618)
QALAVHLALQGESSSEHFLK,
(SEQ ID NO: 619)
AFYNNVLGEYEEYITK,
(SEQ ID NO: 620)
LLNQMDGFDTLHR,
(SEQ ID NO: 621)
GLTEGLHGFHVHEFG,
(SEQ ID NO: 622)
GLTEGLHGFHVHEFGDNTAGCT,
(SEQ ID NO: 623)
GLTEGLHGFHVHEFGDNTAGCT,
(SEQ ID NO: 624)
GLTEGLHGFHVHEFGDNTAGCT,
(SEQ ID NO: 625)
GLTEGLHGFHVHEFGDNTAGCT,
(SEQ ID NO: 626)
GLTEGLHGFHVHEFGDNTAGCT,
(SEQ ID NO: 627)
AADSYFSLLQGFINSLDESTQESK,
(SEQ ID NO: 628)
INPYLLGTMAGGAADCSFWER,
(SEQ ID NO: 629)
QHDLFDSGFGGGAGVETGGK,
(SEQ ID NO: 630)
TTHFVEGGDAGNREDQINR,
(SEQ ID NO: 631)
TTHFVEGGDAGNREDQINR,
(SEQ ID NO: 632)
SQPIAQQPLQGGDHSGNYGYK,
(SEQ ID NO: 633)
GTDGTDNPLSGGDQYQNITVHR,
(SEQ ID NO: 634)
GCITXIGGGDTATCCAK,
(SEQ ID NO: 635)
WGSGGGGGGGGGGGGGGGGGGGGGGGGGGGRKSSSAAA,
(SEQ ID NO: 636)
LAAGSLAAPGGGGGSAGGARP,
(SEQ ID NO: 637)
GSXXXGGGSYNDFGNY,
(SEQ ID NO: 638)
VNAANXSLLGGGGVDGCIHR,
(SEQ ID NO: 639)
FCVGFLEGGKDSCQGDSGGPVVC,
(SEQ ID NO: 640)
LVDGQIFCLHGGLSPSIDTLDHIR,
(SEQ ID NO: 641)
MFXGGLSWDTSKK,
(SEQ ID NO: 642)
DPQELLEGGNQGEGDPQAEGR,
(SEQ ID NO: 643)
NMGGPYGGGNYGPGGSGGSGGYGGR,
(SEQ ID NO: 644)
RGGPGGPGGPGGPMGR,
(SEQ ID NO: 645)
SVLDDWFPLQGGQGQVHLR,
(SEQ ID NO: 646)
IIMEYLGGGSALDLLR,
(SEQ ID NO: 647)
SHFAMMHGGTGFAGIDSSSPEVK,
(SEQ ID NO: 648)
QGFQLTHSLGGGTGSGMGTLLI,
(SEQ ID NO: 649)
MADYLISGGTSYVPDDGLT,
(SEQ ID NO: 650)
VTVAGGVHISGLH,
(SEQ ID NO: 651)
VTVAGGVHISGLHT,
(SEQ ID NO: 652)
VTVAGGVHISGLHTE,
(SEQ ID NO: 653)
YAVSELAGHQTSAESWGTGR,
(SEQ ID NO: 654)
TFQGHTNEVNAIK,
(SEQ ID NO: 655)
GDGPVQGIINFEQK,
(SEQ ID NO: 656)
VTIIGPATVGGIKPGCFK,
(SEQ ID NO: 657)
FSLPGMEHVYGIPEHADNLR,
(SEQ ID NO: 658)
IFVGGIPHNCGETELR,
(SEQ ID NO: 659)
LPPSGAVPVTGIPPHVVK,
(SEQ ID NO: 660)
MDGIVPDIAVGTK,
(SEQ ID NO: 661)
RGIWHNDNK,
(SEQ ID NO: 662)
GKPEIEGKPESEGEPGSETR,
(SEQ ID NO: 663)
YDINAHACVTGKPISQGGIHGR,
(SEQ ID NO: 664)
ELTQQLAQATGKPPQYIAVH,
(SEQ ID NO: 665)
NPKPFLNGLTGKPVMVK,
(SEQ ID NO: 666)
CPSILGGLAPEKDQPK,
(SEQ ID NO: 667)
VASGIPAGWXGLDCGPESSKK,
(SEQ ID NO: 668)
QVLQGLDYLHSK,
(SEQ ID NO: 669)
GALEGLPRPPPPVK,
(SEQ ID NO: 670)
LFIGGLSFETTDESLR,
(SEQ ID NO: 671)
VFVGGLSPDTSEEQIK,
(SEQ ID NO: 672)
MFXGGLSWDTSKK,
(SEQ ID NO: 673)
NVIIWGNHSSTQYPDVNHAK,
(SEQ ID NO: 674)
LLSGLAEGLGGNIEQLVAR,
(SEQ ID NO: 675)
LVINGNPITIFQER,
(SEQ ID NO: 676)
SAAMLGNSEDHTALSR,
(SEQ ID NO: 677)
IFQGNVHNFEK,
(SEQ ID NO: 678)
NNPPTLEGNYSKPLK,
(SEQ ID NO: 679)
MVGPAVIVDKK,
(SEQ ID NO: 680)
MMLGPEGGEGFVVK,
(SEQ ID NO: 681)
SIYEALGGPHDPNVAK,
(SEQ ID NO: 682)
TFQGPNCPATCGR,
(SEQ ID NO: 683)
IMGPNYTPGKK,
(SEQ ID NO: 684)
MVIITGPPEAQFK,
(SEQ ID NO: 685)
AFGLTDDQVSGPPSAPAEDR,
(SEQ ID NO: 686)
TVQGPPTSDDIFER,
(SEQ ID NO: 687)
FVIGGPQGDAGLTGR,
(SEQ ID NO: 688)
IITLXGPTNAIFK,
(SEQ ID NO: 689)
KPPTLIHGQAPSAGLPSQKPK,
(SEQ ID NO: 690)
RGQGGYPGKPR,
(SEQ ID NO: 691)
RPDNFXFGQSGAGNNWAK,
(SEQ ID NO: 692)
GLLALSSALSGQSHLAIK,
(SEQ ID NO: 693)
ALPPVLTTVNGQSPPEHSAPAK,
(SEQ ID NO: 694)
QSGYGGQTKPIFR,
(SEQ ID NO: 695)
LSGQTNIHLSK,
(SEQ ID NO: 696)
VVLMSHLGRPDGVPMPDK,
(SEQ ID NO: 697)
VVLMSHLGRPDGVPMPDKY,
(SEQ ID NO: 698)
QQSIAGSADSKPIDVSR,
(SEQ ID NO: 699)
VTLGPVPEIGGSEAPAPQNK,
(SEQ ID NO: 700)
NFGGSFAGSFGGAGGHAPGVAR,
(SEQ ID NO: 701)
MMDYLQGSGETPQTDVR,
(SEQ ID NO: 702)
DSVWGSGGGQQSVNHLVK,
(SEQ ID NO: 703)
PQVAIICGSGLGGLTDK,
(SEQ ID NO: 704)
PTSSEQGGLEGSGSAAGEGKPALSEEER,
(SEQ ID NO: 705)
TVEQLLTGSPTSPTVEPEKPTR,
(SEQ ID NO: 706)
GCLEGSQGTQALHK,
(SEQ ID NO: 707)
LLAVSAPALQGSRPGETEENVR,
(SEQ ID NO: 708)
IXXGSSGAQGSGGGSTSAHY,
(SEQ ID NO: 709)
VAFTGSTEVGHLIQK,
(SEQ ID NO: 710)
VVVLMGSTSDLGHCEK,
(SEQ ID NO: 711)
MVELLGSYTEDNASQAR,
(SEQ ID NO: 712)
IYWGTATTGKPHVA,
(SEQ ID NO: 713)
IVGFCWGGTAVHHLM,
(SEQ ID NO: 714)
GVVPLAGTDGETTTQGLDGLSER,
(SEQ ID NO: 715)
GXVXFXGTDHIDQWNK,
(SEQ ID NO: 716)
SVSGTDVQEECR,
(SEQ ID NO: 717)
MMLGTEGGEGFVVK,
(SEQ ID NO: 718)
IAFHQDGSLAGTGGLDAFGR,
(SEQ ID NO: 719)
LNFSHGTHEYHAETIK,
(SEQ ID NO: 720)
LVLGTHTSDEQNHLV,
(SEQ ID NO: 721)
ALHWLVLGTHTSDEQNHLVVAR,
(SEQ ID NO: 722)
LSGTIHAGQPVK,
(SEQ ID NO: 723)
IITITGTQDQIQNAQY,
(SEQ ID NO: 724)
GGTSDVEVNEK,
(SEQ ID NO: 725)
VLTGVAGEDAECHAAK,
(SEQ ID NO: 726)
TGGVDTAAVGGVFDVSNADR,
(SEQ ID NO: 727)
FIVDGWHEMDAENPLH,
(SEQ ID NO: 728)
TMFSSEVQFGHAGACANQASETAVAK,
(SEQ ID NO: 729)
PIYDVLQMVGHANRPLQDDEGR,
(SEQ ID NO: 730)
EWAHATIIPK,
(SEQ ID NO: 731)
KHEANNPQLK,
(SEQ ID NO: 732)
MVNHFIAEFK,
(SEQ ID NO: 733)
LVXHFVEEFK,
(SEQ ID NO: 734)
MPFPVNHGASSEDTLLK,
(SEQ ID NO: 735)
NXCWELYCLEHGIQPDGQMPSDK,
(SEQ ID NO: 736)
NXCWELYCLEHGIQPDGQMPSDK,
(SEQ ID NO: 737)
VHAGPFANIAHGNSSIIADR,
(SEQ ID NO: 738)
INQVFHGSCITEGNELTK,
(SEQ ID NO: 739)
FELQHGTEEQQEEVR,
(SEQ ID NO: 740)
EQQEAIEHIDEVQNEIDR,
(SEQ ID NO: 741)
VEALAAALAHISGATSVDQR,
(SEQ ID NO: 742)
RHLAPTGNAPASR,
(SEQ ID NO: 743)
LLTDFCTHLPNLPDSTAK,
(SEQ ID NO: 744)
VDEFVTHNLSFDEINK,
(SEQ ID NO: 745)
ATLELTHNWGTEDDETQSY,
(SEQ ID NO: 746)
EEFTAFLHPEEYDYMK,
(SEQ ID NO: 747)
QXFHPEQLITGK,
(SEQ ID NO: 748)
PVTHNLPTVAHPSQAPSPNQPTK,
(SEQ ID NO: 749)
AXXXXXQHQAGQAPHLG,
(SEQ ID NO: 750)
CNFTDGALVQHQEWDGK,
(SEQ ID NO: 751)
GVLHQFSGTETNK,
(SEQ ID NO: 752)
QIGAVVSHQSSVIPDR,
(SEQ ID NO: 753)
IEPNEVTHSGDTGVETDGR,
(SEQ ID NO: 754)
HYAHTDCPGHADYVK,
(SEQ ID NO: 755)
TICSHVQNMIK,
(SEQ ID NO: 756)
LLGHWEEAAHDLA,
(SEQ ID NO: 757)
TYTIANQFPLNK,
(SEQ ID NO: 758)
NPTXFFDIAVDGEPLGR,
(SEQ ID NO: 759)
LVSIGAEEIVDGNAK,
(SEQ ID NO: 760)
TTDGVYEGVAIGGDRYPGSTF,
(SEQ ID NO: 761)
THINIVVIGHVDSGK,
(SEQ ID NO: 762)
DNDFCGTDMTIGTDSALHR,
(SEQ ID NO: 763)
VLXNMEIGTSLFDEEGAK,
(SEQ ID NO: 764)
VCTLAIIDPGDSDIIR,
(SEQ ID NO: 765)
GCITIIGGGDTATCCAK,
(SEQ ID NO: 766)
TFNQVEIKPEMIGH,
(SEQ ID NO: 767)
CQLEINFNTLQTK,
(SEQ ID NO: 768)
HLEINPDHPIVE,
(SEQ ID NO: 769)
HLEINPDHSIIETLR,
(SEQ ID NO: 770)
VPYLIAGIQHSCQDIGAK,
(SEQ ID NO: 771)
VLSIQSHVIR,
(SEQ ID NO: 772)
ELGITALHIK,
(SEQ ID NO: 773)
LVAIVDPHIK,
(SEQ ID NO: 774)
TLTIVDTGIGMTK,
(SEQ ID NO: 775)
LVAIVDVIDQNR,
(SEQ ID NO: 776)
QIILEKEETEELKR,
(SEQ ID NO: 777)
XKHPDADSLY,
(SEQ ID NO: 778)
CIGKPGGSLDNSEQK,
(SEQ ID NO: 779)
HHIYLEGTLLKPNMVTPGHACTQK,
(SEQ ID NO: 780)
LTQQLAQATGKPPQYIAVH,
(SEQ ID NO: 781)
SSPPELPDVMKPQDSGSSANEQAVQ,
(SEQ ID NO: 782)
LQELEKYPGIQTR,
(SEQ ID NO: 783)
WIGLDLSNGKPR,
(SEQ ID NO: 784)
MPFLELDTNLPANR,
(SEQ ID NO: 785)
ETALLSSGFSLEDPQTHANR,
(SEQ ID NO: 786)
EAFSLFDKDGDGTITTK,
(SEQ ID NO: 787)
YELGRPAANTK,
(SEQ ID NO: 788)
GNPICSLHDQGAGGNGNVLK,
(SEQ ID NO: 789)
VILHLKEDQTEYLEER,
(SEQ ID NO: 790)
IQQLCEDIIQLKPDVVITEK,
(SEQ ID NO: 791)
IQQLCEDIIQLKPDVVITEK,
(SEQ ID NO: 792)
TLNNDIMLIK,
(SEQ ID NO: 793)
NQVALNPQNTVFDAK,
(SEQ ID NO: 794)
NQVALNPQNTVFDAK,
(SEQ ID NO: 795)
STATLAWGVNLPAHTVIIK,
(SEQ ID NO: 796)
EXLELPEDEEEKK,
(SEQ ID NO: 797)
GVNLPGAAVDLPAVSEK,
(SEQ ID NO: 798)
RLPPAAGDEP,
(SEQ ID NO: 799)
LDLPPYETF,
(SEQ ID NO: 800)
DGDSVMVLPTIPEEEAKK,
(SEQ ID NO: 801)
EIVHLQAGQCGNQIGAK,
(SEQ ID NO: 802)
DVSIEDSVISLSGDHCIIGR,
(SEQ ID NO: 803)
SSAPGPLELDLTGDLESFKK,
(SEQ ID NO: 804)
FLEMCNDLLAR,
(SEQ ID NO: 805)
TTGFGMIYDSLDYAK,
(SEQ ID NO: 806)
XMNPTNTVFDAK,
(SEQ ID NO: 807)
EDAMAMVDHCLK,
(SEQ ID NO: 808)
ANXVXSGGXTMYPGIADR,
(SEQ ID NO: 809)
ANXVXSGGXTMYPGIADR,
(SEQ ID NO: 810)
ALQDLENAASGDAAVHQR,
(SEQ ID NO: 811)
DPVTNLNNAFEVAEK,
(SEQ ID NO: 812)
XNAGPNTNGSQFF,
(SEQ ID NO: 813)
NYSVFYYEIQNAPEQACH,
(SEQ ID NO: 814)
ELISNASDALDKIR,
(SEQ ID NO: 815)
YYFNHITNASQWERPSGNSSSGGK,
(SEQ ID NO: 816)
TNDWEDHLAVK,
(SEQ ID NO: 817)
AFHNEAQVNPERK,
(SEQ ID NO: 818)
NCLTNFHGMDLTR,
(SEQ ID NO: 819)
TNVANFPGHSGPIT,
(SEQ ID NO: 820)
ILNNGHAFNVEFDDSQDK,
(SEQ ID NO: 821)
IEQLQNHENEDIYK,
(SEQ ID NO: 822)
PVFVHAGPFANIAHGNSSIIADR,
(SEQ ID NO: 823)
VWYVSNIDGTHIAK,
(SEQ ID NO: 824)
CDEVMQLLLENLGNENVHR,
(SEQ ID NO: 825)
QDQRPLHPVANPHAEISTK,
(SEQ ID NO: 826)
XNPLDAGAAEPI,
(SEQ ID NO: 827)
LIPQLVANVTNPNSTEHMK,
(SEQ ID NO: 828)
SAAMLGNSEDHTALSR,
(SEQ ID NO: 829)
NYQQNYQNSESGEKNEGSESAPEGQAQQR,
(SEQ ID NO: 830)
LGEMWNNTAADDKQPYEK,
(SEQ ID NO: 831)
IMQNTDPHSQEYVEHLK,
(SEQ ID NO: 832)
ILIANTGMDTDKIK,
(SEQ ID NO: 833)
AWVWNTHADFADECPKPELL,
(SEQ ID NO: 834)
DHASIQMNVAEVDKVTGR,
(SEQ ID NO: 835)
ALANVNIGSLIC,
(SEQ ID NO: 836)
EHGXXTNWDDMEK,
(SEQ ID NO: 837)
SAAQAAAQTNSNAAGK,
(SEQ ID NO: 838)
EETFEAAMLGQAEEVVQER,
(SEQ ID NO: 839)
PPYDEQTQAFIDAAQEAR,
(SEQ ID NO: 840)
LEQGQAIDDLMPAQK,
(SEQ ID NO: 841)
SLHQAIEGDTSGDFLK,
(SEQ ID NO: 842)
QLQQAQAAGAEQEVEK,
(SEQ ID NO: 843)
YLEVVLNTLQQASQAQVDK,
(SEQ ID NO: 844)
YLEVVLNTLQQASQAQVDK,
(SEQ ID NO: 845)
FLSELTQQLAQATGKPPQYI,
(SEQ ID NO: 846)
FLSELTQQLAQATGKPPQYIA,
(SEQ ID NO: 847)
FLSELTQQLAQATGKPPQYIAVH,
(SEQ ID NO: 848)
MTSMGQATWSDPHK,
(SEQ ID NO: 849)
EELGLIEQAYDNPHEALSR,
(SEQ ID NO: 850)
SLGTIQQCCDAIDHLCR,
(SEQ ID NO: 851)
AAAAAAQQQQQCGGGGATKPAVSGK,
(SEQ ID NO: 852)
NSCNQCNEPRPEDSR,
(SEQ ID NO: 853)
VLIAFAQYLQQCPFEDHVK,
(SEQ ID NO: 854)
DSLLQDGEFSMDLR,
(SEQ ID NO: 855)
YFLGSIVNFSQDPDVHFK,
(SEQ ID NO: 856)
VFSWLQQEGHLSEEEMAR,
(SEQ ID NO: 857)
VMSQEIQEQLHK,
(SEQ ID NO: 858)
KQEPVKPEEGR,
(SEQ ID NO: 859)
LWYCDLQQESSGIAGILK,
(SEQ ID NO: 860)
KQEYDESGPSIVHR,
(SEQ ID NO: 861)
ETEAICFFVQQFTDMEHNR,
(SEQ ID NO: 862)
VTEQGAELSNEER,
(SEQ ID NO: 863)
AYMGNVLQGGEGQAPTR,
(SEQ ID NO: 864)
AVTEQGHELSNEER,
(SEQ ID NO: 865)
VAHTFVVDVAQGTQVTGR,
(SEQ ID NO: 866)
VGQGYPHDPPK,
(SEQ ID NO: 867)
IYAVEASTMAQHAEVLVK,
(SEQ ID NO: 868)
TLAIYFEVVNQHNAPIPQGGR,
(SEQ ID NO: 869)
ELAQIAGRPTEDEDEKEK,
(SEQ ID NO: 870)
MDEMATTQISKDELDELK,
(SEQ ID NO: 871)
YPHLGQKPGGSDFLR,
(SEQ ID NO: 872)
TMLELLNQLDGFQPNTQVK,
(SEQ ID NO: 873)
ILLELLNQMDGFDQNVNVK,
(SEQ ID NO: 874)
LLNQMDGFDTLHR,
(SEQ ID NO: 875)
FQESAEAILGQNAAYLGELK,
(SEQ ID NO: 876)
HPCFIIAEIGQNHQGDLDVAK,
(SEQ ID NO: 877)
LLQDHPWLLSQNLVVKPDQLIK,
(SEQ ID NO: 878)
ALPAVQQNNLDEDLIRK,
(SEQ ID NO: 879)
ALGQNPTNAEVLK,
(SEQ ID NO: 880)
NYQQNYQNSESGEK,
(SEQ ID NO: 881)
NYQQNYQNSESGEKNEGSESAPEGQAQQR,
(SEQ ID NO: 882)
CGAPSATQPATAETQHIADQVR,
(SEQ ID NO: 883)
QAAAAAAQQQQQCGGGGATKPAVSGK,
(SEQ ID NO: 884)
IDVTDFLSMTQQDSHAPLR,
(SEQ ID NO: 885)
IGSCTQQDVELHVQK,
(SEQ ID NO: 886)
LFPLNQQDVPDKFK,
(SEQ ID NO: 887)
IGQQPQQPGAPPQQDYTK,
(SEQ ID NO: 888)
HQAAAAAAQQQQQCGGGGATKPAVSGK,
(SEQ ID NO: 889)
MFTQQQPQELAR,
(SEQ ID NO: 890)
LQQQQRPEDAEDGAEGGGK,
(SEQ ID NO: 891)
LQQQQRPEDAEDGAEGGGKR,
(SEQ ID NO: 892)
SSEADMECLNQRPPENPDTDK,
(SEQ ID NO: 893)
SSEADMECLNQRPPENPDTDKNVQ,
(SEQ ID NO: 894)
NVNPESQLIQQSEQSESETAGSTK,
(SEQ ID NO: 895)
PDNFXFGQSGAGNNWAK,
(SEQ ID NO: 896)
SQTCEFNMIEQSGPPHEPR,
(SEQ ID NO: 897)
SAVLPPEDMSQSGPSGSHPQGPR,
(SEQ ID NO: 898)
IEFLQSHENQEIYQK,
(SEQ ID NO: 899)
NTVSQSISGDPEIDKK,
(SEQ ID NO: 900)
LLIHQSLAGGIIGVK,
(SEQ ID NO: 901)
MVXYLANLTQSQIALNEK
(SEQ ID NO: 902)
PPKPEPFQFGQSSQKPPVAGGK,,
(SEQ ID NO: 903)
NGNYCVLQMDQSYKPDENEVR,
(SEQ ID NO: 904)
ILVGDVGQTVDDPYATFVK,
(SEQ ID NO: 905)
ADDVDLEQVANETHGHVG,
(SEQ ID NO: 906)
ADDVDLEQVANETHGHVGA,
(SEQ ID NO: 907)
SINFLHQVCHDQTPTTK,
(SEQ ID NO: 908)
CTTVAFTQVNSEDKGALAK,
(SEQ ID NO: 909)
QQLQQVPGLLHR,
(SEQ ID NO: 910)
SQQYPAARPAEP,
(SEQ ID NO: 911)
DFCIQVGRNIIHGSDSVK,
(SEQ ID NO: 912)
VLMSHLGRPDGVPMPDKY,
(SEQ ID NO: 913)
VLMSHLGRPDGVPMPDKYS,
(SEQ ID NO: 914)
AQVARPGGDTIFGK,
(SEQ ID NO: 915)
AQVARPGGDTIFGK,
(SEQ ID NO: 916)
FMSVQRPGPYDRPGTAR,
(SEQ ID NO: 917)
VLVERSAAETVTK,
(SEQ ID NO: 918)
FLPSARSSPASSPE,
(SEQ ID NO: 919)
RPELGSEGLGSAAHGSQPDLR,
(SEQ ID NO: 920)
MPDQGMTSADDFFQGTK,
(SEQ ID NO: 921)
DVPAPSTSADKVESLDVDSEAK,
(SEQ ID NO: 922)
QVCLPVISAENWKPATK,
(SEQ ID NO: 923)
GFGSGDDPYSSAEPHVSGVK,
(SEQ ID NO: 924)
EFGDNTAGCTSAGPHFNPLSR,
(SEQ ID NO: 925)
TYFSCTSAHTSTGDGTAMITR,
(SEQ ID NO: 926)
TYSLGSALRPSTSR,
(SEQ ID NO: 927)
VSDQELQSANASVDDSR,
(SEQ ID NO: 928)
APGSAAPAAGSAPAAAEEK,
(SEQ ID NO: 929)
APGSAAPAAGSAPAAAEEK,
(SEQ ID NO: 930)
APGSAAPAAGSAPAAAEEK,
(SEQ ID NO: 931)
APGSAAPAAGSAPAAAEEKK,
(SEQ ID NO: 932)
NEGSESAPEGQAQQR,
(SEQ ID NO: 933)
QVEPLDPPAGSAPGEHVFVK,
(SEQ ID NO: 934)
PTGEAGPSCSSASDKLPR,
(SEQ ID NO: 935)
YYTSASGDEMVSLK,
(SEQ ID NO: 936)
NQQGAHSALSSASTSSHNLQ,
(SEQ ID NO: 937)
EALLSSAVDHGSDEVK,
(SEQ ID NO: 938)
DYMVEIDILASCDHPNIVK,
(SEQ ID NO: 939)
MESCGIHETTF,
(SEQ ID NO: 940)
QLSSCLPNIVPK,
(SEQ ID NO: 941)
LIXSDGHEFIVK,
(SEQ ID NO: 942)
EIVDGGVILESDPQQVVHR,
(SEQ ID NO: 943)
SLEDALSSDTSGHFR,
(SEQ ID NO: 944)
VGVEAHVDIHSDVPKGANSF,
(SEQ ID NO: 945)
VILGSEAAQQHPEEVR,
(SEQ ID NO: 946)
XSEDKGALAK,
(SEQ ID NO: 947)
GGTSXXSSEGTQHSYSEEEK,
(SEQ ID NO: 948)
CALGGTSELSSEGTQHSYSEEEKY,
(SEQ ID NO: 949)
MDPNIVGSEHYDVAR,
(SEQ ID NO: 950)
SPAPSSVPLGSEKPSNVSQDR,
(SEQ ID NO: 951)
MTQAGVEELESENKIPATQK,
(SEQ ID NO: 952)
MLLDSEQHPCQLK,
(SEQ ID NO: 953)
GLGNVLGGLISGAGGGGGGGGGGGGGGGGGGGGTAMR,
(SEQ ID NO: 954)
IMDDLTEVLCSGAGGVHSGGSGDGAGSGGPGAQNHVK,
(SEQ ID NO: 955)
ATQGAAAAAGSGAGTGGGTASGGTEGGSAESEGAK,
(SEQ ID NO: 956)
LEPAPLDSLCSGASAEEPTSHR,
(SEQ ID NO: 957)
VIGSGCNLDSAR,
(SEQ ID NO: 958)
WXLNSGDGAFYGPK,
(SEQ ID NO: 959)
FFDMAYQGFASGDGDKDAWAVR,
(SEQ ID NO: 960)
VSIEDSVISLSGDHCIIGR,
(SEQ ID NO: 961)
EYLLSGDISEAEHCLK,
(SEQ ID NO: 962)
DDGLFSGDPNWFPK,
(SEQ ID NO: 963)
WQHDLFDSGFGGGAGVETGGK,
(SEQ ID NO: 964)
DSVWGSGGGQQSVNHLVK,
(SEQ ID NO: 965)
PEGPNEAEVTSGKPEQEVPDAEEEK,
(SEQ ID NO: 966)
VQSGNINAAK,
(SEQ ID NO: 967)
YQYGGLNSGRPVTPPR,
(SEQ ID NO: 968)
VLQATVVAVGSGSKGKGGEIQPVSVK,
(SEQ ID NO: 969)
GILFVGSGVSGGEEGAR,
(SEQ ID NO: 970)
IEFLQSHENQEIYQK,
(SEQ ID NO: 971)
LDEVITSHGAIEPDKDNVR,
(SEQ ID NO: 972)
EHPVIESHPDNALEDLR,
(SEQ ID NO: 973)
LIQSHPESAEDLQEK,
(SEQ ID NO: 974)
TIVITSHPGQIVK,
(SEQ ID NO: 975)
IEWLESHQDADIEDFK,
(SEQ ID NO: 976)
GYPHLCSICDLPVHSNK,
(SEQ ID NO: 977)
SEPCALCSLHSIGKIGGAQNR,
(SEQ ID NO: 978)
LQSIGTENTEENR,
(SEQ ID NO: 979)
LFIHESIHDEVVNR,
(SEQ ID NO: 980)
VTFNINNSIPPTFDGEEEPSQGQK,
(SEQ ID NO: 981)
NLNTLCWAIGSISGAMHEEDEKR,
(SEQ ID NO: 982)
EASATNSPCTSKPATPAPSEK,
(SEQ ID NO: 983)
PPNPNCYVCASKPEVTVR,
(SEQ ID NO: 984)
ICSKPVVLPK,
(SEQ ID NO: 985)
QFHFHWGSLDGQGSEHTVDK,
(SEQ ID NO: 986)
QFHFHWGSLDGQGSEHTVDKK,
(SEQ ID NO: 987)
GNPICSLHDQGAGGNGNVLK,
(SEQ ID NO: 988)
EANFTVSSMHGDMPQK,
(SEQ ID NO: 989)
NQLTSNPENTVFDAK,
(SEQ ID NO: 990)
QVLVGSYCVFSNQGGLVHPK,
(SEQ ID NO: 991)
DLQSNVEHLTEK,
(SEQ ID NO: 992)
EEMQSNVEVVHTYR,
(SEQ ID NO: 993)
APVQPQQSPAAAPGGTDEKPSGK,
(SEQ ID NO: 994)
APVQPQQSPAAAPGGTDEKPSGK,
(SEQ ID NO: 995)
NDGPVTIELESPAPGTATSDPK,
(SEQ ID NO: 996)
INSLFLTDLYSPEYPGPSHR,
(SEQ ID NO: 997)
NGSLDSPGKQDTEEDEEEDEKDK,
(SEQ ID NO: 998)
SAAAASAASGSPGPGEGSAGGEKR,
(SEQ ID NO: 999)
SAAAASAASGSPGPGEGSAGGEKR,
(SEQ ID NO: 1000)
NADTDLVSWLSPHDPNSVVTK,
(SEQ ID NO: 1001)
LSPPYSSPQEFAQDVGR,
(SEQ ID NO: 1002)
IIAFVGSPVEDNEKDLVK,
(SEQ ID NO: 1003)
MESQEPTESSQNGK,
(SEQ ID NO: 1004)
AXASQLDCNFLK,
(SEQ ID NO: 1005)
SQGDSISSQLGPIHPPPR,
(SEQ ID NO: 1006)
LGGLLKPTVASQNQNLPVAK,
(SEQ ID NO: 1007)
SSWGMMGMLASQQNQSGPSGNNQNQGNMQR,
(SEQ ID NO: 1008)
DEYLINSQTTEHIVK,
(SEQ ID NO: 1009)
YQLGLAYGYNSQYDEAVAQFSK,
(SEQ ID NO: 1010)
GLLLLSVVVTSRPEAFQPH,
(SEQ ID NO: 1011)
RPASVSSSAAVEHEQR,
(SEQ ID NO: 1012)
FGIVTSSAGTGTTEDTEAK,
(SEQ ID NO: 1013)
FGIVTSSAGTGTTEDTEAKK,
(SEQ ID NO: 1014)
STASAPAAVNSSASADKPLSNMK,
(SEQ ID NO: 1015)
EALLSSAVDHGSDEVK,
(SEQ ID NO: 1016)
VSWLEYESSFSNEEAQK,
(SEQ ID NO: 1017)
IXXGSSGAQGSGGGSTSAHY,
(SEQ ID NO: 1018)
HIGGPPGFASSSGKPGPTVIK,
(SEQ ID NO: 1019)
FEMYEPSELESSHLTDQDNEIR,
(SEQ ID NO: 1020)
SPDDDLGSSNWEAADLGNEER,
(SEQ ID NO: 1021)
GDSQVSSNPTSSPPGEAPAPVSVDSEPS,
(SEQ ID NO: 1022)
FVNGQPRPLESSQVKYLR,
(SEQ ID NO: 1023)
KPLTSSSAAPQRPISTQR,
(SEQ ID NO: 1024)
IHIGGPPGFASSSGKPGPTVIK,
(SEQ ID NO: 1025)
ELVSSSSSGSDSDSEVDKK,
(SEQ ID NO: 1026)
LLDSSTVTHLFK,
(SEQ ID NO: 1027)
PPPAAPPPSSSSVPEAGGPPIKK,
(SEQ ID NO: 1028)
YVELFLNSTAGASGGAYEHR,
(SEQ ID NO: 1029)
SHELSDFGLESTAGEIPVVAIR,
(SEQ ID NO: 1030)
ECEEEAINIQSTAPEEEHESPR,
(SEQ ID NO: 1031)
EGTGSTATSSSSTAGAAGK,
(SEQ ID NO: 1032)
PLHSIISSTESVQGSTSK,
(SEQ ID NO: 1033)
VAFTGSTEVGHLIQK,
(SEQ ID NO: 1034)
LALVTGGEIASTFDHPELVK,
(SEQ ID NO: 1035)
ATIELCSTHANDASALR,
(SEQ ID NO: 1036)
VHITLSTHECAGLSER,
(SEQ ID NO: 1037)
EEEEPQAPQESTPAPPKK,
(SEQ ID NO: 1038)
SITILSTPEGTSAACK,
(SEQ ID NO: 1039)
ETLASSDSFASTQPTHSWK,
(SEQ ID NO: 1040)
VVVLMGSTSDLGHCEK,
(SEQ ID NO: 1041)
VLLSNLSSTSHVPEVDPGSAELQK,
(SEQ ID NO: 1042)
LFDSTTLEHQK,
(SEQ ID NO: 1043)
TQLEGLQSTVTGHVER,
(SEQ ID NO: 1044)
GSESGGSAVDSVAGEHSVSGR,
(SEQ ID NO: 1045)
YEILQSVDDAAIVIK,
(SEQ ID NO: 1046)
NDLSICGTLHSVDQYLNIK,
(SEQ ID NO: 1047)
ILDSVGIEADDDR,
(SEQ ID NO: 1048)
ILDSVGIEADDDRLNK,
(SEQ ID NO: 1049)
IYVASVHQDLSDDDIK,
(SEQ ID NO: 1050)
ELQSVKPQEAPK,
(SEQ ID NO: 1051)
HYTEGAELVDSVLDVVRK,
(SEQ ID NO: 1052)
LAEGSVTSVGSVNPAENFR,
(SEQ ID NO: 1053)
GSPTSLGTWGSWIGPDHDK,
(SEQ ID NO: 1054)
VLNSYWVGEDSTYK,
(SEQ ID NO: 1055)
SLGTADVHFER,
(SEQ ID NO: 1056)
MAGTAFDFENMK,
(SEQ ID NO: 1057)
VLATAFDTTLGGR,
(SEQ ID NO: 1058)
VELFLNSTAGASGGAYEHR,
(SEQ ID NO: 1059)
APPPSGSAVSTAPQQKPIGK,
(SEQ ID NO: 1060)
SQIFSTASDNQPTVTIK,
(SEQ ID NO: 1061)
IYWGTATTGKPHVA,
(SEQ ID NO: 1062)
MMLGTEGGEGFVVK,
(SEQ ID NO: 1063)
FGAVWTGDNTAEWDHLK,
(SEQ ID NO: 1064)
VSHVSTGGGASLELL,
(SEQ ID NO: 1065)
VSHVSTGGGASLELLE,
(SEQ ID NO: 1066)
VSHVSTGGGASLELLEGK,
(SEQ ID NO: 1067)
ILISLATGHREEGGENLDQAR,
(SEQ ID NO: 1068)
TLDQCIQTGVDNPGHPFIK,
(SEQ ID NO: 1069)
SGFTLDDVIQTGVDNPGHPY,
(SEQ ID NO: 1070)
DLTTGYDDSQPDKK,
(SEQ ID NO: 1071)
FFFGTHETAFLGPK,
(SEQ ID NO: 1072)
FPSLLTHNENMVAK,
(SEQ ID NO: 1073)
YEDICPSTHNMDVPNIK,
(SEQ ID NO: 1074)
DYALHWLVLGTHTSDEQNHLVVAR,
(SEQ ID NO: 1075)
FGTINIVHPK,
(SEQ ID NO: 1076)
SMVNTKPEKTEEDSEEVR,
(SEQ ID NO: 1077)
VTLLTPAGATGSGGGTSGDSSKGEDKQDR,
(SEQ ID NO: 1078)
PGETLTEILETPATSEQEAEHQR,
(SEQ ID NO: 1079)
NSVQTPVENSTNSQHQVK,
(SEQ ID NO: 1080)
AXXITPVPGGVGPMTV,
(SEQ ID NO: 1081)
STVLTPMFVETQASQGTLQTR,
(SEQ ID NO: 1082)
TFTTQETITNAETAK,
(SEQ ID NO: 1083)
SPVSTRPLPSASQK,
(SEQ ID NO: 1084)
TNEQWQMSLGTSEDHQHFT,
(SEQ ID NO: 1085)
QEIIXQLDVTTSEYEKEK,
(SEQ ID NO: 1086)
LLAFLLAELGTSGSIDGNNQLVIK,
(SEQ ID NO: 1087)
LXNMEIGTSLFDEEGAK,
(SEQ ID NO: 1088)
AEKPAETPVATSPTATDSTSGDSSR,
(SEQ ID NO: 1089)
LLETTDRPDGHQNNLR,
(SEQ ID NO: 1090)
AQTITSEXXSTTTTTHITK,
(SEQ ID NO: 1091)
ADAVGMSTVPEVIVAR,
(SEQ ID NO: 1092)
IHFPLATYAPVISAEK,
(SEQ ID NO: 1093)
DTXVXXDTYNCDLHFK,
(SEQ ID NO: 1094)
VVIGMDVAASEFFR,
(SEQ ID NO: 1095)
GXXXXXIGLXVADLAESIMK,
(SEQ ID NO: 1096)
ANPQVGVAFPHIK,
(SEQ ID NO: 1097)
PQEAKPQEAAVAPEKPPASDETK,
(SEQ ID NO: 1098)
HFSVEGQLEFR,
(SEQ ID NO: 1099)
VATLGVEVHPLVFH,
(SEQ ID NO: 1100)
HWPFQVINDGDKPK,
(SEQ ID NO: 1101)
LPVPAFNVINGGSHAGNK,
(SEQ ID NO: 1102)
EVANGIESLGVKPDLPPPPSK,
(SEQ ID NO: 1103)
TYYDVLGVKPNATQEELKK,
(SEQ ID NO: 1104)
ETVAVKPTENNEEEFTSK,
(SEQ ID NO: 1105)
SLLVNPEGPTLMR,
(SEQ ID NO: 1106)
NWMNSLGVNPHVNHLY,
(SEQ ID NO: 1107)
HGLLVPNNTTDQELQHIR,
(SEQ ID NO: 1108)
QELEFLEVQEEYIKDEQK,
(SEQ ID NO: 1109)
LEGTLLKPNMVTPGHACTQK,
(SEQ ID NO: 1110)
FVNVVPTFGKK,
(SEQ ID NO: 1111)
EDLVFIFWAPESAPLK,
(SEQ ID NO: 1112)
AIYIDASCLTWEGQQFQGK,
(SEQ ID NO: 1113)
EQPQHPLHVTYAGAAVDELGK,
(SEQ ID NO: 1114)
SPDGHLFQVEYAQEAVKK,
(SEQ ID NO: 1115)
NYKPPAQK,
(SEQ ID NO: 1116)
VYNYNHLMPTR,
(SEQ ID NO: 1117)
LAEAELEYNPEHVSR,
(SEQ ID NO: 1118)
MPYQYPALTPEQK,
(SEQ ID NO: 1119)
TSSANNPNLMYQDECDRR,
(SEQ ID NO: 1120)
VGINYQPPTVVPGGDLAK,
(SEQ ID NO: 1121)
YMACCXLYRGDVVPK,
and
(SEQ ID NO: 1122)
SYCYVSKEELK.

Other cathepsin sensitive sites are known to the skilled artisan or can easily be determined experimentally using digestion assays with no more than routine experimentation.

The mRNA cancer vaccines of the present invention comprise one or more polynucleotides, e.g., polynucleotide constructs, which encode one or more wild type or engineered antigens, including the concatemeric antigens. Exemplary polynucleotides, e.g., polynucleotide constructs, include antigen-encoding mRNAs. In exemplary aspect, polynucleotides of the invention, e.g., antigen-encoding mRNAs, may include at least one chemical modification.

The polynucleotides (e.g., antigen-encoding polynucleotides) can include various substitutions and/or insertions. As used herein in a polynucleotide, the terms “chemical modification” or, as appropriate, “chemically modified” refer to modification with respect to adenosine (A), guanosine (G), uridine (U), thymidine (T) or cytidine (C) ribo- or deoxyribnucleosides in one or more of their position, pattern, percent or population. Generally, herein, these terms are not intended to refer to the ribonucleotide modifications in naturally occurring 5′-terminal mRNA cap moieties. In a polypeptide, the term “modification” refers to a modification as compared to the canonical set of 20 amino acids.

The skilled artisan will appreciate that, except where otherwise noted, polynucleotide sequences set forth in the instant application will recite “T”s in a representative DNA sequence but where the sequence represents RNA, the “T”s would be substituted for “U”s.

The modifications may be various distinct modifications. In some embodiments, the regions may contain one, two, or more (optionally different) nucleoside or nucleotide modifications. In some embodiments, a modified polynucleotide, introduced to a cell or organism may exhibit reduced degradation in the cell or organism, as compared to an unmodified polynucleotide. In some embodiments, a modified polynucleotide, introduced into a call or organism, may exhibit reduced immunogenicity in the cell or organism (e.g., a reduced innate response.)

Modifications of the polynucleotides of the mRNA cancer vaccines include, but are not limited to those listed in detail below. The polynucleotide may comprise modifications which are naturally occurring, non-naturally occurring or the polynucleotide can comprise both naturally and non-naturally occurring modifications.

The polynucleotides of the mRNA cancer vaccines of the invention can include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present invention may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

Non-natural modified nucleotides may be introduced to polynucleotides, e.g., of the mRNA cancer vaccines, or nucleic acids during synthesis or post-synthesis of the chains to achieve desired functions or properties. The modifications may be on internucleotide lineage, the purine or pyrimidine bases, or sugar. The modification may be introduced at the terminal of a chain or anywhere else in the chain; with chemical synthesis or with a polymerase enzyme. Any of the regions of the polynucleotides may be chemically modified.

The present disclosure provides for modified nucleosides and nucleotides. As described herein “nucleoside” is defined as a compound containing a sugar molecule (e.g., a pentose or ribose) or a derivative thereof in combination with an organic base (e.g., a purine or pyrimidine) or a derivative thereof (also referred to herein as “nucleobase”). As described herein, “nucleotide” is defined as a nucleoside including a phosphate group. The modified nucleotides may by synthesized by any useful method, as described herein (e.g., chemically, enzymatically, or recombinantly to include one or more modified or non-natural nucleosides). The polynucleotides may comprise a region or regions of linked nucleosides. Such regions may have variable backbone linkages. The linkages may be standard phosphodiester linkages, in which case the polynucleotides would comprise regions of nucleotides.

The modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into the polynucleotides of the invention.

Modifications of the polynucleotides of the mRNA cancer vaccines which are useful in the present invention include, but are not limited to the following: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl)adenine; N6-cis-hydroxy-isopentenyl-adenosine; a-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′, 2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; a-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-I-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; a-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methyluridine, 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; a-thio-uridine; 1 (aminoalkylamino-carbonylethylenyl)-2(thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 2 (thio)pseudouracil; 2′ deoxy uridine; 2′ fluorouridine; 2-(thio)uracil; 2,4-(dithio)psuedouracil; 2′ methyl, 2′amino, 2′azido, 2′fluro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; P seudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (±)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl}pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2(2-ethoxy)-ethoxy}-ethoxy]-ethoxy)-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluro-cytidine; 2′ methyl, 2′amino, 2′azido, 2′fluro-adenine; 2′methyl, 2′amino, 2′azido, 2′fluro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, an mRNA of the invention includes a combination of one or more of the aforementioned modified nucleobases (e.g., a combination of 2, 3 or 4 of the aforementioned modified nucleobases.)

In some embodiments, the modified nucleobase is pseudouridine (ψ), N1-methylpseudouridine (m¹ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, or 2′-O-methyl uridine. In some embodiments, an mRNA of the invention includes a combination of one or more of the aforementioned modified nucleobases (e.g., a combination of 2, 3 or 4 of the aforementioned modified nucleobases.)

In some embodiments, the modified nucleobase is 1-methyl-pseudouridine (ml), 5-methoxy-uridine (mo⁵U), 5-methyl-cytidine (m⁵C), pseudouridine (ψ), α-thio-guanosine, or α-thio-adenosine. In some embodiments, an mRNA of the invention includes a combination of one or more of the aforementioned modified nucleobases (e.g., a combination of 2, 3 or 4 of the aforementioned modified nucleobases.)

In some embodiments, the mRNA comprises pseudouridine (ψ) and 5-methyl-cytidine (m⁵C). In some embodiments, the mRNA comprises 1-methyl-pseudouridine (m¹ψ). In some embodiments, the mRNA comprises 1-methyl-pseudouridine (m¹ψ) and 5-methyl-cytidine (m⁵C). In some embodiments, the mRNA comprises 2-thiouridine (s²U). In some embodiments, the mRNA comprises 2-thiouridine and 5-methyl-cytidine (m⁵C). In some embodiments, the mRNA comprises 5-methoxy-uridine (mo⁵U). In some embodiments, the mRNA comprises 5-methoxy-uridine (mo⁵U) and 5-methyl-cytidine (m⁵C). In some embodiments, the mRNA comprises 2′-O-methyl uridine. In some embodiments, the mRNA comprises 2′-O-methyl uridine and 5-methyl-cytidine (m⁵C). In some embodiments, the mRNA comprises N6-methyl-adenosine (m⁶A). In some embodiments, the mRNA comprises N6-methyl-adenosine (m⁶A) and 5-methyl-cytidine (m⁵C).

In certain embodiments, an mRNA of the invention is uniformly modified (i.e., fully modified, modified through-out the entire sequence) for a particular modification. For example, an mRNA can be uniformly modified with 5-methyl-cytidine (m⁵C), meaning that all cytosine residues in the mRNA sequence are replaced with 5-methyl-cytidine (m⁵C). Similarly, mRNAs of the invention can be uniformly modified for any type of nucleoside residue present in the sequence by replacement with a modified residue such as those set forth above.

In some embodiments, the modified nucleobase is a modified cytosine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine.

In some embodiments, the modified nucleobase is a modified uridine. Exemplary nucleobases and nucleosides having a modified uridine include 5-cyano uridine or 4′-thio uridine.

In some embodiments, the modified nucleobase is a modified adenine. Exemplary nucleobases and nucleosides having a modified adenine include 7-deaza-adenine, 1-methyl-adenosine (m1A), 2-methyl-adenine (m2A), N6-methyl-adenosine (m6A), and 2,6-Diaminopurine.

In some embodiments, the modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQO), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

The mRNA cancer vaccines are nucleic acid molecules, specifically polynucleotides which, in some embodiments, encode one or more peptides or polypeptides of interest. Such peptides or polypeptides serve as an antigen or antigenic molecule. The term “nucleic acid,” in its broadest sense, includes any compound and/or substance that comprise a polymer of nucleotides. These polymers are often referred to as polynucleotides.

Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or hybrids or combinations thereof.

In one embodiment, the polynucleotides of the present invention is or functions as a messenger RNA (mRNA). As used herein, the term “messenger RNA” (mRNA) refers to any polynucleotide which encodes at least one peptide or polypeptide of interest and which is capable of being translated to produce the encoded peptide polypeptide of interest in vitro, in vivo, in situ or ex vivo.

Traditionally, the basic components of an mRNA molecule include at least a coding region, a 5′UTR, a 3′UTR, a 5′ cap and a poly-A tail. The polynucleotides of the present invention may function as mRNA but are distinguished from wild-type mRNA in their functional and/or structural design features which serve to overcome existing problems of effective polypeptide production using nucleic-acid based therapeutics. It is to be understood that the antigens of the mRNA cancer vaccines of the present invention may be encoded by in vitro translated (IVT) polynucleotides. A “primary construct” refers to a polynucleotide which encodes one or more polypeptides of interest and which retains sufficient structural and/or chemical features to allow the polypeptide of interest encoded therein to be translated.

An “in vitro transcription template (IVT),” as used herein, refers to deoxyribonucleic acid (DNA) suitable for use in an IVT reaction for the production of messenger RNA (mRNA).

In some embodiments, an IVT template encodes a 5′ untranslated region, contains an open reading frame, and encodes a 3′ untranslated region and a polyA tail. The particular nucleotide sequence composition and length of an IVT template will depend on the mRNA of interest encoded by the template.

A “5′ untranslated region (UTR)” refers to a region of an mRNA that is directly upstream (i.e., 5′) from the start codon (i.e., the first codon of an mRNA transcript translated by a ribosome) that does not encode a protein or peptide.

A “3′ untranslated region (UTR)” refers to a region of an mRNA that is directly downstream (i.e., 3′) from the stop codon (i.e., the codon of an mRNA transcript that signals a termination of translation) that does not encode a protein or peptide.

An “open reading frame” is a continuous stretch of DNA beginning with a start codon (e.g., methionine (ATG)), and ending with a stop codon (e.g., TAA, TAG or TGA) and encodes a protein or peptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

In some embodiments, the polynucleotide includes from about 200 to about 3,000 nucleotides (e.g., from 200 to 500, from 200 to 1,000, from 200 to 1,500, from 200 to 3,000, from 500 to 1,000, from 500 to 1,500, from 500 to 2,000, from 500 to 3,000, from 1,000 to 1,500, from 1,000 to 2,000, from 1,000 to 3,000, from 1,500 to 3,000, and from 2,000 to 3,000).

In other aspects, the invention relates to a method for preparing an mRNA cancer vaccine by IVT methods. In vitro transcription (IVT) methods permit template-directed synthesis of RNA molecules of almost any sequence. The size of the RNA molecules that can be synthesized using IVT methods range from short oligonucleotides to long nucleic acid polymers of several thousand bases. IVT methods permit synthesis of large quantities of RNA transcript (e.g., from microgram to milligram quantities) (Beckert et al., Synthesis of RNA by in vitro transcription, Methods Mol Biol. 703:29-41(2011); Rio et al. RNA: A Laboratory Manual. Cold Spring Harbor: Cold Spring Harbor Laboratory Press, 2011, 205-220; Cooper, Geoffery M. The Cell: A Molecular Approach. 4th ed. Washington D.C.: ASM Press, 2007. 262-299). Generally, IVT utilizes a DNA template featuring a promoter sequence upstream of a sequence of interest. The promoter sequence is most commonly of bacteriophage origin (ex. the T7, T3 or SP6 promoter sequence) but many other promotor sequences can be tolerated including those designed de novo. Transcription of the DNA template is typically best achieved by using the RNA polymerase corresponding to the specific bacteriophage promoter sequence. Exemplary RNA polymerases include, but are not limited to T7 RNA polymerase, T3 RNA polymerase, or SP6 RNA polymerase, among others. IVT is generally initiated at a dsDNA but can proceed on a single strand.

It will be appreciated that mRNA vaccines of the present disclosure, e.g., mRNAs encoding the concatameric antigen, may be made using any appropriate synthesis method. For example, in some embodiments, mRNA vaccines of the present disclosure are made using IVT from a single bottom strand DNA as a template and complementary oligonucleotide that serves as promotor. The single bottom strand DNA may act as a DNA template for in vitro transcription of RNA, and may be obtained from, for example, a plasmid, a PCR product, or chemical synthesis. In some embodiments, the single bottom strand DNA is linearized from a circular template. The single bottom strand DNA template generally includes a promoter sequence, e.g., a bacteriophage promoter sequence, to facilitate IVT. Methods of making RNA using a single bottom strand DNA and a top strand promoter complementary oligonucleotide are known in the art. An exemplary method includes, but is not limited to, annealing the DNA bottom strand template with the top strand promoter complementary oligonucleotide (e.g., T7 promoter complementary oligonucleotide, T3 promoter complementary oligonucleotide, or SP6 promoter complementary oligonucleotide), followed by IVT using an RNA polymerase corresponding to the promoter sequence, e.g., aT7 RNA polymerase, a T3 RNA polymerase, or an SP6 RNA polymerase.

IVT methods can also be performed using a double-stranded DNA template. For example, in some embodiments, the double-stranded DNA template is made by extending a complementary oligonucleotide to generate a complementary DNA strand using strand extension techniques available in the art. In some embodiments, a single bottom strand DNA template containing a promoter sequence and sequence encoding one or more epitopes of interest is annealed to a top strand promoter complementary oligonucleotide and subjected to a PCR-like process to extend the top strand to generate a double-stranded DNA template. Alternatively or additionally, a top strand DNA containing a sequence complementary to the bottom strand promoter sequence and complementary to the sequence encoding one or more epitopes of interest is annealed to a bottom strand promoter oligonucleotide and subjected to a PCR-like process to extend the bottom strand to generate a double-stranded DNA template. In some embodiments, the number of PCR-like cycles ranges from 1 to 20 cycles, e.g., 3 to 10 cycles. In some embodiments, a double-stranded DNA template is synthesized wholly or in part by chemical synthesis methods. The double-stranded DNA template can be subjected to in vitro transcription as described herein.

In another aspect, mRNA vaccines of the present disclosure, e.g., mRNAs encoding the concatameric antigen, may be made using two DNA strands that are complementary across an overlapping portion of their sequence, leaving single-stranded overhangs (i.e., sticky ends) when the complementary portions are annealed. These single-stranded overhangs can be made double-stranded by extending using the other strand as a template, thereby generating double-stranded DNA. In some cases, this primer extension method can permit larger ORFs to be incorporated into the template DNA sequence, e.g., as compared to sizes incorporated into the template DNA sequences obtained by top strand DNA synthesis methods. In the primer extension method, a portion of the 3′-end of a first strand (in the 5″-3′ direction) is complementary to a portion the 3′-end of a second strand (in the 3′-5′ direction). In some such embodiments, the single first strand DNA may include a sequence of a promoter (e.g., T7, T3, or SP6), optionally a 5′-UTR, and some or all of an ORF (e.g., a portion of the 5′-end of the ORF). In some embodiments, the single second strand DNA may include complementary sequences for some or all of an ORF (e.g., a portion complementary to the 3′-end of the ORF), and optionally a 3′-UTR, a stop sequence, and/or a poly(A) tail. Methods of making RNA using two synthetic DNA strands may include annealing the two strands with overlapping complementary portions, followed by primer extension using one or more PCR-like cycles to extend the strands to generate a double-stranded DNA template. In some embodiments, the number of PCR-like cycles ranges from 1 to 20 cycles, e.g., 3 to 10 cycles. Such double-stranded DNA can be subjected to in vitro transcription as described herein.

In another aspect, mRNA vaccines of the present disclosure, e.g., mRNAs encoding the concatameric antigen, may be made using synthetic double-stranded linear DNA molecules, such as gBlocks® (Integrated DNA Technologies, Coralville, Iowa), as the double-stranded DNA template. An advantage to such synthetic double-stranded linear DNA molecules is that they provide a longer template from which to generate mRNAs. For example, gBlocks® can range in size from 45-1000 (e.g., 125-750 nucleotides). In some embodiments, a synthetic double-stranded linear DNA template includes a full length 5′-UTR, a full length 3′-UTR, or both. A full length 5′-UTR may be up to 100 nucleotides in length, e.g., about 40-60 nucleotides. A full length 3′-UTR may be up to 300 nucleotides in length, e.g., about 100-150 nucleotides.

To facilitate generation of longer constructs, two or more double-stranded linear DNA molecules and/or gene fragments that are designed with overlapping sequences on the 3′ strands may be assembled together using methods known in art. For example, the Gibson Assembly™ Method (Synthetic Genomics, Inc., La Jolla, CA) may be performed with the use of a mesophilic exonuclease that cleaves bases from the 5′-end of the double-stranded DNA fragments, followed by annealing of the newly formed complementary single-stranded 3′-ends, polymerase-dependent extension to fill in any single-stranded gaps, and finally, covalent joining of the DNA segments by a DNA ligase.

In another aspect, mRNA vaccines of the present disclosure, e.g., mRNAs encoding the concatameric antigen, may be made using chemical synthesis of the RNA. Methods, for instance, involve annealing a first polynucleotide comprising an open reading frame encoding the polypeptide and a second polynucleotide comprising a 5′-UTR to a complementary polynucleotide conjugated to a solid support. The 3′-terminus of the second polynucleotide is then ligated to the 5′-terminus of the first polynucleotide under suitable conditions. Suitable conditions include the use of a DNA Ligase. The ligation reaction produces a first ligation product. The 5′ terminus of a third polynucleotide comprising a 3′-UTR is then ligated to the 3′-terminus of the first ligation product under suitable conditions. Suitable conditions for the second ligation reaction include an RNA Ligase. A second ligation product is produced in the second ligation reaction. The second ligation product is released from the solid support to produce an mRNA encoding a polypeptide of interest. In some embodiments the mRNA is between 30 and 1000 nucleotides.

An mRNA encoding a polypeptide of interest may also be prepared by binding a first polynucleotide comprising an open reading frame encoding the polypeptide to a second polynucleotide comprising 3′-UTR to a complementary polynucleotide conjugated to a solid support. The 5′-terminus of the second polynucleotide is ligated to the 3′-terminus of the first polynucleotide under suitable conditions. The suitable conditions include a DNA Ligase. The method produces a first ligation product. A third polynucleotide comprising a 5′-UTR is ligated to the first ligation product under suitable conditions to produce a second ligation product. The suitable conditions include an RNA Ligase, such as T4 RNA. The second ligation product is released from the solid support to produce an mRNA encoding a polypeptide of interest.

In some embodiments the first polynucleotide features a 5′-triphosphate and a 3′-OH. In other embodiments the second polynucleotide comprises a 3′-OH. In yet other embodiments, the third polynucleotide comprises a 5′-triphosphate and a 3′-OH. The second polynucleotide may also include a 5′-cap structure. The method may also involve the further step of ligating a fourth polynucleotide comprising a poly-A region at the 3′-terminus of the third polynucleotide. The fourth polynucleotide may comprise a 5′-triphosphate.

The method may or may not comprise reverse phase purification. The method may also include a washing step wherein the solid support is washed to remove unreacted polynucleotides. The solid support may be, for instance, a capture resin. In some embodiments the method involves dT purification.

In accordance with the present disclosure, template DNA encoding the mRNA vaccines of the present disclosure includes an open reading frame (ORF) encoding one or more cancer epitopes. In some embodiments, the template DNA includes an ORF of up to 1000 nucleotides, e.g., about 10-350, 30-300 nucleotides or about 50-250 nucleotides. In some embodiments, the template DNA includes an ORF of about 150 nucleotides. In some embodiments, the template DNA includes an ORF of about 200 nucleotides.

In some embodiments, IVT transcripts are purified from the components of the IVT reaction mixture after the reaction takes place. For example, the crude IVT mix may be treated with RNase-free DNase to digest the original template. The mRNA can be purified using methods known in the art, including but not limited to, precipitation using an organic solvent or column based purification method. Commercial kits are available to purify RNA, e.g., MEGACLEAR™ Kit (Ambion, Austin, TX). The mRNA can be quantified using methods known in the art, including but not limited to, commercially available instruments, e.g., NanoDrop. Purified mRNA can be analyzed, for example, by agarose gel electrophoresis to confirm the RNA is the proper size and/or to confirm that no degradation of the RNA has occurred.

The template DNA may include one or more stabilizing elements, including, but not limited to untranslated regions (UTR) at their 5′-end (5′UTR) and/or at their 3′-end (3′UTR), in addition to other structural features, such as a 5′-cap structure or a 3′-poly(A) tail. In some embodiments, the template DNA includes a 5′-UTR of about 1-30 nucleotides, e.g., about 5-25 nucleotides or about 10-20 nucleotides. In some embodiments, the template DNA includes a 5′-UTR of 13 nucleotides. In some embodiments, the template DNA does not include a 5′-UTR. In some embodiments, the template DNA includes a 3′-UTR of about 1-60 nucleotides, e.g., 10-50 nucleotides. In some embodiments, the template DNA includes a 3′-UTR of 40 nucleotides. In some embodiments, the template DNA does not include a 3′-UTR. In some embodiments, the template DNA includes a 3′-poly(A) tail of 1-150 nucleotides, e.g., 10-100 nucleotides, e.g., 30 nucleotides. Such stabilizing elements may be included in the DNA for transcription in the IVT reaction, or may be synthesized separately and added to the resulting RNA generated from the IVT reaction.

A 3′-poly(A) tail may be added to an RNA of the present disclosure. Methods for poly(A) tail addition are well known in the art. Such methods include, but are not limited to poly(A) polymerase catalysis or periodate treatment. Alternatively or additionally, a poly(A) tail can be synthesized separately and then added to the RNA using any appropriate technique, such as click chemistry, orthoclick chemistry, solulink, or other bioconjugate chemistries known to those in the art.

A 7-methyl guanosine (m7G) cap may be added to an RNA of the present disclosure. Methods for m7G cap addition are well known in the art. Examples include, but are not limited to, co-transcriptional incorporation of anti-reverse cap analog (ARCA) using RNA polymerase, such as T7 polymerase. Commercial kits are available for T7 ARCA mRNA generation, such as the HiScribe™ T7 ARCA mRNA kit (New England BioLabs).

According to the present disclosure, two regions or parts of a chimeric polynucleotide may be joined or ligated, for example, using triphosphate chemistry. In some embodiments, a first region or part of 100 nucleotides or less is chemically synthesized with a 5′-monophosphate and terminal 3′-desOH or blocked OH. If the region is longer than 80 nucleotides, it may be synthesized as two or more strands that will subsequently be chemically linked by ligation. If the first region orpart is synthesized as a non-positionally modified region or part using IVT, conversion to the 5′-monophosphate with subsequent capping of the 3′-terminus may follow. Monophosphate protecting groups may be selected from any of those known in the art. A second region or part of the chimeric polynucleotide may be synthesized using either chemical synthesis or IVT methods, e.g., as described herein. IVT methods may include use of an RNA polymerase that can utilize a primer with a modified cap. Alternatively, a cap may be chemically synthesized and coupled to the IVT region or part.

It is noted that for ligation methods, ligation with DNA T4 ligase followed by DNAse treatment (to eliminate the DNA splint required for DNA T4 Ligase activity) should readily prevent the undesirable formation of concatenation products.

The entire chimeric polynucleotide need not be manufactured with a phosphate-sugar backbone. If one of the regions or parts encodes a polypeptide, then it is preferable that such region or part comprise a phosphate-sugar backbone.

Ligation may be performed using any appropriate technique, such as enzymatic ligation, click chemistry, orthoclick chemistry, solulink, or other bioconjugate chemistries known to those in the art. In some embodiments, the ligation is directed by a complementary oligonucleotide splint. In some embodiments, the ligation is performed without a complementary oligonucleotide splint.

In other aspects, the invention relates to kits for preparing an mRNA cancer vaccine by IVT methods. In personalized cancer vaccines, it is important to identify patient specific mutations and vaccinate the patient with one or more neoepitopes. In such vaccines, the antigen(s) encoded by the ORFs of an mRNA will be specific to the patient. The 5′- and 3′-ends of RNAs encoding the antigen(s) may be more broadly applicable, as they include untranslated regions and stabilizing regions that are common to many RNAs. Among other things, the present disclosure provides kits that include one or parts of a chimeric polynucleotide, such as one or more 5′- and/or 3′-regions of RNA, which may be combined with an ORF encoding a patient-specific epitope. For example, a kit may include a polynucleotide containing one or more of a 5′-ORF, a 3′-ORF, and a poly(A) tail. In some embodiments, each polynucleotide component is in an individual container. In other embodiments, more than one polynucleotide component is present together in a single container. In some embodiments, the kit includes a ligase enzyme. In some embodiments, provided kits include instructions for use. In some embodiments, the instructions include an instruction to ligate the epitope encoding ORF to one or more other components from the kit, e.g., 5′-ORF, a 3′-ORF, and/or a poly(A) tail.

Methods for generating personalized cancer vaccines according to the invention involve identification of mutations using techniques such as deep nucleic acid or protein sequencing methods as described herein of tissue samples. In some embodiments an initial identification of mutations in a patient's transcriptome is performed. The data from the patient's transcriptome is compared with sequence information from the patients exome in order to identify patient specific and tumor specific mutations that are expressed. The comparison produces a dataset of putative neoepitopes, referred to as a mutanome. The mutanome may include approximately 100-10,000 candidate mutations per patients. The mutanome is subject to a data probing analysis using a set of inquiries or algorithms to identify an optimal mutation set for generation of a neoantigen vaccine. In some embodiments an mRNA neoantigen vaccine is designed and manufactured. The patient is then treated with the vaccine.

The neoantigen vaccine may be a polycistronic vaccine including multiple neoepitopes or one or more single RNA vaccines or a combination thereof.

In some embodiments the entire method from the initiation of the mutation identification process to the start of patient treatment is achieved in less than 2 months. In other embodiments the whole process is achieved in 7 weeks or less, 6 weeks or less, 5 weeks or less, 4 weeks or less, 3 weeks or less, 2 weeks or less or less than 1 week. In some embodiments the whole method is performed in less than 30 days.

The mutation identification process may involve both transcriptome and exome analysis or only transcriptome or exome analysis. In some embodiments transcriptome analysis is performed first and exome analysis is performed second. The analysis is performed on a biological or tissue sample. In some embodiments a biological or tissue sample is a blood or serum sample. In other embodiments the sample is a tissue bank sample or EBV transformation of B-cells.

Once an mRNA vaccine is synthesized, it is administered to the patient. In some embodiments the vaccine is administered on a schedule for up to two months, up to three months, up to four month, up to five months, up to six months, up to seven months, up to eight months, up to nine months, up to ten months, up to eleven months, up to 1 year, up to 1 and ½ years, up to two years, up to three years, or up to four years. The schedule may be the same or varied. In some embodiments the schedule is weekly for the first 3 weeks and then monthly thereafter.

The vaccine may be administered by any route. In some embodiments the vaccine is administered by an IM or IV route.

At any point in the treatment the patient may be examined to determine whether the mutations in the vaccine are still appropriate. Based on that analysis the vaccine may be adjusted or reconfigured to include one or more different mutations or to remove one or more mutations.

It has been recognized and appreciated that, by analyzing certain properties of cancer associated mutations, optimal neoepitopes may be assessed and/or selected for inclusion in an mRNA vaccine. For example, at a given time, one or more of several properties may be assessed and weighted in order to select a set of neoepitopes for inclusion in a vaccine. A property of a neoepitope or set of neoepitopes may include, for instance, an assessment of gene or transcript-level expression in patient RNA-seq or other nucleic acid analysis, tissue-specific expression in available databases, known oncogenes/tumor suppressors, variant call confidence score, RNA-seq allele-specific expression, conservative vs. non-conservative AA substitution, position of point mutation (Centering Score for increased TCR engagement), position of point mutation (Anchoring Score for differential HLA binding), Selfness: <100% core epitope homology with patient WES data, HLA-A and -B IC50 for 8mers-11mers, HLA-DRB1 IC50 for 15mers-20mers, promiscuity Score (i.e. number of patient HLAs predicted to bind), HLA-C IC50 for 8mers-llmers, HLA-DRB3-5 IC50 for 15mers-20mers, HLA-DQB1/A1 IC50 for 15mers-20mers, HLA-DPB1/A1 IC50 for 15mers-20mers, Class I vs Class II proportion, Diversity of patient HLA-A, —B and DRB1 allotypes covered, proportion of point mutation vs complex epitopes (e.g. frameshifts), and/or pseudo-epitope HLA binding scores.

In some embodiments, the properties of cancer associated mutations used to identify optimal neoepitopes are properties related to the type of mutation, abundance of mutation in patient sample, immunogenicity, lack of self-reactivity, and nature of peptide composition.

The type of mutation should be determined and considered as a factor in determining whether a putative epitope should be included in a vaccine. The type of mutation may vary. In some instances it may be desirable to include multiple different types of mutations in a single vaccine. In other instances a single type of mutation may be more desirable. A value for particular mutation can be weighted and calculated.

The abundance of the mutation in patient sample may also be scored and factored into the decision of whether a putative epitope should be included in a vaccine. Highly abundant mutations may promote a more robust immune response.

The consideration of the immunogenicity is an important component in the selection of optimal neoepitopes for inclusion in a vaccine. Immunogenicity may be assessed for instance, by analyzing the MHC binding capacity of a neoepitope, HLA promiscuity, mutation position, predicted T cell reactivity, actual T cell reactivity, structure leading to particular conformations and resultant solvent exposure, and representation of specific amino acids. Known algorithms such as the NetMHC prediction algorithm can be used to predict capacity of a peptide to bind to common HLA-A and —B alleles. Structural assessment of a MHC bound peptide may also be conducted by in silico 3-dimensional analysis and/or protein docking programs. Use of a predicted epitope structure when bound to a MHC molecule, such as acquired from a Rosetta algorithm, may be used to evaluate the degree of solvent exposure of an amino acid residues of an epitope when the epitope is bound to a MHC molecule. T cell reactivity may be assessed experimentally with epitopes and T cells in vitro. Alternatively T cell reactivity may be assessed using T cell response/sequence datasets.

An important component of a neoepitope included in a vaccine, is a lack of self-reactivity. The putative neoepitopes may be screened to confirm that the epitope is restricted to tumor tissue, for instance, arising as a result of genetic change within malignant cells. Ideally, the epitope should not be present in normal tissue of the patient and thus, self-similar epitopes are filtered out of the dataset.

The nature of peptide composition may also be considered in the epitope design. For instance a score can be provided for each putative epitope on the value of conserved versus non-conserved amino acids found in the epitope.

In some embodiments, the analysis performed by the tools described herein may include comparing different sets of properties acquired at different times from a patient, i.e. prior to and following a therapeutic intervention, from different tissue samples, from different patients having similar tumors, etc. In some embodiments, an average of peak values from one set of properties may be compared with an average of peak values from another set of properties. For example, an average value for HLA binding may be compared between two different sets of distributions. The two sets of distributions may be determined for time durations separated by days, months, or years, for instance.

Moreover, the inventors have recognized and appreciated that such data on properties of cancer mutations may be collected and analyzed using the algorithms described herein. The data is useful for identifying neoepitopes and sets of neoepitopes for the development of personalized cancer vaccines.

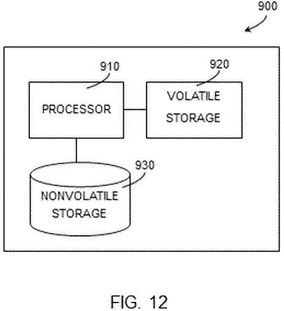

A neoepitope characterization system in accordance with the techniques described herein may take any suitable form, as embodiments are not limited in this respect. An illustrative implementation of a computer system 900 that may be used in connection with some embodiments is shown in FIG. 12. One or more computer systems such as computer system 900 may be used to implement any of the functionality described above. The computer system 900 may include one or more processors 910 and one or more computer-readable storage media (i.e., tangible, non-transitory computer-readable media), e.g., volatile storage 920 and one or more non-volatile storage media 930, which may be formed of any suitable data storage media. The processor 910 may control writing data to and reading data from the volatile storage 920 and the non-volatile storage device 930 in any suitable manner, as embodiments are not limited in this respect. To perform any of the functionality described herein, the processor 910 may execute one or more instructions stored in one or more computer-readable storage media (e.g., volatile storage 920 and/or non-volatile storage 930), which may serve as tangible, non-transitory computer-readable media storing instructions for execution by the processor 910.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation comprises at least one computer-readable storage medium (i.e., at least one tangible, non-transitory computer-readable medium), such as a computer memory (e.g., hard drive, flash memory, processor working memory, etc.), a floppy disk, an optical disk, a magnetic tape, or other tangible, non-transitory computer-readable medium, encoded with a computer program (i.e., a plurality of instructions), which, when executed on one or more processors, performs above-discussed functions. The computer-readable storage medium can be transportable such that the program stored thereon can be loaded onto any computer resource to implement techniques discussed herein. In addition, it should be appreciated that the reference to a computer program which, when executed, performs above-discussed functions, is not limited to an application program running on a host computer. Rather, the term “computer program” is used herein in a generic sense to reference any type of computer code (e.g., software or microcode) that can be employed to program one or more processors to implement above-techniques.

The mRNA cancer vaccines of the invention can be used as therapeutic or prophylactic agents. They are provided for use in medicine and/or for the priming of immune effector cells, e.g., stimulate/transfect PBMCs ex vivo and re-infuse the activated cells. For example, a vaccine described herein can be administered to a subject, wherein the polynucleotides are translated in vivo to produce an antigen. Provided are compositions, methods, kits, and reagents for diagnosis, treatment or prevention of a disease or condition in humans and other mammals. The active therapeutic agents of the invention include the mRNA cancer vaccines, cells containing mRNA cancer vaccines or polypeptides translated from the polynucleotides contained in the mRNA cancer vaccines.

The mRNA cancer vaccines may be induced for translation of a polypeptide (e.g., antigen or immunogen) in a cell, tissue or organism. Such translation can be in vivo, ex vivo, in culture, or in vitro. The cell, tissue or organism is contacted with an effective amount of a composition containing a mRNA cancer vaccine which contains a polynucleotide that has at least one a translatable region encoding the antigen or concatemeric antigen.

An “effective amount” of the mRNA cancer vaccine is provided based, at least in part, on the target tissue, target cell type, means of administration, physical characteristics of the polynucleotide (e.g., size, and extent of modified nucleosides) and other components of the mRNA cancer vaccine, and other determinants. In general, an effective amount of the mRNA cancer vaccine composition provides an induced or boosted immune response as a function of antigen production in the cell, preferably more efficient than a composition containing a corresponding unmodified polynucleotide encoding the same antigen or a peptide antigen. Increased antigen production may be demonstrated by increased cell transfection (i.e., the percentage of cells transfected with the mRNA cancer vaccine), increased protein translation from the polynucleotide, decreased nucleic acid degradation (as demonstrated, e.g., by increased duration of protein translation from a modified polynucleotide), or altered antigen specific immune response of the host cell.

In some embodiments, polynucleotides of the mRNA cancer vaccines and their encoded polypeptides in accordance with the present invention may be used for treatment of cancer.

mRNA cancer vaccines may be administered prophylactically or therapeutically as part of an active immunization scheme to healthy individuals or early in cancer or late stage and/or metastatic cancer. In one embodiment, the effective amount of the polynucleotides of the mRNA cancer vaccines of the invention provided to a cell, a tissue or a subject may be enough for immune activation, and in particular antigen specific immune activation.

The polynucleotides of the mRNA cancer vaccines may be administered with other therapeutic compounds. As a non-limiting example, the prophylactic or therapeutic compound may be an adjuvant or a booster. As used herein, when referring to a composition, such as a vaccine, the term “booster” refers to an extra administration of the composition. A booster (or booster vaccine) may be given after an earlier administration of the composition. The time of administration between the initial administration of the composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years.

In some embodiments, the polynucleotides of the mRNA cancer vaccines may be administered with an anti-cancer therapeutic agent, including but not limited to, a traditional cancer vaccine. The mRNA cancer vaccine and anti-cancer therapeutic can be combined to enhance immune therapeutic responses even further. The mRNA cancer vaccine and other therapeutic agent may be administered simultaneously or sequentially. When the other therapeutic agents are administered simultaneously they can be administered in the same or separate formulations, but are administered at the same time. The other therapeutic agents are administered sequentially with one another and with the mRNA cancer vaccine, when the administration of the other therapeutic agents and the mRNA cancer vaccine is temporally separated. The separation in time between the administration of these compounds may be a matter of minutes or it may be longer, e.g. hours, days, weeks, months. Other therapeutic agents include but are not limited to anti-cancer therapeutic, adjuvants, cytokines, antibodies, antigens, etc.

In one embodiment, the polynucleotides may be administered intramuscularly or intradermally similarly to the administration of vaccines known in the art.

The mRNA cancer vaccines may be utilized in various settings depending on the severity of the cancer or the degree or level of unmet medical need. As a non-limiting example, the mRNA cancer vaccines may be utilized to treat any stage of cancer. The mRNA cancer vaccines have superior properties in that they produce much larger antibody titers, T cell responses and produce responses early than commercially available anti-cancer vaccines. While not wishing to be bound by theory, the inventors hypothesize that the mRNA cancer vaccines, as mRNAs, are better designed to produce the appropriate protein conformation on translation as the mRNA cancer vaccines co-opt natural cellular machinery. Unlike traditional vaccines which are manufactured ex vivo and may trigger unwanted cellular responses, the mRNA cancer vaccines are presented to the cellular system in a more native fashion.

A non-limiting list of cancers that the mRNA cancer vaccines may treat is presented below. Peptide epitopes may be derived from any antigen of these cancers or tumors. Such epitopes are referred to as cancer or tumor antigens. Cancer cells may differentially express cell surface molecules during different phases of tumor progression. For example, a cancer cell may express a cell surface antigen in a benign state, yet down-regulate that particular cell surface antigen upon metastasis. As such, it is envisioned that the tumor or cancer antigen may encompass antigens produced during any stage of cancer progression. The methods of the invention may be adjusted to accommodate for these changes. For instance, several different mRNA vaccines may be generated for a particular patient. For instance a first vaccine may be used at the start of the treatment. At a later time point, a new mRNA vaccine may be generated and administered to the patient to account for different antigens being expressed.

In some embodiments, the tumor antigen is one of the following antigens: CD2, CD19, CD20, CD22, CD27, CD33, CD37, CD38, CD40, CD44, CD47, CD52, CD56, CD70, CD79, CD137, 4-IBB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gplOO, gpA33, GPNMB, ICOS, IGF1R, Integrin av, Integrin αvβ, LAG-3, Lewis Y, Mesothelin, c-MET, MN Carbonic anhydrase IX, MUC1, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, and variants thereof.

Cancers or tumors include but are not limited to neoplasms, malignant tumors, metastases, or any disease or disorder characterized by uncontrolled cell growth such that it would be considered cancerous. The cancer may be a primary or metastatic cancer. Specific cancers that can be treated according to the present invention include, but are not limited to, those listed below (for a review of such disorders, see Fishman et al., 1985, Medicine, 2d Ed., J.B. Lippincott Co., Philadelphia). Cancers include, but are not limited to, biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas, choriocarcinomas; stromal tumors and germ cell tumors; thyroid cancer including thyroid adenocarcinoma and medullar carcinoma; and renal cancer including adenocarcinoma and Wilms' tumor. Commonly encountered cancers include breast, prostate, lung, ovarian, colorectal, and brain cancer.

The polynucleotides contained in the mRNA cancer vaccines of the invention, their regions or parts or subregions may be codon optimized. Codon optimization methods are known in the art and may be useful in efforts to achieve one or more of several goals. These goals include to match codon frequencies in target and host organisms to ensure proper folding, bias GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g. glycosylation sites), add, remove or shuffle protein domains, insert or delete restriction sites, modify ribosome binding sites and mRNA degradation sites, to adjust translational rates to allow the various domains of the protein to fold properly, or to reduce or eliminate problem secondary structures within the polynucleotide. Codon optimization tools, algorithms and services are known in the art, non-limiting examples include services from GeneArt (Life Technologies), DNA2.0 (Menlo Park CA) and/or proprietary methods. In one embodiment, the ORF sequence is optimized using optimization algorithms.

In some embodiments, a codon optimized sequence shares less than 95% sequence identity to a naturally-occurring or wild-type sequence (e.g., a naturally-occurring or wild-type mRNA sequence encoding a polypeptide or protein of interest (e.g., an antigenic protein or polypeptide. In some embodiments, a codon optimized sequence shares less than 90% sequence identity to a naturally-occurring or wild-type sequence (e.g., a naturally-occurring or wild-type mRNA sequence encoding a polypeptide or protein of interest (e.g., an antigenic protein or polypeptide. In some embodiments, a codon optimized sequence shares less than 85% sequence identity to a naturally-occurring or wild-type sequence (e.g., a naturally-occurring or wild-type mRNA sequence encoding a polypeptide or protein of interest (e.g., an antigenic protein or polypeptide. In some embodiments, a codon optimized sequence shares less than 80% sequence identity to a naturally-occurring or wild-type sequence (e.g., a naturally-occurring or wild-type mRNA sequence encoding a polypeptide or protein of interest (e.g., an antigenic protein or polypeptide. In some embodiments, a codon optimized sequence shares less than 75% sequence identity to a naturally-occurring or wild-type sequence (e.g., a naturally-occurring or wild-type mRNA sequence encoding a polypeptide or protein of interest (e.g., an antigenic protein or polypeptide.

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a polypeptide or protein of interest (e.g., an antigenic protein or polypeptide. In some embodiments, a codon optimized sequence shares between 65% and 75 or about 80% sequence identity to a naturally-occurring or wild-type sequence (e.g., a naturally-occurring or wild-type mRNA sequence encoding a polypeptide or protein of interest (e.g., an antigenic protein or polypeptide.

In some embodiments a codon optimized RNA may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

In one embodiment, the polynucleotides of the present invention (e.g., antigen-encoding polynucleotides featured in the mRNA cancer vaccines of the invention) may be quantified in a biological sample or when derived from one or more bodily fluid. As used herein “bodily fluids” include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, the biological sample may be retrieved from an organ selected from the group consisting of lung, heart, pancreas, stomach, intestine, bladder, kidney, ovary, testis, skin, colon, breast, prostate, brain, esophagus, liver, and placenta.

In some instances the polypeptide encoded is larger than 25 amino acids and smaller than about 50 amino acids. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides such as antibodies or insulin and may be associated or linked. Most commonly disulfide linkages are found in multichain polypeptides. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

In some embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine.

“Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation. By “homologs” as it applies to polypeptide sequences means the corresponding sequence of other species having substantial identity to a second sequence of a second species.

“Analogs” is meant to include polypeptide variants which differ by one or more amino acid alterations, e.g., substitutions, additions or deletions of amino acid residues that still maintain one or more of the properties of the parent or starting polypeptide.

The present invention contemplates several types of compositions which are polypeptide based including variants and derivatives. These include substitutional, insertional, deletion and covalent variants and derivatives. The term “derivative” is used synonymously with the term “variant” but generally refers to a molecule that has been modified and/or changed in any way relative to a reference molecule or starting molecule.

As such, polynucleotides encoding peptides or polypeptides containing substitutions, insertions and/or additions, deletions and covalent modifications with respect to reference sequences, in particular the polypeptide sequences disclosed herein, are included within the scope of this invention. For example, sequence tags or amino acids, such as one or more lysines, can be added to the peptide sequences of the invention (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to polypeptides are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Features” when referring to polypeptide or polynucleotide are defined as distinct amino acid sequence-based or nucleotide-based components of a molecule respectively. Features of the polypeptides encoded by the polynucleotides of the present invention include surface manifestations, local conformational shape, folds, loops, half-loops, domains, half-domains, sites, termini or any combination thereof.

As used herein when referring to polypeptides the term “domain” refers to a motif of a polypeptide having one or more identifiable structural or functional characteristics or properties (e.g., binding capacity, serving as a site for protein-protein interactions).

As used herein when referring to polypeptides the terms “site” as it pertains to amino acid based embodiments is used synonymously with “amino acid residue” and “amino acid side chain.” As used herein when referring to polynucleotides the terms “site” as it pertains to nucleotide based embodiments is used synonymously with “nucleotide.” A site represents a position within a peptide or polypeptide or polynucleotide that may be modified, manipulated, altered, derivatized or varied within the polypeptide or polynucleotide based molecules of the present invention.

As used herein the terms “termini” or “terminus” when referring to polypeptides or polynucleotides refers to an extremity of a polypeptide or polynucleotide respectively. Such extremity is not limited only to the first or final site of the polypeptide or polynucleotide but may include additional amino acids or nucleotides in the terminal regions. The polypeptide based molecules of the present invention may be characterized as having both an N-terminus (terminated by an amino acid with a free amino group (NH2)) and a C-terminus (terminated by an amino acid with a free carboxyl group (COOH)). Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

As recognized by those skilled in the art, protein fragments, functional protein domains, and homologous proteins are also considered to be within the scope of polypeptides of interest of this invention. For example, provided herein is any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of about 20, about 30, about 40, about 50, or about 100 amino acids which are about 40%, about 50%, about 60%, about 70%, about 80%, about 90%, about 95%, or about 100% identical to any of the sequences described herein can be utilized in accordance with the invention. In certain embodiments, a polypeptide to be utilized in accordance with the invention includes 2, 3, 4, 5, 6, 7, 8, 9, 10, or more mutations as shown in any of the sequences provided or referenced herein.

Reference molecules (polypeptides or polynucleotides) may share a certain identity with the designed molecules (polypeptides or polynucleotides). The term “identity” as known in the art, refers to a relationship between the sequences of two or more peptides, polypeptides or polynucleotides, as determined by comparing the sequences. In the art, identity also means the degree of sequence relatedness between them as determined by the number of matches between strings of two or more amino acid residues or nucleosides. Identity measures the percent of identical matches between the smaller of two or more sequences with gap alignments (if any) addressed by a particular mathematical model or computer program (i.e., “algorithms”). Identity of related peptides can be readily calculated by known methods. Generally, variants of a particular polynucleotide or polypeptide of the invention will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402.) A general global alignment technique based on dynamic programming is the Needleman-Wunsch algorithm. More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm. Other tools are described herein, specifically in the definition of “Identity” below.

As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids.

As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

In some preferred embodiments of the invention the mRNA cancer vaccines are administered with a T cell activator such as be an immune checkpoint modulator. Immune checkpoint modulators include both stimulatory checkpoint molecules and inhibitory checkpoint molecules i.e., an anti-CTLA4 and anti-PD1 antibody.

Stimulatory checkpoint inhibitors function by promoting the checkpoint process. Several stimulatory checkpoint molecules are members of the tumor necrosis factor (TNF) receptor superfamily—CD27, CD40, OX40, GITR and CD137, while others belong to the B7-CD28 superfamily—CD28 and ICOS. OX40 (CD134), is involved in the expansion of effector and memory T cells. Anti-OX40 monoclonal antibodies have been shown to be effective in treating advanced cancer. MEDI0562 is a humanized OX40 agonist. GITR, Glucocorticoid-Induced TNFR family Related gene, is involved in T cell expansion Several antibodies to GITR have been shown to promote an anti-tumor responses. ICOS, Inducible T-cell costimulator, is important in T cell effector function. CD27 supports antigen-specific expansion of naïve T cells and is involved in the generation of T and B cell memory. Several agonistic anti-CD27 antibodies are in development. CD122 is the Interleukin-2 receptor beta sub-unit. NKTR-214 is a CD122-biased immune-stimulatory cytokine.

Inhibitory checkpoint molecules include but are not limited to PD-1, TIM-3, VISTA, A2AR, B7-H3, B7-H4, BTLA, CTLA-4, IDO, KIR and LAG3. CTLA-4, PD-1 and its ligands are members of the CD28-B7 family of co-signaling molecules that play important roles throughout all stages of T-cell function and other cell functions. CTLA-4, Cytotoxic T-Lymphocyte-Associated protein 4 (CD152), is involved in controlling T cell proliferation.

The PD-1 receptor is expressed on the surface of activated T cells (and B cells) and, under normal circumstances, binds to its ligands (PD-Li and PD-L2) that are expressed on the surface of antigen-presenting cells, such as dendritic cells or macrophages. This interaction sends a signal into the T cell and inhibits it. Cancer cells take advantage of this system by driving high levels of expression of PD-L1 on their surface. This allows them to gain control of the PD-1 pathway and switch off T cells expressing PD-1 that may enter the tumor microenvironment, thus suppressing the anticancer immune response. Pembrolizumab (formerly MK-3475 and lambrolizumab, trade name Keytruda) is a human antibody used in cancer immunotherapy. It targets the PD-1 receptor.

IDO, Indoleamine 2,3-dioxygenase, is a tryptophan catabolic enzyme, which suppresses T and NK cells, generates and activates Tregs and myeloid-derived suppressor cells, and promotes tumor angiogenesis. TIM-3, T-cell Immunoglobulin domain and Mucin domain 3, acts as a negative regulator of Thl/Tcl function by triggering cell death upon interaction with its ligand, galectin-9. VISTA, V-domain Ig suppressor of T cell activation.

The checkpoint inhibitor is a molecule such as a monoclonal antibody, a humanized antibody, a fully human antibody, a fusion protein or a combination thereof or a small molecule. For instance, the checkpoint inhibitor inhibits a checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. Ligands of checkpoint proteins include but are not limited to CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands. In some embodiments the anti-PD-1 antibody is BMS-936558 (nivolumab). In other embodiments the anti-CTLA-4 antibody is ipilimumab (trade name Yervoy, formerly known as MDX-010 and MDX-101).

In some preferred embodiments the cancer therapeutic agents, including the checkpoint modulators, are delivered in the form of mRNA encoding the cancer therapeutic agents, e.g anti-PD1, cytokines, chemokines or stimulatory receptors/ligands (e.g. OX40.

In some embodiments the cancer therapeutic agent is a targeted therapy. The targeted therapy may be a BRAF inhibitor such as vemurafenib (PLX4032) or dabrafenib. The BRAF inhibitor may be PLX 4032, PLX 4720, PLX 4734, GDC-0879, PLX 4032, PLX-4720, PLX 4734 and Sorafenib Tosylate. BRAF is a human gene that makes a protein called B-Raf, also referred to as proto-oncogene B-Raf and v-Raf murine sarcoma viral oncogene homolog Bl. The B-Raf protein is involved in sending signals inside cells, which are involved in directing cell growth. Vemurafenib, a BRAF inhibitor, was approved by FDA for treatment of late-stage melanoma.

The T-cell therapeutic agent in other embodiments is OX40L. OX40 is a member of the tumor necrosis factor/nerve growth factor receptor (TNFR/NGFR) family. OX40 may play a role in T-cell activation as well as regulation of differentiation, proliferation or apoptosis of normal and malignant lymphoid cells.

In other embodiments the cancer therapeutic agent is a cytokine. In yet other embodiments the cancer therapeutic agent is a vaccine comprising a population based tumor specific antigen.

The present invention provides pharmaceutical compositions including mRNA cancer vaccines and mRNA cancer vaccine compositions and/or complexes optionally in combination with one or more pharmaceutically acceptable excipients.

The vaccines may be formulated or administered alone or in conjunction with one or more other components. For instance the vaccine compositions of the invention may comprise other components including, but not limited to, adjuvants. Optionally the vaccine is free of adjuvants.

The present invention provides mRNA cancer vaccines and mRNA cancer vaccine pharmaceutical compositions and complexes optionally in combination with one or more pharmaceutically acceptable excipients. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety).

In some embodiments, compositions are administered to humans, human patients or subjects. For the purposes of the present disclosure, the phrase “active ingredient” generally refers to the mRNA cancer vaccines or the polynucleotides contained therein, e.g., antigen-encoding polynucleotides, for example, mRNAs, to be delivered as described herein.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

The mRNA cancer vaccines of the invention can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with mRNA cancer vaccines (e.g., for transplantation into a subject), hyaluronidase, nanoparticle mimics and combinations thereof.

### Stabilizing Elements

Naturally-occurring eukaryotic mRNA molecules have been found to contain stabilizing elements, including, but not limited to untranslated regions (UTR) at their 5′-end (5′UTR) and/or at their 3′-end (3′UTR), in addition to other structural features, such as a 5′-cap structure or a 3′-poly(A) tail. Both the 5′UTR and the 3′UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing. The 3′-poly(A) tail is typically a stretch of adenine nucleotides added to the 3′-end of the transcribed mRNA. It can comprise up to about 400 adenine nucleotides. In some embodiments the length of the 3′-poly(A) tail may be an essential element with respect to the stability of the individual mRNA.

In some embodiments the RNA vaccine may include one or more stabilizing elements. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it is peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the RNA vaccines include a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, P-Galactosidase, EGFP), or a marker or selection protein (e.g. alpha-Globin, Galactokinase and Xanthine:guanine phosphoribosyl transferase (GPT)).

In some embodiments, the combination of a poly(A) sequence or polyadenylation signal and at least one histone stem-loop, even though both represent alternative mechanisms in nature, acts synergistically to increase the protein expression beyond the level observed with either of the individual elements. It has been found that the synergistic effect of the combination of poly(A) and at least one histone stem-loop does not depend on the order of the elements or the length of the poly(A) sequence.

In some embodiments, the RNA vaccine does not comprise a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. Ideally, the inventive nucleic acid does not include an intron.

In some embodiments, the RNA vaccine may or may not contain a enhancer and/or promoter sequence, which may be modified or unmodified or which may be activated or inactivated. In some embodiments, the histone stem-loop is generally derived from histone genes, and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures, but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In other embodiments the RNA vaccine may have one or more AU-rich sequences removed. These sequences, sometimes referred to as AURES are destabilizing sequences found in the 3′UTR. The AURES may be removed from the RNA vaccines. Alternatively the AURES may remain in the RNA vaccine.

In one embodiment, the mRNA cancer vaccines may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Pub. No. WO2012013326 or US Patent Pub. No. US20130142818; each of which is herein incorporated by reference in its entirety. In another embodiment, the mRNA cancer vaccines may be formulated in a lipid-polycation complex which may further include a non-cationic lipid such as, but not limited to, cholesterol or dioleoyl phosphatidylethanolamine (DOPE).

The liposome formulation may be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010 28:172-176; herein incorporated by reference in its entirety), the liposome formulation was composed of 57.1% cationic lipid, 7.1% dipalmitoylphosphatidylcholine, 34.3% cholesterol, and 1.4% PEG-c-DMA. As another example, changing the composition of the cationic lipid could more effectively deliver siRNA to various antigen presenting cells (Basha et al. Mol Ther. 2011 19:2186-2200; herein incorporated by reference in its entirety). In some embodiments, liposome formulations may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to mRNA in liposomes may be from about 5:1 to about 20:1, from about 10:1 to about 25:1, from about 15:1 to about 30:1 and/or at least 30:1.

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain from about 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of PEG-c-DOMG (R-3-[(ω-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In another embodiment the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In one embodiment, the mRNA cancer vaccine formulation comprising the polynucleotide is a nanoparticle which may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In another aspect, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in US Patent Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In one embodiment, the lipid nanoparticle formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of about 20-60% cationic lipid:5-25% neutral lipid:25-55% sterol; 0.5-15% PEG-lipid.

In one embodiment, the formulation includes from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In one embodiment, the formulation includes from about 0.5% to about 15% on a molar basis of the neutral lipid e.g., from about 3 to about 12%, from about 5 to about 10% or about 15%, about 10%, or about 7.5% on a molar basis. Exemplary neutral lipids include, but are not limited to, DSPC, POPC, DPPC, DOPE and SM. In one embodiment, the formulation includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In one embodiment, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis. In one embodiment, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Exemplary PEG-modified lipids include, but are not limited to, PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in its entirety)

In one embodiment, the formulations of the inventions include 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In one embodiment, the formulations of the inventions include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In one embodiment, the formulations of the inventions include 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In one embodiment, the formulations of the inventions include about 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.5% of the neutral lipid, about 31% of the sterol, and about 1.5% of the PEG or PEG-modified lipid on a molar basis.

In one embodiment, the formulations of the inventions include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 38.5% of the sterol, and about 1.5% of the PEG or PEG-modified lipid on a molar basis.

In one embodiment, the formulations of the inventions include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 35% of the sterol, about 4.5% or about 5% of the PEG or PEG-modified lipid, and about 0.5% of the targeting lipid on a molar basis.

In one embodiment, the formulations of the inventions include about 40% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 15% of the neutral lipid, about 40% of the sterol, and about 5% of the PEG or PEG-modified lipid on a molar basis.

In one embodiment, the formulations of the inventions include about 57.2% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.1% of the neutral lipid, about 34.3% of the sterol, and about 1.4% of the PEG or PEG-modified lipid on a molar basis.

In one embodiment, the formulations of the inventions include about 57.5% of a cationic lipid selected from the PEG lipid is PEG-cDMA (PEG-cDMA is further discussed in Reyes et al. (J. Controlled Release, 107, 276-287 (2005), the contents of which are herein incorporated by reference in its entirety), about 7.5% of the neutral lipid, about 31.5% of the sterol, and about 3.5% of the PEG or PEG-modified lipid on a molar basis.

In preferred embodiments, lipid nanoparticle formulation consists essentially of a lipid mixture in molar ratios of about 20-70% cationic lipid:5-45% neutral lipid:20-55% cholesterol: 0.5-15% PEG-modified lipid; more preferably in a molar ratio of about 20-60% cationic lipid:5-25% neutral lipid:25-55% cholesterol: 0.5-15% PEG-modified lipid.

In particular embodiments, the molar lipid ratio is approximately 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-cDMA).

Exemplary lipid nanoparticle compositions and methods of making same are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578 (the contents of each of which are incorporated herein by reference in their entirety).

In one embodiment, the lipid nanoparticle formulations described herein may comprise a cationic lipid, a PEG lipid and a structural lipid and optionally comprise a non-cationic lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In one embodiment, the cationic lipid may be any cationic lipid described herein such as, but not limited to, DLin-KC2-DMA, DLin-MC3-DMA and L319.

In one embodiment, the lipid nanoparticle formulations described herein may be 4 component lipid nanoparticles. The lipid nanoparticle may comprise a cationic lipid, a non-cationic lipid, a PEG lipid and a structural lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In one embodiment, the cationic lipid may be any cationic lipid described herein such as, but not limited to, DLin-KC2-DMA, DLin-MC3-DMA and L319.

In one embodiment, the lipid nanoparticle formulations described herein may comprise a cationic lipid, a non-cationic lipid, a PEG lipid and a structural lipid. As a non-limiting example, the lipid nanoparticle comprise about 50% of the cationic lipid DLin-KC2-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DMG and about 38.5% of the structural lipid cholesterol. As yet another non-limiting example, the lipid nanoparticle comprise about 55% of the cationic lipid L319, about 10% of the non-cationic lipid DSPC, about 2.5% of the PEG lipid PEG-DMG and about 32.5% of the structural lipid cholesterol.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In one embodiment, the mRNA cancer vaccine composition may comprise the polynucleotide described herein, formulated in a lipid nanoparticle comprising MC3, Cholesterol, DSPC and PEG2000-DMG, the buffer trisodium citrate, sucrose and water for injection. As a non-limiting example, the composition comprises: 2.0 mg/mL of drug substance (e.g., polynucleotides encoding H10N8 influenza virus), 21.8 mg/mL of MC3, 10.1 mg/mL of cholesterol, 5.4 mg/mL of DSPC, 2.7 mg/mL of PEG2000-DMG, 5.16 mg/mL of trisodium citrate, 71 mg/mL of sucrose and about 1.0 mL of water for injection.

The RNA vaccines of the invention can be formulated using one or more liposomes, lipoplexes, or lipid nanoparticles. In one embodiment, pharmaceutical compositions of RNA vaccines include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, the contents of each of which are herein incorporated by reference in its entirety.

In one embodiment, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, WA), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, PA).

In one embodiment, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; all of which are incorporated herein in their entireties). The original manufacture method by Wheeler et al. was a detergent dialysis method, which was later improved by Jeffs et al. and is referred to as the spontaneous vesicle formation method. The liposome formulations are composed of 3 to 4 lipid components in addition to the polynucleotide. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In some embodiments, liposome formulations may comprise from about about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In a preferred embodiment, formulations may comprise a percentage of cholesterol selected from the group consisting of 28.5%, 31.5%, 33.5%, 36.5%, 37.0%, 38.5%, 39.0% and 43.5%. In some embodiments, formulations may comprise from about 5.0% to about 10.0% DSPC and/or from about 7.0% to about 15.0% DSPC.

In one embodiment, pharmaceutical compositions may include liposomes which may be formed to deliver polynucleotides which may encode at least one immunogen (antigen) or any other polypeptide of interest. The RNA vaccine may be encapsulated by the liposome and/or it may be contained in an aqueous core which may then be encapsulated by the liposome (see International Pub. Nos. WO2012031046, WO2012031043, WO2012030901 and WO2012006378 and US Patent Publication No. US20130189351, US20130195969 and US20130202684; the contents of each of which are herein incorporated by reference in their entirety).

In another embodiment, liposomes may be formulated for targeted delivery. As a non-limiting example, the liposome may be formulated for targeted delivery to the liver. The liposome used for targeted delivery may include, but is not limited to, the liposomes described in and methods of making liposomes described in US Patent Publication No. US20130195967, the contents of which are herein incorporated by reference in its entirety.

In another embodiment, the polynucleotide which may encode an immunogen (antigen) may be formulated in a cationic oil-in-water emulsion where the emulsion particle comprises an oil core and a cationic lipid which can interact with the polynucleotide anchoring the molecule to the emulsion particle (see International Pub. No. WO2012006380; herein incorporated by reference in its entirety).

In one embodiment, the RNA vaccines may be formulated in a water-in-oil emulsion comprising a continuous hydrophobic phase in which the hydrophilic phase is dispersed. As a non-limiting example, the emulsion may be made by the methods described in International Publication No. WO201087791, the contents of which are herein incorporated by reference in its entirety.

In another embodiment, the lipid formulation may include at least cationic lipid, a lipid which may enhance transfection and a least one lipid which contains a hydrophilic head group linked to a lipid moiety (International Pub. No. WO2011076807 and U.S. Pub. No. 20110200582; the contents of each of which is herein incorporated by reference in their entirety). In another embodiment, the polynucleotides encoding an immunogen may be formulated in a lipid vesicle which may have crosslinks between functionalized lipid bilayers (see U.S. Pub. No. 20120177724, the contents of which is herein incorporated by reference in its entirety).

In one embodiment, the polylnucleotides may be formulated in a lipsome as described in International Patent Publication No. WO2013086526, the contents of which is herein incorporated by reference in its entirety. The RNA vaccines may be encapsulated in a liposome using reverse pH gradients and/or optimized internal buffer compositions as described in International Patent Publication No. WO2013086526, the contents of which is herein incorporated by reference in its entirety.

In one embodiment, the RNA vaccine pharmaceutical compositions may be formulated in liposomes such as, but not limited to, DiLa2 liposomes (Marina Biotech, Bothell, WA), SMARTICLES® (Marina Biotech, Bothell, WA), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In one embodiment, the cationic lipid may be a low molecular weight cationic lipid such as those described in US Patent Application No. 20130090372, the contents of which are herein incorporated by reference in its entirety.

In one embodiment, the RNA vaccines may be formulated in a lipid vesicle which may have crosslinks between functionalized lipid bilayers.

In one embodiment, the RNA vaccines may be formulated in a liposome comprising a cationic lipid. The liposome may have a molar ratio of nitrogen atoms in the cationic lipid to the phophates in the RNA (N:P ratio) of between 1:1 and 20:1 as described in International Publication No. WO2013006825, herein incorporated by reference in its entirety. In another embodiment, the liposome may have a N:P ratio of greater than 20:1 or less than 1:1.

In one embodiment, the RNA vaccines may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Pub. No. WO2012013326 or US Patent Pub. No. US20130142818; each of which is herein incorporated by reference in its entirety. In another embodiment, the RNA vaccines may be formulated in a lipid-polycation complex which may further include a non-cationic lipid such as, but not limited to, cholesterol or dioleoyl phosphatidylethanolamine (DOPE).

In one embodiment, the RNA vaccines may be formulated in an aminoalcohol lipidoid. Aminoalcohol lipidoids which may be used in the present invention may be prepared by the methods described in U.S. Pat. No. 8,450,298, herein incorporated by reference in its entirety.

The liposome formulation may be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010 28:172-176; herein incorporated by reference in its entirety), the liposome formulation was composed of 57.1% cationic lipid, 7.1% dipalmitoylphosphatidylcholine, 34.3% cholesterol, and 1.4% PEG-c-DMA. As another example, changing the composition of the cationic lipid could more effectively deliver siRNA to various antigen presenting cells (Basha et al. Mol Ther. 2011 19:2186-2200; herein incorporated by reference in its entirety). In some embodiments, liposome formulations may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to mRNA in liposomes may be from about about 5:1 to about 20:1, from about 10:1 to about 25:1, from about 15:1 to about 30:1 and/or at least 30:1.

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain from about 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of PEG-c-DOMG (R-3-[(ω-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In another embodiment the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In one embodiment, the RNA vaccines may be formulated in a lipid nanoparticle such as those described in International Publication No. WO2012170930, the contents of which is herein incorporated by reference in its entirety.

In one embodiment, the RNA vaccine formulation comprising the polynucleotide is a nanoparticle which may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In another aspect, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in US Patent Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In one embodiment, the lipid nanoparticle formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of about 20-60% cationic lipid:5-25% neutral lipid:25-55% sterol; 0.5-15% PEG-lipid.

In one embodiment, the formulation includes from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In one embodiment, the formulation includes from about 0.5% to about 15% on a molar basis of the neutral lipid e.g., from about 3 to about 12%, from about 5 to about 10% or about 15%, about 10%, or about 7.5% on a molar basis. Exemplary neutral lipids include, but are not limited to, DSPC, POPC, DPPC, DOPE and SM. In one embodiment, the formulation includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In one embodiment, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis. In one embodiment, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Exemplary PEG-modified lipids include, but are not limited to, PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in its entirety)

In one embodiment, the formulations of the inventions include 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In one embodiment, the formulations of the inventions include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In one embodiment, the formulations of the inventions include 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In one embodiment, the formulations of the inventions include about 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.5% of the neutral lipid, about 31% of the sterol, and about 1.5% of the PEG or PEG-modified lipid on a molar basis.

In one embodiment, the formulations of the inventions include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 38.5% of the sterol, and about 1.5% of the PEG or PEG-modified lipid on a molar basis.

In one embodiment, the formulations of the inventions include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 35% of the sterol, about 4.5% or about 5% of the PEG or PEG-modified lipid, and about 0.5% of the targeting lipid on a molar basis.

In one embodiment, the formulations of the inventions include about 40% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 15% of the neutral lipid, about 40% of the sterol, and about 5% of the PEG or PEG-modified lipid on a molar basis.

In one embodiment, the formulations of the inventions include about 57.2% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.1% of the neutral lipid, about 34.3% of the sterol, and about 1.4% of the PEG or PEG-modified lipid on a molar basis.

In one embodiment, the formulations of the inventions include about 57.5% of a cationic lipid selected from the PEG lipid is PEG-cDMA (PEG-cDMA is further discussed in Reyes et al. (J. Controlled Release, 107, 276-287 (2005), the contents of which are herein incorporated by reference in its entirety), about 7.5% of the neutral lipid, about 31.5% of the sterol, and about 3.5% of the PEG or PEG-modified lipid on a molar basis.

In preferred embodiments, lipid nanoparticle formulation consists essentially of a lipid mixture in molar ratios of about 20-70% cationic lipid:5-45% neutral lipid:20-55% cholesterol: 0.5-15% PEG-modified lipid; more preferably in a molar ratio of about 20-60% cationic lipid:5-25% neutral lipid:25-55% cholesterol: 0.5-15% PEG-modified lipid.

In particular embodiments, the molar lipid ratio is approximately 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-cDMA).

Exemplary lipid nanoparticle compositions and methods of making same are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578 (the contents of each of which are incorporated herein by reference in their entirety).

In one embodiment, the lipid nanoparticle formulations described herein may comprise a cationic lipid, a PEG lipid and a structural lipid and optionally comprise a non-cationic lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In one embodiment, the cationic lipid may be any cationic lipid described herein such as, but not limited to, DLin-KC2-DMA, DLin-MC3-DMA and L319.

In one embodiment, the lipid nanoparticle formulations described herein may be 4 component lipid nanoparticles. The lipid nanoparticle may comprise a cationic lipid, a non-cationic lipid, a PEG lipid and a structural lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In one embodiment, the cationic lipid may be any cationic lipid described herein such as, but not limited to, DLin-KC2-DMA, DLin-MC3-DMA and L319.

In one embodiment, the lipid nanoparticle formulations described herein may comprise a cationic lipid, a non-cationic lipid, a PEG lipid and a structural lipid. As a non-limiting example, the lipid nanoparticle comprise about 50% of the cationic lipid DLin-KC2-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DMG and about 38.5% of the structural lipid cholesterol. As yet another non-limiting example, the lipid nanoparticle comprise about 55% of the cationic lipid L319, about 10% of the non-cationic lipid DSPC, about 2.5% of the PEG lipid PEG-DMG and about 32.5% of the structural lipid cholesterol.

In one embodiment, the cationic lipid may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865, WO2008103276, WO2013086373 and WO2013086354, U.S. Pat. Nos. 7,893,302, 7,404,969, 8,283,333, and 8,466,122 and US Patent Publication No. US20100036115, US20120202871, US20130064894, US20130129785, US20130150625, US20130178541 and US20130225836; the contents of each of which are herein incorporated by reference in their entirety. In another embodiment, the cationic lipid may be selected from, but not limited to, formula A described in International Publication Nos. WO2012040184, WO2011153120, WO2011149733, WO2011090965, WO2011043913, WO2011022460, WO2012061259, WO2012054365, WO2012044638 and WO2013116126 or US Patent Publication No. US20130178541 and US20130225836; the contents of each of which is herein incorporated by reference in their entirety. In yet another embodiment, the cationic lipid may be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula I-VI of US Patent Publication No. US20100036115, formula I of US Patent Publication No US20130123338; each of which is herein incorporated by reference in their entirety. As a non-limiting example, the cationic lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N5N-dimethylpentacosa-16, 19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21 Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21 Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine,N,N-dimethyl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)—N,N-dimethylnonacosa-11,20,2-trien-10-amine or a pharmaceutically acceptable salt or stereoisomer thereof.

In one embodiment, the lipid may be a cleavable lipid such as those described in International Publication No. WO2012170889, herein incorporated by reference in its entirety.

In another embodiment, the lipid may be a cationic lipid such as, but not limited to, Formula (I) of U.S. Patent Application No. US20130064894, the contents of which are herein incorporated by reference in its entirety.

In one embodiment, the cationic lipid may be synthesized by methods known in the art and/or as described in International Publication Nos. WO2012040184, WO2011153120, WO2011149733, WO2011090965, WO2011043913, WO2011022460, WO2012061259, WO2012054365, WO2012044638, WO2010080724, WO201021865, WO2013086373 and WO2013086354; the contents of each of which are herein incorporated by reference in their entirety.

In another embodiment, the cationic lipid may be a trialkyl cationic lipid. Non-limiting examples of trialkyl cationic lipids and methods of making and using the trialkyl cationic lipids are described in International Patent Publication No. WO2013126803, the contents of which are herein incorporated by reference in its entirety.

In one embodiment, the LNP formulations of the RNA vaccines may contain PEG-c-DOMG at 3% lipid molar ratio. In another embodiment, the LNP formulations of the RNA vaccines may contain PEG-c-DOMG at 1.5% lipid molar ratio.

In one embodiment, the pharmaceutical compositions of the RNA vaccines may include at least one of the PEGylated lipids described in International Publication No. WO2012099755, the contents of which is herein incorporated by reference in its entirety.

In one embodiment, the LNP formulation may contain PEG-DMG 2000 (1,2-dimyristoyl-sn-glycero-3-phophoethanolamine-N-[methoxy(polyethylene glycol)-2000). In one embodiment, the LNP formulation may contain PEG-DMG 2000, a cationic lipid known in the art and at least one other component. In another embodiment, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48 (see e.g., Geall et al., Nonviral delivery of self-amplifying RNA vaccines, PNAS 2012; PMID: 22908294; herein incorporated by reference in its entirety).

In one embodiment, the LNP formulation may be formulated by the methods described in International Publication Nos. WO2011127255 or WO2008103276, the contents of each of which is herein incorporated by reference in their entirety. As a non-limiting example, the RNA vaccines described herein may be encapsulated in LNP formulations as described in WO2011127255 and/or WO2008103276; each of which is herein incorporated by reference in their entirety.

In one embodiment, the RNA vaccines described herein may be formulated in a nanoparticle to be delivered by a parenteral route as described in U.S. Pub. No. US20120207845; the contents of which are herein incorporated by reference in its entirety.

In one embodiment, the RNA vaccines may be formulated in a lipid nanoparticle made by the methods described in US Patent Publication No US20130156845 or International Publication No WO2013093648 or WO2012024526, each of which is herein incorporated by reference in its entirety.

The lipid nanoparticles described herein may be made in a sterile environment by the system and/or methods described in US Patent Publication No. US20130164400, herein incorporated by reference in its entirety.

In one embodiment, the LNP formulation may be formulated in a nanoparticle such as a nucleic acid-lipid particle described in U.S. Pat. No. 8,492,359, the contents of which are herein incorporated by reference in its entirety. As a non-limiting example, the lipid particle may comprise one or more active agents or therapeutic agents; one or more cationic lipids comprising from about 50 mol % to about 85 mol % of the total lipid present in the particle; one or more non-cationic lipids comprising from about 13 mol % to about 49.5 mol % of the total lipid present in the particle; and one or more conjugated lipids that inhibit aggregation of particles comprising from about 0.5 mol % to about 2 mol % of the total lipid present in the particle. The nucleic acid in the nanoparticle may be the polynucleotides described herein and/or are known in the art.

In one embodiment, the LNP formulation may be formulated by the methods described in International Publication Nos. WO2011127255 or WO2008103276, the contents of each of which are herein incorporated by reference in their entirety. As a non-limiting example, modified RNA described herein may be encapsulated in LNP formulations as described in WO2011127255 and/or WO2008103276; the contents of each of which are herein incorporated by reference in their entirety.

In one embodiment, LNP formulations described herein may comprise a polycationic composition. As a non-limiting example, the polycationic composition may be selected from formula 1-60 of US Patent Publication No. US20050222064; the content of which is herein incorporated by reference in its entirety. In another embodiment, the LNP formulations comprising a polycationic composition may be used for the delivery of the modified RNA described herein in vivo and/or in vitro.

In one embodiment, the LNP formulations described herein may additionally comprise a permeability enhancer molecule. Non-limiting permeability enhancer molecules are described in US Patent Publication No. US20050222064; the content of which is herein incorporated by reference in its entirety.

In one embodiment, the RNA vaccine pharmaceutical compositions may be formulated in liposomes such as, but not limited to, DiLa2 liposomes (Marina Biotech, Bothell, WA), SMARTICLES® (Marina Biotech, Bothell, WA), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In one embodiment, the RNA vaccines may be formulated in a lyophilized gel-phase liposomal composition as described in US Publication No. US2012060293, herein incorporated by reference in its entirety.

In some embodiments the RNA vaccine may be associated with a cationic or polycationic compounds, including protamine, nucleoline, spermine or spermidine, or other cationic peptides or proteins, such as poly-L-lysine (PLL), polyarginine, basic polypeptides, cell penetrating peptides (CPPs), including HIV-binding peptides, HIV-1 Tat (HIV), Tat-derived peptides, Penetratin, VP22 derived or analog peptides, Pestivirus Erns, HSV, VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides (particularly from Drosophila antennapedia), pAntp, plsl, FGF, Lactoferrin, Transportan, Buforin-2, Bac715-24, SynB, SynB(1), pVEC, hCT-derived peptides, SAP, histones, cationic polysaccharides, for example chitosan, polybrene, cationic polymers, e.g. polyethyleneimine (PEI), cationic lipids, e.g. DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS: Dioctadecylamidoglicylspermin, DIMRI: Dimyristooxypropyl dimethyl hydroxyethyl ammonium bromide, DOTAP: dioleoyloxy-3-(trimethylammonio)propane, DC-6-14: O,O-ditetradecanoyl-N-.alpha.-trimethylammonioacetyl)diethanolamine chloride, CLIP 1: rac-[(2,3-dioctadecyloxypropyl)(2-hydroxyethyl)]-dimethylammonium chloride, CLIP6: rac-[2(2,3-dihexadecyloxypropyloxymethyloxy)ethyl]-trimethylammonium, CLIP9: rac-[2(2,3-dihexadecyloxypropyloxysuccinyloxy)ethyl]-trimethylammo-nium, oligofectamine, or cationic or polycationic polymers, e.g. modified polyaminoacids, such as beta-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., modified polybetaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole); etc.

In other embodiments the RNA vaccine is not associated with a cationic or polycationic compounds.

The nanoparticle formulations may comprise a phosphate conjugate. The phosphate conjugate may increase in vivo circulation times and/or increase the targeted delivery of the nanoparticle. Phosphate conjugates for use with the present invention may be made by the methods described in International Application No. WO2013033438 or US Patent Publication No. US20130196948, the contents of each of which are herein incorporated by reference in its entirety. As a non-limiting example, the phosphate conjugates may include a compound of any one of the formulas described in International Application No. WO2013033438, herein incorporated by reference in its entirety.

The nanoparticle formulation may comprise a polymer conjugate. The polymer conjugate may be a water soluble conjugate. The polymer conjugate may have a structure as described in U.S. Patent Application No. 20130059360, the contents of which are herein incorporated by reference in its entirety. In one aspect, polymer conjugates with the polynucleotides of the present invention may be made using the methods and/or segmented polymeric reagents described in U.S. Patent Application No. 20130072709, herein incorporated by reference in its entirety. In another aspect, the polymer conjugate may have pendant side groups comprising ring moieties such as, but not limited to, the polymer conjugates described in US Patent Publication No. US20130196948, the contents of which is herein incorporated by reference in its entirety.

The nanoparticle formulations may comprise a conjugate to enhance the delivery of nanoparticles of the present invention in a subject. Further, the conjugate may inhibit phagocytic clearance of the nanoparticles in a subject. In one aspect, the conjugate may be a “self” peptide designed from the human membrane protein CD47 (e.g., the “self” particles described by Rodriguez et al (Science 2013 339, 971-975), herein incorporated by reference in its entirety). As shown by Rodriguez et al. the self peptides delayed macrophage-mediated clearance of nanoparticles which enhanced delivery of the nanoparticles. In another aspect, the conjugate may be the membrane protein CD47 (e.g., see Rodriguez et al. Science 2013 339, 971-975, herein incorporated by reference in its entirety). Rodriguez et al. showed that, similarly to “self” peptides, CD47 can increase the circulating particle ratio in a subject as compared to scrambled peptides and PEG coated nanoparticles.

In one embodiment, the RNA vaccines of the present invention are formulated in nanoparticles which comprise a conjugate to enhance the delivery of the nanoparticles of the present invention in a subject. The conjugate may be the CD47 membrane or the conjugate may be derived from the CD47 membrane protein, such as the “self” peptide described previously. In another aspect the nanoparticle may comprise PEG and a conjugate of CD47 or a derivative thereof. In yet another aspect, the nanoparticle may comprise both the “self” peptide described above and the membrane protein CD47.

In another aspect, a “self” peptide and/or CD47 protein may be conjugated to a virus-like particle or pseudovirion, as described herein for delivery of the RNA vaccines of the present invention.

In another embodiment, RNA vaccine pharmaceutical compositions comprising the polynucleotides of the present invention and a conjugate which may have a degradable linkage. Non-limiting examples of conjugates include an aromatic moiety comprising an ionizable hydrogen atom, a spacer moiety, and a water-soluble polymer. As a non-limiting example, pharmaceutical compositions comprising a conjugate with a degradable linkage and methods for delivering such pharmaceutical compositions are described in US Patent Publication No. US20130184443, the contents of which are herein incorporated by reference in its entirety.

The nanoparticle formulations may be a carbohydrate nanoparticle comprising a carbohydrate carrier and a RNA vaccine. As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; the contents of which are herein incorporated by reference in its entirety).

Nanoparticle formulations of the present invention may be coated with a surfactant or polymer in order to improve the delivery of the particle. In one embodiment, the nanoparticle may be coated with a hydrophilic coating such as, but not limited to, PEG coatings and/or coatings that have a neutral surface charge. The hydrophilic coatings may help to deliver nanoparticles with larger payloads such as, but not limited to, RNA vaccines within the central nervous system. As a non-limiting example nanoparticles comprising a hydrophilic coating and methods of making such nanoparticles are described in US Patent Publication No. US20130183244, the contents of which are herein incorporated by reference in its entirety.

In one embodiment, the lipid nanoparticles of the present invention may be hydrophilic polymer particles. Non-limiting examples of hydrophilic polymer particles and methods of making hydrophilic polymer particles are described in US Patent Publication No. US20130210991, the contents of which are herein incorporated by reference in its entirety.

In another embodiment, the lipid nanoparticles of the present invention may be hydrophobic polymer particles.

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In one embodiment, the internal ester linkage may be located on either side of the saturated carbon.

In one embodiment, an immune response may be elicited by delivering a lipid nanoparticle which may include a nanospecies, a polymer and an immunogen. (U.S. Publication No. 20120189700 and International Publication No. WO2012099805; each of which is herein incorporated by reference in their entirety). The polymer may encapsulate the nanospecies or partially encapsulate the nanospecies. The immunogen may be a recombinant protein, a modified RNA and/or a polynucleotide described herein. In one embodiment, the lipid nanoparticle may be formulated for use in a vaccine such as, but not limited to, against a pathogen.

Lipid nanoparticles may be engineered to alter the surface properties of particles so the lipid nanoparticles may penetrate the mucosal barrier. Mucus is located on mucosal tissue such as, but not limted to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosla tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104(5):1482-487; Lai et al. Adv Drug Deliv Rev. 2009 61(2): 158-171; each of which is herein incorporated by reference in their entirety). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Patent Publication No. WO2013110028, the contents of each of which are herein incorporated by reference in its entirety.

The lipid nanoparticle engineered to penetrate mucus may comprise a polymeric material (i.e. a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material may be biodegradable and/or biocompatible. Non-limiting examples of biocompatible polymers are described in International Patent Publication No. WO2013116804, the contents of which are herein incorporated by reference in its entirety. The polymeric material may additionally be irradiated. As a non-limiting example, the polymeric material may be gamma irradiated (See e.g., International App. No. WO201282165, herein incorporated by reference in its entirety). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), PEG-PLGA-PEG and trimethylene carbonate, polyvinylpyrrolidone. The lipid nanoparticle may be coated or associated with a co-polymer such as, but not limited to, a block co-polymer (such as a branched polyether-polyamide block copolymer described in International Publication No. WO2013012476, herein incorporated by reference in its entirety), and (poly(ethylene glycol))-(poly(propylene oxide))-(poly(ethylene glycol)) triblock copolymer (see e.g., US Publication 20120121718 and US Publication 20100003337 and U.S. Pat. No. 8,263,665; each of which is herein incorporated by reference in their entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate 25 human mucus (Yang et al. Angew. Chem. Int. Ed. 2011 50:2597-2600; the contents of which are herein incorporated by reference in its entirety). A non-limiting scalable method to produce nanoparticles which can penetrate human mucus is described by Xu et al. (See e.g., J Control Release 2013, 170(2):279-86; the contents of which are herein incorporated by reference in its entirety).

The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

The lipid nanoparticle engineered to penetrate mucus may include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 dornase alfa, neltenexine, erdosteine) and various DNases including rhDNase. The surface altering agent may be embedded or enmeshed in the particle's surface or disposed (e.g., by coating, adsorption, covalent linkage, or other process) on the surface of the lipid nanoparticle. (see e.g., US Publication 20100215580 and US Publication 20080166414 and US20130164343; the contents of each of which is herein incorporated by reference in their entirety).

In one embodiment, the mucus penetrating lipid nanoparticles may comprise at least one polynucleotide described herein. The polynucleotide may be encapsulated in the lipid nanoparticle and/or disposed on the surface of the paricle. The polynucleotide may be covalently coupled to the lipid nanoparticle. Formulations of mucus penetrating lipid nanoparticles may comprise a plurality of nanoparticles. Further, the formulations may contain particles which may interact with the mucus and alter the structural and/or adhesive properties of the surrounding mucus to decrease mucoadhesion which may increase the delivery of the mucus penetrating lipid nanoparticles to the mucosal tissue.

In another embodiment, the mucus penetrating lipid nanoparticles may be a hypotonic formulation comprising a mucosal penetration enhancing coating. The formulation may be hypotonice for the epithelium to which it is being delivered. Non-limiting examples of hypotonic formulations may be found in International Patent Publication No. WO2013110028, the contents of which are herein incorporated by reference in its entirety.

In one embodiment, in order to enhance the delivery through the mucosal barrier the RNA vaccine formulation may comprise or be a hypotonic solution. Hypotonic solutions were found to increase the rate at which mucoinert particles such as, but not limited to, mucus-penetrating particles, were able to reach the vaginal epithelial surface (See e.g., Ensign et al. Biomaterials 2013 34(28):6922-9; the contents of which is herein incorporated by reference in its entirety).

In one embodiment, the RNA vaccine is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, MA), and polyethylenimine (PEI) or protamine-based targeted and non-targeted delivery of nucleic acids acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; all of which are incorporated herein by reference in its entirety).

In one embodiment such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133; all of which are incorporated herein by reference in its entirety). One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18:1357-1364; herein incorporated by reference in its entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133; all of which are incorporated herein by reference in its entirety).

In one embodiment, the RNA vaccine is formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In a further embodiment, the lipid nanoparticle may be a self-assembly lipid-polymer nanoparticle (see Zhang et al., ACS Nano, 2008, 2 (8), pp 1696-1702; the contents of which are herein incorporated by reference in its entirety). As a non-limiting example, the SLN may be the SLN described in International Patent Publication No. WO2013105101, the contents of which are herein incorporated by reference in its entirety. As another non-limiting example, the SLN may be made by the methods or processes described in International Patent Publication No. WO2013105101, the contents of which are herein incorporated by reference in its entirety.

Liposomes, lipoplexes, or lipid nanoparticles may be used to improve the efficacy of polynucleotides directed protein production as these formulations may be able to increase cell transfection by the RNA vaccine; and/or increase the translation of encoded protein. One such example involves the use of lipid encapsulation to enable the effective systemic delivery of polyplex plasmid DNA (Heyes et al., Mol Ther. 2007 15:713-720; herein incorporated by reference in its entirety). The liposomes, lipoplexes, or lipid nanoparticles may also be used to increase the stability of the polynucleotide.

In one embodiment, the RNA vaccines of the present invention can be formulated for controlled release and/or targeted delivery. As used herein, “controlled release” refers to a pharmaceutical composition or compound release profile that conforms to a particular pattern of release to effect a therapeutic outcome. In one embodiment, the RRNA vaccines may be encapsulated into a delivery agent described herein and/or known in the art for controlled release and/or targeted delivery. As used herein, the term “encapsulate” means to enclose, surround or encase. As it relates to the formulation of the compounds of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the invention may be enclosed, surrounded or encased within the delivery agent. “Partially encapsulated” means that less than 10, 10, 20, 30, 40 50 or less of the pharmaceutical composition or compound of the invention may be enclosed, surrounded or encased within the delivery agent. Advantageously, encapsulation may be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the delivery agent.

In one embodiment, the controlled release formulation may include, but is not limited to, tri-block co-polymers. As a non-limiting example, the formulation may include two different types of tri-block co-polymers (International Pub. No. WO2012131104 and WO2012131106; the contents of each of which is herein incorporated by reference in its entirety).

In another embodiment, the RNA vaccines may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, FL), HYLENEX® (Halozyme Therapeutics, San Diego CA), surgical sealants such as fibrinogen polymers (Ethicon Inc. Cornelia, GA), TISSELL® (Baxter International, Inc Deerfield, IL), PEG-based sealants, and COSEAL® (Baxter International, Inc Deerfield, IL).

In another embodiment, the lipid nanoparticle may be encapsulated into any polymer known in the art which may form a gel when injected into a subject. As another non-limiting example, the lipid nanoparticle may be encapsulated into a polymer matrix which may be biodegradable.

In one embodiment, the the RNA vaccine formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In one embodiment, the RNA vaccine controlled release and/or targeted delivery formulation may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In one embodiment, the RNA vaccine controlled release and/or targeted delivery formulation comprising at least one polynucleotide may comprise at least one PEG and/or PEG related polymer derivatives as described in U.S. Pat. No. 8,404,222, herein incorporated by reference in its entirety.

In another embodiment, the RNA vaccine controlled release delivery formulation comprising at least one polynucleotide may be the controlled release polymer system described in US20130130348, herein incorporated by reference in its entirety.

In one embodiment, the the RNA vaccines of the present invention may be encapsulated in a therapeutic nanoparticle, referred to herein as “therapeutic nanoparticle RRNA vaccines.” Therapeutic nanoparticles may be formulated by methods described herein and known in the art such as, but not limited to, International Pub Nos. WO2010005740, WO2010030763, WO2010005721, WO2010005723, WO2012054923, US Pub. Nos. US20110262491, US20100104645, US20100087337, US20100068285, US20110274759, US20100068286, US20120288541, US20130123351 and US20130230567 and U.S. Pat. Nos. 8,206,747, 8,293,276, 8,318,208 and 8,318,211; the contents of each of which are herein incorporated by reference in their entirety. In another embodiment, therapeutic polymer nanoparticles may be identified by the methods described in US Pub No. US20120140790, the contents of which is herein incorporated by reference in its entirety.

In one embodiment, the therapeutic nanoparticle RNA vaccine may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the the polynucleotides of the present invention (see International Pub No. 2010075072 and US Pub No. US20100216804, US20110217377 and US20120201859, each of which is herein incorporated by reference in their entirety). In another non-limiting example, the sustained release formulation may comprise agents which permit persistent bioavailability such as, but not limited to, crystals, macromolecular gels and/or particulate suspensions (see US Patent Publication No US20130150295, the contents of which is herein incorporated by reference in its entirety).

In one embodiment, the therapeutic nanoparticle RNA vaccines may be formulated to be target specific. As a non-limiting example, the thereapeutic nanoparticles may include a corticosteroid (see International Pub. No. WO2011084518; herein incorporated by reference in its entirety). As a non-limiting example, the therapeutic nanoparticles may be formulated in nanoparticles described in International Pub No. WO2008121949, WO2010005726, WO2010005725, WO2011084521 and US Pub No. US20100069426, US20120004293 and US20100104655, each of which is herein incorporated by reference in their entirety.

In one embodiment, the nanoparticles of the present invention may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In one embodiment, the therapeutic nanoparticle comprises a diblock copolymer. In one embodiment, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof. In another embodiment, the diblock copolymer may comprise the diblock copolymers described in European Patent Publication No. the contents of which are herein incorporated by reference in its entirety. In yet another embodiment, the diblock copolymer may be a high-X diblock copolymer such as those described in International Patent Publication No. WO2013120052, the contents of which are herein incorporated by reference in its entirety.

As a non-limiting example the therapeutic nanoparticle comprises a PLGA-PEG block copolymer (see US Pub. No. US20120004293 and U.S. Pat. No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968 and International Publication No. WO2012166923, the contents of each of which are herein incorporated by reference in its entirety). In yet another non-limiting example, the therapeutic nanoparticle is a stealth nanoparticle or a target-specific stealth nanoparticle as described in US Patent Publication No. US20130172406, the contents of which are herein incorporated by reference in its entirety.

In one embodiment, the therapeutic nanoparticle may comprise a multiblock copolymer (See e.g., U.S. Pat. Nos. 8,263,665 and 8,287,910 and US Patent Pub. No. US20130195987; the contents of each of which are herein incorporated by reference in its entirety).

In yet another non-limiting example, the lipid nanoparticle comprises the block copolymer PEG-PLGA-PEG (see e.g., the thermosensitive hydrogel (PEG-PLGA-PEG) was used as a TGF-beta1 gene delivery vehicle in Lee et al. Thermosensitive Hydrogel as a Tgf-β1 Gene Delivery Vehicle Enhances Diabetic Wound Healing. Pharmaceutical Research, 2003 20(12): 1995-2000; as a controlled gene delivery system in Li et al. Controlled Gene Delivery System Based on Thermosensitive Biodegradable Hydrogel. Pharmaceutical Research 2003 20(6):884-888; and Chang et al., Non-ionic amphiphilic biodegradable PEG-PLGA-PEG copolymer enhances gene delivery efficiency in rat skeletal muscle. J Controlled Release. 2007 118:245-253; each of which is herein incorporated by reference in its entirety). The RNA vaccines of the present invention may be formulated in lipid nanoparticles comprising the PEG-PLGA-PEG block copolymer.

In one embodiment, the therapeutic nanoparticle may comprise a multiblock copolymer (See e.g., U.S. Pat. Nos. 8,263,665 and 8,287,910 and US Patent Pub. No. US20130195987; the contents of each of which are herein incorporated by reference in its entirety).

In one embodiment, the block copolymers described herein may be included in a polyion complex comprising a non-polymeric micelle and the block copolymer. (See e.g., U.S. Pub. No. 20120076836; herein incorporated by reference in its entirety).

In one embodiment, the therapeutic nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the therapeutic nanoparticles may comprise at least one poly(vinyl ester) polymer. The poly(vinyl ester) polymer may be a copolymer such as a random copolymer. As a non-limiting example, the random copolymer may have a structure such as those described in International Application No. WO2013032829 or US Patent Publication No US20130121954, the contents of which are herein incorporated by reference in its entirety. In one aspect, the poly(vinyl ester) polymers may be conjugated to the polynucleotides described herein. In another aspect, the poly(vinyl ester) polymer which may be used in the present invention may be those described in, herein incorporated by reference in its entirety.

In one embodiment, the therapeutic nanoparticle may comprise at least one diblock copolymer. The diblock copolymer may be, but it not limited to, a poly(lactic) acid-poly(ethylene)glycol copolymer (see e.g., International Patent Publication No. WO2013044219; herein incorporated by reference in its entirety). As a non-limiting example, the therapeutic nanoparticle may be used to treat cancer (see International publication No. WO2013044219; herein incorporated by reference in its entirety).

In one embodiment, the therapeutic nanoparticles may comprise at least one cationic polymer described herein and/or known in the art.

In one embodiment, the therapeutic nanoparticles may comprise at least one amine-containing polymer such as, but not limited to polylysine, polyethylene imine, poly(amidoamine) dendrimers, poly(beta-amino esters) (See e.g., U.S. Pat. No. 8,287,849; herein incorporated by reference in its entirety) and combinations thereof.

In another embodiment, the nanoparticles described herein may comprise an amine cationic lipid such as those described in International Patent Application No. WO2013059496, the contents of which are herein incorporated by reference in its entirety. In one aspect the cationic lipids may have an amino-amine or an amino-amide moiety.

In one embodiment, the therapeutic nanoparticles may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In another embodiment, the therapeutic nanoparticle may include a conjugation of at least one targeting ligand. The targeting ligand may be any ligand known in the art such as, but not limited to, a monoclonal antibody. (Kirpotin et al, Cancer Res. 2006 66:6732-6740; herein incorporated by reference in its entirety).

In one embodiment, the therapeutic nanoparticle may be formulated in an aqueous solution which may be used to target cancer (see International Pub No. WO2011084513 and US Pub No. US20110294717, each of which is herein incorporated by reference in their entirety).

In one embodiment, the therapeutic nanoparticle RNA vaccines, e.g., therapeutic nanoparticles comprising at least one RNA vaccine may be formulated using the methods described by Podobinski et al in U.S. Pat. No. 8,404,799, the contents of which are herein incorporated by reference in its entirety.

In one embodiment, the RNA vaccines may be encapsulated in, linked to and/or associated with synthetic nanocarriers. Synthetic nanocarriers include, but are not limited to, those described in International Pub. Nos. WO2010005740, WO2010030763, WO201213501, WO2012149252, WO2012149255, WO2012149259, WO2012149265, WO2012149268, WO2012149282, WO2012149301, WO2012149393, WO2012149405, WO2012149411, WO2012149454 and WO2013019669, and US Pub. Nos. US20110262491, US20100104645, US20100087337 and US20120244222, each of which is herein incorporated by reference in their entirety. The synthetic nanocarriers may be formulated using methods known in the art and/or described herein. As a non-limiting example, the synthetic nanocarriers may be formulated by the methods described in International Pub Nos. WO2010005740, WO2010030763 and WO201213501 and US Pub. Nos. US20110262491, US20100104645, US20100087337 and US2012024422, each of which is herein incorporated by reference in their entirety. In another embodiment, the synthetic nanocarrier formulations may be lyophilized by methods described in International Pub. No. WO2011072218 and U.S. Pat. No. 8,211,473; the content of each of which is herein incorporated by reference in their entirety. In yet another embodiment, formulations of the present invention, including, but not limited to, synthetic nanocarriers, may be lyophilized or reconstituted by the methods described in US Patent Publication No. US20130230568, the contents of which are herein incorporated by reference in its entirety.

In one embodiment, the synthetic nanocarriers may contain reactive groups to release the polynucleotides described herein (see International Pub. No. WO20120952552 and US Pub No. US20120171229, each of which is herein incorporated by reference in their entirety).

In one embodiment, the synthetic nanocarriers may contain an immunostimulatory agent to enhance the immune response from delivery of the synthetic nanocarrier. As a non-limiting example, the synthetic nanocarrier may comprise a Th1 immunostimulatory agent which may enhance a Th1-based response of the immune system (see International Pub No. WO2010123569 and US Pub. No. US20110223201, each of which is herein incorporated by reference in its entirety).

In one embodiment, the synthetic nanocarriers may be formulated for targeted release. In one embodiment, the synthetic nanocarrier is formulated to release the polynucleotides at a specified pH and/or after a desired time interval. As a non-limiting example, the synthetic nanoparticle may be formulated to release the RNA vaccines after 24 hours and/or at a pH of 4.5 (see International Pub. Nos. WO2010138193 and WO2010138194 and US Pub Nos. US20110020388 and US20110027217, each of which is herein incorporated by reference in their entireties).

In one embodiment, the synthetic nanocarriers may be formulated for controlled and/or sustained release of the polynucleotides described herein. As a non-limiting example, the synthetic nanocarriers for sustained release may be formulated by methods known in the art, described herein and/or as described in International Pub No. WO2010138192 and US Pub No. 20100303850, each of which is herein incorporated by reference in their entirety.

In one embodiment, the RNA vaccine may be formulated for controlled and/or sustained release wherein the formulation comprises at least one polymer that is a crystalline side chain (CYSC) polymer. CYSC polymers are described in U.S. Pat. No. 8,399,007, herein incorporated by reference in its entirety.

In one embodiment, the synthetic nanocarrier may be formulated for use as a vaccine. In one embodiment, the synthetic nanocarrier may encapsulate at least one polynucleotide which encode at least one antigen. As a non-limiting example, the synthetic nanocarrier may include at least one antigen and an excipient for a vaccine dosage form (see International Pub No. WO2011150264 and US Pub No. US20110293723, each of which is herein incorporated by reference in their entirety). As another non-limiting example, a vaccine dosage form may include at least two synthetic nanocarriers with the same or different antigens and an excipient (see International Pub No. WO2011150249 and US Pub No. US20110293701, each of which is herein incorporated by reference in their entirety). The vaccine dosage form may be selected by methods described herein, known in the art and/or described in International Pub No. WO2011150258 and US Pub No. US20120027806, each of which is herein incorporated by reference in their entirety).

In one embodiment, the synthetic nanocarrier may comprise at least one polynucleotide which encodes at least one adjuvant. As non-limiting example, the adjuvant may comprise dimethyldioctadecylammonium-bromide, dimethyldioctadecylammonium-chloride, dimethyldioctadecylammonium-phosphate or dimethyldioctadecylammonium-acetate (DDA) and an apolar fraction or part of said apolar fraction of a total lipid extract of a mycobacterium (See e.g, U.S. Pat. No. 8,241,610; herein incorporated by reference in its entirety). In another embodiment, the synthetic nanocarrier may comprise at least one polynucleotide and an adjuvant. As a non-limiting example, the synthetic nanocarrier comprising and adjuvant may be formulated by the methods described in International Pub No. WO2011150240 and US Pub No. US20110293700, each of which is herein incorporated by reference in its entirety.

In one embodiment, the synthetic nanocarrier may encapsulate at least one polynucleotide which encodes a peptide, fragment or region from a virus. As a non-limiting example, the synthetic nanocarrier may include, but is not limited to, the nanocarriers described in International Pub No. WO2012024621, WO201202629, WO2012024632 and US Pub No. US20120064110, US20120058153 and US20120058154, each of which is herein incorporated by reference in their entirety.

In one embodiment, the synthetic nanocarrier may be coupled to a polynucleotide which may be able to trigger a humoral and/or cytotoxic T lymphocyte (CTL) response (See e.g., International Publication No. WO2013019669, herein incorporated by reference in its entirety).

In one embodiment, the RNA vaccine may be encapsulated in, linked to and/or associated with zwitterionic lipids. Non-limiting examples of zwitterionic lipids and methods of using zwitterionic lipids are described in US Patent Publication No. US20130216607, the contents of which are herein incorporated by reference in its entirety. In one aspect, the zwitterionic lipids may be used in the liposomes and lipid nanoparticles described herein.

In one embodiment, the RNA vaccine may be formulated in colloid nanocarriers as described in US Patent Publication No. US20130197100, the contents of which are herein incorporated by reference in its entirety.

In one embodiment, the nanoparticle may be optimized for oral administration. The nanoparticle may comprise at least one cationic biopolymer such as, but not limited to, chitosan or a derivative thereof. As a non-limiting example, the nanoparticle may be formulated by the methods described in U.S. Pub. No. 20120282343; herein incorporated by reference in its entirety.

In some embodiments, LNPs comprise the lipid KL52 (an amino-lipid disclosed in U.S. Application Publication No. 2012/0295832 expressly incorporated herein by reference in its entirety). Activity and/or safety (as measured by examining one or more of ALT/AST, white blood cell count and cytokine induction) of LNP administration may be improved by incorporation of such lipids. LNPs comprising KL52 may be administered intravenously and/or in one or more doses. In some embodiments, administration of LNPs comprising KL52 results in equal or improved mRNA and/or protein expression as compared to LNPs comprising MC3.

In some embodiments, RNA vaccine may be delivered using smaller LNPs. Such particles may comprise a diameter from below 0.1 um up to 100 nm such as, but not limited to, less than 0.1 um, less than 1.0 um, less than 5 um, less than 10 um, less than 15 um, less than 20 um, less than 25 um, less than 30 um, less than 35 um, less than 40 um, less than 50 um, less than 55 um, less than 60 um, less than 65 um, less than 70 um, less than 75 um, less than 80 um, less than 85 um, less than 90 um, less than 95 um, less than 100 um, less than 125 um, less than 150 um, less than 175 um, less than 200 um, less than 225 um, less than 250 um, less than 275 um, less than 300 um, less than 325 um, less than 350 um, less than 375 um, less than 400 um, less than 425 um, less than 450 um, less than 475 um, less than 500 um, less than 525 um, less than 550 um, less than 575 um, less than 600 um, less than 625 um, less than 650 um, less than 675 um, less than 700 um, less than 725 um, less than 750 um, less than 775 um, less than 800 um, less than 825 um, less than 850 um, less than 875 um, less than 900 um, less than 925 um, less than 950 um, less than 975 um.

In another embodiment, RNA vaccines may be delivered using smaller LNPs which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nM, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm.

In some embodiments, such LNPs are synthesized using methods comprising microfluidic mixers. Exemplary microfluidic mixers may include, but are not limited to a slit interdigitial micromixer including, but not limited to those manufactured by Microinnova (Allerheiligen bei Wildon, Austria) and/or a staggered herringbone micromixer (SHM) (Zhigaltsev, I. V. et al., Bottom-up design and synthesis of limit size lipid nanoparticle systems with aqueous and triglyceride cores using millisecond microfluidic mixing have been published (Langmuir. 2012. 28:3633-40; Belliveau, N. M. et al., Microfluidic synthesis of highly potent limit-size lipid nanoparticles for in vivo delivery of siRNA. Molecular Therapy-Nucleic Acids. 2012. 1:e37; Chen, D. et al., Rapid discovery of potent siRNA-containing lipid nanoparticles enabled by controlled microfluidic formulation. J Am Chem Soc. 2012. 134(16):6948-51; each of which is herein incorporated by reference in its entirety). In some embodiments, methods of LNP generation comprising SHM, further comprise the mixing of at least two input streams wherein mixing occurs by microstructure-induced chaotic advection (MICA). According to this method, fluid streams flow through channels present in a herringbone pattern causing rotational flow and folding the fluids around each other. This method may also comprise a surface for fluid mixing wherein the surface changes orientations during fluid cycling. Methods of generating LNPs using SHM include those disclosed in U.S. Application Publication Nos. 2004/0262223 and 2012/0276209, each of which is expressly incorporated herein by reference in their entirety.

In one embodiment, the RNA vaccine of the present invention may be formulated in lipid nanoparticles created using a micromixer such as, but not limited to, a Slit Interdigital Microstructured Mixer (SIMM-V2) or a Standard Slit Interdigital Micro Mixer (SSIMM) or Caterpillar (CPMM) or Impinging-jet (IJMM) from the Institut fir Mikrotechnik Mainz GmbH, Mainz Germany).

In one embodiment, the RNA vaccines of the present invention may be formulated in lipid nanoparticles created using microfluidic technology (see Whitesides, George M. The Origins and the Future of Microfluidics. Nature, 2006 442: 368-373; and Abraham et al. Chaotic Mixer for Microchannels. Science, 2002 295: 647-651; each of which is herein incorporated by reference in its entirety). As a non-limiting example, controlled microfluidic formulation includes a passive method for mixing streams of steady pressure-driven flows in micro channels at a low Reynolds number (See e.g., Abraham et al. Chaotic Mixer for Microchannels. Science, 2002 295: 647-651; which is herein incorporated by reference in its entirety).

In one embodiment, the RNA vaccines of the present invention may be formulated in lipid nanoparticles created using a micromixer chip such as, but not limited to, those from Harvard Apparatus (Holliston, MA) or Dolomite Microfluidics (Royston, UK). A micromixer chip can be used for rapid mixing of two or more fluid streams with a split and recombine mechanism.

In one embodiment, the RNA vaccines of the invention may be formulated for delivery using the drug encapsulating microspheres described in International Patent Publication No. WO2013063468 or U.S. Pat. No. 8,440,614, each of which is herein incorporated by reference in its entirety. The microspheres may comprise a compound of the formula (I), (II), (III), (IV), (V) or (VI) as described in International Patent Publication No. WO2013063468, the contents of which are herein incorporated by reference in its entirety. In another aspect, the amino acid, peptide, polypeptide, lipids (APPL) are useful in delivering the RNA vaccines of the invention to cells (see International Patent Publication No. WO2013063468, the contents of which is herein incorporated by reference in its entirety).

In one embodiment, the RNA vaccines of the invention may be formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the lipid nanoparticles may have a diameter from about 10 to 500 nm.

In one embodiment, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In one aspect, the lipid nanoparticle may be a limit size lipid nanoparticle described in International Patent Publication No. WO2013059922, the contents of which are herein incorporated by reference in its entirety. The limit size lipid nanoparticle may comprise a lipid bilayer surrounding an aqueous core or a hydrophobic core; where the lipid bilayer may comprise a phospholipid such as, but not limited to, diacylphosphatidylcholine, a diacylphosphatidylethanolamine, a ceramide, a sphingomyelin, a dihydrosphingomyelin, a cephalin, a cerebroside, a C8-C20 fatty acid diacylphophatidylcholine, and 1-palmitoyl-2-oleoyl phosphatidylcholine (POPC). In another aspect the limit size lipid nanoparticle may comprise a polyethylene glycol-lipid such as, but not limited to, DLPE-PEG, DMPE-PEG, DPPC-PEG and DSPE-PEG.

In one embodiment, the RNA vaccines may be delivered, localized and/or concentrated in a specific location using the delivery methods described in International Patent Publication No. WO2013063530, the contents of which are herein incorporated by reference in its entirety. As a non-limiting example, a subject may be administered an empty polymeric particle prior to, simultaneously with or after delivering the RNA vaccines to the subject. The empty polymeric particle undergoes a change in volume once in contact with the subject and becomes lodged, embedded, immobilized or entrapped at a specific location in the subject.

In one embodiment, the RNA vaccines may be formulated in an active substance release system (See e.g., US Patent Publication No. US20130102545, the contents of which is herein incorporated by reference in its entirety). The active substance release system may comprise 1) at least one nanoparticle bonded to an oligonucleotide inhibitor strand which is hybridized with a catalytically active nucleic acid and 2) a compound bonded to at least one substrate molecule bonded to a therapeutically active substance (e.g., polynucleotides described herein), where the therapeutically active substance is released by the cleavage of the substrate molecule by the catalytically active nucleic acid.

In one embodiment, the RNA vaccines may be formulated in a nanoparticle comprising an inner core comprising a non-cellular material and an outer surface comprising a cellular membrane. The cellular membrane may be derived from a cell or a membrane derived from a virus. As a non-limiting example, the nanoparticle may be made by the methods described in International Patent Publication No. WO2013052167, herein incorporated by reference in its entirety. As another non-limiting example, the nanoparticle described in International Patent Publication No. WO2013052167, herein incorporated by reference in its entirety, may be used to deliver the RNA vaccines described herein.

In one embodiment, the RNA vaccines may be formulated in porous nanoparticle-supported lipid bilayers (protocells). Protocells are described in International Patent Publication No. WO2013056132, the contents of which are herein incorporated by reference in its entirety.

In one embodiment, the RNA vaccines described herein may be formulated in polymeric nanoparticles as described in or made by the methods described in U.S. Pat. Nos. 8,420,123 and 8,518,963 and European Patent No. EP2073848B1, the contents of each of which are herein incorporated by reference in their entirety. As a non-limiting example, the polymeric nanoparticle may have a high glass transition temperature such as the nanoparticles described in or nanoparticles made by the methods described in U.S. Pat. No. 8,518,963, the contents of which are herein incorporated by reference in its entirety. As another non-limiting example, the polymer nanoparticle for oral and parenteral formulations may be made by the methods described in European Patent No. EP2073848B1, the contents of which are herein incorporated by reference in its entirety.

In another embodiment, the RNA vaccines described herein may be formulated in nanoparticles used in imaging. The nanoparticles may be liposome nanoparticles such as those described in US Patent Publication No US20130129636, herein incorporated by reference in its entirety. As a non-limiting example, the liposome may comprise gadolinium(III)2-{4,7-bis-carboxymethyl-10-[(N,N-distearylamidomethyl-N′-amido-methyl]-1,4,7,10-tetra-azacyclododec-1-yl}-acetic acid and a neutral, fully saturated phospholipid component (see e.g., US Patent Publication No US20130129636, the contents of which is herein incorporated by reference in its entirety).

In one embodiment, the nanoparticles which may be used in the present invention are formed by the methods described in U.S. Patent Application No. US20130130348, the contents of which is herein incorporated by reference in its entirety.

The nanoparticles of the present invention may further include nutrients such as, but not limited to, those which deficiencies can lead to health hazards from anemia to neural tube defects (see e.g, the nanoparticles described in International Patent Publication No WO2013072929, the contents of which is herein incorporated by reference in its entirety). As a non-limiting example, the nutrient may be iron in the form of ferrous, ferric salts or elemental iron, iodine, folic acid, vitamins or micronutrients.

In one embodiment, the RNA vaccines of the present invention may be formulated in a swellable nanoparticle. The swellable nanoparticle may be, but is not limited to, those described in U.S. Pat. No. 8,440,231, the contents of which is herein incorporated by reference in its entirety. As a non-limiting embodiment, the swellable nanoparticle may be used for delivery of the RNA vaccines of the present invention to the pulmonary system (see e.g., U.S. Pat. No. 8,440,231, the contents of which is herein incorporated by reference in its entirety).

The RNA vaccines of the present invention may be formulated in polyanhydride nanoparticles such as, but not limited to, those described in U.S. Pat. No. 8,449,916, the contents of which is herein incorporated by reference in its entirety.

The nanoparticles and microparticles of the present invention may be geometrically engineered to modulate macrophage and/or the immune response. In one aspect, the geometrically engineered particles may have varied shapes, sizes and/or surface charges in order to incorporated the polynucleotides of the present invention for targeted delivery such as, but not limited to, pulmonary delivery (see e.g., International Publication No WO2013082111, the contents of which is herein incorporated by reference in its entirety). Other physical features the geometrically engineering particles may have include, but are not limited to, fenestrations, angled arms, asymmetry and surface roughness, charge which can alter the interactions with cells and tissues. As a non-limiting example, nanoparticles of the present invention may be made by the methods described in International Publication No WO2013082111, the contents of which is herein incorporated by reference in its entirety.

In one embodiment, the nanoparticles of the present invention may be water soluble nanoparticles such as, but not limited to, those described in International Publication No. WO2013090601, the contents of which is herein incorporated by reference in its entirety. The nanoparticles may be inorganic nanoparticles which have a compact and zwitterionic ligand in order to exhibit good water solubility. The nanoparticles may also have small hydrodynamic diameters (HD), stability with respect to time, pH, and salinity and a low level of non-specific protein binding.

In one embodiment the nanoparticles of the present invention may be developed by the methods described in US Patent Publication No. US20130172406, the contents of which are herein incorporated by reference in its entirety.

In one embodiment, the nanoparticles of the present invention are stealth nanoparticles or target-specific stealth nanoparticles such as, but not limited to, those described in US Patent Publication No. US20130172406; the contents of which is herein incorporated by reference in its entirety. The nanoparticles of the present invention may be made by the methods described in US Patent Publication No. US20130172406, the contents of which are herein incorporated by reference in its entirety.

In another embodiment, the stealth or target-specific stealth nanoparticles may comprise a polymeric matrix. The polymeric matrix may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates or combinations thereof.

In one embodiment, the nanoparticle may be a nanoparticle-nucleic acid hybrid structure having a high density nucleic acid layer. As a non-limiting example, the nanoparticle-nucleic acid hybrid structure may made by the methods described in US Patent Publication No. US20130171646, the contents of which are herein incorporated by reference in its entirety. The nanoparticle may comprise a nucleic acid such as, but not limited to, polynucleotides described herein and/or known in the art.

At least one of the nanoparticles of the present invention may be embedded in in the core a nanostructure or coated with a low density porous 3-D structure or coating which is capable of carrying or associating with at least one payload within or on the surface of the nanostructure. Non-limiting examples of the nanostructures comprising at least one nanoparticle are described in International Patent Publication No. WO2013123523, the contents of which are herein incorporated by reference in its entirety.

The mRNA cancer vaccines of the present invention may be administered by any route which results in a therapeutically effective outcome. These include, but are not limited, to intradermal, intramuscular, and/or subcutaneous administration. The present invention provides methods comprising administering mRNA cancer vaccines and in accordance with the invention to a subject in need thereof. The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect (see e.g., the range of unit doses described in International Publication No WO2013078199, herein incorporated by reference in its entirety). The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

A mRNA cancer vaccine pharmaceutical composition described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

In some embodiments, the RNA (e.g., mRNA) vaccine compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see e.g., the range of unit doses described in International Publication No WO2013078199, herein incorporated by reference in its entirety). The desired dosage may be delivered three times a day, two times a day, once a day, every other day, every third day, every week, every two weeks, every three weeks, every four weeks, every 2 months, every three months, every 6 months, etc. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In some embodiments, the RNA vaccine compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg. In some embodiments, the RNA vaccine compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, the RNA vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, a the RNA vaccine composition may be administered three or four times.

In some embodiments, the RNA vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

In some embodiments the RNA vaccine for use in a method of vaccinating a subject is administered the subject a single dosage of between 10 μg/kg and 400 μg/kg of the nucleic acid vaccine in an effective amount to vaccinate the subject. In some embodiments the RNA vaccine for use in a method of vaccinating a subject is administered the subject a single dosage of between 10 μg and 400 μg of the nucleic acid vaccine in an effective amount to vaccinate the subject.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

### Example 1. Manufacture of Polynucleotides

According to the present invention, the manufacture of polynucleotides and or parts or regions thereof may be accomplished utilizing the methods taught in U.S. Ser. No. 61/800,049 filed Mar. 15, 2013 entitled “Manufacturing Methods for Production of RNA Transcripts” (Attorney Docket number M500), the contents of which is incorporated herein by reference in its entirety.

Purification methods may include those taught in U.S. Provisional Patent Application No. 61/799,872, U.S. Provisional Patent Application No. 61/794,842, U.S. Provisional Patent Application 61/800,326, each of which is incorporated herein by reference in its entirety.

Detection and characterization methods of the polynucleotides may be performed as taught in U.S. Provisional Patent Application No. 61/799,780 and U.S. Provisional Patent Application No. 61/798,945, each of which is incorporated herein by reference in its entirety.

Characterization of the polynucleotides of the invention may be accomplished using a procedure selected from the group consisting of polynucleotide mapping, reverse transcriptase sequencing, charge distribution analysis, and detection of RNA impurities, wherein characterizing comprises determining the RNA transcript sequence, determining the purity of the RNA transcript, or determining the charge heterogeneity of the RNA transcript. Such methods are taught in, for example, U.S. Provisional Patent Application No. 61/799,905 and U.S. Provisional Patent Application No. 61/800,110, the contents of each of which is incorporated herein by reference in its entirety.

### Example 2. Chimeric Polynucleotide Synthesis

### INTRODUCTION

According to the present invention, two regions or parts of a chimeric polynucleotide may be joined or ligated using triphosphate chemistry.

According to this method, a first region or part of 100 nucleotides or less is chemically synthesized with a 5′ monophosphate and terminal 3′desOH or blocked OH. If the region is longer than 80 nucleotides, it may be synthesized as two strands for ligation.

If the first region or part is synthesized as a non-positionally modified region or part using in vitro transcription (IVT), conversion the 5′monophosphate with subsequent capping of the 3′ terminus may follow.

Monophosphate protecting groups may be selected from any of those known in the art.

The second region or part of the chimeric polynucleotide may be synthesized using either chemical synthesis or IVT methods. IVT methods may include an RNA polymerase that can utilize a primer with a modified cap. Alternatively, a cap of up to 130 nucleotides may be chemically synthesized and coupled to the IVT region or part.

It is noted that for ligation methods, ligation with DNA T4 ligase, followed by treatment with DNAse should readily avoid concatenation.

The entire chimeric polynucleotide need not be manufactured with a phosphate-sugar backbone. If one of the regions or parts encodes a polypeptide, then it is preferable that such region or part comprise a phosphate-sugar backbone.

Ligation is then performed using any known click chemistry, orthoclick chemistry, solulink, or other bioconjugate chemistries known to those in the art.

### Synthetic Route

The chimeric polynucleotide is made using a series of starting segments. Such segments include:
(a) Capped and protected 5′ segment comprising a normal 3′OH (SEG. 1)
(b) 5′ triphosphate segment which may include the coding region of a polypeptide and comprising a normal 3′OH (SEG. 2)
(c) 5′ monophosphate segment for the 3′ end of the chimeric polynucleotide (e.g., the tail) comprising cordycepin or no 3′OH (SEG. 3)

After synthesis (chemical or IVT), segment 3 (SEG. 3) is treated with cordycepin and then with pyrophosphatase to create the 5′monophosphate.

Segment 2 (SEG. 2) is then ligated to SEG. 3 using RNA ligase. The ligated polynucleotide is then purified and treated with pyrophosphatase to cleave the diphosphate. The treated SEG.2-SEG. 3 construct is then purified and SEG. 1 is ligated to the 5′ terminus. A further purification step of the chimeric polynucleotide may be performed.

Where the chimeric polynucleotide encodes a polypeptide, the ligated or joined segments may be represented as: 5′UTR (SEG. 1), open reading frame or ORF (SEG. 2) and 3′UTR+PolyA (SEG. 3).

The yields of each step may be as much as 90-95%.

### Example 3: PCR for cDNA Production

PCR procedures for the preparation of cDNA are performed using 2×KAPA HIFI™ HotStart ReadyMix by Kapa Biosystems (Woburn, MA). This system includes 2×KAPA ReadyMix12.5 μl; Forward Primer (10 uM) 0.75 μl; Reverse Primer (10 uM) 0.75 μl; Template cDNA−100 ng; and dH₂O diluted to 25.0 μl. The reaction conditions are at 95° C. for 5 min. and 25 cycles of 98° C. for 20 sec, then 58° C. for 15 sec, then 72° C. for 45 sec, then 72° C. for 5 min. then 4° C. to termination.

The reaction is cleaned up using Invitrogen's PURELINK™ PCR Micro Kit (Carlsbad, CA) per manufacturer's instructions (up to 5 μg). Larger reactions will require a cleanup using a product with a larger capacity. Following the cleanup, the cDNA is quantified using the NANODROP™ and analyzed by agarose gel electrophoresis to confirm the cDNA is the expected size. The cDNA is then submitted for sequencing analysis before proceeding to the in vitro transcription reaction.

### Example 4. In Vitro Transcription (IVT)

The in vitro transcription reaction generates polynucleotides containing uniformly modified polynucleotides. Such uniformly modified polynucleotides may comprise a region or part of the polynucleotides of the invention. The input nucleotide triphosphate (NTP) mix is made in-house using natural and un-natural NTPs.

A typical in vitro transcription reaction includes the following:

| 1 | Template cDNA | 1.0 | μg |
|---|---|---|---|
| 2 | 10x transcription buffer (400 mM Tris-HCl | 2.0 | μl |
| | pH 8.0, 190 mM MgCl₂, 50 mM DTT, 10 mM | | |
| | Spermidine) | | |
| 3 | Custom NTPs (25 mM each) | 7.2 | μl |
| 4 | RNase Inhibitor | 20 | U |
| 5 | T7 RNA polymerase | 3000 | U |
| 6 | dH₂0 | Up to 20.0 μl. and | |
| 7 | Incubation at 37° C. for 3 hr-5 hrs. | | |

The crude IVT mix may be stored at 4° C. overnight for cleanup the next day. 1 U of RNase-free DNase is then used to digest the original template. After 15 minutes of incubation at 37° C., the mRNA is purified using Ambion's MEGACLEAR™ Kit (Austin, TX) following the manufacturer's instructions. This kit can purify up to 500 μg of RNA. Following the cleanup, the RNA is quantified using the NanoDrop and analyzed by agarose gel electrophoresis to confirm the RNA is the proper size and that no degradation of the RNA has occurred.

### Example 5. In Vivo Immunogeniticy Assay with mRNA Cancer Vaccines

An MC38 immunogenicity study using mRNA vaccines in mice was performed. mRNA antigens: three MC38 neoepitopes Adpgk, Dpagtl, Reps1 having formats: 25mer, TMG, secreted CD40L-TMG fusion protein) were generated. The positive control was a benchmark comparison to 25-mer peptide immunization+anti-CD40+ poly(I:C) (Yadav et al, Nature 2015). Mice were immunized on days 0, 7, and 14. A readout was measured on Days 3, 10, and 17; followed by MC38 challenge on day 21 and sacrifice on day 35.

Characterization of the epitope-specific T cell population was made by frequency of antigen-specific T cell population by dextramers staining. A cytokine profile was generated: Intracellular cytokine staining (IFNγ, TNFα, IL-2) and ELISPOT (upon MC38 mutant peptide stimulation). The following memory and T cell differentiation markers: CD44, CD62L, IL7R, KLRG1, CD122 and exhaustion markers: PD1, Lag3, Tim3, 2B4 were used.

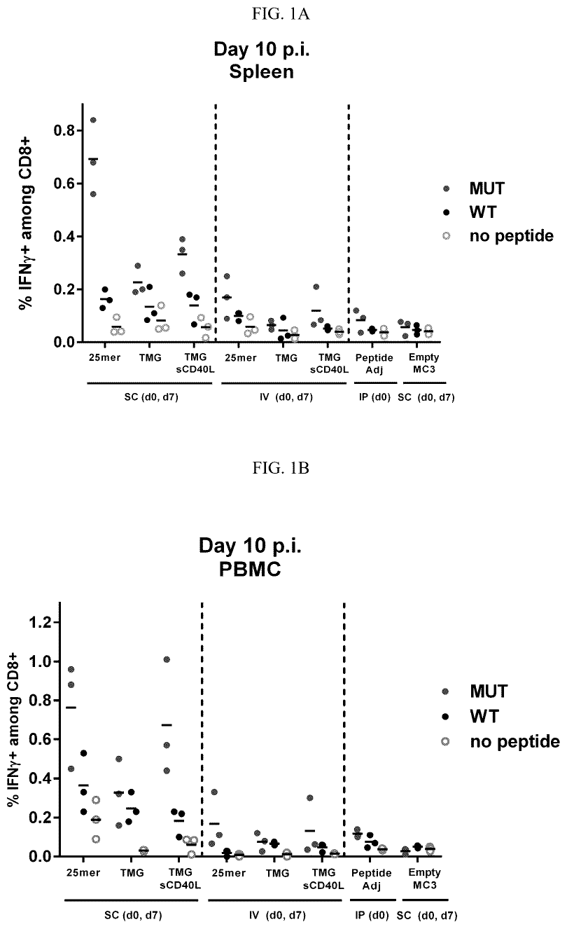

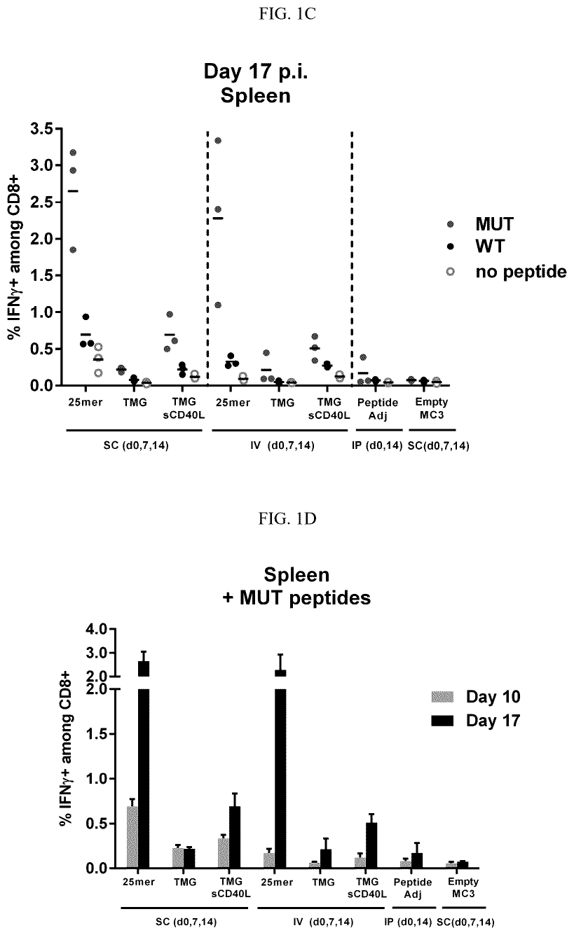

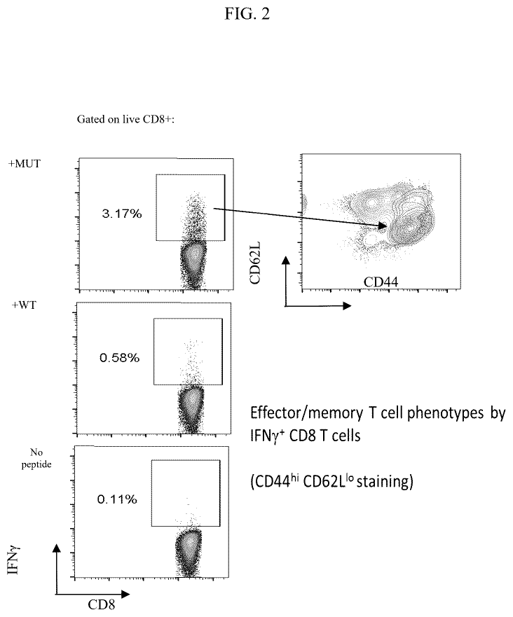

The results showing that mRNA vaccine induced an antigen specific CD8 response are shown in FIG. 1. Results showing that mRNA vaccines induced antigen specific effector/memory CD8 T cells are shown in FIG. 2.

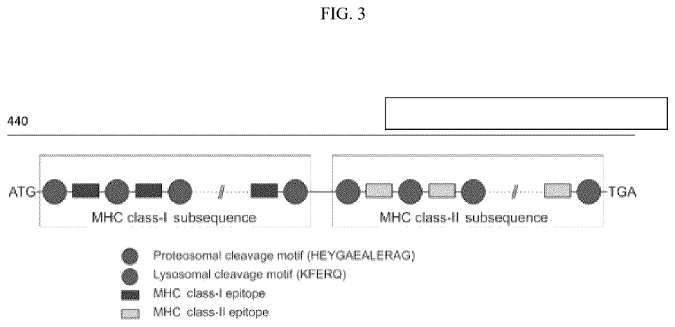

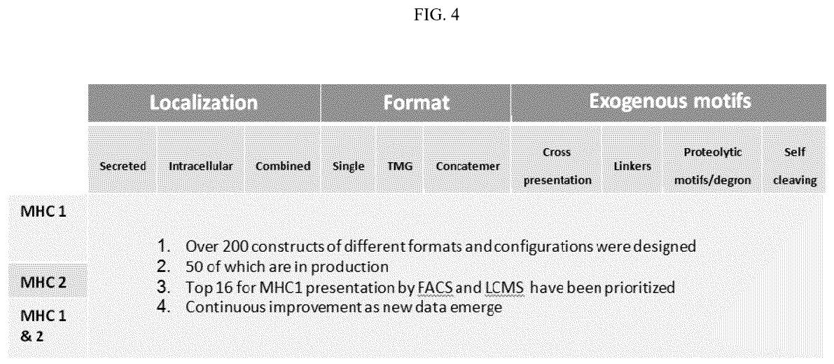

Some of the considerations for antigen designs include MHC classes, Expression localization, Polypeptide format and configuration, and Potency enhancing motifs. A multi-factorial consideration of antigen design of mRNA-based neoepitopes is shown in FIGS. 3 (schematic) and 4 (table).

### Example 6. Method Development of FACS-Based MHC-Presentation

Objective: Validation of FACS-based assay of mRNA encoded epitopes in MCF7 (HLA*201). The mRNA used was a combination a concatemer of four different epitopes: mut.gp100(T209M)+mut.tyrpsoinase(N271D)+mut.CDK4(R24C)+mut.MART1(A27L) TMG.G25 (1/2)\hat{ }3.nPEST seq: control mRNA of tandem minigene of three repeats of mut.gp100(T209M). Protein production was detected using an Anti-mut.MART1(A27L) TCRmer-PE and Anti-HLA antibodies.

The method involved: MCF7 transfected with 250 ng mRNA using LF2000; Peptide-pulsed control preparation: MCF7 were left un-pulsed or pulsed with synthetic peptides in serum-free RPMI for 3 h at 37 C; and FACS analysis with anti-HLA and TCRmer (specific for mutant MART1-HLA*201 complex) at ˜20 h.

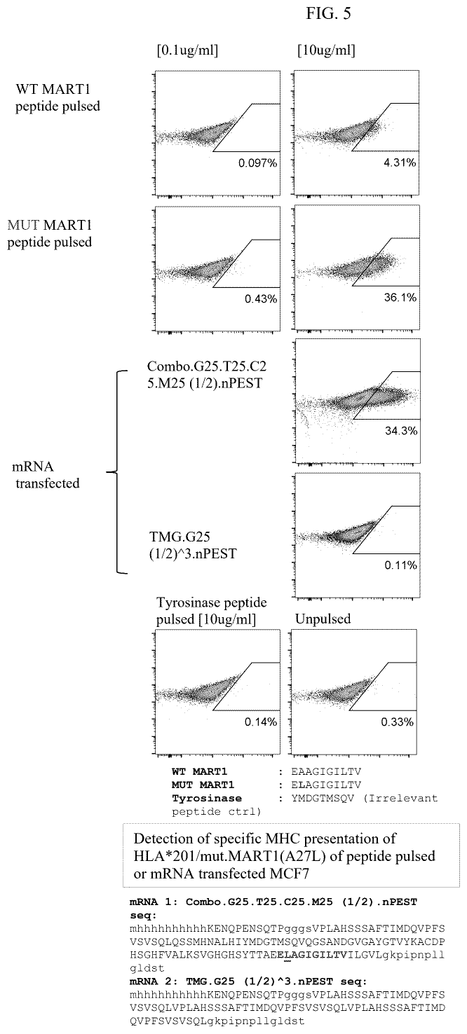

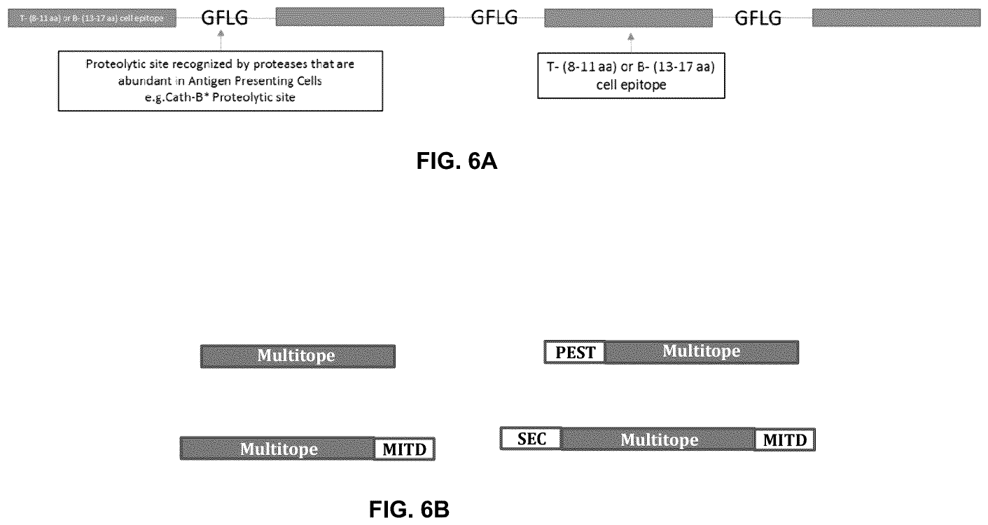

The data is shown in FIG. 5. Specific MHC1/mut.MART1peptide presentation by anti-mut.MART1TCRmer was detected on MCF7 cells.

### Example 7. T Cell Response Elicited with mRNA Encoding Concatamers of 20 Epitopes

mRNA concatamers induced both class I and class II T cell responses. CA60 encodes 20 epitopes derived from the mutanome of a patient. It includes 5 murine class II epitopes, 10 murine class I epitopes, a murine positive control (SIINFEKL (SEQ ID NO: 6), derived from ovalbumin), and 4 human (HLA-A2) epitopes (not shown). Mice were immunized with 10 ug mRNA twice (prime+boost at day 14) and spleen cells were analyzed at day 21 by flow cytometry.

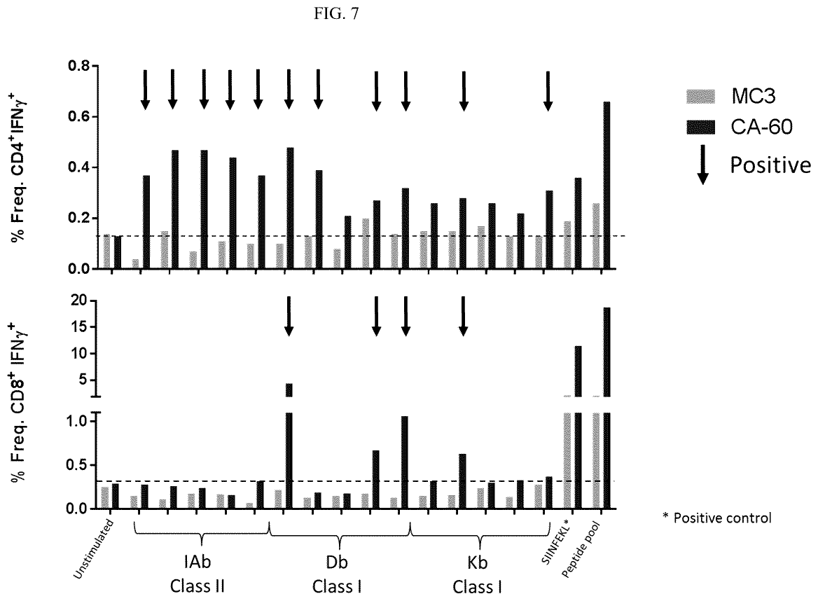

The data are shown in FIG. 7. Four out of ten Class I epitopes and five out of five class II epitopes were immunogenic. The epitopes showed responses two-fold over the unstimulated control. Some Class I predicted epitopes showed some level of cross presentation.

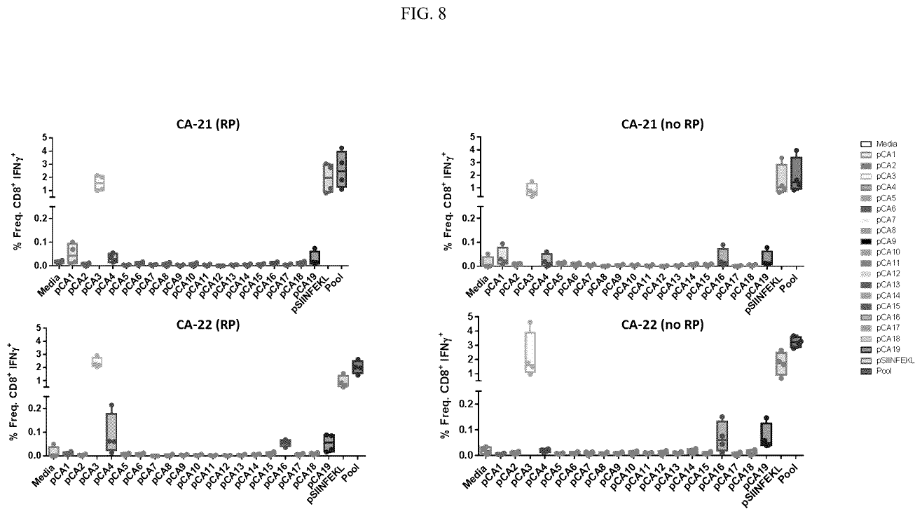

No differences were observed between RP and no-RP for known A2 concatamers. The same 5 out of 19 epitopes that showed responses in ELISpot after restimulation have CD8+IFNg+ specific responses. Data are shown in FIG. 8.

### Example 8. Epitopes are Immunogenic Irrespective of Position within mRNA Concatamer

The epitopes were immunogenic irrespective of their position within the mRNA. CA80 and CA81 encode the same 20 epitopes known to elicit T cell responses. They include 5 class II epitopes, 10 murine class I epitopes, a murine positive control (SIINFEKL (SEQ ID NO: 6), derived from ovalbumin), and 4 human (HLA-A2) epitopes (not shown). CA80 and CA81 differ only in the relative positions of the different epitopes. Mice were immunized with 10 ug mRNA twice (prime+boost at day 14) and spleen cells were analyzed at day 21 by flow cytometry.

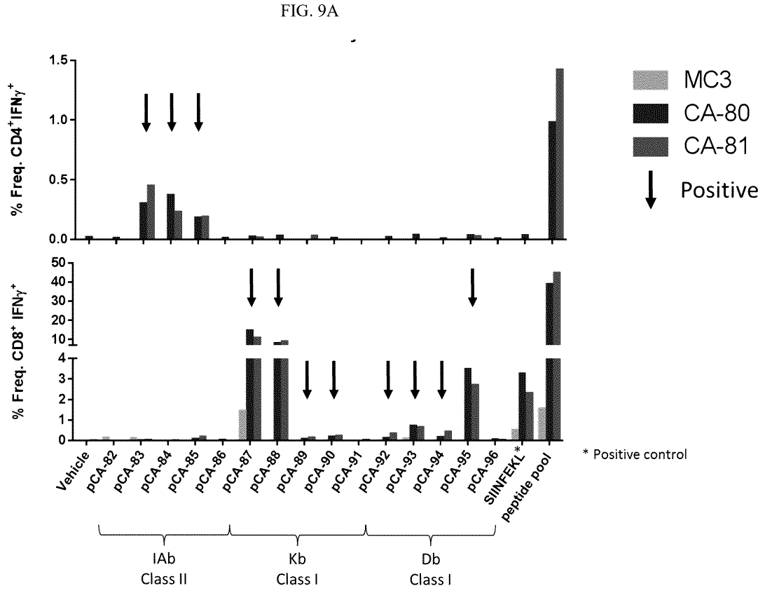

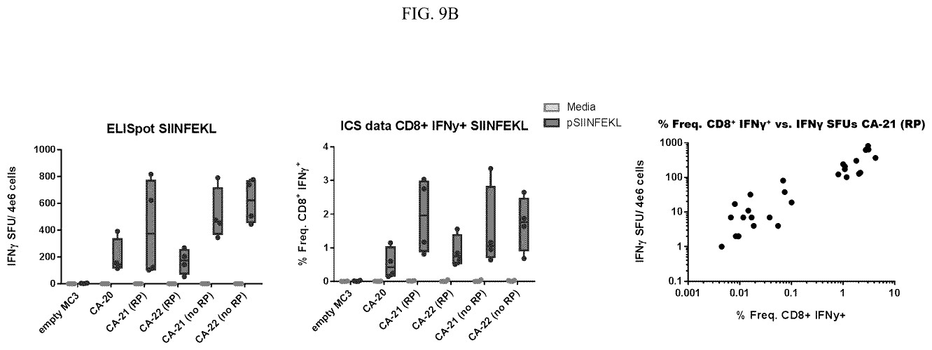

The data are shown in FIG. 9A. Eight out of 10 class I epitopes and three out of five class II epitopes were immunogenic. The epitopes showed responses eight-fold over the unstimulated control. The same level of immunogenicity was observed irrespective of the position within the mRNA. FIG. 9B shows that there is a strong correlation (R squared=0.78) between percent frequency of CD8+IFNg+ cells and interferon-gamma spot forming units (SFUs) in ELISpot assays.

### Example 9. Phase I, Open-Label Study to Assess Safety, Tolerability, and Immunogenicity of mRNA Vaccine in Patients with Solid Tumors

A phase I, open-label study to assess the safety, tolerability, and immunogenicity of mRNA 4379 alone in patients with resected solid tumors, and in combination with 5 pembrolizumab (a humanized anti-PD-1 antibody) in patients with unresectable solid tumors is performed.

Objectives: Primary: safety & tolerability of mRNA-4379 in patients with resected solid tumors (Part A) & mRNA-4379+ pembrolizumab in patients with unresectable solid tumors (Part B)

Secondary: Part A: RFS in patients with resected solid tumors treated.

Part B: ORR, DOR, PFS & OS in patients with unresectable solid tumors (pembro label)

Exploratory Study Objectives: Immunogenicity

Methodology: Two-part, open-label, 3+3 dose-escalation: fixed dose of either 0.1 mg, 0.2 mg or 0.4 mg of mRNA-4379 administered via intramuscular (IM) injections once during 21-day cycles for a maximum of 4 doses over 4 cycles.

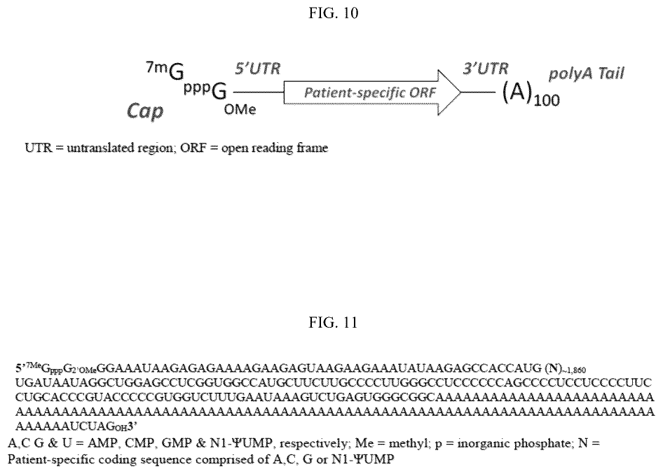

A schematic of the mRNA component of mRNA-4379 is shown in FIG. 10. mRNA-4379 contains a canonical dinucleotide mammalian cap 1 structure at the 5′ end comprised of a 7-methyl guanosine linked in a 5′-5′ triphosphate configuration to the penultimate nucleotide that is methylated at the 2′ position of the ribose sugar (Kozak, 1991; Fechter and Brownlee, 2005). The cap structure is required for initiation of translation. Following the cap structure is the 48-nt 5′ untranslated region (5′ UTR) that has been optimized to facilitate initiation of translation. The 5′ UTR ends at the AUG methionine start codon encoding the first amino acid of the protein coding region, or open reading frame (ORF), of mRNA-4379 which will be uniquely defined for each patient. The ORF of mRNA-4379 ends with the three mammalian stop codons linked in a row (5′-UGA-UAA-UAG-3′ (SEQ ID NO: 1132)) that start a common, pre-specified 3′ UTR nt sequence that has been optimized to promote mRNA stabilization. mRNA-4379 ends with an approximately 100-nt adenosine homopolymer, the polyA tail, which is required for mRNA stabilization and protein translation. Both the cap structure at the 5′ end and the polyA tail at the 3′ end are required for mRNA-4379 to be translated by the cellular translational machinery. RNA lacking either the 5′ cap or the 3′ polyA tail cannot be translated and therefore will not produce protein. Any degradant of mRNA-4379 lacking either the cap 1 structure on the 5′ end or the polyA tail on the 3′ end would not produce any protein.

An example of the general molecular sequence of mRNA-4379 is provided in FIG. 11, in which the patient specific coding region is depicted by reference as (N). The nucleosides in mRNA-4379 are chemically identical to naturally-occurring mammalian mRNA nucleosides, with the exception that the uridine nucleoside normally present in mammalian mRNA is fully replaced with N1-methyl-pseudouridine, a naturally-occurring pyrimidine base present in mammalian tRNAs (Rozenski, Crain et al. 1999; Kariko, Buckstein et al. 2005). This nucleoside is included in mRNA-4379 in place of the normal uridine base to minimize the indiscriminate recognition of mRNA-4379 by pathogen-associated molecular pattern (PAMP) receptors (e.g.,

Toll-like receptors (TLR), Desmet and Ishii, 2012).

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

1-77. (canceled)

78. A personalized messenger ribonucleic acid (mRNA) cancer vaccine for treating a cancer in a patient, comprising:
a mRNA which comprises an open reading frame (ORF) encoding 5-100 peptide epitopes, and
a lipid nanoparticle which comprises 20-60 mol % ionizable cationic lipid, 5-25 mol % non-cationic lipid, 25-55 mol % sterol, and 0.5-15% PEG-modified lipid;
wherein the peptide epitopes are selected by a process comprising:
(a) identifying neoepitopes that are expressed in a tumor sample from the patient but not expressed in a normal tissue of the patient, and
(b) selecting peptide epitopes to be encoded by the mRNA ORF which have the following characteristics:
(i) the peptide epitopes are selected from the identified neoepitopes;
(ii) the peptide epitopes lack self-reactivity;
(iii) each peptide epitope contains at least one of the following types of mutations: an insertion, a deletion, a substitution, and a frameshift mutation;
(iv) at least one of the peptide epitopes is an MHC class I epitope and at least one of the peptide epitopes is an MHC class II epitope,
wherein, in the ORF, the peptide epitopes are connected to one another directly or via a linker; and
wherein the mRNA comprises N1-methylpseudouridine.

79. The personalized mRNA cancer vaccine of claim 78, wherein at least two of the peptide epitopes comprise a mutation of the same type.

80. The personalized mRNA cancer vaccine of claim 79, wherein each of the peptide epitopes comprises a mutation of the same type.

81. The personalized mRNA cancer vaccine of claim 78, wherein one or more of the peptide epitopes comprises a mutation that is abundant in the tumor sample.

82. The personalized mRNA cancer vaccine of claim 78, wherein one or more of the peptide epitopes comprise a mutation that is highly abundant in the tumor sample.

83. The personalized mRNA cancer vaccine of claim 78, wherein the peptide epitopes are representative of one or more of an exome of the tumor sample and a transcriptome of the tumor sample.

84. The personalized mRNA cancer vaccine of claim 78, wherein one or more of the peptide epitopes comprise a conservative amino acid substitution.

85. The personalized mRNA cancer vaccine of claim 78, wherein each of the peptide epitopes comprises a conservative amino acid substitution.

86. The personalized mRNA cancer vaccine of claim 78, wherein one or more of the peptide epitopes comprise a non-conservative amino acid substitution.

87. The personalized mRNA cancer vaccine of claim 78, wherein each of the peptide epitopes comprises a non-conservative amino acid substitution.

88. The personalized mRNA cancer vaccine of claim 78, wherein the ORF encodes fewer than 100 peptide epitopes.

89. The personalized mRNA cancer vaccine of claim 78, wherein the ORF encodes 5-50 peptide epitopes.

90. The personalized mRNA cancer vaccine of claim 78, wherein the ORF encodes 5-40 peptide epitopes.

91. The personalized mRNA cancer vaccine of claim 78, wherein each peptide epitope is 9-29 amino acids in length.

92. The personalized mRNA cancer vaccine of claim 78, wherein at least 30% of the peptide epitopes are MHC class I epitopes.

93. The personalized mRNA cancer vaccine of claim 78, wherein at least 50% of the peptide epitopes are MHC class I epitopes.

94. The personalized mRNA cancer vaccine of claim 78, wherein the mRNA is modified with N1-methylpseudouridine throughout the entire sequence.

95. The personalized mRNA cancer vaccine of claim 78, wherein at least two of the peptide epitopes are connected to one another via a linker.

96. The personalized mRNA cancer vaccine of claim 78, wherein at least two of the peptide epitopes are connected directly to one another without a linker.

97. The personalized mRNA cancer vaccine of claim 78, wherein the ORF encodes 5-50 different peptide epitopes.

98. The personalized mRNA cancer vaccine of claim 78, wherein 3 or more of the peptide epitopes have the same amino acid sequence.

99. The personalized mRNA cancer vaccine of claim 78, wherein the selected peptide epitopes exhibit T cell reactivity.

100. The personalized mRNA cancer vaccine of claim 99, wherein the process further comprises, prior to step (b), identifying neoepitopes that exhibit T cell reactivity.

101. The personalized mRNA cancer vaccine of claim 78, wherein the lipid nanoparticle comprises about 50 mol % ionizable cationic lipid, about 10 mol % non-cationic lipid, about 38.5 mol % sterol, and about 1.5 mol % PEG-modified lipids.

102. The personalized mRNA cancer vaccine of claim 101, wherein:
the ORF encodes 5-40 peptide epitopes;
at least 50% of the peptide epitopes are MHC class I epitopes; and
the mRNA further comprises a 5′ terminal cap.

103. The personalized mRNA cancer vaccine of claim 102, further having one or more features selected from:
(a) two or more of the peptide epitopes are connected directly to one another;
(b) two or more of the peptide epitopes are connected to one another through a linker that is not cleavage sensitive;
(c) at least one of the peptide epitopes comprises 9-29 amino acids; and
(d) the ORF encodes 9 or more peptide epitopes.

104. The personalized mRNA cancer vaccine of claim 103, having each of features (a)-(d).

105. A method of treating a cancer in a patient comprising administering the personalized mRNA cancer vaccine of claim 78 to the patient.

106. A method of treating a cancer in a patient comprising administering the personalized mRNA cancer vaccine of claim 103 to the patient.

107. The method of claim 106, further comprising administering an immune checkpoint modulator to the patient.

108. The method of claim 107, wherein the personalized mRNA cancer vaccine and the immune checkpoint modulator are administered to the patient simultaneously.

109. The method of claim 107, wherein the personalized mRNA cancer vaccine and the immune checkpoint modulator are administered to the patient sequentially.

110. The method of claim 107, wherein the immune checkpoint modulator is an anti-PD1 antibody.

111. The method of claim 110, wherein the anti-PD1 antibody is pembrolizumab.

112. The method of claim 111, wherein the patient is a human patient and has a resected solid tumor.

113. The method of claim 111, wherein the patient is a human patient and has an unresectable solid tumor.

114. A method of treating a cancer in a patient comprising administering the personalized mRNA cancer vaccine of claim 103 to the patient and administering pembrolizumab to the patient.

115. A method of preparing a personalized messenger ribonucleic acid (mRNA) cancer vaccine for treating a cancer in a patient, the personalized mRNA vaccine comprising a mRNA which comprises an open reading frame (ORF) encoding 5-100 peptide epitopes, the method comprising selecting the peptide epitopes by a process comprising:
(a) identifying neoepitopes that are expressed in a tumor sample from the patient but not expressed in a normal tissue of the patient, and
(b) selecting peptide epitopes to be encoded by the mRNA ORF which have the following characteristics:
(i) the peptide epitopes are selected from the identified neoepitopes;
(ii) the peptide epitopes lack self-reactivity;
(iii) each peptide epitope contains at least one of the following types of mutations: an insertion, a deletion, a substitution, and a frameshift mutation;
(iv) at least one of the peptide epitopes is an MHC class I epitope and at least one of the peptide epitopes is an MHC class II epitope,
wherein, in the ORF, the peptide epitopes are connected to one another directly or via a linker; and
wherein the mRNA comprises N1-methylpseudouridine.

116. The method of claim 115, further comprising preparing the mRNA.

117. The method of claim 116, further comprising formulating the mRNA with a lipid nanoparticle, wherein the lipid nanoparticle comprises 20-60 mol % ionizable cationic lipid, 5-25 mol % non-cationic lipid, 25-55 mol % sterol, and 0.5-15% PEG-modified lipid.

118. A method of selecting a set of peptide epitopes for use in a personalized messenger ribonucleic acid (mRNA) cancer vaccine for treating a cancer in a patient, the personalized mRNA vaccine comprising an mRNA comprising an open reading frame (ORF) encoding 5-100 peptide epitopes, the method comprising:
(a) identifying neoepitopes that are expressed in a tumor sample from the patient but not expressed in a normal tissue of the patient, and
(b) selecting peptide epitopes to be encoded by the mRNA ORF which have the following characteristics:
(i) the peptide epitopes are selected from the identified neoepitopes;
(ii) the peptide epitopes lack self-reactivity;
(iii) each peptide epitope contains at least one of the following types of mutations: an insertion, a deletion, a substitution, and a frameshift mutation;
(iv) at least one of the peptide epitopes is an MHC class I epitope and at least one of the peptide epitopes is an MHC class II epitope,
wherein, in the ORF, the peptide epitopes are connected to one another directly or via a linker; and
wherein the mRNA comprises N1-methylpseudouridine.

119. The method of claim 118, wherein the mRNA is modified with N1-methylpseudouridine throughout the entire sequence.

120. A method of vaccinating a patient having a cancer with a personalized mRNA cancer vaccine comprising an mRNA comprising an open reading frame (ORF) encoding 5-100 peptide epitopes, the method comprising:
(a) identifying neoepitopes that are expressed in a tumor sample from the patient but not expressed in a normal tissue of the patient, and
(b) selecting peptide epitopes to be encoded by the mRNA ORF which have the following characteristics:
(i) the peptide epitopes are selected from the identified neoepitopes;
(ii) the peptide epitopes lack self-reactivity;
(iii) each peptide epitope contains at least one of the following types of mutations: an insertion, a deletion, a substitution, and a frameshift mutation;
(iv) at least one of the peptide epitopes is an MHC class I epitope and at least one of the peptide epitopes is an MHC class II epitope,
(c) preparing the mRNA, wherein, in the ORF, the peptide epitopes are connected to one another directly or via a linker, and wherein the mRNA comprises N1-methylpseudouridine;
(d) formulating the mRNA with a lipid nanoparticle to obtain the personalized mRNA cancer vaccine, wherein the lipid nanoparticle comprises 20-60 mol % ionizable cationic lipid, 5-25 mol % non-cationic lipid, 25-55 mol % sterol, and 0.5-15% PEG-modified lipid, and
(e) administering the personalized mRNA cancer vaccine to the patient.

121. The method of claim 120, wherein the mRNA is modified with N1-methylpseudouridine throughout the entire sequence.


SEQUENCE LISTING (1132 sequences)

SEQ ID NO: 1 - protein, 4 aa, synthetic construct
  REGION 1..4: note = Synthetic Polypeptide
GFLG

SEQ ID NO: 2 - protein, 4 aa, synthetic construct
  REGION 1..4: note = Synthetic Polypeptide
KVSR

SEQ ID NO: 3 - protein, 5 aa, synthetic construct
  REGION 1..5: note = Synthetic Polypeptide
TVGLR

SEQ ID NO: 4 - protein, 5 aa, synthetic construct
  REGION 1..5: note = Synthetic Polypeptide
PMGLP

SEQ ID NO: 5 - protein, 5 aa, synthetic construct
  REGION 1..5: note = Synthetic Polypeptide
PMGAP

SEQ ID NO: 6 - protein, 8 aa, synthetic construct
  REGION 1..8: note = Synthetic Polypeptide
SIINFEKL

SEQ ID NO: 7 - DNA, 42 bp, synthetic construct
  misc_feature 1..42: note = Synthetic Polynucleotide
  source 1..42: mol_type = other DNA
aagcttagcggccgcaccatgcgggtcacggcgccccgaacc

SEQ ID NO: 8 - DNA, 30 bp, synthetic construct
  misc_feature 1..30: note = Synthetic Polynucleotide
  source 1..30: mol_type = other DNA
ctgcagggagccggcccaggtctcggtcag

SEQ ID NO: 9 - DNA, 33 bp, synthetic construct
  misc_feature 1..33: note = Synthetic Polynucleotide
  source 1..33: mol_type = other DNA
ggatccatcgtgggcattgttgctggcctggct

SEQ ID NO: 10 - DNA, 42 bp, synthetic construct
  misc_feature 1..42: note = Synthetic Polynucleotide
  source 1..42: mol_type = other DNA
gaattcagtctcgagtcaagctgtgagagacacatcagagcc

SEQ ID NO: 11 - protein, 76 aa, Homo sapiens [sequence omitted]

SEQ ID NO: 12 - protein, 41 aa, synthetic construct
  REGION 1..41: note = Synthetic Polypeptide
EGAMVAATQGAAAAAGSGAGTGGGTASGGTEGGSAESEGAK

SEQ ID NO: 13 - protein, 39 aa, synthetic construct
  REGION 1..39: note = Synthetic Polypeptide
AMVAATQGAAAAAGSGAGTGGGTASGGTEGGSAESEGAK

SEQ ID NO: 14 - protein, 26 aa, synthetic construct
  REGION 1..26: note = Synthetic Polypeptide
GGGGGGGGAGAAGGGGGGENYDDPHK

SEQ ID NO: 15 - protein, 38 aa, synthetic construct
  REGION 1..38: note = Synthetic Polypeptide
MVAATQGAAAAAGSGAGTGGGTASGGTEGGSAESEGAK

SEQ ID NO: 16 - protein, 17 aa, synthetic construct
  REGION 1..17: note = Synthetic Polypeptide
QLLCGAAIGTHEDDKYR

SEQ ID NO: 17 - protein, 22 aa, synthetic construct
  REGION 1..22: note = Synthetic Polypeptide
FSHHFEDADNIYIFLELCSRKS

SEQ ID NO: 18 - protein, 15 aa, synthetic construct
  REGION 1..15: note = Synthetic Polypeptide
  VARIANT 2: note = misc_feature - Xaa can be any naturally occurring amino acid
YXLVGAGAIGCELLK

SEQ ID NO: 19 - protein, 26 aa, synthetic construct
  REGION 1..26: note = Synthetic Polypeptide
IPESCSFGYHAGGWGKPPVDETGKPL

SEQ ID NO: 20 - protein, 36 aa, synthetic construct
  REGION 1..36: note = Synthetic Polypeptide
VAATQGAAAAAGSGAGTGGGTASGGTEGGSASEGAK

SEQ ID NO: 21 - protein, 22 aa, synthetic construct
  REGION 1..22: note = Synthetic Polypeptide
SEADIEGPLPAKDIHLDLPSNN

SEQ ID NO: 22 - protein, 16 aa, synthetic construct
  REGION 1..16: note = Synthetic Polypeptide
HFNALGGWGELQNSVK

SEQ ID NO: 23 - protein, 12 aa, synthetic construct
  REGION 1..12: note = Synthetic Polypeptide
FAQALGLTEAVK

SEQ ID NO: 24 - protein, 17 aa, synthetic construct
  REGION 1..17: note = Synthetic Polypeptide
TSVLAAANPIESQWNPK

SEQ ID NO: 25 - protein, 16 aa, synthetic construct
  REGION 1..16: note = Synthetic Polypeptide
QLLQANPILESFGNAK

SEQ ID NO: 26 - protein, 16 aa, synthetic construct
  REGION 1..16: note = Synthetic Polypeptide
TSILAAANPISGHYDR

SEQ ID NO: 27 - protein, 15 aa, synthetic construct
  REGION 1..15: note = Synthetic Polypeptide
  VARIANT 2..3: note = misc_feature - Xaa can be any naturally occurring amino acid
IXXANPLLEAFGNAK

SEQ ID NO: 28 - protein, 17 aa, synthetic construct
  REGION 1..17: note = Synthetic Polypeptide
LYGAQFHPEVGLTENGK

SEQ ID NO: 29 - protein, 22 aa, synthetic construct
  REGION 1..22: note = Synthetic Polypeptide
PQGQAPPLSQAQGHPGIQTPQR

SEQ ID NO: 30 - protein, 27 aa, synthetic construct
  REGION 1..27: note = Synthetic Polypeptide
AAASAAAASAASGSPGPGEGSAGGEKR

SEQ ID NO: 31 - protein, 16 aa, synthetic construct
  REGION 1..16: note = Synthetic Polypeptide
  VARIANT 2..4: note = misc_feature - Xaa can be any naturally occurring amino acid
IXXXFLGASLKDEVLK

SEQ ID NO: 32 - protein, 21 aa, synthetic construct
  REGION 1..21: note = Synthetic Polypeptide
LTISPDYAYGATGHPGIIPPH

SEQ ID NO: 33 - protein, 22 aa, synthetic construct
  REGION 1..22: note = Synthetic Polypeptide
LTISPDYAYGATGHPGIIPPHA

SEQ ID NO: 34 - protein, 19 aa, synthetic construct
  REGION 1..19: note = Synthetic Polypeptide
ILISLATGHREEGGENLDQ

SEQ ID NO: 35 - protein, 27 aa, synthetic construct
  REGION 1..27: note = Synthetic Polypeptide
LSELTQQLAQATGKPPQYIAVHVVPDQ

SEQ ID NO: 36 - protein, 28 aa, synthetic construct
  REGION 1..28: note = Synthetic Polypeptide
LSELTQQLAQATGKPPQYIAVHVVPDQL

SEQ ID NO: 37 - protein, 19 aa, synthetic construct
  REGION 1..19: note = Synthetic Polypeptide
DATNVGDEGGFAPNILENK

SEQ ID NO: 38 - protein, 14 aa, synthetic construct
  REGION 1..14: note = Synthetic Polypeptide
ILAQATSDLVNAIK

SEQ ID NO: 39 - protein, 15 aa, synthetic construct
  REGION 1..15: note = Synthetic Polypeptide
  VARIANT 2..3: note = misc_feature - Xaa can be any naturally occurring amino acid
  VARIANT 5: note = misc_feature - Xaa can be any naturally occurring amino acid
VXXVXQHAVGIVVNK

SEQ ID NO: 40 - protein, 23 aa, synthetic construct
  REGION 1..23: note = Synthetic Polypeptide
GSLAEAVGSPPPAATPTPTPPTR

SEQ ID NO: 41 - protein, 19 aa, synthetic construct
  REGION 1..19: note = Synthetic Polypeptide
  VARIANT 2: note = misc_feature - Xaa can be any naturally occurring amino acid
SXGLPVGAVINCADNTGAK

SEQ ID NO: 42 - protein, 23 aa, synthetic construct
  REGION 1..23: note = Synthetic Polypeptide
YCFSEMAPVCAVVGGILAQEIVK

SEQ ID NO: 43 - protein, 20 aa, synthetic construct
  REGION 1..20: note = Synthetic Polypeptide
HVYGYSMAYGPAQHAISTEK

SEQ ID NO: 44 - protein, 20 aa, synthetic construct
  REGION 1..20: note = Synthetic Polypeptide
LWQLSKPRPGCSVLGPLPLL

SEQ ID NO: 45 - protein, 18 aa, synthetic construct
  REGION 1..18: note = Synthetic Polypeptide
MILIQDGSQNTNVDKPLR

SEQ ID NO: 46 - protein, 22 aa, synthetic construct
  REGION 1..22: note = Synthetic Polypeptide
TYSMVVVPLYDTLGPGAIRYII

SEQ ID NO: 47 - protein, 22 aa, synthetic construct
  REGION 1..22: note = Synthetic Polypeptide
HFAMMHGGTGFAGIDSSSPEVK

SEQ ID NO: 48 - protein, 21 aa, synthetic construct
  REGION 1..21: note = Synthetic Polypeptide
  VARIANT 2: note = misc_feature - Xaa can be any naturally occurring amino acid
GXLKPGMVVTFAPVNVTTEVK

SEQ ID NO: 49 - protein, 15 aa, synthetic construct
  REGION 1..15: note = Synthetic Polypeptide
FNALFAQGNYSEAAK

SEQ ID NO: 50 - protein, 24 aa, synthetic construct
  REGION 1..24: note = Synthetic Polypeptide
GPIHIGGPPGFASSSGKPGPTVIK

SEQ ID NO: 51 - protein, 15 aa, synthetic construct
  REGION 1..15: note = Synthetic Polypeptide
GFGFVTFDDHDPVDK

SEQ ID NO: 52 - protein, 20 aa, synthetic construct
  REGION 1..20: note = Synthetic Polypeptide
DQGSCGSCWAFGAVEAISDR

SEQ ID NO: 53 - protein, 21 aa, synthetic construct
  REGION 1..21: note = Synthetic Polypeptide
  VARIANT 2: note = misc_feature - Xaa can be any naturally occurring amino acid
GXNFGFGDSRGGGGNFGPGPG

SEQ ID NO: 54 - protein, 19 aa, synthetic construct
  REGION 1..19: note = Synthetic Polypeptide
HDLFDSGFGGGAGVETGGK

SEQ ID NO: 55 - protein, 15 aa, synthetic construct
  REGION 1..15: note = Synthetic Polypeptide
CYLFGGLANDSEDPK

SEQ ID NO: 56 - protein, 20 aa, synthetic construct
  REGION 1..20: note = Synthetic Polypeptide
TTEDSVMLNGFGTVVNALGK

SEQ ID NO: 57 - protein, 20 aa, synthetic construct
  REGION 1..20: note = Synthetic Polypeptide
LTEGLHGFHVHEFGDNTAGC

SEQ ID NO: 58 - protein, 11 aa, synthetic construct
  REGION 1..11: note = Synthetic Polypeptide
GYAFIEYEHER

SEQ ID NO: 59 - protein, 13 aa, synthetic construct
  REGION 1..13: note = Synthetic Polypeptide
MFIGGLSWDTSKK

SEQ ID NO: 60 - protein, 13 aa, synthetic construct
  REGION 1..13: note = Synthetic Polypeptide
MFIGGLSWDTTKK

SEQ ID NO: 61 - protein, 12 aa, synthetic construct
  REGION 1..12: note = Synthetic Polypeptide
SMGFIGHYLDQK

SEQ ID NO: 62 - protein, 12 aa, synthetic construct
  REGION 1..12: note = Synthetic Polypeptide
SMGFIGHYLDQK

SEQ ID NO: 63 - protein, 21 aa, synthetic construct
  REGION 1..21: note = Synthetic Polypeptide
  VARIANT 3: note = misc_feature - Xaa can be any naturally occurring amino acid
ALXGGIGFIHHNCTPEFQANE

SEQ ID NO: 64 - protein, 19 aa, synthetic construct
  REGION 1..19: note = Synthetic Polypeptide
NLQSTFSGFGFINSENVFK

SEQ ID NO: 65 - protein, 15 aa, synthetic construct
  REGION 1..15: note = Synthetic Polypeptide
GFCFITYTDEEPVKK

SEQ ID NO: 66 - protein, 12 aa, synthetic construct
  REGION 1..12: note = Synthetic Polypeptide
MPMFIVNTNVPR

SEQ ID NO: 67 - protein, 19 aa, synthetic construct
  REGION 1..19: note = Synthetic Polypeptide
VSEIFVELQGFLAAEQDIR

SEQ ID NO: 68 - protein, 11 aa, synthetic construct
  REGION 1..11: note = Synthetic Polypeptide
GFCFLEYEDHK

SEQ ID NO: 69 - protein, 15 aa, synthetic construct
  REGION 1..15: note = Synthetic Polypeptide
QAVSMFLGAVEEAKK

SEQ ID NO: 70 - protein, 17 aa, synthetic construct
  REGION 1..17: note = Synthetic Polypeptide
  VARIANT 3: note = misc_feature - Xaa can be any naturally occurring amino acid
KPXKPMQFLGDEETVRK

SEQ ID NO: 71 - protein, 22 aa, synthetic construct
  REGION 1..22: note = Synthetic Polypeptide
GAAEPHTIAAFLGGAAAQEVIK

SEQ ID NO: 72 - protein, 17 aa, synthetic construct
  REGION 1..17: note = Synthetic Polypeptide
MIPCDFLIPVQTQHPIR

SEQ ID NO: 73 - protein, 19 aa, synthetic construct
  REGION 1..19: note = Synthetic Polypeptide
QGAPTSFLPPEASQLKPDR

SEQ ID NO: 74 - protein, 14 aa, synthetic construct
  REGION 1..14: note = Synthetic Polypeptide
STGGAPTFNVTVTK

SEQ ID NO: 75 - protein, 13 aa, synthetic construct
  REGION 1..13: note = Synthetic Polypeptide
MVYMFQYDSTHGK

SEQ ID NO: 76 - protein, 22 aa, synthetic construct
  REGION 1..22: note = Synthetic Polypeptide
HFPMTHGNTGFSGIESSSPEVK

SEQ ID NO: 77 - protein, 12 aa, synthetic construct
  REGION 1..12: note = Synthetic Polypeptide
AVAFSPVTELKK

SEQ ID NO: 78 - protein, 22 aa, synthetic construct
  REGION 1..22: note = Synthetic Polypeptide
GFGFVTFSSMAEVDAAMAARPH

SEQ ID NO: 79 - protein, 15 aa, synthetic construct
  REGION 1..15: note = Synthetic Polypeptide
TCGFDFTGAVEDISK

SEQ ID NO: 80 - protein, 18 aa, synthetic construct
  REGION 1..18: note = Synthetic Polypeptide
EYSGLSDGYGFTTDLFGR

SEQ ID NO: 81 - protein, 23 aa, synthetic construct
  REGION 1..23: note = Synthetic Polypeptide
  VARIANT 5: note = misc_feature - Xaa can be any naturally occurring amino acid
GQHVXGSPFQFTVGPLGEGGAHK

SEQ ID NO: 82 - protein, 14 aa, synthetic construct
  REGION 1..14: note = Synthetic Polypeptide
GFGFVDFNSEEDAK

SEQ ID NO: 83 - protein, 10 aa, synthetic construct
  REGION 1..10: note = Synthetic Polypeptide
  VARIANT 2: note = misc_feature - Xaa can be any naturally occurring amino acid
FXFVEFEDPR

SEQ ID NO: 84 - protein, 10 aa, synthetic construct
  REGION 1..10: note = Synthetic Polypeptide
  VARIANT 2: note = misc_feature - Xaa can be any naturally occurring amino acid
FXFVEFEDPR

SEQ ID NO: 85 - protein, 17 aa, synthetic construct
  REGION 1..17: note = Synthetic Polypeptide
IELFVGGELIDPADDRK

SEQ ID NO: 86 - protein, 13 aa, synthetic construct
  REGION 1..13: note = Synthetic Polypeptide
MFVGGLSWDTSKK

SEQ ID NO: 87 - protein, 16 aa, synthetic construct
  REGION 1..16: note = Synthetic Polypeptide
AFSAFVGQMHQQGILK

SEQ ID NO: 88 - protein, 17 aa, synthetic construct
  REGION 1..17: note = Synthetic Polypeptide
GILFVGSGVSGGEEGAR

SEQ ID NO: 89 - protein, 18 aa, synthetic construct
  REGION 1..18: note = Synthetic Polypeptide
IIAFVGSPVEDNEKDLVK

SEQ ID NO: 90 - protein, 10 aa, synthetic construct
  REGION 1..10: note = Synthetic Polypeptide
DYAFVHFEDR

SEQ ID NO: 91 - protein, 15 aa, synthetic construct
  REGION 1..15: note = Synthetic Polypeptide
GYAFVHFETQEAADK

SEQ ID NO: 92 - protein, 15 aa, synthetic construct
  REGION 1..15: note = Synthetic Polypeptide
GYGFVHFETQEAAER

SEQ ID NO: 93 - protein, 10 aa, synthetic construct
  REGION 1..10: note = Synthetic Polypeptide
NYGFVHIEDK

SEQ ID NO: 94 - protein, 18 aa, synthetic construct
  REGION 1..18: note = Synthetic Polypeptide
ITLPVDFVTADKFDENAK

SEQ ID NO: 95 - protein, 15 aa, synthetic construct
  REGION 1..15: note = Synthetic Polypeptide
GFGFVTFDDHDPVDK

SEQ ID NO: 96 - protein, 17 aa, synthetic construct
  REGION 1..17: note = Synthetic Polypeptide
LPNFGFVVFDDSEPVQK

SEQ ID NO: 97 - protein, 15 aa, synthetic construct
  REGION 1..15: note = Synthetic Polypeptide
GFGFVYFQNHDAADK

SEQ ID NO: 98 - protein, 11 aa, synthetic construct
  REGION 1..11: note = Synthetic Polypeptide
YQFWDTQPVPK

SEQ ID NO: 99 - protein, 17 aa, synthetic construct
  REGION 1..17: note = Synthetic Polypeptide
QLLCGAAIGTHEDDKYR

SEQ ID NO: 100 - protein, 17 aa, synthetic construct
  REGION 1..17: note = Synthetic Polypeptide
QLLCGAAIGTHEDDKYR

SEQ ID NO: 101 - protein, 26 aa, synthetic construct
  REGION 1..26: note = Synthetic Polypeptide
PPAGGGGGAGGAGGGPPPGPPGAGDR

SEQ ID NO: 102 - protein, 21 aa, synthetic construct
  REGION 1..21: note = Synthetic Polypeptide
FGGSFAGSFGGAGGHAPGVAR

SEQ ID NO: 103 - protein, 26 aa, synthetic construct
  REGION 1..26: note = Synthetic Polypeptide
CNPIISGLYQGAGGPGPGGFGAQGPK

SEQ ID NO: 104 - protein, 22 aa, synthetic construct
  REGION 1..22: note = Synthetic Polypeptide
PGLNLPPPIGGAGPPLGLPKPK

SEQ ID NO: 105 - protein, 25 aa, synthetic construct
  REGION 1..25: note = Synthetic Polypeptide
  VARIANT 3: note = misc_feature - Xaa can be any naturally occurring amino acid
QPXVDGFLVGGASLKPEFVDIINAK

SEQ ID NO: 106 - protein, 15 aa, synthetic construct
  REGION 1..15: note = Synthetic Polypeptide
VTGDHIPTPQDLPQR

SEQ ID NO: 107 - protein, 12 aa, synthetic construct
  REGION 1..12: note = Synthetic Polypeptide
YGGELVPHFPAR

SEQ ID NO: 108 - protein, 18 aa, synthetic construct
  REGION 1..18: note = Synthetic Polypeptide
YQGAGGPGPGGFGAQGPK

SEQ ID NO: 109 - protein, 19 aa, synthetic construct
  REGION 1..19: note = Synthetic Polypeptide
EYFGGFGEVESIELPMDNK

SEQ ID NO: 110 - protein, 15 aa, synthetic construct
  REGION 1..15: note = Synthetic Polypeptide
ALVLGGFAHMDTETK

SEQ ID NO: 111 - protein, 18 aa, synthetic construct
  REGION 1..18: note = Synthetic Polypeptide
VSHVSTGGGASLELLEGK

SEQ ID NO: 112 - protein, 19 aa, synthetic construct
  REGION 1..19: note = Synthetic Polypeptide
AEGGGGGGRPGAPAAGDGK

SEQ ID NO: 113 - protein, 17 aa, synthetic construct
  REGION 1..17: note = Synthetic Polypeptide
RGGGGGGSGGIGYPYPR

SEQ ID NO: 114 - protein, 23 aa, synthetic construct
  REGION 1..23: note = Synthetic Polypeptide
NMGGPYGGGNYGPGGSGGSGGYG

SEQ ID NO: 115 - protein, 21 aa, synthetic construct
  REGION 1..21: note = Synthetic Polypeptide
GTGGVDTAATGGVFDISNLDR

SEQ ID NO: 116 - protein, 16 aa, synthetic construct
  REGION 1..16: note = Synthetic Polypeptide
HFNALGGWGELQNSVK

SEQ ID NO: 117 - protein, 25 aa, synthetic construct
  REGION 1..25: note = Synthetic Polypeptide
PESCSFGYHAGGWGKPPVDETGKPL

SEQ ID NO: 118 - protein, 15 aa, synthetic construct
  REGION 1..15: note = Synthetic Polypeptide
SSLPNFCGIFNHLER

SEQ ID NO: 119 - protein, 19 aa, synthetic construct
  REGION 1..19: note = Synthetic Polypeptide
  VARIANT 5: note = misc_feature - Xaa can be any naturally occurring amino acid
AMALXGGIGFIHHNCTPEF

SEQ ID NO: 120 - protein, 23 aa, synthetic construct
  REGION 1..23: note = Synthetic Polypeptide
  VARIANT 5: note = misc_feature - Xaa can be any naturally occurring amino acid
AMALXGGIGFIHHNCTPEFQANE

SEQ ID NO: 121 - protein, 22 aa, synthetic construct
  REGION 1..22: note = Synthetic Polypeptide
EWIKPIMFSGGIGSMEADHISK

SEQ ID NO: 122 - protein, 14 aa, synthetic construct
  REGION 1..14: note = Synthetic Polypeptide
GDGPVQGIINFEQK

SEQ ID NO: 123 - protein, 19 aa, synthetic construct
  REGION 1..19: note = Synthetic Polypeptide
EMAPVCAVVGGILAQEIVK

SEQ ID NO: 124 - protein, 14 aa, synthetic construct
  REGION 1..14: note = Synthetic Polypeptide
LAFHGILLHGLEDR

SEQ ID NO: 125 - protein, 14 aa, synthetic construct
  REGION 1..14: note = Synthetic Polypeptide
MGVVAGILVQNVLK

SEQ ID NO: 126 - protein, 20 aa, synthetic construct
  REGION 1..20: note = Synthetic Polypeptide
FTASAGIQVVGDDLTVTNPK

SEQ ID NO: 127 - protein, 21 aa, synthetic construct
  REGION 1..21: note = Synthetic Polypeptide
TPYQIACGISQGLADNTVIAK

SEQ ID NO: 128 - protein, 16 aa, synthetic construct
  REGION 1..16: note = Synthetic Polypeptide
YPIEHGIVTNWDDMEK

SEQ ID NO: 129 - protein, 21 aa, synthetic construct
  REGION 1..21: note = Synthetic Polypeptide
  VARIANT 10: note = misc_feature - Xaa can be any naturally occurring amino acid
VASGIPAGWXGLDCGPESSKK

SEQ ID NO: 130 - protein, 16 aa, synthetic construct
  REGION 1..16: note = Synthetic Polypeptide
LFVGGLDWSTTQETLR

SEQ ID NO: 131 - protein, 16 aa, synthetic construct
  REGION 1..16: note = Synthetic Polypeptide
HGGSLGLGLAAMGTAR

SEQ ID NO: 132 - protein, 16 aa, synthetic construct
  REGION 1..16: note = Synthetic Polypeptide
IFVGGLSANTVVEDVK

SEQ ID NO: 133 - protein, 16 aa, synthetic construct
  REGION 1..16: note = Synthetic Polypeptide
LFIGGLSFETTDDSLR

SEQ ID NO: 134 - protein, 16 aa, synthetic construct
  REGION 1..16: note = Synthetic Polypeptide
LFIGGLSFETTDESLR

SEQ ID NO: 135 - protein, 16 aa, synthetic construct
  REGION 1..16: note = Synthetic Polypeptide
LFIGGLSFETTEESLR

SEQ ID NO: 136 - protein, 13 aa, synthetic construct
  REGION 1..13: note = Synthetic Polypeptide
  VARIANT 3: note = misc_feature - Xaa can be any naturally occurring amino acid
MFXGGLSWDTSKK

SEQ ID NO: 137 - protein, 13 aa, synthetic construct
  REGION 1..13: note = Synthetic Polypeptide
  VARIANT 3: note = misc_feature - Xaa can be any naturally occurring amino acid
MFXGGLSWDTSKK

SEQ ID NO: 138 - protein, 13 aa, synthetic construct
  REGION 1..13: note = Synthetic Polypeptide
  VARIANT 3: note = misc_feature - Xaa can be any naturally occurring amino acid
MFXGGLSWDTSKK

SEQ ID NO: 139 - protein, 15 aa, synthetic construct
  REGION 1..15: note = Synthetic Polypeptide
DAVSGMGVIVHIIEK

SEQ ID NO: 140 - protein, 10 aa, synthetic construct
  REGION 1..10: note = Synthetic Polypeptide
GGNFGFGDSR

SEQ ID NO: 141 - protein, 26 aa, synthetic construct
  REGION 1..26: note = Synthetic Polypeptide
GTTGSGAGSGGPGGLTSAAPAGGDKK

SEQ ID NO: 142 - protein, 23 aa, synthetic construct
  REGION 1..23: note = Synthetic Polypeptide
IISGLYQGAGGPGPGGFGAQGPK

SEQ ID NO: 143 - protein, 23 aa, synthetic construct
  REGION 1..23: note = Synthetic Polypeptide
IISGLYQGAGGPGPGGFGAQGPK

SEQ ID NO: 144 - protein, 22 aa, synthetic construct
  REGION 1..22: note = Synthetic Polypeptide
GGGLLIGGQAWDWANQGEDERV

SEQ ID NO: 145 - protein, 23 aa, synthetic construct
  REGION 1..23: note = Synthetic Polypeptide
GNFGGSFAGSFGGAGGHAPGVAR

SEQ ID NO: 146 - protein, 22 aa, synthetic construct
  REGION 1..22: note = Synthetic Polypeptide
NFGGSFAGSFGGAGGHAPGVAR

SEQ ID NO: 147 - protein, 22 aa, synthetic construct
  REGION 1..22: note = Synthetic Polypeptide
NFGGSFAGSFGGAGGHAPGVAR

SEQ ID NO: 148 - protein, 22 aa, synthetic construct
  REGION 1..22: note = Synthetic Polypeptide
NFGGSFAGSFGGAGGHAPGVAR

SEQ ID NO: 149 - protein, 33 aa, synthetic construct
  REGION 1..33: note = Synthetic Polypeptide
SAADTKPGTTGSGAGSGGPGGLTSAAPAGGDKK

SEQ ID NO: 150 - protein, 36 aa, synthetic construct
  REGION 1..36: note = Synthetic Polypeptide
AATQGAAAAAGSGAGTGGGTASGGTEGGSAESEGAK

SEQ ID NO: 151 - protein, 19 aa, synthetic construct
  REGION 1..19: note = Synthetic Polypeptide
GSSGGSGAKPSDAASEAAR

SEQ ID NO: 152 - protein, 21 aa, synthetic construct
  REGION 1..21: note = Synthetic Polypeptide
IQFHFHWGSLDGQGSEHTVDK

SEQ ID NO: 153 - protein, 18 aa, synthetic construct
  REGION 1..18: note = Synthetic Polypeptide
MILIQDGSQNTNVDKPLR

SEQ ID NO: 154 - protein, 10 aa, synthetic construct
  REGION 1..10: note = Synthetic Polypeptide
KGTFTDDLHK

SEQ ID NO: 155 - protein, 25 aa, synthetic construct
  REGION 1..25: note = Synthetic Polypeptide
QQSHFAMMHGGTGFAGIDSSSPEVK

SEQ ID NO: 156 - protein, 20 aa, synthetic construct
  REGION 1..20: note = Synthetic Polypeptide
VAVLISGTGSNLQALIDSTR

SEQ ID NO: 157 - protein, 16 aa, synthetic construct
  REGION 1..16: note = Synthetic Polypeptide
FLAAGTHLGGTNLDFQ

SEQ ID NO: 158 - protein, 14 aa, synthetic construct
  REGION 1..14: note = Synthetic Polypeptide
LVLGTHTSDEQNHL

SEQ ID NO: 159 - protein, 20 aa, synthetic construct
  REGION 1..20: note = Synthetic Polypeptide
TGGVDTAATGGVFDISNLDR

SEQ ID NO: 160 - protein, 20 aa, synthetic construct
  REGION 1..20: note = Synthetic Polypeptide
TGGVDTAAVGGVFDVSNADR

SEQ ID NO: 161 - protein, 18 aa, synthetic construct
  REGION 1..18: note = Synthetic Polypeptide
  VARIANT 3: note = misc_feature - Xaa can be any naturally occurring amino acid
AVXIVAAGVGEFEAGISK

SEQ ID NO: 162 - protein, 20 aa, synthetic construct
  REGION 1..20: note = Synthetic Polypeptide
EILTLLQGVHQGAGFQDIPK

SEQ ID NO: 163 - protein, 17 aa, synthetic construct
  REGION 1..17: note = Synthetic Polypeptide
MKPLMGVIYVPLTDKEK

SEQ ID NO: 164 - protein, 20 aa, synthetic construct
  REGION 1..20: note = Synthetic Polypeptide
  VARIANT 5: note = misc_feature - Xaa can be any naturally occurring amino acid
ECISXHVGQAGVQIGNACWE

SEQ ID NO: 165 - protein, 16 aa, synthetic construct
  REGION 1..16: note = Synthetic Polypeptide
HFNALGGWGELQNSVK

SEQ ID NO: 166 - protein, 24 aa, synthetic construct
  REGION 1..24: note = Synthetic Polypeptide
ESCSFGYHAGGWGKPPVDETGKPL

SEQ ID NO: 167 - protein, 9 aa, synthetic construct
  REGION 1..9: note = Synthetic Polypeptide
AGYVTHLMK

SEQ ID NO: 168 - protein, 26 aa, synthetic construct
  REGION 1..26: note = Synthetic Polypeptide
TMFSSEVQFGHAGACANQASETAVAK

SEQ ID NO: 169 - protein, 17 aa, synthetic construct
  REGION 1..17: note = Synthetic Polypeptide
MPFPVNHGASSEDTLLK

SEQ ID NO: 170 - protein, 18 aa, synthetic construct
  REGION 1..18: note = Synthetic Polypeptide
FFLHHLIAEIHTAEIRAT

SEQ ID NO: 171 - protein, 17 aa, synthetic construct
  REGION 1..17: note = Synthetic Polypeptide
  VARIANT 2: note = misc_feature - Xaa can be any naturally occurring amino acid
  VARIANT 5: note = misc_feature - Xaa can be any naturally occurring amino acid
NXSAXQVLIEHIGNLDR

SEQ ID NO: 172 - protein, 15 aa, synthetic construct
  REGION 1..15: note = Synthetic Polypeptide
GGYVLHIGTIYGDLK

SEQ ID NO: 173 - protein, 12 aa, synthetic construct
  REGION 1..12: note = Synthetic Polypeptide
  VARIANT 2: note = misc_feature - Xaa can be any naturally occurring amino acid
DXHLGGEDFDNR

SEQ ID NO: 174 - protein, 20 aa, synthetic construct
  REGION 1..20: note = Synthetic Polypeptide
GILGPPPPSFHLGGPAVGPR

SEQ ID NO: 175 - protein, 19 aa, synthetic construct
  REGION 1..19: note = Synthetic Polypeptide
PTPPPTLHLVPEPAAPPPP

SEQ ID NO: 176 - protein, 25 aa, synthetic construct
  REGION 1..25: note = Synthetic Polypeptide
YGPQYGHPPPPPPPPEYGPHADSPV

SEQ ID NO: 177 - protein, 17 aa, synthetic construct
  REGION 1..17: note = Synthetic Polypeptide
KHSGPNSADSANDGFVR

SEQ ID NO: 178 - protein, 16 aa, synthetic construct
  REGION 1..16: note = Synthetic Polypeptide
RPELLTHSTTEVTQPR

SEQ ID NO: 179 - protein, 17 aa, synthetic construct
  REGION 1..17: note = Synthetic Polypeptide
  VARIANT 2: note = misc_feature - Xaa can be any naturally occurring amino acid
LXGHVGFDSLPDQLVNK

SEQ ID NO: 180 - protein, 19 aa, synthetic construct
  REGION 1..19: note = Synthetic Polypeptide
AASATQTIAAAQHAASTPK

SEQ ID NO: 181 - protein, 14 aa, synthetic construct
  REGION 1..14: note = Synthetic Polypeptide
CLTQSGIAGGYKPF

SEQ ID NO: 182 - protein, 18 aa, synthetic construct
  REGION 1..18: note = Synthetic Polypeptide
ELAQIAGRPTEDEDEKEK

SEQ ID NO: 183 - protein, 16 aa, synthetic construct
  REGION 1..16: note = Synthetic Polypeptide
AITIAGVPQSVTECVK

SEQ ID NO: 184 - protein, 12 aa, synthetic construct
  REGION 1..12: note = Synthetic Polypeptide
GLCAIAQAESLR

SEQ ID NO: 185 - protein, 21 aa, synthetic construct
  REGION 1..21: note = Synthetic Polypeptide
KPTALIGVAAIGGAFSEQILK

SEQ ID NO: 186 - protein, 21 aa, synthetic construct
  REGION 1..21: note = Synthetic Polypeptide
DYMNVQCHACIGGTNVGEDIR

SEQ ID NO: 187 - protein, 23 aa, synthetic construct
  REGION 1..23: note = Synthetic Polypeptide
NTQNFQSLHNIGSVVQHSEGKPL

SEQ ID NO: 188 - protein, 22 aa, synthetic construct
  REGION 1..22: note = Synthetic Polypeptide
LKPPTLIHGQAPSAGLPSQKPK

SEQ ID NO: 189 - protein, 13 aa, synthetic construct
  REGION 1..13: note = Synthetic Polypeptide
VLIIGGGDGGVLR

SEQ ID NO: 190 - protein, 17 aa, synthetic construct
  REGION 1..17: note = Synthetic Polypeptide
GCITIIGGGDTATCCAK

SEQ ID NO: 191 - protein, 17 aa, synthetic construct
  REGION 1..17: note = Synthetic Polypeptide
GRPSETGIIGIIDPECR

SEQ ID NO: 192 - protein, 20 aa, synthetic construct
  REGION 1..20: note = Synthetic Polypeptide
EAFGWHAIIVDGHSVEELCK

SEQ ID NO: 193 - protein, 17 aa, synthetic construct
  REGION 1..17: note = Synthetic Polypeptide
LAAAILGGVDQIHIKPG

SEQ ID NO: 194 - protein, 14 aa, synthetic construct
  REGION 1..14: note = Synthetic Polypeptide
LYSILGTTLKDEGK

SEQ ID NO: 195 - protein, 18 aa, synthetic construct
  REGION 1..18: note = Synthetic Polypeptide
MILIQDGSQNTNVDKPLR

SEQ ID NO: 196 - protein, 17 aa, synthetic construct
  REGION 1..17: note = Synthetic Polypeptide
LAMQEFMILPVGAANFR

SEQ ID NO: 197 - protein, 18 aa, synthetic construct
  REGION 1..18: note = Synthetic Polypeptide
VPYLIAGIQHSCQDIGAK

SEQ ID NO: 198 - protein, 18 aa, synthetic construct
  REGION 1..18: note = Synthetic Polypeptide
TVAGGVHISGLHTESAPR

SEQ ID NO: 199 - protein, 20 aa, synthetic construct
  REGION 1..20: note = Synthetic Polypeptide
VAVLISGTGSNLQALIDSTR

SEQ ID NO: 200 - protein, 25 aa, synthetic construct
  REGION 1..25: note = Synthetic Polypeptide
GITAIGGTSTISSEGTQHSYSEEEK

SEQ ID NO: 201 - protein, 18 aa, synthetic construct
  REGION 1..18: note = Synthetic Polypeptide
AGVSISVVHGNLSEEAAK

SEQ ID NO: 202 - protein, 26 aa, synthetic construct
  REGION 1..26: note = Synthetic Polypeptide
HVTQAHVQTGITAAPPPHPGAPHPPQ

SEQ ID NO: 203 - protein, 21 aa, synthetic construct
  REGION 1..21: note = Synthetic Polypeptide
AGLFLPGSVGITDPCESGNFR

SEQ ID NO: 204 - protein, 16 aa, synthetic construct
  REGION 1..16: note = Synthetic Polypeptide
AFAHITGGGLLENIPR

SEQ ID NO: 205 - protein, 12 aa, synthetic construct
  REGION 1..12: note = Synthetic Polypeptide
ILAQITGTEHLK

SEQ ID NO: 206 - protein, 16 aa, synthetic construct
  REGION 1..16: note = Synthetic Polypeptide
  VARIANT 3: note = misc_feature - Xaa can be any naturally occurring amino acid
TFXNITPAEVGVLVGK

SEQ ID NO: 207 - protein, 15 aa, synthetic construct
  REGION 1..15: note = Synthetic Polypeptide
HSSGIVADLSEQSLK

SEQ ID NO: 208 - protein, 23 aa, synthetic construct
  REGION 1..23: note = Synthetic Polypeptide
EDGNEEDKENQGDETQGQQPPQR

SEQ ID NO: 209 - protein, 22 aa, synthetic construct
  REGION 1..22: note = Synthetic Polypeptide
PGPSGITIPGKPGAQGVPGPPG

SEQ ID NO: 210 - protein, 22 aa, synthetic construct
  REGION 1..22: note = Synthetic Polypeptide
GLTKPAALAAAPAKPGGAGGSK

SEQ ID NO: 211 - protein, 13 aa, synthetic construct
  REGION 1..13: note = Synthetic Polypeptide
LGAQLADLHLDNK

SEQ ID NO: 212 - protein, 18 aa, synthetic construct
  REGION 1..18: note = Synthetic Polypeptide
SLVASLAEPDFVVTDFAK

SEQ ID NO: 213 - protein, 19 aa, synthetic construct
  REGION 1..19: note = Synthetic Polypeptide
MSLPLLAGGVADDINTNKK

SEQ ID NO: 214 - protein, 22 aa, synthetic construct
  REGION 1..22: note = Synthetic Polypeptide
QPYAVSELAGHQTSAESWGTGR

SEQ ID NO: 215 - protein, 14 aa, synthetic construct
  REGION 1..14: note = Synthetic Polypeptide
VTVAGLAGKDPVQC

SEQ ID NO: 216 - protein, 13 aa, synthetic construct
  REGION 1..13: note = Synthetic Polypeptide
IITLAGPTNAIFK

SEQ ID NO: 217 - protein, 14 aa, synthetic construct
  REGION 1..14: note = Synthetic Polypeptide
STHGLAILGPENPK

SEQ ID NO: 218 - protein, 15 aa, synthetic construct
  REGION 1..15: note = Synthetic Polypeptide
ASAELALGENSEVLK

SEQ ID NO: 219 - protein, 19 aa, synthetic construct
  REGION 1..19: note = Synthetic Polypeptide
ILISLATGHREEGGENLDQ

SEQ ID NO: 220 - protein, 18 aa, synthetic construct
  REGION 1..18: note = Synthetic Polypeptide
AMSRPFGVALLFGGVDEK

SEQ ID NO: 221 - protein, 21 aa, synthetic construct
  REGION 1..21: note = Synthetic Polypeptide
LQATAHAQAQLGCPVIIHPGR

SEQ ID NO: 222 - protein, 16 aa, synthetic construct
  REGION 1..16: note = Synthetic Polypeptide
ILAGLGFDPEMQNRPT

SEQ ID NO: 223 - protein, 32 aa, synthetic construct
  REGION 1..32: note = Synthetic Polypeptide
PERPQQLPHGLGGIGMGLGPGGQPIDANHLNK

SEQ ID NO: 224 - protein, 24 aa, synthetic construct
  REGION 1..24: note = Synthetic Polypeptide
QLMQLIGPAGLGGLGGLGALTGPG

SEQ ID NO: 225 - protein, 16 aa, synthetic construct
  REGION 1..16: note = Synthetic Polypeptide
HFNALGGWGELQNSVK

SEQ ID NO: 226 - protein, 11 aa, synthetic construct
  REGION 1..11: note = Synthetic Polypeptide
MGAGLGHGMDR

SEQ ID NO: 227 - protein, 22 aa, synthetic construct
  REGION 1..22: note = Synthetic Polypeptide
THMTAIVGMALGHRPIPNQPPT

SEQ ID NO: 228 - protein, 25 aa, synthetic construct
  REGION 1..25: note = Synthetic Polypeptide
PHGLGGIGMGLGPGGQPIDANHLNK

SEQ ID NO: 229 - protein, 19 aa, synthetic construct
  REGION 1..19: note = Synthetic Polypeptide
ASQGDSISSQLGPIHPPPR

SEQ ID NO: 230 - protein, 18 aa, synthetic construct
  REGION 1..18: note = Synthetic Polypeptide
VWQLGSSSPNFTLEGHEK

SEQ ID NO: 231 - protein, 12 aa, synthetic construct
  REGION 1..12: note = Synthetic Polypeptide
YVATLGVEVHPL

SEQ ID NO: 232 - protein, 13 aa, synthetic construct
  REGION 1..13: note = Synthetic Polypeptide
KLIADYSPDDIFN

SEQ ID NO: 233 - protein, 13 aa, synthetic construct
  REGION 1..13: note = Synthetic Polypeptide
  VARIANT 2: note = misc_feature - Xaa can be any naturally occurring amino acid
TXGLIFVVDSNDR

SEQ ID NO: 234 - protein, 17 aa, synthetic construct
  REGION 1..17: note = Synthetic Polypeptide
VPEFQFLIGDEAATHLK

SEQ ID NO: 235 - protein, 18 aa, synthetic construct
  REGION 1..18: note = Synthetic Polypeptide
CNINLLPLPDPIPSGLME

SEQ ID NO: 236 - protein, 22 aa, synthetic construct
  REGION 1..22: note = Synthetic Polypeptide
LITEMVALNPDFKPPADYKPPA

SEQ ID NO: 237 - protein, 15 aa, synthetic construct
  REGION 1..15: note = Synthetic Polypeptide
NQVALNPQNTVFDAK

SEQ ID NO: 238 - protein, 15 aa, synthetic construct
  REGION 1..15: note = Synthetic Polypeptide
GLLKPGLNVVLEGPK

SEQ ID NO: 239 - protein, 17 aa, synthetic construct
  REGION 1..17: note = Synthetic Polypeptide
GVNLPGAAVDLPAVSEK

SEQ ID NO: 240 - protein, 20 aa, synthetic construct
  REGION 1..20: note = Synthetic Polypeptide
  VARIANT 3: note = misc_feature - Xaa can be any naturally occurring amino acid
ISXGLPVGAVINCADNTGAK

SEQ ID NO: 241 - protein, 14 aa, synthetic construct
  REGION 1..14: note = Synthetic Polypeptide
GQVCLPVISAENWK

SEQ ID NO: 242 - protein, 20 aa, synthetic construct
  REGION 1..20: note = Synthetic Polypeptide
EILTLLQGVHQGAGFQDIPK

SEQ ID NO: 243 - protein, 20 aa, synthetic construct
  REGION 1..20: note = Synthetic Polypeptide
NNQFQALLQYADPVSAQHAK

SEQ ID NO: 244 - protein, 16 aa, synthetic construct
  REGION 1..16: note = Synthetic Polypeptide
LFIGGLSFETTDDSLR

SEQ ID NO: 245 - protein, 14 aa, synthetic construct
  REGION 1..14: note = Synthetic Polypeptide
AIQLSGAEQLEALK

SEQ ID NO: 246 - protein, 20 aa, synthetic construct
  REGION 1..20: note = Synthetic Polypeptide
DVSIEDSVISLSGDHCIIGR

SEQ ID NO: 247 - protein, 16 aa, synthetic construct
  REGION 1..16: note = Synthetic Polypeptide
EYLLSGDISEAEHCLK

SEQ ID NO: 248 - protein, 20 aa, synthetic construct
  REGION 1..20: note = Synthetic Polypeptide
VVISSDGQFALSGSWDGTLR

SEQ ID NO: 249 - protein, 17 aa, synthetic construct
  REGION 1..17: note = Synthetic Polypeptide
VHEQLAALSQGPISKPK

SEQ ID NO: 250 - protein, 23 aa, synthetic construct
  REGION 1..23: note = Synthetic Polypeptide
  VARIANT 3: note = misc_feature - Xaa can be any naturally occurring amino acid
  VARIANT 5..6: note = misc_feature - Xaa can be any naturally occurring amino acid
LVXLXXETALLSSGFSLEDPQTH

SEQ ID NO: 251 - protein, 17 aa, synthetic construct
  REGION 1..17: note = Synthetic Polypeptide
GPDGLTAFEATDNQAIK

SEQ ID NO: 252 - protein, 18 aa, synthetic construct
  REGION 1..18: note = Synthetic Polypeptide
ALYWLSGLTCTEQNFISK

SEQ ID NO: 253 - protein, 13 aa, synthetic construct
  REGION 1..13: note = Synthetic Polypeptide
IITLTGPTNAIFK

SEQ ID NO: 254 - protein, 13 aa, synthetic construct
  REGION 1..13: note = Synthetic Polypeptide
LATQLTGPVMPVR

SEQ ID NO: 255 - protein, 14 aa, synthetic construct
  REGION 1..14: note = Synthetic Polypeptide
FPSLLTHNENMVAK

SEQ ID NO: 256 - protein, 19 aa, synthetic construct
  REGION 1..19: note = Synthetic Polypeptide
  VARIANT 3: note = misc_feature - Xaa can be any naturally occurring amino acid
  VARIANT 5: note = misc_feature - Xaa can be any naturally occurring amino acid
  VARIANT 9: note = misc_feature - Xaa can be any naturally occurring amino acid
LEXLXTINXGLTSIANLPK

SEQ ID NO: 257 - protein, 15 aa, synthetic construct
  REGION 1..15: note = Synthetic Polypeptide
ALLLLLVGGVDQSPR

SEQ ID NO: 258 - protein, 18 aa, synthetic construct
  REGION 1..18: note = Synthetic Polypeptide
GKPVGLVGVTELSDAQKK

SEQ ID NO: 259 - protein, 15 aa, synthetic construct
  REGION 1..15: note = Synthetic Polypeptide
VNVAGLVLAGSADFK

SEQ ID NO: 260 - protein, 19 aa, synthetic construct
  REGION 1..19: note = Synthetic Polypeptide
QGYIGAALVLGGVDVTGPH

SEQ ID NO: 261 - protein, 14 aa, synthetic construct
  REGION 1..14: note = Synthetic Polypeptide
LYTLVLTDPDAPSR

SEQ ID NO: 262 - protein, 14 aa, synthetic construct
  REGION 1..14: note = Synthetic Polypeptide
AQIHDLVLVGGSTR

SEQ ID NO: 263 - protein, 20 aa, synthetic construct
  REGION 1..20: note = Synthetic Polypeptide
LNHVAAGLVSPSLKSDTSSK

SEQ ID NO: 264 - protein, 16 aa, synthetic construct
  REGION 1..16: note = Synthetic Polypeptide
IEVGLVVGNSQVAFEK

SEQ ID NO: 265 - protein, 21 aa, synthetic construct
  REGION 1..21: note = Synthetic Polypeptide
GYHQSASEHGLVVIAPDTSPR

SEQ ID NO: 266 - protein, 21 aa, synthetic construct
  REGION 1..21: note = Synthetic Polypeptide
GYHQSASEHGLVVIAPDTSPR

SEQ ID NO: 267 - protein, 20 aa, synthetic construct
  REGION 1..20: note = Synthetic Polypeptide
QDHPWLLSQNLVVKPDQLIK

SEQ ID NO: 268 - protein, 15 aa, synthetic construct
  REGION 1..15: note = Synthetic Polypeptide
MGLAMGGGGGASFDR

SEQ ID NO: 269 - protein, 27 aa, synthetic construct
  REGION 1..27: note = Synthetic Polypeptide
QLPHGLGGIGMGLGPGGQPIDANHLNK

SEQ ID NO: 270 - protein, 16 aa, synthetic construct
  REGION 1..16: note = Synthetic Polypeptide
VVVLMGSTSDLGHCEK

SEQ ID NO: 271 - protein, 15 aa, synthetic construct
  REGION 1..15: note = Synthetic Polypeptide
MALIQMGSVEEAVQA

SEQ ID NO: 272 - protein, 15 aa, synthetic construct
  REGION 1..15: note = Synthetic Polypeptide
TTGFGMIYDSLDYAK

SEQ ID NO: 273 - protein, 16 aa, synthetic construct
  REGION 1..16: note = Synthetic Polypeptide
WLLAEMLGDLSDSQLK

SEQ ID NO: 274 - protein, 17 aa, synthetic construct
  REGION 1..17: note = Synthetic Polypeptide
QAQYLGMSCDGPFKPDH

SEQ ID NO: 275 - protein, 13 aa, synthetic construct
  REGION 1..13: note = Synthetic Polypeptide
AHSSMVGVNLPQK

SEQ ID NO: 276 - protein, 16 aa, synthetic construct
  REGION 1..16: note = Synthetic Polypeptide
SGPVVAMVWEGLNVVK

SEQ ID NO: 277 - protein, 24 aa, synthetic construct
  REGION 1..24: note = Synthetic Polypeptide
VNTQNFQSLHNIGSVVQHSEGKPL

SEQ ID NO: 278 - protein, 12 aa, synthetic construct
  REGION 1..12: note = Synthetic Polypeptide
LYVSNLGIGHTR

SEQ ID NO: 279 - protein, 17 aa, synthetic construct
  REGION 1..17: note = Synthetic Polypeptide
VYVGNLGNNGNKTELER

SEQ ID NO: 280 - protein, 18 aa, synthetic construct
  REGION 1..18: note = Synthetic Polypeptide
IVDLLQMLEMNMAIAFPA

SEQ ID NO: 281 - protein, 16 aa, synthetic construct
  REGION 1..16: note = Synthetic Polypeptide
VLAQNSGFDLQETLVK

SEQ ID NO: 282 - protein, 25 aa, synthetic construct
  REGION 1..25: note = Synthetic Polypeptide
QQSHFPMTHGNTGFSGIESSSPEVK

SEQ ID NO: 283 - protein, 14 aa, synthetic construct
  REGION 1..14: note = Synthetic Polypeptide
ILIANTGMDTDKIK

SEQ ID NO: 284 - protein, 12 aa, synthetic construct
  REGION 1..12: note = Synthetic Polypeptide
NNTVTPGGKPNK

SEQ ID NO: 285 - protein, 19 aa, synthetic construct
  REGION 1..19: note = Synthetic Polypeptide
VVNVANVGAVPSGQDNIHR

SEQ ID NO: 286 - protein, 17 aa, synthetic construct
  REGION 1..17: note = Synthetic Polypeptide
MPFPVNHGASSEDTLLK

SEQ ID NO: 287 - protein, 9 aa, synthetic construct
  REGION 1..9: note = Synthetic Polypeptide
RPKDPGHPY

SEQ ID NO: 288 - protein, 15 aa, synthetic construct
  REGION 1..15: note = Synthetic Polypeptide
ELDIMEPKVPDDIYK

SEQ ID NO: 289 - protein, 22 aa, synthetic construct
  REGION 1..22: note = Synthetic Polypeptide
AETSQQEASEGGDPASPALSLS

SEQ ID NO: 290 - protein, 16 aa, synthetic construct
  REGION 1..16: note = Synthetic Polypeptide
LLAAQNPLSQADRPHQ

SEQ ID NO: 291 - protein, 17 aa, synthetic construct
  REGION 1..17: note = Synthetic Polypeptide
  VARIANT 5: note = misc_feature - Xaa can be any naturally occurring amino acid
PDNFXFGQSGAGNNWAK

SEQ ID NO: 292 - protein, 16 aa, synthetic construct
  REGION 1..16: note = Synthetic Polypeptide
MIAGQVLDINLAAEPK

SEQ ID NO: 293 - protein, 22 aa, synthetic construct
  REGION 1..22: note = Synthetic Polypeptide
IILNSHSPAGSAAISQQDFHPK

SEQ ID NO: 294 - protein, 26 aa, synthetic construct
  REGION 1..26: note = Synthetic Polypeptide
GAVAVSAAPGSAAPAAGSAPAAAEEK

SEQ ID NO: 295 - protein, 47 aa, synthetic construct
  REGION 1..47: note = Synthetic Polypeptide
SAAGAAGSAGGSSGAAGAAGGGAGAGTRPGDGGTASAGAAGPGAATK

SEQ ID NO: 296 - protein, 20 aa, synthetic construct
  REGION 1..20: note = Synthetic Polypeptide
FTASAGIQVVGDDLTVTNPK

SEQ ID NO: 297 - protein, 20 aa, synthetic construct
  REGION 1..20: note = Synthetic Polypeptide
FGIVTSSAGTGTTEDTEAKK

SEQ ID NO: 298 - protein, 24 aa, synthetic construct
  REGION 1..24: note = Synthetic Polypeptide
SLYQSAGVAPESFEYIEAHGTGTK

SEQ ID NO: 299 - protein, 13 aa, synthetic construct
  REGION 1..13: note = Synthetic Polypeptide
VSEIDEMFEARKM

SEQ ID NO: 300 - protein, 18 aa, synthetic construct
  REGION 1..18: note = Synthetic Polypeptide
FGGSFAGSFGGAGGHAPG

SEQ ID NO: 301 - protein, 21 aa, synthetic construct
  REGION 1..21: note = Synthetic Polypeptide
FGGSFAGSFGGAGGHAPGVAR

SEQ ID NO: 302 - protein, 21 aa, synthetic construct
  REGION 1..21: note = Synthetic Polypeptide
FGGSFAGSFGGAGGHAPGVAR

SEQ ID NO: 303 - protein, 21 aa, synthetic construct
  REGION 1..21: note = Synthetic Polypeptide
FGGSFAGSFGGAGGHAPGVAR

SEQ ID NO: 304 - protein, 32 aa, synthetic construct
  REGION 1..32: note = Synthetic Polypeptide
AADTKPGTTGSGAGSGGPGGLTSAAPAGGDKK

SEQ ID NO: 305 - protein, 35 aa, synthetic construct
  REGION 1..35: note = Synthetic Polypeptide
ATQGAAAAAGSGAGTGGGTASGGTEGGSAESEGAK

SEQ ID NO: 306 - protein, 15 aa, synthetic construct
  REGION 1..15: note = Synthetic Polypeptide
EIELIGSGGFGQVFK

SEQ ID NO: 307 - protein, 28 aa, synthetic construct
  REGION 1..28: note = Synthetic Polypeptide
KPGTTGSGAGSGGPGGLTSAAPAGGDKK

SEQ ID NO: 308 - protein, 20 aa, synthetic construct
  REGION 1..20: note = Synthetic Polypeptide
  VARIANT 5: note = misc_feature - Xaa can be any naturally occurring amino acid
  VARIANT 7: note = misc_feature - Xaa can be any naturally occurring amino acid
LYANXVXSGGTTMYPGIADR

SEQ ID NO: 309 - protein, 10 aa, synthetic construct
  REGION 1..10: note = Synthetic Polypeptide
RSGKYDLDFK

SEQ ID NO: 310 - protein, 16 aa, synthetic construct
  REGION 1..16: note = Synthetic Polypeptide
HDGYGSHGPLLPLPSR

SEQ ID NO: 311 - protein, 13 aa, synthetic construct
  REGION 1..13: note = Synthetic Polypeptide
SLFSSIGEVESAK

SEQ ID NO: 312 - protein, 13 aa, synthetic construct
  REGION 1..13: note = Synthetic Polypeptide
LQSIGTENTEENR

SEQ ID NO: 313 - protein, 18 aa, synthetic construct
  REGION 1..18: note = Synthetic Polypeptide
SLVASLAEPDFVVTDFAK

SEQ ID NO: 314 - protein, 21 aa, synthetic construct
  REGION 1..21: note = Synthetic Polypeptide
AEPMGEKPVGSLAGIGEVLGK

SEQ ID NO: 315 - protein, 15 aa, synthetic construct
  REGION 1..15: note = Synthetic Polypeptide
VQEAINSLGGSVFPK

SEQ ID NO: 316 - protein, 24 aa, synthetic construct
  REGION 1..24: note = Synthetic Polypeptide
SAAAASAASGSPGPGEGSAGGEKR

SEQ ID NO: 317 - protein, 19 aa, synthetic construct
  REGION 1..19: note = Synthetic Polypeptide
QTIDNSQGAYQEAFDISKK

SEQ ID NO: 318 - protein, 19 aa, synthetic construct
  REGION 1..19: note = Synthetic Polypeptide
FGIVTSSAGTGTTEDTEAK

SEQ ID NO: 319 - protein, 20 aa, synthetic construct
  REGION 1..20: note = Synthetic Polypeptide
FGIVTSSAGTGTTEDTEAKK

SEQ ID NO: 320 - protein, 12 aa, synthetic construct
  REGION 1..12: note = Synthetic Polypeptide
  VARIANT 1: note = misc_feature - Xaa can be any naturally occurring amino acid
XSSFDLDYDFQR

SEQ ID NO: 321 - protein, 21 aa, synthetic construct
  REGION 1..21: note = Synthetic Polypeptide
FQAGTSKPLHSSGINVNAAPF

SEQ ID NO: 322 - protein, 21 aa, synthetic construct
  REGION 1..21: note = Synthetic Polypeptide
HIGGPPGFASSSGKPGPTVIK

SEQ ID NO: 323 - protein, 21 aa, synthetic construct
  REGION 1..21: note = Synthetic Polypeptide
  VARIANT 1: note = misc_feature - Xaa can be any naturally occurring amino acid
XSSGPGASSGTSGDHGELVVR

SEQ ID NO: 324 - protein, 19 aa, synthetic construct
  REGION 1..19: note = Synthetic Polypeptide
ELVSSSSSGSDSDSEVDKK

SEQ ID NO: 325 - protein, 12 aa, synthetic construct
  REGION 1..12: note = Synthetic Polypeptide
MDSTEPPYSQKR

SEQ ID NO: 326 - protein, 16 aa, synthetic construct
  REGION 1..16: note = Synthetic Polypeptide
VVVLMGSTSDLGHCEK

SEQ ID NO: 327 - protein, 16 aa, synthetic construct
  REGION 1..16: note = Synthetic Polypeptide
ILDSVGIEADDDRLNK

SEQ ID NO: 328 - protein, 16 aa, synthetic construct
  REGION 1..16: note = Synthetic Polypeptide
STQPISSVGKPASVIK

SEQ ID NO: 329 - protein, 14 aa, synthetic construct
  REGION 1..14: note = Synthetic Polypeptide
ALQSVGQIVGEVLK

SEQ ID NO: 330 - protein, 22 aa, synthetic construct
  REGION 1..22: note = Synthetic Polypeptide
VSSLAEGSVTSVGSVNPAENFR

SEQ ID NO: 331 - protein, 16 aa, synthetic construct
  REGION 1..16: note = Synthetic Polypeptide
TGSISSSVSVPAKPER

SEQ ID NO: 332 - protein, 19 aa, synthetic construct
  REGION 1..19: note = Synthetic Polypeptide
  VARIANT 2..7: note = misc_feature - Xaa can be any naturally occurring amino acid
YXXXXXXYSQSYGGYENQK

SEQ ID NO: 333 - protein, 13 aa, synthetic construct
  REGION 1..13: note = Synthetic Polypeptide
IYWGTATTGKPHV

SEQ ID NO: 334 - protein, 11 aa, synthetic construct
  REGION 1..11: note = Synthetic Polypeptide
MVQTAVVPVKK

SEQ ID NO: 335 - protein, 14 aa, synthetic construct
  REGION 1..14: note = Synthetic Polypeptide
MMLGTEGGEGFVVK

SEQ ID NO: 336 - protein, 13 aa, synthetic construct
  REGION 1..13: note = Synthetic Polypeptide
  VARIANT 1: note = misc_feature - Xaa can be any naturally occurring amino acid
XTFIAIKPDGVQR

SEQ ID NO: 337 - protein, 18 aa, synthetic construct
  REGION 1..18: note = Synthetic Polypeptide
VSHVSTGGGASLELLEGK

SEQ ID NO: 338 - protein, 31 aa, synthetic construct
  REGION 1..31: note = Synthetic Polypeptide
AAAAAGSGAGTGGGTASGGTEGGSAESEGAK

SEQ ID NO: 339 - protein, 21 aa, synthetic construct
  REGION 1..21: note = Synthetic Polypeptide
TIGNSCGTIGLIHAVANNQDK

SEQ ID NO: 340 - protein, 16 aa, synthetic construct
  REGION 1..16: note = Synthetic Polypeptide
TGEEIFGTIGMRPNAK

SEQ ID NO: 341 - protein, 20 aa, synthetic construct
  REGION 1..20: note = Synthetic Polypeptide
TTQFSCTLGEKFEETTADGR

SEQ ID NO: 342 - protein, 11 aa, synthetic construct
  REGION 1..11: note = Synthetic Polypeptide
GCTATLGNFAK

SEQ ID NO: 343 - protein, 12 aa, synthetic construct
  REGION 1..12: note = Synthetic Polypeptide
YVATLGVEVHPL

SEQ ID NO: 344 - protein, 15 aa, synthetic construct
  REGION 1..15: note = Synthetic Polypeptide
LAATNALLNSLEFTK

SEQ ID NO: 345 - protein, 18 aa, synthetic construct
  REGION 1..18: note = Synthetic Polypeptide
GPGASSGTSGDHGELVVR

SEQ ID NO: 346 - protein, 10 aa, synthetic construct
  REGION 1..10: note = Synthetic Polypeptide
STTTGHLIYK

SEQ ID NO: 347 - protein, 35 aa, synthetic construct
  REGION 1..35: note = Synthetic Polypeptide
ALSAADTKPGTTGSGAGSGGPGGLTSAAPAGGDKK

SEQ ID NO: 348 - protein, 10 aa, synthetic construct
  REGION 1..10: note = Synthetic Polypeptide
STTTGHLIYK

SEQ ID NO: 349 - protein, 18 aa, synthetic construct
  REGION 1..18: note = Synthetic Polypeptide
VTIIGPATVGGIKPGCFK

SEQ ID NO: 350 - protein, 22 aa, synthetic construct
  REGION 1..22: note = Synthetic Polypeptide
TVVFSHPPIGTVGLTEDEAIHK

SEQ ID NO: 351 - protein, 19 aa, synthetic construct
  REGION 1..19: note = Synthetic Polypeptide
GSPTSLGTWGSWIGPDHDK

SEQ ID NO: 352 - protein, 20 aa, synthetic construct
  REGION 1..20: note = Synthetic Polypeptide
GSPTSLGTWGSWIGPDHDKF

SEQ ID NO: 353 - protein, 16 aa, synthetic construct
  REGION 1..16: note = Synthetic Polypeptide
IHFPLATYAPVISAEK

SEQ ID NO: 354 - protein, 13 aa, synthetic construct
  REGION 1..13: note = Synthetic Polypeptide
ANPQVGVAFPHIK

SEQ ID NO: 355 - protein, 15 aa, synthetic construct
  REGION 1..15: note = Synthetic Polypeptide
TCTTVAFTQVNSEDK

SEQ ID NO: 356 - protein, 16 aa, synthetic construct
  REGION 1..16: note = Synthetic Polypeptide
VLTGVAGEDAECHAAK

SEQ ID NO: 357 - protein, 20 aa, synthetic construct
  REGION 1..20: note = Synthetic Polypeptide
NIPPYFVALVPQEEELDDQK

SEQ ID NO: 358 - protein, 19 aa, synthetic construct
  REGION 1..19: note = Synthetic Polypeptide
GQETAVAPSLVAPALNKPK

SEQ ID NO: 359 - protein, 20 aa, synthetic construct
  REGION 1..20: note = Synthetic Polypeptide
QGQETAVAPSLVAPALNKPK

SEQ ID NO: 360 - protein, 20 aa, synthetic construct
  REGION 1..20: note = Synthetic Polypeptide
GFVTFSSMAEVDAAMAARPH

SEQ ID NO: 361 - protein, 17 aa, synthetic construct
  REGION 1..17: note = Synthetic Polypeptide
VDYYTTTPALVFGKPVR

SEQ ID NO: 362 - protein, 17 aa, synthetic construct
  REGION 1..17: note = Synthetic Polypeptide
VDYYTTTPALVFGKPVR

SEQ ID NO: 363 - protein, 27 aa, synthetic construct
  REGION 1..27: note = Synthetic Polypeptide
  VARIANT 5: note = misc_feature - Xaa can be any naturally occurring amino acid
ASQPXVDGFLVGGASLKPEFVDIINAK

SEQ ID NO: 364 - protein, 17 aa, synthetic construct
  REGION 1..17: note = Synthetic Polypeptide
TIIGPATVGGIKPGCFK

SEQ ID NO: 365 - protein, 19 aa, synthetic construct
  REGION 1..19: note = Synthetic Polypeptide
GGVDTAAVGGVFDVSNADR

SEQ ID NO: 366 - protein, 11 aa, synthetic construct
  REGION 1..11: note = Synthetic Polypeptide
TTVHAITATQK

SEQ ID NO: 367 - protein, 20 aa, synthetic construct
  REGION 1..20: note = Synthetic Polypeptide
EEVRPQDTVSVIGGVAGGSK

SEQ ID NO: 368 - protein, 11 aa, synthetic construct
  REGION 1..11: note = Synthetic Polypeptide
QVIGTGSFFPK

SEQ ID NO: 369 - protein, 16 aa, synthetic construct
  REGION 1..16: note = Synthetic Polypeptide
ASGNYATVISHNPETK

SEQ ID NO: 370 - protein, 17 aa, synthetic construct
  REGION 1..17: note = Synthetic Polypeptide
MKPLMGVIYVPLTDKEK

SEQ ID NO: 371 - protein, 16 aa, synthetic construct
  REGION 1..16: note = Synthetic Polypeptide
FSVCVLGDQQHCDEAK

SEQ ID NO: 372 - protein, 21 aa, synthetic construct
  REGION 1..21: note = Synthetic Polypeptide
ENAFCNLAAIVPDSVGRHSPA

SEQ ID NO: 373 - protein, 20 aa, synthetic construct
  REGION 1..20: note = Synthetic Polypeptide
AYVGNLPFNTVQGDIDAIFK

SEQ ID NO: 374 - protein, 15 aa, synthetic construct
  REGION 1..15: note = Synthetic Polypeptide
TLTTVQGIADDYDKK

SEQ ID NO: 375 - protein, 19 aa, synthetic construct
  REGION 1..19: note = Synthetic Polypeptide
  VARIANT 4: note = misc_feature - Xaa can be any naturally occurring amino acid
CISXHVGQAGVQIGNACWE

SEQ ID NO: 376 - protein, 23 aa, synthetic construct
  REGION 1..23: note = Synthetic Polypeptide
THALQWPSLTVQWLPEVTKPEGK

SEQ ID NO: 377 - protein, 32 aa, synthetic construct
  REGION 1..32: note = Synthetic Polypeptide
ASVPAGGAVAVSAAPGSAAPAAGSAPAAAEEK

SEQ ID NO: 378 - protein, 14 aa, synthetic construct
  REGION 1..14: note = Synthetic Polypeptide
YEEVSVSGFEEFHR

SEQ ID NO: 379 - protein, 17 aa, synthetic construct
  REGION 1..17: note = Synthetic Polypeptide
CMTTVSWDGDKLQCVQK

SEQ ID NO: 380 - protein, 16 aa, synthetic construct
  REGION 1..16: note = Synthetic Polypeptide
MHGGGPTVTAGLPLPK

SEQ ID NO: 381 - protein, 20 aa, synthetic construct
  REGION 1..20: note = Synthetic Polypeptide
LALVTGGEIASTFDHPELVK

SEQ ID NO: 382 - protein, 20 aa, synthetic construct
  REGION 1..20: note = Synthetic Polypeptide
LEGTLLKPNMVTPGHACTQK

SEQ ID NO: 383 - protein, 11 aa, synthetic construct
  REGION 1..11: note = Synthetic Polypeptide
  VARIANT 1: note = misc_feature - Xaa can be any naturally occurring amino acid
XVVESAYEVIK

SEQ ID NO: 384 - protein, 15 aa, synthetic construct
  REGION 1..15: note = Synthetic Polypeptide
ILAQVVGDVDTSLPR

SEQ ID NO: 385 - protein, 22 aa, synthetic construct
  REGION 1..22: note = Synthetic Polypeptide
CFSEMAPVCAVVGGILAQEIVK

SEQ ID NO: 386 - protein, 20 aa, synthetic construct
  REGION 1..20: note = Synthetic Polypeptide
  VARIANT 7: note = misc_feature - Xaa can be any naturally occurring amino acid
ETEDTFXADLVVGLCTGQIK

SEQ ID NO: 387 - protein, 14 aa, synthetic construct
  REGION 1..14: note = Synthetic Polypeptide
EGPAVVGQFIQDVK

SEQ ID NO: 388 - protein, 19 aa, synthetic construct
  REGION 1..19: note = Synthetic Polypeptide
MLISGYALNCVVGSQGMPK

SEQ ID NO: 389 - protein, 14 aa, synthetic construct
  REGION 1..14: note = Synthetic Polypeptide
HWPFMVVNDAGRPK

SEQ ID NO: 390 - protein, 16 aa, synthetic construct
  REGION 1..16: note = Synthetic Polypeptide
SGPVVAMVWEGLNVVK

SEQ ID NO: 391 - protein, 18 aa, synthetic construct
  REGION 1..18: note = Synthetic Polypeptide
ALQDEWDAVMLHSFTLRQ

SEQ ID NO: 392 - protein, 14 aa, synthetic construct
  REGION 1..14: note = Synthetic Polypeptide
EYFSWEGAFQHVGK

SEQ ID NO: 393 - protein, 22 aa, synthetic construct
  REGION 1..22: note = Synthetic Polypeptide
ATVASGIPAGWMGLDCGPESSK

SEQ ID NO: 394 - protein, 23 aa, synthetic construct
  REGION 1..23: note = Synthetic Polypeptide
ATVASGIPAGWMGLDCGPESSKK

SEQ ID NO: 395 - protein, 20 aa, synthetic construct
  REGION 1..20: note = Synthetic Polypeptide
DCAFYDPTHAWSGGLDHQLK

SEQ ID NO: 396 - protein, 18 aa, synthetic construct
  REGION 1..18: note = Synthetic Polypeptide
QFQALLQYADPVSAQHAK

SEQ ID NO: 397 - protein, 21 aa, synthetic construct
  REGION 1..21: note = Synthetic Polypeptide
EQPQHPLHVTYAGAAVDELGK

SEQ ID NO: 398 - protein, 12 aa, synthetic construct
  REGION 1..12: note = Synthetic Polypeptide
TFSYAGFEMQPK

SEQ ID NO: 399 - protein, 21 aa, synthetic construct
  REGION 1..21: note = Synthetic Polypeptide
GYIWNYGAIPQTWEDPGHNDK

SEQ ID NO: 400 - protein, 23 aa, synthetic construct
  REGION 1..23: note = Synthetic Polypeptide
DYTGYNNYYGYGDYSNQQSGYGK

SEQ ID NO: 401 - protein, 13 aa, synthetic construct
  REGION 1..13: note = Synthetic Polypeptide
QSGYGGQTKPIFR

SEQ ID NO: 402 - protein, 20 aa, synthetic construct
  REGION 1..20: note = Synthetic Polypeptide
VPLIESGTAGYLGQVTTIKK

SEQ ID NO: 403 - protein, 20 aa, synthetic construct
  REGION 1..20: note = Synthetic Polypeptide
GILGYTEHQVVSSDFNSDTH

SEQ ID NO: 404 - protein, 22 aa, synthetic construct
  REGION 1..22: note = Synthetic Polypeptide
GILGYTEHQVVSSDFNSDTHSS

SEQ ID NO: 405 - protein, 16 aa, synthetic construct
  REGION 1..16: note = Synthetic Polypeptide
  VARIANT 5: note = misc_feature - Xaa can be any naturally occurring amino acid
QTCVXHYTGMLEDGKK

SEQ ID NO: 406 - protein, 19 aa, synthetic construct
  REGION 1..19: note = Synthetic Polypeptide
  VARIANT 5: note = misc_feature - Xaa can be any naturally occurring amino acid
QTCVXHYTGMLEDGKKFDS

SEQ ID NO: 407 - protein, 9 aa, synthetic construct
  REGION 1..9: note = Synthetic Polypeptide
  VARIANT 2: note = misc_feature - Xaa can be any naturally occurring amino acid
AXYVTHLMK

SEQ ID NO: 408 - protein, 5 aa, synthetic construct
  REGION 1..5: note = Synthetic Polypeptide
KVSVR

SEQ ID NO: 409 - protein, 19 aa, synthetic construct
  REGION 1..19: note = Synthetic Polypeptide
MDLAAAAEPGAGSQHLEVR

SEQ ID NO: 410 - protein, 41 aa, synthetic construct; identical to SEQ ID NO: 12
  REGION 1..41: note = Synthetic Polypeptide

SEQ ID NO: 411 - protein, 18 aa, synthetic construct
  REGION 1..18: note = Synthetic Polypeptide
GTSFDAAATSGGSASSEK

SEQ ID NO: 412 - protein, 39 aa, synthetic construct; identical to SEQ ID NO: 13
  REGION 1..39: note = Synthetic Polypeptide

SEQ ID NO: 413 - protein, 14 aa, synthetic construct
  REGION 1..14: note = Synthetic Polypeptide
GILAADESTGSIAK

SEQ ID NO: 414 - protein, 29 aa, synthetic construct
  REGION 1..29: note = Synthetic Polypeptide
PAAPALSAADTKPGTTGSGAGSGGPGGLT

SEQ ID NO: 415 - protein, 38 aa, synthetic construct; identical to SEQ ID NO: 15
  REGION 1..38: note = Synthetic Polypeptide

SEQ ID NO: 416 - protein, 18 aa, synthetic construct
  REGION 1..18: note = Synthetic Polypeptide
SSIQATTAAGSGHPTSCC

SEQ ID NO: 417 - protein, 18 aa, synthetic construct
  REGION 1..18: note = Synthetic Polypeptide
NEAIQAAHDAVAQEGQCR

SEQ ID NO: 418 - protein, 22 aa, synthetic construct
  REGION 1..22: note = Synthetic Polypeptide
QFGLPAEAVEAANKGDVEAFAK

SEQ ID NO: 419 - protein, 21 aa, synthetic construct
  REGION 1..21: note = Synthetic Polypeptide
LVIPNTLAVNAAQDSTDLVAK

SEQ ID NO: 420 - protein, 16 aa, synthetic construct
  REGION 1..16: note = Synthetic Polypeptide
EALAAMNAAQVKPLGK

SEQ ID NO: 421 - protein, 21 aa, synthetic construct
  REGION 1..21: note = Synthetic Polypeptide
APRPPVSAASGRPQDDTDSSR

SEQ ID NO: 422 - protein, 25 aa, synthetic construct
  REGION 1..25: note = Synthetic Polypeptide
GDPQEAKPQEAAVAPEKPPASDETK

SEQ ID NO: 423 - protein, 16 aa, synthetic construct
  REGION 1..16: note = Synthetic Polypeptide
EGDMIVCAAYAHELPK

SEQ ID NO: 424 - protein, 14 aa, synthetic construct
  REGION 1..14: note = Synthetic Polypeptide
GILAADESTGSIAK

SEQ ID NO: 425 - protein, 19 aa, synthetic construct
  REGION 1..19: note = Synthetic Polypeptide
PEEACSFILSADFPALVVK

SEQ ID NO: 426 - protein, 24 aa, synthetic construct
  REGION 1..24: note = Synthetic Polypeptide
GWNAYIDNLMADGTCQDAAIVGYK

SEQ ID NO: 427 - protein, 14 aa, synthetic construct
  REGION 1..14: note = Synthetic Polypeptide
YLAADKDGNVTCER

SEQ ID NO: 428 - protein, 16 aa, synthetic construct
  REGION 1..16: note = Synthetic Polypeptide
LPVDFVTADKFDENAK

SEQ ID NO: 429 - protein, 13 aa, synthetic construct
  REGION 1..13: note = Synthetic Polypeptide
  VARIANT 2: note = misc_feature - Xaa can be any naturally occurring amino acid
TXEAEAAHGTVTR

SEQ ID NO: 430 - protein, 12 aa, synthetic construct
  REGION 1..12: note = Synthetic Polypeptide
TVFAEHISDECK

SEQ ID NO: 431 - protein, 13 aa, synthetic construct
  REGION 1..13: note = Synthetic Polypeptide
TVFAEHISDECKR

SEQ ID NO: 432 - protein, 16 aa, synthetic construct
  REGION 1..16: note = Synthetic Polypeptide
DLEAEHVEVEDTTLNR

SEQ ID NO: 433 - protein, 11 aa, synthetic construct
  REGION 1..11: note = Synthetic Polypeptide
CAEIAHNVSSK

SEQ ID NO: 434 - protein, 28 aa, synthetic construct
  REGION 1..28: note = Synthetic Polypeptide
EAAAAGGGVGAGAGGGCGPGGADSSKPR

SEQ ID NO: 435 - protein, 15 aa, synthetic construct
  REGION 1..15: note = Synthetic Polypeptide
  VARIANT 2: note = misc_feature - Xaa can be any naturally occurring amino acid
YXLVGAGAIGCELLK

SEQ ID NO: 436 - protein, 15 aa, synthetic construct
  REGION 1..15: note = Synthetic Polypeptide
LIYAGKILNDDTALK

SEQ ID NO: 437 - protein, 20 aa, synthetic construct
  REGION 1..20: note = Synthetic Polypeptide
FGDNTAGCTSAGPHFNPLSR

SEQ ID NO: 438 - protein, 13 aa, synthetic construct
  REGION 1..13: note = Synthetic Polypeptide
IITLAGPTNAIFK

SEQ ID NO: 439 - protein, 17 aa, synthetic construct
  REGION 1..17: note = Synthetic Polypeptide
  VARIANT 5: note = misc_feature - Xaa can be any naturally occurring amino acid
EIVHXQAGQCGNQIGAK

SEQ ID NO: 440 - protein, 19 aa, synthetic construct
  REGION 1..19: note = Synthetic Polypeptide
AICAGPTALLAHEIGFGSK

SEQ ID NO: 441 - protein, 18 aa, synthetic construct
  REGION 1..18: note = Synthetic Polypeptide
SHEHSPSDLEAHFVPLVK

SEQ ID NO: 442 - protein, 12 aa, synthetic construct
  REGION 1..12: note = Synthetic Polypeptide
ELQAHGADELLK

SEQ ID NO: 443 - protein, 18 aa, synthetic construct
  REGION 1..18: note = Synthetic Polypeptide
SWADLVNAHVVPGSGVVK

SEQ ID NO: 444 - protein, 12 aa, synthetic construct
  REGION 1..12: note = Synthetic Polypeptide
TFIAIKPDGVQR

SEQ ID NO: 445 - protein, 21 aa, synthetic construct
  REGION 1..21: note = Synthetic Polypeptide
GYIWNYGAIPQTWEDPGHNDK

SEQ ID NO: 446 - protein, 15 aa, synthetic construct
  REGION 1..15: note = Synthetic Polypeptide
  VARIANT 5..6: note = misc_feature - Xaa can be any naturally occurring amino acid
ATATXXAKPQITNPK

SEQ ID NO: 447 - protein, 19 aa, synthetic construct
  REGION 1..19: note = Synthetic Polypeptide
TTETAQHAQGAKPQVQPQK

SEQ ID NO: 448 - protein, 18 aa, synthetic construct
  REGION 1..18: note = Synthetic Polypeptide
VASYLLAALGGNSSPSAK

SEQ ID NO: 449 - protein, 14 aa, synthetic construct
  REGION 1..14: note = Synthetic Polypeptide
IALPAPRGSGTASD

SEQ ID NO: 450 - protein, 14 aa, synthetic construct
  REGION 1..14: note = Synthetic Polypeptide
QIGNVAALPGIVHR

SEQ ID NO: 451 - protein, 21 aa, synthetic construct
  REGION 1..21: note = Synthetic Polypeptide
DGTVLCELINALYPEGQAPVK

SEQ ID NO: 452 - protein, 22 aa, synthetic construct
  REGION 1..22: note = Synthetic Polypeptide
DGTVLCELINALYPEGQAPVKK

SEQ ID NO: 453 - protein, 14 aa, synthetic construct
  REGION 1..14: note = Synthetic Polypeptide
DFTVSAMHGDMDQK

SEQ ID NO: 454 - protein, 16 aa, synthetic construct
  REGION 1..16: note = Synthetic Polypeptide
LVTDCVAAMNPDAVLR

SEQ ID NO: 455 - protein, 14 aa, synthetic construct
  REGION 1..14: note = Synthetic Polypeptide
HELQANCYEEVKDR

SEQ ID NO: 456 - protein, 25 aa, synthetic construct
  REGION 1..25: note = Synthetic Polypeptide
CSLQAAAILDANDAHQTETSSSQVK

SEQ ID NO: 457 - protein, 20 aa, synthetic construct
  REGION 1..20: note = Synthetic Polypeptide
SGLGRPQLQGAPAAEPMAVP

SEQ ID NO: 458 - protein, 18 aa, synthetic construct
  REGION 1..18: note = Synthetic Polypeptide
QETAVAPSLVAPALNKPK

SEQ ID NO: 459 - protein, 15 aa, synthetic construct
  REGION 1..15: note = Synthetic Polypeptide
  VARIANT 3: note = misc_feature - Xaa can be any naturally occurring amino acid
AQXAAPASVPAQAPK

SEQ ID NO: 460 - protein, 21 aa, synthetic construct
  REGION 1..21: note = Synthetic Polypeptide
GETIFVTAPHEATAGIIGVNR

SEQ ID NO: 461 - protein, 18 aa, synthetic construct
  REGION 1..18: note = Synthetic Polypeptide
EYSSELNAPSQESDSHPR

SEQ ID NO: 462 - protein, 20 aa, synthetic construct
  REGION 1..20: note = Synthetic Polypeptide
DQVTAQEIFQDNHEDGPTAK

SEQ ID NO: 463 - protein, 19 aa, synthetic construct
  REGION 1..19: note = Synthetic Polypeptide
LHEEEIQELQAQIQEQHVQ

SEQ ID NO: 464 - protein, 18 aa, synthetic construct
  REGION 1..18: note = Synthetic Polypeptide
QQQRPLEAQPSAPGHSVK

SEQ ID NO: 465 - protein, 22 aa, synthetic construct
  REGION 1..22: note = Synthetic Polypeptide
AAHTANFLLNASGSTSTPAPSR

SEQ ID NO: 466 - protein, 16 aa, synthetic construct
  REGION 1..16: note = Synthetic Polypeptide
  VARIANT 2..4: note = misc_feature - Xaa can be any naturally occurring amino acid
IXXXFLGASLKDEVLK

SEQ ID NO: 467 - protein, 24 aa, synthetic construct
  REGION 1..24: note = Synthetic Polypeptide
NVEEADAAMAASPHAVDGNTVELK

SEQ ID NO: 468 - protein, 16 aa, synthetic construct
  REGION 1..16: note = Synthetic Polypeptide
ALLVTASQCQQPAENK

SEQ ID NO: 469 - protein, 31 aa, synthetic construct
  REGION 1..31: note = Synthetic Polypeptide
QSSWGMMGMLASQQNQSGPSGNNQNQGNMQR

SEQ ID NO: 470 - protein, 16 aa, synthetic construct
  REGION 1..16: note = Synthetic Polypeptide
LPPGFSASSTVEKPSK

SEQ ID NO: 471 - protein, 16 aa, synthetic construct
  REGION 1..16: note = Synthetic Polypeptide
AAVPSGASTGIYEALE

SEQ ID NO: 472 - protein, 16 aa, synthetic construct
  REGION 1..16: note = Synthetic Polypeptide
LNCQVIGASVDSHFCH

SEQ ID NO: 473 - protein, 20 aa, synthetic construct
  REGION 1..20: note = Synthetic Polypeptide
GANQYTFHLEATENPGALIK

SEQ ID NO: 474 - protein, 22 aa, synthetic construct
  REGION 1..22: note = Synthetic Polypeptide
LSELTQQLAQATGKPPQYIAVH

SEQ ID NO: 475 - protein, 45 aa, synthetic construct
  REGION 1..45: note = Synthetic Polypeptide
AGEQEGAMVAATQGAAAAAGSGAGTGGGTASGGTEGGSAESEGAK

SEQ ID NO: 476 - protein, 19 aa, synthetic construct
  REGION 1..19: note = Synthetic Polypeptide
ISSIQATTAAGSGHPTSCC

SEQ ID NO: 477 - protein, 13 aa, synthetic construct
  REGION 1..13: note = Synthetic Polypeptide
GLGATTHPTAAVK

SEQ ID NO: 478 - protein, 17 aa, synthetic construct
  REGION 1..17: note = Synthetic Polypeptide
IEPPPLDAVIEAEHTLR

SEQ ID NO: 479 - protein, 19 aa, synthetic construct
  REGION 1..19: note = Synthetic Polypeptide
  VARIANT 2: note = misc_feature - Xaa can be any naturally occurring amino acid
SXGLPVGAVINCADNTGAK

SEQ ID NO: 480 - protein, 18 aa, synthetic construct
  REGION 1..18: note = Synthetic Polypeptide
VNVANVGAVPSGQDNIHR

SEQ ID NO: 481 - protein, 13 aa, synthetic construct
  REGION 1..13: note = Synthetic Polypeptide
QFLECAQNQGDIK

SEQ ID NO: 482 - protein, 20 aa, synthetic construct
  REGION 1..20: note = Synthetic Polypeptide
GGLTDEAALSCCSDADPSTK

SEQ ID NO: 483 - protein, 11 aa, synthetic construct
  REGION 1..11: note = Synthetic Polypeptide
ILSCGEVIHVK

SEQ ID NO: 484 - protein, 14 aa, synthetic construct
  REGION 1..14: note = Synthetic Polypeptide
AIVDCGFEHPSEVQ

SEQ ID NO: 485 - protein, 14 aa, synthetic construct
  REGION 1..14: note = Synthetic Polypeptide
HYYEVSCHDQGLCR

SEQ ID NO: 486 - protein, 15 aa, synthetic construct
  REGION 1..15: note = Synthetic Polypeptide
MLVQCMQDQEHPSIR

SEQ ID NO: 487 - protein, 15 aa, synthetic construct
  REGION 1..15: note = Synthetic Polypeptide
DVVICPDASLEDAKK

SEQ ID NO: 488 - protein, 16 aa, synthetic construct
  REGION 1..16: note = Synthetic Polypeptide
SGYAFVDCPDEHWAMK

SEQ ID NO: 489 - protein, 19 aa, synthetic construct
  REGION 1..19: note = Synthetic Polypeptide
FVLCPECENPETDLHVNPK

SEQ ID NO: 490 - protein, 14 aa, synthetic construct
  REGION 1..14: note = Synthetic Polypeptide
IAILTCPFEPPKPK

SEQ ID NO: 491 - protein, 14 aa, synthetic construct
  REGION 1..14: note = Synthetic Polypeptide
IAILTCPFEPPKPK

SEQ ID NO: 492 - protein, 25 aa, synthetic construct
  REGION 1..25: note = Synthetic Polypeptide
AATEQYHQVLCPGPSQDDPLHPLNK

SEQ ID NO: 493 - protein, 14 aa, synthetic construct
  REGION 1..14: note = Synthetic Polypeptide
AIVICPTDEDLKDR

SEQ ID NO: 494 - protein, 19 aa, synthetic construct
  REGION 1..19: note = Synthetic Polypeptide
ATAHAQAQLGCPVIIHPGR

SEQ ID NO: 495 - protein, 14 aa, synthetic construct
  REGION 1..14: note = Synthetic Polypeptide
  VARIANT 5: note = misc_feature - Xaa can be any naturally occurring amino acid
IIPGXMCQGGDFTR

SEQ ID NO: 496 - protein, 14 aa, synthetic construct
  REGION 1..14: note = Synthetic Polypeptide
  VARIANT 5: note = misc_feature - Xaa can be any naturally occurring amino acid
IIPGXMCQGGDFTR

SEQ ID NO: 497 - protein, 19 aa, synthetic construct
  REGION 1..19: note = Synthetic Polypeptide
PALYWLSGLTCTEQNFISK

SEQ ID NO: 498 - protein, 17 aa, synthetic construct
  REGION 1..17: note = Synthetic Polypeptide
MTVGCVAGDEESYEVFK

SEQ ID NO: 499 - protein, 17 aa, synthetic construct
  REGION 1..17: note = Synthetic Polypeptide
ATVAFCDAQSTQEIHEK

SEQ ID NO: 500 - protein, 16 aa, synthetic construct
  REGION 1..16: note = Synthetic Polypeptide
NYGILADATEQVGQHK

SEQ ID NO: 501 - protein, 10 aa, synthetic construct
  REGION 1..10: note = Synthetic Polypeptide
LQDCEGLIVR

SEQ ID NO: 502 - protein, 20 aa, synthetic construct
  REGION 1..20: note = Synthetic Polypeptide
  VARIANT 7: note = misc_feature - Xaa can be any naturally occurring amino acid
  VARIANT 10: note = misc_feature - Xaa can be any naturally occurring amino acid
QISAGYXPVXDCHTAHIACK

SEQ ID NO: 503 - protein, 20 aa, synthetic construct
  REGION 1..20: note = Synthetic Polypeptide
  VARIANT 7: note = misc_feature - Xaa can be any naturally occurring amino acid
  VARIANT 10: note = misc_feature - Xaa can be any naturally occurring amino acid
QISAGYXPVXDCHTAHIACK

SEQ ID NO: 504 - protein, 19 aa, synthetic construct
  REGION 1..19: note = Synthetic Polypeptide
TGEPCCDWVGDEGAGHFVK

SEQ ID NO: 505 - protein, 19 aa, synthetic construct
  REGION 1..19: note = Synthetic Polypeptide
DATNVGDEGGFAPNILENK

SEQ ID NO: 506 - protein, 13 aa, synthetic construct
  REGION 1..13: note = Synthetic Polypeptide
NILDFPQHVSPSK

SEQ ID NO: 507 - protein, 14 aa, synthetic construct
  REGION 1..14: note = Synthetic Polypeptide
DHVVSDFSEHGSLK

SEQ ID NO: 508 - protein, 18 aa, synthetic construct
  REGION 1..18: note = Synthetic Polypeptide
ADNELSPECLDGAQHFLK

SEQ ID NO: 509 - protein, 18 aa, synthetic construct
  REGION 1..18: note = Synthetic Polypeptide
IQTLGYFPVGDGDFPHQK

SEQ ID NO: 510 - protein, 19 aa, synthetic construct
  REGION 1..19: note = Synthetic Polypeptide
  VARIANT 2: note = misc_feature - Xaa can be any naturally occurring amino acid
  VARIANT 5..7: note = misc_feature - Xaa can be any naturally occurring amino acid
DXQEXXXFLLDGLHEDLNR

SEQ ID NO: 511 - protein, 18 aa, synthetic construct
  REGION 1..18: note = Synthetic Polypeptide
MILIQDGSQNTNVDKPLR

SEQ ID NO: 512 - protein, 15 aa, synthetic construct
  REGION 1..15: note = Synthetic Polypeptide
FTISDHPQPIDPLLK

SEQ ID NO: 513 - protein, 18 aa, synthetic construct
  REGION 1..18: note = Synthetic Polypeptide
  VARIANT 5: note = misc_feature - Xaa can be any naturally occurring amino acid
VNPTXFFDIAVDGEPLGR

SEQ ID NO: 514 - protein, 17 aa, synthetic construct
  REGION 1..17: note = Synthetic Polypeptide
YEDICPSTHNMDVPNIK

SEQ ID NO: 515 - protein, 20 aa, synthetic construct
  REGION 1..20: note = Synthetic Polypeptide
NGSIYNPEVLDITEETLHSR

SEQ ID NO: 516 - protein, 13 aa, synthetic construct
  REGION 1..13: note = Synthetic Polypeptide
QHIVNDMNPGNLH

SEQ ID NO: 517 - protein, 13 aa, synthetic construct
  REGION 1..13: note = Synthetic Polypeptide
MLLDSEQHPCQLK

SEQ ID NO: 518 - protein, 13 aa, synthetic construct
  REGION 1..13: note = Synthetic Polypeptide
EGLMLDSHEELYK

SEQ ID NO: 519 - protein, 15 aa, synthetic construct
  REGION 1..15: note = Synthetic Polypeptide
TAGDTHLGGEDFDNR

SEQ ID NO: 520 - protein, 21 aa, synthetic construct
  REGION 1..21: note = Synthetic Polypeptide
PGGLLLGDVAPNFEANTTVGR

SEQ ID NO: 521 - protein, 18 aa, synthetic construct
  REGION 1..18: note = Synthetic Polypeptide
NDGATILSMMDVDHQIAK

SEQ ID NO: 522 - protein, 19 aa, synthetic construct
  REGION 1..19: note = Synthetic Polypeptide
GEEGLTLNLEDVQPHDLGK

SEQ ID NO: 523 - protein, 18 aa, synthetic construct
  REGION 1..18: note = Synthetic Polypeptide
TIDNSQGAYQEAFDISKK

SEQ ID NO: 524 - protein, 18 aa, synthetic construct
  REGION 1..18: note = Synthetic Polypeptide
IVGFFDDSFSEAHSEFLK

SEQ ID NO: 525 - protein, 21 aa, synthetic construct
  REGION 1..21: note = Synthetic Polypeptide
QEEASGVALGEAPDHSYESLR

SEQ ID NO: 526 - protein, 14 aa, synthetic construct
  REGION 1..14: note = Synthetic Polypeptide
DPVQEAWAEDVDLR

SEQ ID NO: 527 - protein, 16 aa, synthetic construct
  REGION 1..16: note = Synthetic Polypeptide
PMIYICGECHTENEIK

SEQ ID NO: 528 - protein, 15 aa, synthetic construct
  REGION 1..15: note = Synthetic Polypeptide
HMSEFMECNLNELVK

SEQ ID NO: 529 - protein, 15 aa, synthetic construct
  REGION 1..15: note = Synthetic Polypeptide
MGYAEEAPYDAIHVG

SEQ ID NO: 530 - protein, 14 aa, synthetic construct
  REGION 1..14: note = Synthetic Polypeptide
MADQLTEEQIAEFK

SEQ ID NO: 531 - protein, 12 aa, synthetic construct
  REGION 1..12: note = Synthetic Polypeptide
YLAEFATGNDRK

SEQ ID NO: 532 - protein, 17 aa, synthetic construct
  REGION 1..17: note = Synthetic Polypeptide
LAELEEFINGPNNAHIQ

SEQ ID NO: 533 - protein, 9 aa, synthetic construct
  REGION 1..9: note = Synthetic Polypeptide
  VARIANT 1: note = misc_feature - Xaa can be any naturally occurring amino acid
XEFTDHLVK

SEQ ID NO: 534 - protein, 23 aa, synthetic construct
  REGION 1..23: note = Synthetic Polypeptide
FCVGFLEGGKDSCQGDSGGPVVC

SEQ ID NO: 535 - protein, 15 aa, synthetic construct
  REGION 1..15: note = Synthetic Polypeptide
LLAEGHPDPDAELQR

SEQ ID NO: 536 - protein, 10 aa, synthetic construct
  REGION 1..10: note = Synthetic Polypeptide
GLTEGLHGFH

SEQ ID NO: 537 - protein, 10 aa, synthetic construct
  REGION 1..10: note = Synthetic Polypeptide
GLTEGLHGFH

SEQ ID NO: 538 - protein, 19 aa, synthetic construct
  REGION 1..19: note = Synthetic Polypeptide
FVHWYVGEGMEEGEFSEAR

SEQ ID NO: 539 - protein, 13 aa, synthetic construct
  REGION 1..13: note = Synthetic Polypeptide
MLLHEGQHPAQLR

SEQ ID NO: 540 - protein, 18 aa, synthetic construct
  REGION 1..18: note = Synthetic Polypeptide
YHGYTFANLGEHEFVEEK

SEQ ID NO: 541 - protein, 12 aa, synthetic construct
  REGION 1..12: note = Synthetic Polypeptide
TVFAEHISDECK

SEQ ID NO: 542 - protein, 15 aa, synthetic construct
  REGION 1..15: note = Synthetic Polypeptide
VILEEHSTCENEVSK

SEQ ID NO: 543 - protein, 15 aa, synthetic construct
  REGION 1..15: note = Synthetic Polypeptide
LLTEIHGGAGGPSGR

SEQ ID NO: 544 - protein, 16 aa, synthetic construct
  REGION 1..16: note = Synthetic Polypeptide
AHLMEIQVNGGTVAEK

SEQ ID NO: 545 - protein, 18 aa, synthetic construct
  REGION 1..18: note = Synthetic Polypeptide
ISWLDANTLAEKDEFEHK

SEQ ID NO: 546 - protein, 18 aa, synthetic construct
  REGION 1..18: note = Synthetic Polypeptide
ISWLDANTLAEKDEFEHK

SEQ ID NO: 547 - protein, 12 aa, synthetic construct
  REGION 1..12: note = Synthetic Polypeptide
  VARIANT 1: note = misc_feature - Xaa can be any naturally occurring amino acid
XEKFEDENFILK

SEQ ID NO: 548 - protein, 22 aa, synthetic construct
  REGION 1..22: note = Synthetic Polypeptide
SAVEAGSEVSEKPGQEAPVLPK

SEQ ID NO: 549 - protein, 22 aa, synthetic construct
  REGION 1..22: note = Synthetic Polypeptide
SAVEAGSEVSEKPGQEAPVLPK

SEQ ID NO: 550 - protein, 13 aa, synthetic construct
  REGION 1..13: note = Synthetic Polypeptide
ILNEKPTTDEPEK

SEQ ID NO: 551 - protein, 14 aa, synthetic construct
  REGION 1..14: note = Synthetic Polypeptide
YLAEKYEWDVAEAR

SEQ ID NO: 552 - protein, 15 aa, synthetic construct
  REGION 1..15: note = Synthetic Polypeptide
  VARIANT 6: note = misc_feature - Xaa can be any naturally occurring amino acid
CLELFXELAEDKENY

SEQ ID NO: 553 - protein, 16 aa, synthetic construct
  REGION 1..16: note = Synthetic Polypeptide
  VARIANT 6: note = misc_feature - Xaa can be any naturally occurring amino acid
CLELFXELAEDKENYK

SEQ ID NO: 554 - protein, 11 aa, synthetic construct
  REGION 1..11: note = Synthetic Polypeptide
MEELHNQEVQK

SEQ ID NO: 555 - protein, 26 aa, synthetic construct
  REGION 1..26: note = Synthetic Polypeptide
GVNVAGVSLQELNPEMGTDNDSENWK

SEQ ID NO: 556 - protein, 16 aa, synthetic construct
  REGION 1..16: note = Synthetic Polypeptide
ASDIAMTELPPTHPIR

SEQ ID NO: 557 - protein, 16 aa, synthetic construct
  REGION 1..16: note = Synthetic Polypeptide
VVVAENFDEIVNNENK

SEQ ID NO: 558 - protein, 16 aa, synthetic construct
  REGION 1..16: note = Synthetic Polypeptide
  VARIANT 2: note = misc_feature - Xaa can be any naturally occurring amino acid
IXEGCEEPATHNALAK

SEQ ID NO: 559 - protein, 13 aa, synthetic construct
  REGION 1..13: note = Synthetic Polypeptide
VTEQGAELSNEER

SEQ ID NO: 560 - protein, 14 aa, synthetic construct
  REGION 1..14: note = Synthetic Polypeptide
AVTEQGHELSNEER

SEQ ID NO: 561 - protein, 21 aa, synthetic construct
  REGION 1..21: note = Synthetic Polypeptide
QVDQEEPHVEEQQQQTPAENK

SEQ ID NO: 562 - protein, 21 aa, synthetic construct
  REGION 1..21: note = Synthetic Polypeptide
QVDQEEPHVEEQQQQTPAENK

SEQ ID NO: 563 - protein, 14 aa, synthetic construct
  REGION 1..14: note = Synthetic Polypeptide
VVFEQTKVIADNVK

SEQ ID NO: 564 - protein, 23 aa, synthetic construct
  REGION 1..23: note = Synthetic Polypeptide
NIFVGENILEESENLHNADQPLR

SEQ ID NO: 565 - protein, 14 aa, synthetic construct
  REGION 1..14: note = Synthetic Polypeptide
LFIHESIHDEVVNR

SEQ ID NO: 566 - protein, 25 aa, synthetic construct
  REGION 1..25: note = Synthetic Polypeptide
VTNGIEEPLEESSHEPEPEPESETK

SEQ ID NO: 567 - protein, 12 aa, synthetic construct
  REGION 1..12: note = Synthetic Polypeptide
GIVEESVTGVHR

SEQ ID NO: 568 - protein, 18 aa, synthetic construct
  REGION 1..18: note = Synthetic Polypeptide
QCPSVVSLLSESYNPHVR

SEQ ID NO: 569 - protein, 14 aa, synthetic construct
  REGION 1..14: note = Synthetic Polypeptide
ASLQETHFDSTQTK

SEQ ID NO: 570 - protein, 10 aa, synthetic construct
  REGION 1..10: note = Synthetic Polypeptide
TFGETHPFTK

SEQ ID NO: 571 - protein, 17 aa, synthetic construct
  REGION 1..17: note = Synthetic Polypeptide
VMLGETNPADSKPGTIR

SEQ ID NO: 572 - protein, 17 aa, synthetic construct
  REGION 1..17: note = Synthetic Polypeptide
VMLGETNPADSKPGTIR

SEQ ID NO: 573 - protein, 17 aa, synthetic construct
  REGION 1..17: note = Synthetic Polypeptide
VMLGETNPADSKPGTIR

SEQ ID NO: 574 - protein, 18 aa, synthetic construct
  REGION 1..18: note = Synthetic Polypeptide
GADFLVTEVENGGSLGSK

SEQ ID NO: 575 - protein, 21 aa, synthetic construct
  REGION 1..21: note = Synthetic Polypeptide
LPTEAYISVEEVHDDGTPTSK

SEQ ID NO: 576 - protein, 21 aa, synthetic construct
  REGION 1..21: note = Synthetic Polypeptide
LPTEAYISVEEVHDDGTPTSK

SEQ ID NO: 577 - protein, 18 aa, synthetic construct
  REGION 1..18: note = Synthetic Polypeptide
MEEVPHDCPGADSAQAGR

SEQ ID NO: 578 - protein, 15 aa, synthetic construct
  REGION 1..15: note = Synthetic Polypeptide
VDENCVGFDHTVKPV

SEQ ID NO: 579 - protein, 21 aa, synthetic construct
  REGION 1..21: note = Synthetic Polypeptide
VHVVPDQLMAFGGSSEPCALC

SEQ ID NO: 580 - protein, 15 aa, synthetic construct
  REGION 1..15: note = Synthetic Polypeptide
IWCFGPDGTGPNILT

SEQ ID NO: 581 - protein, 10 aa, synthetic construct
  REGION 1..10: note = Synthetic Polypeptide
  VARIANT 3: note = misc_feature - Xaa can be any naturally occurring amino acid
YVXFGPHAGK

SEQ ID NO: 582 - protein, 18 aa, synthetic construct
  REGION 1..18: note = Synthetic Polypeptide
EFAGFQCQIQFGPHNEQK

SEQ ID NO: 583 - protein, 17 aa, synthetic construct
  REGION 1..17: note = Synthetic Polypeptide
  VARIANT 3: note = misc_feature - Xaa can be any naturally occurring amino acid
KPXKPMQFLGDEETVRK

SEQ ID NO: 584 - protein, 13 aa, synthetic construct
  REGION 1..13: note = Synthetic Polypeptide
MVYMFQYDSTHGK

SEQ ID NO: 585 - protein, 15 aa, synthetic construct
  REGION 1..15: note = Synthetic Polypeptide
EELGFRPEYSASQLK

SEQ ID NO: 586 - protein, 10 aa, synthetic construct
  REGION 1..10: note = Synthetic Polypeptide
HLEFSHDQYR

SEQ ID NO: 587 - protein, 15 aa, synthetic construct
  REGION 1..15: note = Synthetic Polypeptide
TCGFDFTGAVEDISK

SEQ ID NO: 588 - protein, 14 aa, synthetic construct
  REGION 1..14: note = Synthetic Polypeptide
GFGFVDFNSEEDAK

SEQ ID NO: 589 - protein, 14 aa, synthetic construct
  REGION 1..14: note = Synthetic Polypeptide
GFGFVDFNSEEDAK

SEQ ID NO: 590 - protein, 10 aa, synthetic construct
  REGION 1..10: note = Synthetic Polypeptide
NYGFVHIEDK

SEQ ID NO: 591 - protein, 15 aa, synthetic construct
  REGION 1..15: note = Synthetic Polypeptide
GFGFVTFDDHDPVDK

SEQ ID NO: 592 - protein, 17 aa, synthetic construct
  REGION 1..17: note = Synthetic Polypeptide
LPNFGFVVFDDSEPVQK

SEQ ID NO: 593 - protein, 15 aa, synthetic construct
  REGION 1..15: note = Synthetic Polypeptide
QLLCGAAIGTHEDDK

SEQ ID NO: 594 - protein, 17 aa, synthetic construct
  REGION 1..17: note = Synthetic Polypeptide
QLLCGAAIGTHEDDKYR

SEQ ID NO: 595 - protein, 16 aa, synthetic construct
  REGION 1..16: note = Synthetic Polypeptide
MTNGFSGADLTEICQR

SEQ ID NO: 596 - protein, 22 aa, synthetic construct
  REGION 1..22: note = Synthetic Polypeptide
VQGEVMEGADNQGAGEQGRPVR

SEQ ID NO: 597 - protein, 21 aa, synthetic construct
  REGION 1..21: note = Synthetic Polypeptide
MGGHGYGGAGDASSGFHGGHF

SEQ ID NO: 598 - protein, 36 aa, synthetic construct
  REGION 1..36: note = Synthetic Polypeptide
LGNVLGGLISGAGGGGGGGGGGGGGGGGGGGGTAMR

SEQ ID NO: 599 - protein, 21 aa, synthetic construct
  REGION 1..21: note = Synthetic Polypeptide
FGGSFAGSFGGAGGHAPGVAR

SEQ ID NO: 600 - protein, 21 aa, synthetic construct
  REGION 1..21: note = Synthetic Polypeptide
FGGSFAGSFGGAGGHAPGVAR

SEQ ID NO: 601 - protein, 15 aa, synthetic construct
  REGION 1..15: note = Synthetic Polypeptide
VLVVGAGGIGCELLK

SEQ ID NO: 602 - protein, 20 aa, synthetic construct
  REGION 1..20: note = Synthetic Polypeptide
VTADHGPAVSGAHNTIICAR

SEQ ID NO: 603 - protein, 14 aa, synthetic construct
  REGION 1..14: note = Synthetic Polypeptide
CEALAGAPLDNAPK

SEQ ID NO: 604 - protein, 14 aa, synthetic construct
  REGION 1..14: note = Synthetic Polypeptide
CEALAGAPLDNAPK

SEQ ID NO: 605 - protein, 14 aa, synthetic construct
  REGION 1..14: note = Synthetic Polypeptide
STGGAPTFNVTVTK

SEQ ID NO: 606 - protein, 14 aa, synthetic construct
  REGION 1..14: note = Synthetic Polypeptide
KGCDVVVIPAGVPR

SEQ ID NO: 607 - protein, 15 aa, synthetic construct
  REGION 1..15: note = Synthetic Polypeptide
FSPAGVEGCPALPHK

SEQ ID NO: 608 - protein, 13 aa, synthetic construct
  REGION 1..13: note = Synthetic Polypeptide
HSSLAGCQIINYR

SEQ ID NO: 609 - protein, 19 aa, synthetic construct
  REGION 1..19: note = Synthetic Polypeptide
SSEVGYDAMAGDFVNMVEK

SEQ ID NO: 610 - protein, 18 aa, synthetic construct
  REGION 1..18: note = Synthetic Polypeptide
SIEDSVISLSGDHCIIGR

SEQ ID NO: 611 - protein, 18 aa, synthetic construct
  REGION 1..18: note = Synthetic Polypeptide
SIEDSVISLSGDHCIIGR

SEQ ID NO: 612 - protein, 15 aa, synthetic construct
  REGION 1..15: note = Synthetic Polypeptide
VTGDHIPTPQDLPQR

SEQ ID NO: 613 - protein, 15 aa, synthetic construct
  REGION 1..15: note = Synthetic Polypeptide
VTGDHIPTPQDLPQR

SEQ ID NO: 614 - protein, 17 aa, synthetic construct
  REGION 1..17: note = Synthetic Polypeptide
NGDTFLGGEDFDQALLR

SEQ ID NO: 615 - protein, 16 aa, synthetic construct
  REGION 1..16: note = Synthetic Polypeptide
IVYICCGEDHTAALTK

SEQ ID NO: 616 - protein, 17 aa, synthetic construct
  REGION 1..17: note = Synthetic Polypeptide
MVDGNVSGEFTDLVPEK

SEQ ID NO: 617 - protein, 12 aa, synthetic construct
  REGION 1..12: note = Synthetic Polypeptide
MAAQGEPQVQFK

SEQ ID NO: 618 - protein, 20 aa, synthetic construct
  REGION 1..20: note = Synthetic Polypeptide
QALAVHLALQGESSSEHFLK

SEQ ID NO: 619 - protein, 16 aa, synthetic construct
  REGION 1..16: note = Synthetic Polypeptide
AFYNNVLGEYEEYITK

SEQ ID NO: 620 - protein, 13 aa, synthetic construct
  REGION 1..13: note = Synthetic Polypeptide
LLNQMDGFDTLHR

SEQ ID NO: 621 - protein, 15 aa, synthetic construct
  REGION 1..15: note = Synthetic Polypeptide
GLTEGLHGFHVHEFG

SEQ ID NO: 622 - protein, 22 aa, synthetic construct
  REGION 1..22: note = Synthetic Polypeptide
GLTEGLHGFHVHEFGDNTAGCT

SEQ ID NO: 623 - protein, 22 aa, synthetic construct
  REGION 1..22: note = Synthetic Polypeptide
GLTEGLHGFHVHEFGDNTAGCT

SEQ ID NO: 624 - protein, 22 aa, synthetic construct
  REGION 1..22: note = Synthetic Polypeptide
GLTEGLHGFHVHEFGDNTAGCT

SEQ ID NO: 625 - protein, 22 aa, synthetic construct
  REGION 1..22: note = Synthetic Polypeptide
GLTEGLHGFHVHEFGDNTAGCT

SEQ ID NO: 626 - protein, 22 aa, synthetic construct
  REGION 1..22: note = Synthetic Polypeptide
GLTEGLHGFHVHEFGDNTAGCT

SEQ ID NO: 627 - protein, 24 aa, synthetic construct
  REGION 1..24: note = Synthetic Polypeptide
AADSYFSLLQGFINSLDESTQESK

SEQ ID NO: 628 - protein, 21 aa, synthetic construct
  REGION 1..21: note = Synthetic Polypeptide
INPYLLGTMAGGAADCSFWER

SEQ ID NO: 629 - protein, 20 aa, synthetic construct
  REGION 1..20: note = Synthetic Polypeptide
QHDLFDSGFGGGAGVETGGK

SEQ ID NO: 630 - protein, 19 aa, synthetic construct
  REGION 1..19: note = Synthetic Polypeptide
TTHFVEGGDAGNREDQINR

SEQ ID NO: 631 - protein, 19 aa, synthetic construct
  REGION 1..19: note = Synthetic Polypeptide
TTHFVEGGDAGNREDQINR

SEQ ID NO: 632 - protein, 21 aa, synthetic construct
  REGION 1..21: note = Synthetic Polypeptide
SQPIAQQPLQGGDHSGNYGYK

SEQ ID NO: 633 - protein, 22 aa, synthetic construct
  REGION 1..22: note = Synthetic Polypeptide
GTDGTDNPLSGGDQYQNITVHR

SEQ ID NO: 634 - protein, 17 aa, synthetic construct
  REGION 1..17: note = Synthetic Polypeptide
  VARIANT 5: note = misc_feature - Xaa can be any naturally occurring amino acid
GCITXIGGGDTATCCAK

SEQ ID NO: 635 - protein, 38 aa, synthetic construct
  REGION 1..38: note = Synthetic Polypeptide
WGSGGGGGGGGGGGGGGGGGGGGGGGGGGGRKSSSAAA

SEQ ID NO: 636 - protein, 21 aa, synthetic construct
  REGION 1..21: note = Synthetic Polypeptide
LAAGSLAAPGGGGGSAGGARP

SEQ ID NO: 637 - protein, 16 aa, synthetic construct
  REGION 1..16: note = Synthetic Polypeptide
  VARIANT 3..5: note = misc_feature - Xaa can be any naturally occurring amino acid
GSXXXGGGSYNDFGNY

SEQ ID NO: 638 - protein, 20 aa, synthetic construct
  REGION 1..20: note = Synthetic Polypeptide
  VARIANT 6: note = misc_feature - Xaa can be any naturally occurring amino acid
VNAANXSLLGGGGVDGCIHR

SEQ ID NO: 639 - protein, 23 aa, synthetic construct
  REGION 1..23: note = Synthetic Polypeptide
FCVGFLEGGKDSCQGDSGGPVVC

SEQ ID NO: 640 - protein, 24 aa, synthetic construct
  REGION 1..24: note = Synthetic Polypeptide
LVDGQIFCLHGGLSPSIDTLDHIR

SEQ ID NO: 641 - protein, 13 aa, synthetic construct
  REGION 1..13: note = Synthetic Polypeptide
  VARIANT 3: note = misc_feature - Xaa can be any naturally occurring amino acid
MFXGGLSWDTSKK

SEQ ID NO: 642 - protein, 21 aa, synthetic construct
  REGION 1..21: note = Synthetic Polypeptide
DPQELLEGGNQGEGDPQAEGR

SEQ ID NO: 643 - protein, 25 aa, synthetic construct
  REGION 1..25: note = Synthetic Polypeptide
NMGGPYGGGNYGPGGSGGSGGYGGR

SEQ ID NO: 644 - protein, 16 aa, synthetic construct
  REGION 1..16: note = Synthetic Polypeptide
RGGPGGPGGPGGPMGR

SEQ ID NO: 645 - protein, 19 aa, synthetic construct
  REGION 1..19: note = Synthetic Polypeptide
SVLDDWFPLQGGQGQVHLR

SEQ ID NO: 646 - protein, 16 aa, synthetic construct
  REGION 1..16: note = Synthetic Polypeptide
IIMEYLGGGSALDLLR

SEQ ID NO: 647 - protein, 23 aa, synthetic construct
  REGION 1..23: note = Synthetic Polypeptide
SHFAMMHGGTGFAGIDSSSPEVK

SEQ ID NO: 648 - protein, 22 aa, synthetic construct
  REGION 1..22: note = Synthetic Polypeptide
QGFQLTHSLGGGTGSGMGTLLI

SEQ ID NO: 649 - protein, 19 aa, synthetic construct
  REGION 1..19: note = Synthetic Polypeptide
MADYLISGGTSYVPDDGLT

SEQ ID NO: 650 - protein, 13 aa, synthetic construct
  REGION 1..13: note = Synthetic Polypeptide
VTVAGGVHISGLH

SEQ ID NO: 651 - protein, 14 aa, synthetic construct
  REGION 1..14: note = Synthetic Polypeptide
VTVAGGVHISGLHT

SEQ ID NO: 652 - protein, 15 aa, synthetic construct
  REGION 1..15: note = Synthetic Polypeptide
VTVAGGVHISGLHTE

SEQ ID NO: 653 - protein, 20 aa, synthetic construct
  REGION 1..20: note = Synthetic Polypeptide
YAVSELAGHQTSAESWGTGR

SEQ ID NO: 654 - protein, 13 aa, synthetic construct
  REGION 1..13: note = Synthetic Polypeptide
TFQGHTNEVNAIK

SEQ ID NO: 655 - protein, 14 aa, synthetic construct
  REGION 1..14: note = Synthetic Polypeptide
GDGPVQGIINFEQK

SEQ ID NO: 656 - protein, 18 aa, synthetic construct
  REGION 1..18: note = Synthetic Polypeptide
VTIIGPATVGGIKPGCFK

SEQ ID NO: 657 - protein, 20 aa, synthetic construct
  REGION 1..20: note = Synthetic Polypeptide
FSLPGMEHVYGIPEHADNLR

SEQ ID NO: 658 - protein, 16 aa, synthetic construct
  REGION 1..16: note = Synthetic Polypeptide
IFVGGIPHNCGETELR

SEQ ID NO: 659 - protein, 18 aa, synthetic construct
  REGION 1..18: note = Synthetic Polypeptide
LPPSGAVPVTGIPPHVVK

SEQ ID NO: 660 - protein, 13 aa, synthetic construct
  REGION 1..13: note = Synthetic Polypeptide
MDGIVPDIAVGTK

SEQ ID NO: 661 - protein, 9 aa, synthetic construct
  REGION 1..9: note = Synthetic Polypeptide
RGIWHNDNK

SEQ ID NO: 662 - protein, 20 aa, synthetic construct
  REGION 1..20: note = Synthetic Polypeptide
GKPEIEGKPESEGEPGSETR

SEQ ID NO: 663 - protein, 22 aa, synthetic construct
  REGION 1..22: note = Synthetic Polypeptide
YDINAHACVTGKPISQGGIHGR

SEQ ID NO: 664 - protein, 20 aa, synthetic construct
  REGION 1..20: note = Synthetic Polypeptide
ELTQQLAQATGKPPQYIAVH

SEQ ID NO: 665 - protein, 17 aa, synthetic construct
  REGION 1..17: note = Synthetic Polypeptide
NPKPFLNGLTGKPVMVK

SEQ ID NO: 666 - protein, 16 aa, synthetic construct
  REGION 1..16: note = Synthetic Polypeptide
CPSILGGLAPEKDQPK

SEQ ID NO: 667 - protein, 21 aa, synthetic construct
  REGION 1..21: note = Synthetic Polypeptide
  VARIANT 10: note = misc_feature - Xaa can be any naturally occurring amino acid
VASGIPAGWXGLDCGPESSKK

SEQ ID NO: 668 - protein, 12 aa, synthetic construct
  REGION 1..12: note = Synthetic Polypeptide
QVLQGLDYLHSK

SEQ ID NO: 669 - protein, 14 aa, synthetic construct
  REGION 1..14: note = Synthetic Polypeptide
GALEGLPRPPPPVK

SEQ ID NO: 670 - protein, 16 aa, synthetic construct
  REGION 1..16: note = Synthetic Polypeptide
LFIGGLSFETTDESLR

SEQ ID NO: 671 - protein, 16 aa, synthetic construct
  REGION 1..16: note = Synthetic Polypeptide
VFVGGLSPDTSEEQIK

SEQ ID NO: 672 - protein, 13 aa, synthetic construct
  REGION 1..13: note = Synthetic Polypeptide
  VARIANT 3: note = misc_feature - Xaa can be any naturally occurring amino acid
MFXGGLSWDTSKK

SEQ ID NO: 673 - protein, 20 aa, synthetic construct
  REGION 1..20: note = Synthetic Polypeptide
NVIIWGNHSSTQYPDVNHAK

SEQ ID NO: 674 - protein, 19 aa, synthetic construct
  REGION 1..19: note = Synthetic Polypeptide
LLSGLAEGLGGNIEQLVAR

SEQ ID NO: 675 - protein, 14 aa, synthetic construct
  REGION 1..14: note = Synthetic Polypeptide
LVINGNPITIFQER

SEQ ID NO: 676 - protein, 16 aa, synthetic construct
  REGION 1..16: note = Synthetic Polypeptide
SAAMLGNSEDHTALSR

SEQ ID NO: 677 - protein, 11 aa, synthetic construct
  REGION 1..11: note = Synthetic Polypeptide
IFQGNVHNFEK

SEQ ID NO: 678 - protein, 15 aa, synthetic construct
  REGION 1..15: note = Synthetic Polypeptide
NNPPTLEGNYSKPLK

SEQ ID NO: 679 - protein, 11 aa, synthetic construct
  REGION 1..11: note = Synthetic Polypeptide
MVGPAVIVDKK

SEQ ID NO: 680 - protein, 14 aa, synthetic construct
  REGION 1..14: note = Synthetic Polypeptide
MMLGPEGGEGFVVK

SEQ ID NO: 681 - protein, 16 aa, synthetic construct
  REGION 1..16: note = Synthetic Polypeptide
SIYEALGGPHDPNVAK

SEQ ID NO: 682 - protein, 13 aa, synthetic construct
  REGION 1..13: note = Synthetic Polypeptide
TFQGPNCPATCGR

SEQ ID NO: 683 - protein, 11 aa, synthetic construct
  REGION 1..11: note = Synthetic Polypeptide
IMGPNYTPGKK

SEQ ID NO: 684 - protein, 13 aa, synthetic construct
  REGION 1..13: note = Synthetic Polypeptide
MVIITGPPEAQFK

SEQ ID NO: 685 - protein, 20 aa, synthetic construct
  REGION 1..20: note = Synthetic Polypeptide
AFGLTDDQVSGPPSAPAEDR

SEQ ID NO: 686 - protein, 14 aa, synthetic construct
  REGION 1..14: note = Synthetic Polypeptide
TVQGPPTSDDIFER

SEQ ID NO: 687 - protein, 15 aa, synthetic construct
  REGION 1..15: note = Synthetic Polypeptide
FVIGGPQGDAGLTGR

SEQ ID NO: 688 - protein, 13 aa, synthetic construct
  REGION 1..13: note = Synthetic Polypeptide
  VARIANT 5: note = misc_feature - Xaa can be any naturally occurring amino acid
IITLXGPTNAIFK

SEQ ID NO: 689 - protein, 21 aa, synthetic construct
  REGION 1..21: note = Synthetic Polypeptide
KPPTLIHGQAPSAGLPSQKPK

SEQ ID NO: 690 - protein, 11 aa, synthetic construct
  REGION 1..11: note = Synthetic Polypeptide
RGQGGYPGKPR

SEQ ID NO: 691 - protein, 18 aa, synthetic construct
  REGION 1..18: note = Synthetic Polypeptide
  VARIANT 6: note = misc_feature - Xaa can be any naturally occurring amino acid
RPDNFXFGQSGAGNNWAK

SEQ ID NO: 692 - protein, 18 aa, synthetic construct
  REGION 1..18: note = Synthetic Polypeptide
GLLALSSALSGQSHLAIK

SEQ ID NO: 693 - protein, 22 aa, synthetic construct
  REGION 1..22: note = Synthetic Polypeptide
ALPPVLTTVNGQSPPEHSAPAK

SEQ ID NO: 694 - protein, 13 aa, synthetic construct
  REGION 1..13: note = Synthetic Polypeptide
QSGYGGQTKPIFR

SEQ ID NO: 695 - protein, 11 aa, synthetic construct
  REGION 1..11: note = Synthetic Polypeptide
LSGQTNIHLSK

SEQ ID NO: 696 - protein, 18 aa, synthetic construct
  REGION 1..18: note = Synthetic Polypeptide
VVLMSHLGRPDGVPMPDK

SEQ ID NO: 697 - protein, 19 aa, synthetic construct
  REGION 1..19: note = Synthetic Polypeptide
VVLMSHLGRPDGVPMPDKY

SEQ ID NO: 698 - protein, 17 aa, synthetic construct
  REGION 1..17: note = Synthetic Polypeptide
QQSIAGSADSKPIDVSR

SEQ ID NO: 699 - protein, 20 aa, synthetic construct
  REGION 1..20: note = Synthetic Polypeptide
VTLGPVPEIGGSEAPAPQNK

SEQ ID NO: 700 - protein, 22 aa, synthetic construct
  REGION 1..22: note = Synthetic Polypeptide
NFGGSFAGSFGGAGGHAPGVAR

SEQ ID NO: 701 - protein, 17 aa, synthetic construct
  REGION 1..17: note = Synthetic Polypeptide
MMDYLQGSGETPQTDVR

SEQ ID NO: 702 - protein, 18 aa, synthetic construct
  REGION 1..18: note = Synthetic Polypeptide
DSVWGSGGGQQSVNHLVK

SEQ ID NO: 703 - protein, 17 aa, synthetic construct
  REGION 1..17: note = Synthetic Polypeptide
PQVAIICGSGLGGLTDK

SEQ ID NO: 704 - protein, 28 aa, synthetic construct
  REGION 1..28: note = Synthetic Polypeptide
PTSSEQGGLEGSGSAAGEGKPALSEEER

SEQ ID NO: 705 - protein, 22 aa, synthetic construct
  REGION 1..22: note = Synthetic Polypeptide
TVEQLLTGSPTSPTVEPEKPTR

SEQ ID NO: 706 - protein, 14 aa, synthetic construct
  REGION 1..14: note = Synthetic Polypeptide
GCLEGSQGTQALHK

SEQ ID NO: 707 - protein, 22 aa, synthetic construct
  REGION 1..22: note = Synthetic Polypeptide
LLAVSAPALQGSRPGETEENVR

SEQ ID NO: 708 - protein, 20 aa, synthetic construct
  REGION 1..20: note = Synthetic Polypeptide
  VARIANT 2..3: note = misc_feature - Xaa can be any naturally occurring amino acid
IXXGSSGAQGSGGGSTSAHY

SEQ ID NO: 709 - protein, 15 aa, synthetic construct
  REGION 1..15: note = Synthetic Polypeptide
VAFTGSTEVGHLIQK

SEQ ID NO: 710 - protein, 16 aa, synthetic construct
  REGION 1..16: note = Synthetic Polypeptide
VVVLMGSTSDLGHCEK

SEQ ID NO: 711 - protein, 17 aa, synthetic construct
  REGION 1..17: note = Synthetic Polypeptide
MVELLGSYTEDNASQAR

SEQ ID NO: 712 - protein, 14 aa, synthetic construct
  REGION 1..14: note = Synthetic Polypeptide
IYWGTATTGKPHVA

SEQ ID NO: 713 - protein, 15 aa, synthetic construct
  REGION 1..15: note = Synthetic Polypeptide
IVGFCWGGTAVHHLM

SEQ ID NO: 714 - protein, 23 aa, synthetic construct
  REGION 1..23: note = Synthetic Polypeptide
GVVPLAGTDGETTTQGLDGLSER

SEQ ID NO: 715 - protein, 16 aa, synthetic construct
  REGION 1..16: note = Synthetic Polypeptide
  VARIANT 2: note = misc_feature - Xaa can be any naturally occurring amino acid
  VARIANT 4: note = misc_feature - Xaa can be any naturally occurring amino acid
  VARIANT 6: note = misc_feature - Xaa can be any naturally occurring amino acid
GXVXFXGTDHIDQWNK

SEQ ID NO: 716 - protein, 12 aa, synthetic construct
  REGION 1..12: note = Synthetic Polypeptide
SVSGTDVQEECR

SEQ ID NO: 717 - protein, 14 aa, synthetic construct
  REGION 1..14: note = Synthetic Polypeptide
MMLGTEGGEGFVVK

SEQ ID NO: 718 - protein, 20 aa, synthetic construct
  REGION 1..20: note = Synthetic Polypeptide
IAFHQDGSLAGTGGLDAFGR

SEQ ID NO: 719 - protein, 16 aa, synthetic construct
  REGION 1..16: note = Synthetic Polypeptide
LNFSHGTHEYHAETIK

SEQ ID NO: 720 - protein, 15 aa, synthetic construct
  REGION 1..15: note = Synthetic Polypeptide
LVLGTHTSDEQNHLV

SEQ ID NO: 721 - protein, 22 aa, synthetic construct
  REGION 1..22: note = Synthetic Polypeptide
ALHWLVLGTHTSDEQNHLVVAR

SEQ ID NO: 722 - protein, 13 aa, synthetic construct
  REGION 1..13: note = Synthetic Polypeptide
VLSGTIHAGQPVK

SEQ ID NO: 723 - protein, 16 aa, synthetic construct
  REGION 1..16: note = Synthetic Polypeptide
IITITGTQDQIQNAQY

SEQ ID NO: 724 - protein, 11 aa, synthetic construct
  REGION 1..11: note = Synthetic Polypeptide
GGTSDVEVNEK

SEQ ID NO: 725 - protein, 16 aa, synthetic construct
  REGION 1..16: note = Synthetic Polypeptide
VLTGVAGEDAECHAAK

SEQ ID NO: 726 - protein, 20 aa, synthetic construct
  REGION 1..20: note = Synthetic Polypeptide
TGGVDTAAVGGVFDVSNADR

SEQ ID NO: 727 - protein, 16 aa, synthetic construct
  REGION 1..16: note = Synthetic Polypeptide
FIVDGWHEMDAENPLH

SEQ ID NO: 728 - protein, 26 aa, synthetic construct
  REGION 1..26: note = Synthetic Polypeptide
TMFSSEVQFGHAGACANQASETAVAK

SEQ ID NO: 729 - protein, 22 aa, synthetic construct
  REGION 1..22: note = Synthetic Polypeptide
PIYDVLQMVGHANRPLQDDEGR

SEQ ID NO: 730 - protein, 10 aa, synthetic construct
  REGION 1..10: note = Synthetic Polypeptide
EWAHATIIPK

SEQ ID NO: 731 - protein, 10 aa, synthetic construct
  REGION 1..10: note = Synthetic Polypeptide
KHEANNPQLK

SEQ ID NO: 732 - protein, 10 aa, synthetic construct
  REGION 1..10: note = Synthetic Polypeptide
MVNHFIAEFK

SEQ ID NO: 733 - protein, 10 aa, synthetic construct
  REGION 1..10: note = Synthetic Polypeptide
  VARIANT 3: note = misc_feature - Xaa can be any naturally occurring amino acid
LVXHFVEEFK

SEQ ID NO: 734 - protein, 17 aa, synthetic construct
  REGION 1..17: note = Synthetic Polypeptide
MPFPVNHGASSEDTLLK

SEQ ID NO: 735 - protein, 23 aa, synthetic construct
  REGION 1..23: note = Synthetic Polypeptide
  VARIANT 2: note = misc_feature - Xaa can be any naturally occurring amino acid
NXCWELYCLEHGIQPDGQMPSDK

SEQ ID NO: 736 - protein, 23 aa, synthetic construct
  REGION 1..23: note = Synthetic Polypeptide
  VARIANT 2: note = misc_feature - Xaa can be any naturally occurring amino acid
NXCWELYCLEHGIQPDGQMPSDK

SEQ ID NO: 737 - protein, 20 aa, synthetic construct
  REGION 1..20: note = Synthetic Polypeptide
VHAGPFANIAHGNSSIIADR

SEQ ID NO: 738 - protein, 18 aa, synthetic construct
  REGION 1..18: note = Synthetic Polypeptide
INQVFHGSCITEGNELTK

SEQ ID NO: 739 - protein, 15 aa, synthetic construct
  REGION 1..15: note = Synthetic Polypeptide
FELQHGTEEQQEEVR

SEQ ID NO: 740 - protein, 18 aa, synthetic construct
  REGION 1..18: note = Synthetic Polypeptide
EQQEAIEHIDEVQNEIDR

SEQ ID NO: 741 - protein, 21 aa, synthetic construct
  REGION 1..21: note = Synthetic Polypeptide
AVEALAAALAHISGATSVDQR

SEQ ID NO: 742 - protein, 13 aa, synthetic construct
  REGION 1..13: note = Synthetic Polypeptide
RHLAPTGNAPASR

SEQ ID NO: 743 - protein, 18 aa, synthetic construct
  REGION 1..18: note = Synthetic Polypeptide
LLTDFCTHLPNLPDSTAK

SEQ ID NO: 744 - protein, 16 aa, synthetic construct
  REGION 1..16: note = Synthetic Polypeptide
VDEFVTHNLSFDEINK

SEQ ID NO: 745 - protein, 19 aa, synthetic construct
  REGION 1..19: note = Synthetic Polypeptide
ATLELTHNWGTEDDETQSY

SEQ ID NO: 746 - protein, 16 aa, synthetic construct
  REGION 1..16: note = Synthetic Polypeptide
EEFTAFLHPEEYDYMK

SEQ ID NO: 747 - protein, 12 aa, synthetic construct
  REGION 1..12: note = Synthetic Polypeptide
  VARIANT 2: note = misc_feature - Xaa can be any naturally occurring amino acid
QXFHPEQLITGK

SEQ ID NO: 748 - protein, 23 aa, synthetic construct
  REGION 1..23: note = Synthetic Polypeptide
PVTHNLPTVAHPSQAPSPNQPTK

SEQ ID NO: 749 - protein, 17 aa, synthetic construct
  REGION 1..17: note = Synthetic Polypeptide
  VARIANT 2..6: note = misc_feature - Xaa can be any naturally occurring amino acid
AXXXXXQHQAGQAPHLG

SEQ ID NO: 750 - protein, 17 aa, synthetic construct
  REGION 1..17: note = Synthetic Polypeptide
CNFTDGALVQHQEWDGK

SEQ ID NO: 751 - protein, 13 aa, synthetic construct
  REGION 1..13: note = Synthetic Polypeptide
GVLHQFSGTETNK

SEQ ID NO: 752 - protein, 16 aa, synthetic construct
  REGION 1..16: note = Synthetic Polypeptide
QIGAVVSHQSSVIPDR

SEQ ID NO: 753 - protein, 19 aa, synthetic construct
  REGION 1..19: note = Synthetic Polypeptide
IEPNEVTHSGDTGVETDGR

SEQ ID NO: 754 - protein, 15 aa, synthetic construct
  REGION 1..15: note = Synthetic Polypeptide
HYAHTDCPGHADYVK

SEQ ID NO: 755 - protein, 11 aa, synthetic construct
  REGION 1..11: note = Synthetic Polypeptide
TICSHVQNMIK

SEQ ID NO: 756 - protein, 13 aa, synthetic construct
  REGION 1..13: note = Synthetic Polypeptide
LLGHWEEAAHDLA

SEQ ID NO: 757 - protein, 12 aa, synthetic construct
  REGION 1..12: note = Synthetic Polypeptide
TYTIANQFPLNK

SEQ ID NO: 758 - protein, 17 aa, synthetic construct
  REGION 1..17: note = Synthetic Polypeptide
  VARIANT 4: note = misc_feature - Xaa can be any naturally occurring amino acid
NPTXFFDIAVDGEPLGR

SEQ ID NO: 759 - protein, 15 aa, synthetic construct
  REGION 1..15: note = Synthetic Polypeptide
LVSIGAEEIVDGNAK

SEQ ID NO: 760 - protein, 21 aa, synthetic construct
  REGION 1..21: note = Synthetic Polypeptide
TTDGVYEGVAIGGDRYPGSTF

SEQ ID NO: 761 - protein, 15 aa, synthetic construct
  REGION 1..15: note = Synthetic Polypeptide
THINIVVIGHVDSGK

SEQ ID NO: 762 - protein, 19 aa, synthetic construct
  REGION 1..19: note = Synthetic Polypeptide
DNDFCGTDMTIGTDSALHR

SEQ ID NO: 763 - protein, 18 aa, synthetic construct
  REGION 1..18: note = Synthetic Polypeptide
  VARIANT 3: note = misc_feature - Xaa can be any naturally occurring amino acid
VLXNMEIGTSLFDEEGAK

SEQ ID NO: 764 - protein, 16 aa, synthetic construct
  REGION 1..16: note = Synthetic Polypeptide
VCTLAIIDPGDSDIIR

SEQ ID NO: 765 - protein, 17 aa, synthetic construct
  REGION 1..17: note = Synthetic Polypeptide
GCITIIGGGDTATCCAK

SEQ ID NO: 766 - protein, 14 aa, synthetic construct
  REGION 1..14: note = Synthetic Polypeptide
TFNQVEIKPEMIGH

SEQ ID NO: 767 - protein, 13 aa, synthetic construct
  REGION 1..13: note = Synthetic Polypeptide
CQLEINFNTLQTK

SEQ ID NO: 768 - protein, 12 aa, synthetic construct
  REGION 1..12: note = Synthetic Polypeptide
HLEINPDHPIVE

SEQ ID NO: 769 - protein, 15 aa, synthetic construct
  REGION 1..15: note = Synthetic Polypeptide
HLEINPDHSIIETLR

SEQ ID NO: 770 - protein, 18 aa, synthetic construct
  REGION 1..18: note = Synthetic Polypeptide
VPYLIAGIQHSCQDIGAK

SEQ ID NO: 771 - protein, 10 aa, synthetic construct
  REGION 1..10: note = Synthetic Polypeptide
VLSIQSHVIR

SEQ ID NO: 772 - protein, 10 aa, synthetic construct
  REGION 1..10: note = Synthetic Polypeptide
ELGITALHIK

SEQ ID NO: 773 - protein, 10 aa, synthetic construct
  REGION 1..10: note = Synthetic Polypeptide
LVAIVDPHIK

SEQ ID NO: 774 - protein, 13 aa, synthetic construct
  REGION 1..13: note = Synthetic Polypeptide
TLTIVDTGIGMTK

SEQ ID NO: 775 - protein, 12 aa, synthetic construct
  REGION 1..12: note = Synthetic Polypeptide
LVAIVDVIDQNR

SEQ ID NO: 776 - protein, 14 aa, synthetic construct
  REGION 1..14: note = Synthetic Polypeptide
QIILEKEETEELKR

SEQ ID NO: 777 - protein, 10 aa, synthetic construct
  REGION 1..10: note = Synthetic Polypeptide
  VARIANT 1: note = misc_feature - Xaa can be any naturally occurring amino acid
XKHPDADSLY

SEQ ID NO: 778 - protein, 15 aa, synthetic construct
  REGION 1..15: note = Synthetic Polypeptide
CIGKPGGSLDNSEQK

SEQ ID NO: 779 - protein, 24 aa, synthetic construct
  REGION 1..24: note = Synthetic Polypeptide
HHIYLEGTLLKPNMVTPGHACTQK

SEQ ID NO: 780 - protein, 19 aa, synthetic construct
  REGION 1..19: note = Synthetic Polypeptide
LTQQLAQATGKPPQYIAVH

SEQ ID NO: 781 - protein, 25 aa, synthetic construct
  REGION 1..25: note = Synthetic Polypeptide
SSPPELPDVMKPQDSGSSANEQAVQ

SEQ ID NO: 782 - protein, 13 aa, synthetic construct
  REGION 1..13: note = Synthetic Polypeptide
LQELEKYPGIQTR

SEQ ID NO: 783 - protein, 12 aa, synthetic construct
  REGION 1..12: note = Synthetic Polypeptide
WIGLDLSNGKPR

SEQ ID NO: 784 - protein, 14 aa, synthetic construct
  REGION 1..14: note = Synthetic Polypeptide
MPFLELDTNLPANR

SEQ ID NO: 785 - protein, 20 aa, synthetic construct
  REGION 1..20: note = Synthetic Polypeptide
ETALLSSGFSLEDPQTHANR

SEQ ID NO: 786 - protein, 17 aa, synthetic construct
  REGION 1..17: note = Synthetic Polypeptide
EAFSLFDKDGDGTITTK

SEQ ID NO: 787 - protein, 11 aa, synthetic construct
  REGION 1..11: note = Synthetic Polypeptide
YELGRPAANTK

SEQ ID NO: 788 - protein, 20 aa, synthetic construct
  REGION 1..20: note = Synthetic Polypeptide
GNPICSLHDQGAGGNGNVLK

SEQ ID NO: 789 - protein, 16 aa, synthetic construct
  REGION 1..16: note = Synthetic Polypeptide
VILHLKEDQTEYLEER

SEQ ID NO: 790 - protein, 20 aa, synthetic construct
  REGION 1..20: note = Synthetic Polypeptide
IQQLCEDIIQLKPDVVITEK

SEQ ID NO: 791 - protein, 20 aa, synthetic construct
  REGION 1..20: note = Synthetic Polypeptide
IQQLCEDIIQLKPDVVITEK

SEQ ID NO: 792 - protein, 10 aa, synthetic construct
  REGION 1..10: note = Synthetic Polypeptide
TLNNDIMLIK

SEQ ID NO: 793 - protein, 15 aa, synthetic construct
  REGION 1..15: note = Synthetic Polypeptide
NQVALNPQNTVFDAK

SEQ ID NO: 794 - protein, 15 aa, synthetic construct
  REGION 1..15: note = Synthetic Polypeptide
NQVALNPQNTVFDAK

SEQ ID NO: 795 - protein, 19 aa, synthetic construct
  REGION 1..19: note = Synthetic Polypeptide
STATLAWGVNLPAHTVIIK

SEQ ID NO: 796 - protein, 13 aa, synthetic construct
  REGION 1..13: note = Synthetic Polypeptide
  VARIANT 2: note = misc_feature - Xaa can be any naturally occurring amino acid
EXLELPEDEEEKK

SEQ ID NO: 797 - protein, 17 aa, synthetic construct
  REGION 1..17: note = Synthetic Polypeptide
GVNLPGAAVDLPAVSEK

SEQ ID NO: 798 - protein, 10 aa, synthetic construct
  REGION 1..10: note = Synthetic Polypeptide
RLPPAAGDEP

SEQ ID NO: 799 - protein, 9 aa, synthetic construct
  REGION 1..9: note = Synthetic Polypeptide
LDLPPYETF

SEQ ID NO: 800 - protein, 18 aa, synthetic construct
  REGION 1..18: note = Synthetic Polypeptide
DGDSVMVLPTIPEEEAKK

SEQ ID NO: 801 - protein, 17 aa, synthetic construct
  REGION 1..17: note = Synthetic Polypeptide
EIVHLQAGQCGNQIGAK

SEQ ID NO: 802 - protein, 20 aa, synthetic construct
  REGION 1..20: note = Synthetic Polypeptide
DVSIEDSVISLSGDHCIIGR

SEQ ID NO: 803 - protein, 20 aa, synthetic construct
  REGION 1..20: note = Synthetic Polypeptide
SSAPGPLELDLTGDLESFKK

SEQ ID NO: 804 - protein, 11 aa, synthetic construct
  REGION 1..11: note = Synthetic Polypeptide
FLEMCNDLLAR

SEQ ID NO: 805 - protein, 15 aa, synthetic construct
  REGION 1..15: note = Synthetic Polypeptide
TTGFGMIYDSLDYAK

SEQ ID NO: 806 - protein, 12 aa, synthetic construct
  REGION 1..12: note = Synthetic Polypeptide
  VARIANT 1: note = misc_feature - Xaa can be any naturally occurring amino acid
XMNPTNTVFDAK

SEQ ID NO: 807 - protein, 12 aa, synthetic construct
  REGION 1..12: note = Synthetic Polypeptide
EDAMAMVDHCLK

SEQ ID NO: 808 - protein, 18 aa, synthetic construct
  REGION 1..18: note = Synthetic Polypeptide
  VARIANT 3: note = misc_feature - Xaa can be any naturally occurring amino acid
  VARIANT 5: note = misc_feature - Xaa can be any naturally occurring amino acid
  VARIANT 9: note = misc_feature - Xaa can be any naturally occurring amino acid
ANXVXSGGXTMYPGIADR

SEQ ID NO: 809 - protein, 18 aa, synthetic construct
  REGION 1..18: note = Synthetic Polypeptide
  VARIANT 3: note = misc_feature - Xaa can be any naturally occurring amino acid
  VARIANT 5: note = misc_feature - Xaa can be any naturally occurring amino acid
  VARIANT 9: note = misc_feature - Xaa can be any naturally occurring amino acid
ANXVXSGGXTMYPGIADR

SEQ ID NO: 810 - protein, 18 aa, synthetic construct
  REGION 1..18: note = Synthetic Polypeptide
ALQDLENAASGDAAVHQR

SEQ ID NO: 811 - protein, 15 aa, synthetic construct
  REGION 1..15: note = Synthetic Polypeptide
DPVTNLNNAFEVAEK

SEQ ID NO: 812 - protein, 13 aa, synthetic construct
  REGION 1..13: note = Synthetic Polypeptide
  VARIANT 1: note = misc_feature - Xaa can be any naturally occurring amino acid
XNAGPNTNGSQFF

SEQ ID NO: 813 - protein, 18 aa, synthetic construct
  REGION 1..18: note = Synthetic Polypeptide
NYSVFYYEIQNAPEQACH

SEQ ID NO: 814 - protein, 14 aa, synthetic construct
  REGION 1..14: note = Synthetic Polypeptide
ELISNASDALDKIR

SEQ ID NO: 815 - protein, 24 aa, synthetic construct
  REGION 1..24: note = Synthetic Polypeptide
YYFNHITNASQWERPSGNSSSGGK

SEQ ID NO: 816 - protein, 11 aa, synthetic construct
  REGION 1..11: note = Synthetic Polypeptide
TNDWEDHLAVK

SEQ ID NO: 817 - protein, 13 aa, synthetic construct
  REGION 1..13: note = Synthetic Polypeptide
AFHNEAQVNPERK

SEQ ID NO: 818 - protein, 13 aa, synthetic construct
  REGION 1..13: note = Synthetic Polypeptide
NCLTNFHGMDLTR

SEQ ID NO: 819 - protein, 14 aa, synthetic construct
  REGION 1..14: note = Synthetic Polypeptide
TNVANFPGHSGPIT

SEQ ID NO: 820 - protein, 18 aa, synthetic construct
  REGION 1..18: note = Synthetic Polypeptide
ILNNGHAFNVEFDDSQDK

SEQ ID NO: 821 - protein, 14 aa, synthetic construct
  REGION 1..14: note = Synthetic Polypeptide
IEQLQNHENEDIYK

SEQ ID NO: 822 - protein, 23 aa, synthetic construct
  REGION 1..23: note = Synthetic Polypeptide
PVFVHAGPFANIAHGNSSIIADR

SEQ ID NO: 823 - protein, 14 aa, synthetic construct
  REGION 1..14: note = Synthetic Polypeptide
VWYVSNIDGTHIAK

SEQ ID NO: 824 - protein, 19 aa, synthetic construct
  REGION 1..19: note = Synthetic Polypeptide
CDEVMQLLLENLGNENVHR

SEQ ID NO: 825 - protein, 19 aa, synthetic construct
  REGION 1..19: note = Synthetic Polypeptide
QDQRPLHPVANPHAEISTK

SEQ ID NO: 826 - protein, 12 aa, synthetic construct
  REGION 1..12: note = Synthetic Polypeptide
  VARIANT 1: note = misc_feature - Xaa can be any naturally occurring amino acid
XNPLDAGAAEPI

SEQ ID NO: 827 - protein, 19 aa, synthetic construct
  REGION 1..19: note = Synthetic Polypeptide
LIPQLVANVTNPNSTEHMK

SEQ ID NO: 828 - protein, 16 aa, synthetic construct
  REGION 1..16: note = Synthetic Polypeptide
SAAMLGNSEDHTALSR

SEQ ID NO: 829 - protein, 29 aa, synthetic construct
  REGION 1..29: note = Synthetic Polypeptide
NYQQNYQNSESGEKNEGSESAPEGQAQQR

SEQ ID NO: 830 - protein, 18 aa, synthetic construct
  REGION 1..18: note = Synthetic Polypeptide
LGEMWNNTAADDKQPYEK

SEQ ID NO: 831 - protein, 17 aa, synthetic construct
  REGION 1..17: note = Synthetic Polypeptide
IMQNTDPHSQEYVEHLK

SEQ ID NO: 832 - protein, 14 aa, synthetic construct
  REGION 1..14: note = Synthetic Polypeptide
ILIANTGMDTDKIK

SEQ ID NO: 833 - protein, 20 aa, synthetic construct
  REGION 1..20: note = Synthetic Polypeptide
AWVWNTHADFADECPKPELL

SEQ ID NO: 834 - protein, 18 aa, synthetic construct
  REGION 1..18: note = Synthetic Polypeptide
DHASIQMNVAEVDKVTGR

SEQ ID NO: 835 - protein, 12 aa, synthetic construct
  REGION 1..12: note = Synthetic Polypeptide
ALANVNIGSLIC

SEQ ID NO: 836 - protein, 13 aa, synthetic construct
  REGION 1..13: note = Synthetic Polypeptide
  VARIANT 4..5: note = misc_feature - Xaa can be any naturally occurring amino acid
EHGXXTNWDDMEK

SEQ ID NO: 837 - protein, 16 aa, synthetic construct
  REGION 1..16: note = Synthetic Polypeptide
SAAQAAAQTNSNAAGK

SEQ ID NO: 838 - protein, 19 aa, synthetic construct
  REGION 1..19: note = Synthetic Polypeptide
EETFEAAMLGQAEEVVQER

SEQ ID NO: 839 - protein, 18 aa, synthetic construct
  REGION 1..18: note = Synthetic Polypeptide
PPYDEQTQAFIDAAQEAR

SEQ ID NO: 840 - protein, 15 aa, synthetic construct
  REGION 1..15: note = Synthetic Polypeptide
LEQGQAIDDLMPAQK

SEQ ID NO: 841 - protein, 16 aa, synthetic construct
  REGION 1..16: note = Synthetic Polypeptide
SLHQAIEGDTSGDFLK

SEQ ID NO: 842 - protein, 16 aa, synthetic construct
  REGION 1..16: note = Synthetic Polypeptide
QLQQAQAAGAEQEVEK

SEQ ID NO: 843 - protein, 19 aa, synthetic construct
  REGION 1..19: note = Synthetic Polypeptide
YLEVVLNTLQQASQAQVDK

SEQ ID NO: 844 - protein, 19 aa, synthetic construct
  REGION 1..19: note = Synthetic Polypeptide
YLEVVLNTLQQASQAQVDK

SEQ ID NO: 845 - protein, 20 aa, synthetic construct
  REGION 1..20: note = Synthetic Polypeptide
FLSELTQQLAQATGKPPQYI

SEQ ID NO: 846 - protein, 21 aa, synthetic construct
  REGION 1..21: note = Synthetic Polypeptide
FLSELTQQLAQATGKPPQYIA

SEQ ID NO: 847 - protein, 23 aa, synthetic construct
  REGION 1..23: note = Synthetic Polypeptide
FLSELTQQLAQATGKPPQYIAVH

SEQ ID NO: 848 - protein, 14 aa, synthetic construct
  REGION 1..14: note = Synthetic Polypeptide
MTSMGQATWSDPHK

SEQ ID NO: 849 - protein, 19 aa, synthetic construct
  REGION 1..19: note = Synthetic Polypeptide
EELGLIEQAYDNPHEALSR

SEQ ID NO: 850 - protein, 17 aa, synthetic construct
  REGION 1..17: note = Synthetic Polypeptide
SLGTIQQCCDAIDHLCR

SEQ ID NO: 851 - protein, 25 aa, synthetic construct
  REGION 1..25: note = Synthetic Polypeptide
AAAAAAQQQQQCGGGGATKPAVSGK

SEQ ID NO: 852 - protein, 15 aa, synthetic construct
  REGION 1..15: note = Synthetic Polypeptide
NSCNQCNEPRPEDSR

SEQ ID NO: 853 - protein, 19 aa, synthetic construct
  REGION 1..19: note = Synthetic Polypeptide
VLIAFAQYLQQCPFEDHVK

SEQ ID NO: 854 - protein, 14 aa, synthetic construct
  REGION 1..14: note = Synthetic Polypeptide
DSLLQDGEFSMDLR

SEQ ID NO: 855 - protein, 18 aa, synthetic construct
  REGION 1..18: note = Synthetic Polypeptide
YFLGSIVNFSQDPDVHFK

SEQ ID NO: 856 - protein, 18 aa, synthetic construct
  REGION 1..18: note = Synthetic Polypeptide
VFSWLQQEGHLSEEEMAR

SEQ ID NO: 857 - protein, 12 aa, synthetic construct
  REGION 1..12: note = Synthetic Polypeptide
VMSQEIQEQLHK

SEQ ID NO: 858 - protein, 11 aa, synthetic construct
  REGION 1..11: note = Synthetic Polypeptide
KQEPVKPEEGR

SEQ ID NO: 859 - protein, 18 aa, synthetic construct
  REGION 1..18: note = Synthetic Polypeptide
LWYCDLQQESSGIAGILK

SEQ ID NO: 860 - protein, 14 aa, synthetic construct
  REGION 1..14: note = Synthetic Polypeptide
KQEYDESGPSIVHR

SEQ ID NO: 861 - protein, 19 aa, synthetic construct
  REGION 1..19: note = Synthetic Polypeptide
ETEAICFFVQQFTDMEHNR

SEQ ID NO: 862 - protein, 13 aa, synthetic construct
  REGION 1..13: note = Synthetic Polypeptide
VTEQGAELSNEER

SEQ ID NO: 863 - protein, 17 aa, synthetic construct
  REGION 1..17: note = Synthetic Polypeptide
AYMGNVLQGGEGQAPTR

SEQ ID NO: 864 - protein, 14 aa, synthetic construct
  REGION 1..14: note = Synthetic Polypeptide
AVTEQGHELSNEER

SEQ ID NO: 865 - protein, 18 aa, synthetic construct
  REGION 1..18: note = Synthetic Polypeptide
VAHTFVVDVAQGTQVTGR

SEQ ID NO: 866 - protein, 11 aa, synthetic construct
  REGION 1..11: note = Synthetic Polypeptide
VGQGYPHDPPK

SEQ ID NO: 867 - protein, 18 aa, synthetic construct
  REGION 1..18: note = Synthetic Polypeptide
IYAVEASTMAQHAEVLVK

SEQ ID NO: 868 - protein, 21 aa, synthetic construct
  REGION 1..21: note = Synthetic Polypeptide
TLAIYFEVVNQHNAPIPQGGR

SEQ ID NO: 869 - protein, 18 aa, synthetic construct
  REGION 1..18: note = Synthetic Polypeptide
ELAQIAGRPTEDEDEKEK

SEQ ID NO: 870 - protein, 18 aa, synthetic construct
  REGION 1..18: note = Synthetic Polypeptide
MDEMATTQISKDELDELK

SEQ ID NO: 871 - protein, 15 aa, synthetic construct
  REGION 1..15: note = Synthetic Polypeptide
YPHLGQKPGGSDFLR

SEQ ID NO: 872 - protein, 19 aa, synthetic construct
  REGION 1..19: note = Synthetic Polypeptide
TMLELLNQLDGFQPNTQVK

SEQ ID NO: 873 - protein, 19 aa, synthetic construct
  REGION 1..19: note = Synthetic Polypeptide
ILLELLNQMDGFDQNVNVK

SEQ ID NO: 874 - protein, 13 aa, synthetic construct
  REGION 1..13: note = Synthetic Polypeptide
LLNQMDGFDTLHR

SEQ ID NO: 875 - protein, 20 aa, synthetic construct
  REGION 1..20: note = Synthetic Polypeptide
FQESAEAILGQNAAYLGELK

SEQ ID NO: 876 - protein, 21 aa, synthetic construct
  REGION 1..21: note = Synthetic Polypeptide
HPCFIIAEIGQNHQGDLDVAK

SEQ ID NO: 877 - protein, 22 aa, synthetic construct
  REGION 1..22: note = Synthetic Polypeptide
LLQDHPWLLSQNLVVKPDQLIK

SEQ ID NO: 878 - protein, 17 aa, synthetic construct
  REGION 1..17: note = Synthetic Polypeptide
ALPAVQQNNLDEDLIRK

SEQ ID NO: 879 - protein, 13 aa, synthetic construct
  REGION 1..13: note = Synthetic Polypeptide
ALGQNPTNAEVLK

SEQ ID NO: 880 - protein, 14 aa, synthetic construct
  REGION 1..14: note = Synthetic Polypeptide
NYQQNYQNSESGEK

SEQ ID NO: 881 - protein, 29 aa, synthetic construct
  REGION 1..29: note = Synthetic Polypeptide
NYQQNYQNSESGEKNEGSESAPEGQAQQR

SEQ ID NO: 882 - protein, 22 aa, synthetic construct
  REGION 1..22: note = Synthetic Polypeptide
CGAPSATQPATAETQHIADQVR

SEQ ID NO: 883 - protein, 26 aa, synthetic construct
  REGION 1..26: note = Synthetic Polypeptide
QAAAAAAQQQQQCGGGGATKPAVSGK

SEQ ID NO: 884 - protein, 19 aa, synthetic construct
  REGION 1..19: note = Synthetic Polypeptide
IDVTDFLSMTQQDSHAPLR

SEQ ID NO: 885 - protein, 15 aa, synthetic construct
  REGION 1..15: note = Synthetic Polypeptide
IGSCTQQDVELHVQK

SEQ ID NO: 886 - protein, 14 aa, synthetic construct
  REGION 1..14: note = Synthetic Polypeptide
LFPLNQQDVPDKFK

SEQ ID NO: 887 - protein, 18 aa, synthetic construct
  REGION 1..18: note = Synthetic Polypeptide
IGQQPQQPGAPPQQDYTK

SEQ ID NO: 888 - protein, 27 aa, synthetic construct
  REGION 1..27: note = Synthetic Polypeptide
HQAAAAAAQQQQQCGGGGATKPAVSGK

SEQ ID NO: 889 - protein, 12 aa, synthetic construct
  REGION 1..12: note = Synthetic Polypeptide
MFTQQQPQELAR

SEQ ID NO: 890 - protein, 19 aa, synthetic construct
  REGION 1..19: note = Synthetic Polypeptide
LQQQQRPEDAEDGAEGGGK

SEQ ID NO: 891 - protein, 20 aa, synthetic construct
  REGION 1..20: note = Synthetic Polypeptide
LQQQQRPEDAEDGAEGGGKR

SEQ ID NO: 892 - protein, 21 aa, synthetic construct
  REGION 1..21: note = Synthetic Polypeptide
SSEADMECLNQRPPENPDTDK

SEQ ID NO: 893 - protein, 24 aa, synthetic construct
  REGION 1..24: note = Synthetic Polypeptide
SSEADMECLNQRPPENPDTDKNVQ

SEQ ID NO: 894 - protein, 24 aa, synthetic construct
  REGION 1..24: note = Synthetic Polypeptide
NVNPESQLIQQSEQSESETAGSTK

SEQ ID NO: 895 - protein, 17 aa, synthetic construct
  REGION 1..17: note = Synthetic Polypeptide
  VARIANT 5: note = misc_feature - Xaa can be any naturally occurring amino acid
PDNFXFGQSGAGNNWAK

SEQ ID NO: 896 - protein, 19 aa, synthetic construct
  REGION 1..19: note = Synthetic Polypeptide
SQTCEFNMIEQSGPPHEPR

SEQ ID NO: 897 - protein, 23 aa, synthetic construct
  REGION 1..23: note = Synthetic Polypeptide
SAVLPPEDMSQSGPSGSHPQGPR

SEQ ID NO: 898 - protein, 15 aa, synthetic construct
  REGION 1..15: note = Synthetic Polypeptide
IEFLQSHENQEIYQK

SEQ ID NO: 899 - protein, 16 aa, synthetic construct
  REGION 1..16: note = Synthetic Polypeptide
NTVSQSISGDPEIDKK

SEQ ID NO: 900 - protein, 15 aa, synthetic construct
  REGION 1..15: note = Synthetic Polypeptide
LLIHQSLAGGIIGVK

SEQ ID NO: 901 - protein, 18 aa, synthetic construct
  REGION 1..18: note = Synthetic Polypeptide
  VARIANT 3: note = misc_feature - Xaa can be any naturally occurring amino acid
MVXYLANLTQSQIALNEK

SEQ ID NO: 902 - protein, 22 aa, synthetic construct
  REGION 1..22: note = Synthetic Polypeptide
PPKPEPFQFGQSSQKPPVAGGK

SEQ ID NO: 903 - protein, 21 aa, synthetic construct
  REGION 1..21: note = Synthetic Polypeptide
NGNYCVLQMDQSYKPDENEVR

SEQ ID NO: 904 - protein, 19 aa, synthetic construct
  REGION 1..19: note = Synthetic Polypeptide
ILVGDVGQTVDDPYATFVK

SEQ ID NO: 905 - protein, 18 aa, synthetic construct
  REGION 1..18: note = Synthetic Polypeptide
ADDVDLEQVANETHGHVG

SEQ ID NO: 906 - protein, 19 aa, synthetic construct
  REGION 1..19: note = Synthetic Polypeptide
ADDVDLEQVANETHGHVGA

SEQ ID NO: 907 - protein, 17 aa, synthetic construct
  REGION 1..17: note = Synthetic Polypeptide
SINFLHQVCHDQTPTTK

SEQ ID NO: 908 - protein, 19 aa, synthetic construct
  REGION 1..19: note = Synthetic Polypeptide
CTTVAFTQVNSEDKGALAK

SEQ ID NO: 909 - protein, 12 aa, synthetic construct
  REGION 1..12: note = Synthetic Polypeptide
QQLQQVPGLLHR

SEQ ID NO: 910 - protein, 12 aa, synthetic construct
  REGION 1..12: note = Synthetic Polypeptide
SQQYPAARPAEP

SEQ ID NO: 911 - protein, 18 aa, synthetic construct
  REGION 1..18: note = Synthetic Polypeptide
DFCIQVGRNIIHGSDSVK

SEQ ID NO: 912 - protein, 18 aa, synthetic construct
  REGION 1..18: note = Synthetic Polypeptide
VLMSHLGRPDGVPMPDKY

SEQ ID NO: 913 - protein, 19 aa, synthetic construct
  REGION 1..19: note = Synthetic Polypeptide
VLMSHLGRPDGVPMPDKYS

SEQ ID NO: 914 - protein, 14 aa, synthetic construct
  REGION 1..14: note = Synthetic Polypeptide
AQVARPGGDTIFGK

SEQ ID NO: 915 - protein, 14 aa, synthetic construct
  REGION 1..14: note = Synthetic Polypeptide
AQVARPGGDTIFGK

SEQ ID NO: 916 - protein, 17 aa, synthetic construct
  REGION 1..17: note = Synthetic Polypeptide
FMSVQRPGPYDRPGTAR

SEQ ID NO: 917 - protein, 13 aa, synthetic construct
  REGION 1..13: note = Synthetic Polypeptide
VLVERSAAETVTK

SEQ ID NO: 918 - protein, 14 aa, synthetic construct
  REGION 1..14: note = Synthetic Polypeptide
FLPSARSSPASSPE

SEQ ID NO: 919 - protein, 21 aa, synthetic construct
  REGION 1..21: note = Synthetic Polypeptide
RPELGSEGLGSAAHGSQPDLR

SEQ ID NO: 920 - protein, 17 aa, synthetic construct
  REGION 1..17: note = Synthetic Polypeptide
MPDQGMTSADDFFQGTK

SEQ ID NO: 921 - protein, 22 aa, synthetic construct
  REGION 1..22: note = Synthetic Polypeptide
DVPAPSTSADKVESLDVDSEAK

SEQ ID NO: 922 - protein, 17 aa, synthetic construct
  REGION 1..17: note = Synthetic Polypeptide
QVCLPVISAENWKPATK

SEQ ID NO: 923 - protein, 20 aa, synthetic construct
  REGION 1..20: note = Synthetic Polypeptide
GFGSGDDPYSSAEPHVSGVK

SEQ ID NO: 924 - protein, 21 aa, synthetic construct
  REGION 1..21: note = Synthetic Polypeptide
EFGDNTAGCTSAGPHFNPLSR

SEQ ID NO: 925 - protein, 21 aa, synthetic construct
  REGION 1..21: note = Synthetic Polypeptide
TYFSCTSAHTSTGDGTAMITR

SEQ ID NO: 926 - protein, 14 aa, synthetic construct
  REGION 1..14: note = Synthetic Polypeptide
TYSLGSALRPSTSR

SEQ ID NO: 927 - protein, 17 aa, synthetic construct
  REGION 1..17: note = Synthetic Polypeptide
VSDQELQSANASVDDSR

SEQ ID NO: 928 - protein, 19 aa, synthetic construct
  REGION 1..19: note = Synthetic Polypeptide
APGSAAPAAGSAPAAAEEK

SEQ ID NO: 929 - protein, 19 aa, synthetic construct
  REGION 1..19: note = Synthetic Polypeptide
APGSAAPAAGSAPAAAEEK

SEQ ID NO: 930 - protein, 19 aa, synthetic construct
  REGION 1..19: note = Synthetic Polypeptide
APGSAAPAAGSAPAAAEEK

SEQ ID NO: 931 - protein, 20 aa, synthetic construct
  REGION 1..20: note = Synthetic Polypeptide
APGSAAPAAGSAPAAAEEKK

SEQ ID NO: 932 - protein, 15 aa, synthetic construct
  REGION 1..15: note = Synthetic Polypeptide
NEGSESAPEGQAQQR

SEQ ID NO: 933 - protein, 20 aa, synthetic construct
  REGION 1..20: note = Synthetic Polypeptide
QVEPLDPPAGSAPGEHVFVK

SEQ ID NO: 934 - protein, 18 aa, synthetic construct
  REGION 1..18: note = Synthetic Polypeptide
PTGEAGPSCSSASDKLPR

SEQ ID NO: 935 - protein, 14 aa, synthetic construct
  REGION 1..14: note = Synthetic Polypeptide
YYTSASGDEMVSLK

SEQ ID NO: 936 - protein, 20 aa, synthetic construct
  REGION 1..20: note = Synthetic Polypeptide
NQQGAHSALSSASTSSHNLQ

SEQ ID NO: 937 - protein, 16 aa, synthetic construct
  REGION 1..16: note = Synthetic Polypeptide
EALLSSAVDHGSDEVK

SEQ ID NO: 938 - protein, 19 aa, synthetic construct
  REGION 1..19: note = Synthetic Polypeptide
DYMVEIDILASCDHPNIVK

SEQ ID NO: 939 - protein, 11 aa, synthetic construct
  REGION 1..11: note = Synthetic Polypeptide
MESCGIHETTF

SEQ ID NO: 940 - protein, 12 aa, synthetic construct
  REGION 1..12: note = Synthetic Polypeptide
QLSSCLPNIVPK

SEQ ID NO: 941 - protein, 12 aa, synthetic construct
  REGION 1..12: note = Synthetic Polypeptide
  VARIANT 3: note = misc_feature - Xaa can be any naturally occurring amino acid
LIXSDGHEFIVK

SEQ ID NO: 942 - protein, 19 aa, synthetic construct
  REGION 1..19: note = Synthetic Polypeptide
EIVDGGVILESDPQQVVHR

SEQ ID NO: 943 - protein, 15 aa, synthetic construct
  REGION 1..15: note = Synthetic Polypeptide
SLEDALSSDTSGHFR

SEQ ID NO: 944 - protein, 20 aa, synthetic construct
  REGION 1..20: note = Synthetic Polypeptide
VGVEAHVDIHSDVPKGANSF

SEQ ID NO: 945 - protein, 16 aa, synthetic construct
  REGION 1..16: note = Synthetic Polypeptide
VILGSEAAQQHPEEVR

SEQ ID NO: 946 - protein, 10 aa, synthetic construct
  REGION 1..10: note = Synthetic Polypeptide
  VARIANT 1: note = misc_feature - Xaa can be any naturally occurring amino acid
XSEDKGALAK

SEQ ID NO: 947 - protein, 20 aa, synthetic construct
  REGION 1..20: note = Synthetic Polypeptide
  VARIANT 5..6: note = misc_feature - Xaa can be any naturally occurring amino acid
GGTSXXSSEGTQHSYSEEEK

SEQ ID NO: 948 - protein, 24 aa, synthetic construct
  REGION 1..24: note = Synthetic Polypeptide
CALGGTSELSSEGTQHSYSEEEKY

SEQ ID NO: 949 - protein, 15 aa, synthetic construct
  REGION 1..15: note = Synthetic Polypeptide
MDPNIVGSEHYDVAR

SEQ ID NO: 950 - protein, 21 aa, synthetic construct
  REGION 1..21: note = Synthetic Polypeptide
SPAPSSVPLGSEKPSNVSQDR

SEQ ID NO: 951 - protein, 20 aa, synthetic construct
  REGION 1..20: note = Synthetic Polypeptide
MTQAGVEELESENKIPATQK

SEQ ID NO: 952 - protein, 13 aa, synthetic construct
  REGION 1..13: note = Synthetic Polypeptide
MLLDSEQHPCQLK

SEQ ID NO: 953 - protein, 37 aa, synthetic construct
  REGION 1..37: note = Synthetic Polypeptide
GLGNVLGGLISGAGGGGGGGGGGGGGGGGGGGGTAMR

SEQ ID NO: 954 - protein, 37 aa, synthetic construct
  REGION 1..37: note = Synthetic Polypeptide
IMDDLTEVLCSGAGGVHSGGSGDGAGSGGPGAQNHVK

SEQ ID NO: 955 - protein, 35 aa, synthetic construct; identical to SEQ ID NO: 305
  REGION 1..35: note = Synthetic Polypeptide

SEQ ID NO: 956 - protein, 22 aa, synthetic construct
  REGION 1..22: note = Synthetic Polypeptide
LEPAPLDSLCSGASAEEPTSHR

SEQ ID NO: 957 - protein, 12 aa, synthetic construct
  REGION 1..12: note = Synthetic Polypeptide
VIGSGCNLDSAR

SEQ ID NO: 958 - protein, 14 aa, synthetic construct
  REGION 1..14: note = Synthetic Polypeptide
  VARIANT 2: note = misc_feature - Xaa can be any naturally occurring amino acid
WXLNSGDGAFYGPK

SEQ ID NO: 959 - protein, 22 aa, synthetic construct
  REGION 1..22: note = Synthetic Polypeptide
FFDMAYQGFASGDGDKDAWAVR

SEQ ID NO: 960 - protein, 19 aa, synthetic construct
  REGION 1..19: note = Synthetic Polypeptide
VSIEDSVISLSGDHCIIGR

SEQ ID NO: 961 - protein, 16 aa, synthetic construct
  REGION 1..16: note = Synthetic Polypeptide
EYLLSGDISEAEHCLK

SEQ ID NO: 962 - protein, 14 aa, synthetic construct
  REGION 1..14: note = Synthetic Polypeptide
DDGLFSGDPNWFPK

SEQ ID NO: 963 - protein, 21 aa, synthetic construct
  REGION 1..21: note = Synthetic Polypeptide
WQHDLFDSGFGGGAGVETGGK

SEQ ID NO: 964 - protein, 18 aa, synthetic construct
  REGION 1..18: note = Synthetic Polypeptide
DSVWGSGGGQQSVNHLVK

SEQ ID NO: 965 - protein, 25 aa, synthetic construct
  REGION 1..25: note = Synthetic Polypeptide
PEGPNEAEVTSGKPEQEVPDAEEEK

SEQ ID NO: 966 - protein, 10 aa, synthetic construct
  REGION 1..10: note = Synthetic Polypeptide
VQSGNINAAK

SEQ ID NO: 967 - protein, 16 aa, synthetic construct
  REGION 1..16: note = Synthetic Polypeptide
YQYGGLNSGRPVTPPR

SEQ ID NO: 968 - protein, 26 aa, synthetic construct
  REGION 1..26: note = Synthetic Polypeptide
VLQATVVAVGSGSKGKGGEIQPVSVK

SEQ ID NO: 969 - protein, 17 aa, synthetic construct
  REGION 1..17: note = Synthetic Polypeptide
GILFVGSGVSGGEEGAR

SEQ ID NO: 970 - protein, 15 aa, synthetic construct
  REGION 1..15: note = Synthetic Polypeptide
IEFLQSHENQEIYQK

SEQ ID NO: 971 - protein, 19 aa, synthetic construct
  REGION 1..19: note = Synthetic Polypeptide
LDEVITSHGAIEPDKDNVR

SEQ ID NO: 972 - protein, 17 aa, synthetic construct
  REGION 1..17: note = Synthetic Polypeptide
EHPVIESHPDNALEDLR

SEQ ID NO: 973 - protein, 15 aa, synthetic construct
  REGION 1..15: note = Synthetic Polypeptide
LIQSHPESAEDLQEK

SEQ ID NO: 974 - protein, 13 aa, synthetic construct
  REGION 1..13: note = Synthetic Polypeptide
TIVITSHPGQIVK

SEQ ID NO: 975 - protein, 16 aa, synthetic construct
  REGION 1..16: note = Synthetic Polypeptide
IEWLESHQDADIEDFK

SEQ ID NO: 976 - protein, 17 aa, synthetic construct
  REGION 1..17: note = Synthetic Polypeptide
GYPHLCSICDLPVHSNK

SEQ ID NO: 977 - protein, 21 aa, synthetic construct
  REGION 1..21: note = Synthetic Polypeptide
SEPCALCSLHSIGKIGGAQNR

SEQ ID NO: 978 - protein, 13 aa, synthetic construct
  REGION 1..13: note = Synthetic Polypeptide
LQSIGTENTEENR

SEQ ID NO: 979 - protein, 14 aa, synthetic construct
  REGION 1..14: note = Synthetic Polypeptide
LFIHESIHDEVVNR

SEQ ID NO: 980 - protein, 24 aa, synthetic construct
  REGION 1..24: note = Synthetic Polypeptide
VTFNINNSIPPTFDGEEEPSQGQK

SEQ ID NO: 981 - protein, 23 aa, synthetic construct
  REGION 1..23: note = Synthetic Polypeptide
NLNTLCWAIGSISGAMHEEDEKR

SEQ ID NO: 982 - protein, 21 aa, synthetic construct
  REGION 1..21: note = Synthetic Polypeptide
EASATNSPCTSKPATPAPSEK

SEQ ID NO: 983 - protein, 18 aa, synthetic construct
  REGION 1..18: note = Synthetic Polypeptide
PPNPNCYVCASKPEVTVR

SEQ ID NO: 984 - protein, 10 aa, synthetic construct
  REGION 1..10: note = Synthetic Polypeptide
ICSKPVVLPK

SEQ ID NO: 985 - protein, 20 aa, synthetic construct
  REGION 1..20: note = Synthetic Polypeptide
QFHFHWGSLDGQGSEHTVDK

SEQ ID NO: 986 - protein, 21 aa, synthetic construct
  REGION 1..21: note = Synthetic Polypeptide
QFHFHWGSLDGQGSEHTVDKK

SEQ ID NO: 987 - protein, 20 aa, synthetic construct
  REGION 1..20: note = Synthetic Polypeptide
GNPICSLHDQGAGGNGNVLK

SEQ ID NO: 988 - protein, 16 aa, synthetic construct
  REGION 1..16: note = Synthetic Polypeptide
EANFTVSSMHGDMPQK

SEQ ID NO: 989 - protein, 15 aa, synthetic construct
  REGION 1..15: note = Synthetic Polypeptide
NQLTSNPENTVFDAK

SEQ ID NO: 990 - protein, 20 aa, synthetic construct
  REGION 1..20: note = Synthetic Polypeptide
QVLVGSYCVFSNQGGLVHPK

SEQ ID NO: 991 - protein, 12 aa, synthetic construct
  REGION 1..12: note = Synthetic Polypeptide
DLQSNVEHLTEK

SEQ ID NO: 992 - protein, 14 aa, synthetic construct
  REGION 1..14: note = Synthetic Polypeptide
EEMQSNVEVVHTYR

SEQ ID NO: 993 - protein, 23 aa, synthetic construct
  REGION 1..23: note = Synthetic Polypeptide
APVQPQQSPAAAPGGTDEKPSGK

SEQ ID NO: 994 - protein, 23 aa, synthetic construct
  REGION 1..23: note = Synthetic Polypeptide
APVQPQQSPAAAPGGTDEKPSGK

SEQ ID NO: 995 - protein, 22 aa, synthetic construct
  REGION 1..22: note = Synthetic Polypeptide
NDGPVTIELESPAPGTATSDPK

SEQ ID NO: 996 - protein, 20 aa, synthetic construct
  REGION 1..20: note = Synthetic Polypeptide
INSLFLTDLYSPEYPGPSHR

SEQ ID NO: 997 - protein, 23 aa, synthetic construct
  REGION 1..23: note = Synthetic Polypeptide
NGSLDSPGKQDTEEDEEEDEKDK

SEQ ID NO: 998 - protein, 24 aa, synthetic construct
  REGION 1..24: note = Synthetic Polypeptide
SAAAASAASGSPGPGEGSAGGEKR

SEQ ID NO: 999 - protein, 24 aa, synthetic construct
  REGION 1..24: note = Synthetic Polypeptide
SAAAASAASGSPGPGEGSAGGEKR

SEQ ID NO: 1000 - protein, 21 aa, synthetic construct
  REGION 1..21: note = Synthetic Polypeptide
NADTDLVSWLSPHDPNSVVTK

SEQ ID NO: 1001 - protein, 17 aa, synthetic construct
  REGION 1..17: note = Synthetic Polypeptide
LSPPYSSPQEFAQDVGR

SEQ ID NO: 1002 - protein, 18 aa, synthetic construct
  REGION 1..18: note = Synthetic Polypeptide
IIAFVGSPVEDNEKDLVK

SEQ ID NO: 1003 - protein, 14 aa, synthetic construct
  REGION 1..14: note = Synthetic Polypeptide
MESQEPTESSQNGK

SEQ ID NO: 1004 - protein, 12 aa, synthetic construct
  REGION 1..12: note = Synthetic Polypeptide
  VARIANT 2: note = misc_feature - Xaa can be any naturally occurring amino acid
AXASQLDCNFLK

SEQ ID NO: 1005 - protein, 18 aa, synthetic construct
  REGION 1..18: note = Synthetic Polypeptide
SQGDSISSQLGPIHPPPR

SEQ ID NO: 1006 - protein, 20 aa, synthetic construct
  REGION 1..20: note = Synthetic Polypeptide
LGGLLKPTVASQNQNLPVAK

SEQ ID NO: 1007 - protein, 30 aa, synthetic construct
  REGION 1..30: note = Synthetic Polypeptide
SSWGMMGMLASQQNQSGPSGNNQNQGNMQR

SEQ ID NO: 1008 - protein, 15 aa, synthetic construct
  REGION 1..15: note = Synthetic Polypeptide
DEYLINSQTTEHIVK

SEQ ID NO: 1009 - protein, 22 aa, synthetic construct
  REGION 1..22: note = Synthetic Polypeptide
YQLGLAYGYNSQYDEAVAQFSK

SEQ ID NO: 1010 - protein, 19 aa, synthetic construct
  REGION 1..19: note = Synthetic Polypeptide
GLLLLSVVVTSRPEAFQPH

SEQ ID NO: 1011 - protein, 16 aa, synthetic construct
  REGION 1..16: note = Synthetic Polypeptide
RPASVSSSAAVEHEQR

SEQ ID NO: 1012 - protein, 19 aa, synthetic construct
  REGION 1..19: note = Synthetic Polypeptide
FGIVTSSAGTGTTEDTEAK

SEQ ID NO: 1013 - protein, 20 aa, synthetic construct
  REGION 1..20: note = Synthetic Polypeptide
FGIVTSSAGTGTTEDTEAKK

SEQ ID NO: 1014 - protein, 23 aa, synthetic construct
  REGION 1..23: note = Synthetic Polypeptide
STASAPAAVNSSASADKPLSNMK

SEQ ID NO: 1015 - protein, 16 aa, synthetic construct
  REGION 1..16: note = Synthetic Polypeptide
EALLSSAVDHGSDEVK

SEQ ID NO: 1016 - protein, 17 aa, synthetic construct
  REGION 1..17: note = Synthetic Polypeptide
VSWLEYESSFSNEEAQK

SEQ ID NO: 1017 - protein, 20 aa, synthetic construct
  REGION 1..20: note = Synthetic Polypeptide
  VARIANT 2..3: note = misc_feature - Xaa can be any naturally occurring amino acid
IXXGSSGAQGSGGGSTSAHY

SEQ ID NO: 1018 - protein, 21 aa, synthetic construct
  REGION 1..21: note = Synthetic Polypeptide
HIGGPPGFASSSGKPGPTVIK

SEQ ID NO: 1019 - protein, 22 aa, synthetic construct
  REGION 1..22: note = Synthetic Polypeptide
FEMYEPSELESSHLTDQDNEIR

SEQ ID NO: 1020 - protein, 21 aa, synthetic construct
  REGION 1..21: note = Synthetic Polypeptide
SPDDDLGSSNWEAADLGNEER

SEQ ID NO: 1021 - protein, 28 aa, synthetic construct
  REGION 1..28: note = Synthetic Polypeptide
GDSQVSSNPTSSPPGEAPAPVSVDSEPS

SEQ ID NO: 1022 - protein, 18 aa, synthetic construct
  REGION 1..18: note = Synthetic Polypeptide
FVNGQPRPLESSQVKYLR

SEQ ID NO: 1023 - protein, 18 aa, synthetic construct
  REGION 1..18: note = Synthetic Polypeptide
KPLTSSSAAPQRPISTQR

SEQ ID NO: 1024 - protein, 22 aa, synthetic construct
  REGION 1..22: note = Synthetic Polypeptide
IHIGGPPGFASSSGKPGPTVIK

SEQ ID NO: 1025 - protein, 19 aa, synthetic construct
  REGION 1..19: note = Synthetic Polypeptide
ELVSSSSSGSDSDSEVDKK

SEQ ID NO: 1026 - protein, 12 aa, synthetic construct
  REGION 1..12: note = Synthetic Polypeptide
LLDSSTVTHLFK

SEQ ID NO: 1027 - protein, 23 aa, synthetic construct
  REGION 1..23: note = Synthetic Polypeptide
PPPAAPPPSSSSVPEAGGPPIKK

SEQ ID NO: 1028 - protein, 20 aa, synthetic construct
  REGION 1..20: note = Synthetic Polypeptide
YVELFLNSTAGASGGAYEHR

SEQ ID NO: 1029 - protein, 22 aa, synthetic construct
  REGION 1..22: note = Synthetic Polypeptide
SHELSDFGLESTAGEIPVVAIR

SEQ ID NO: 1030 - protein, 22 aa, synthetic construct
  REGION 1..22: note = Synthetic Polypeptide
ECEEEAINIQSTAPEEEHESPR

SEQ ID NO: 1031 - protein, 19 aa, synthetic construct
  REGION 1..19: note = Synthetic Polypeptide
EGTGSTATSSSSTAGAAGK

SEQ ID NO: 1032 - protein, 18 aa, synthetic construct
  REGION 1..18: note = Synthetic Polypeptide
PLHSIISSTESVQGSTSK

SEQ ID NO: 1033 - protein, 15 aa, synthetic construct
  REGION 1..15: note = Synthetic Polypeptide
VAFTGSTEVGHLIQK

SEQ ID NO: 1034 - protein, 20 aa, synthetic construct
  REGION 1..20: note = Synthetic Polypeptide
LALVTGGEIASTFDHPELVK

SEQ ID NO: 1035 - protein, 17 aa, synthetic construct
  REGION 1..17: note = Synthetic Polypeptide
ATIELCSTHANDASALR

SEQ ID NO: 1036 - protein, 16 aa, synthetic construct
  REGION 1..16: note = Synthetic Polypeptide
VHITLSTHECAGLSER

SEQ ID NO: 1037 - protein, 18 aa, synthetic construct
  REGION 1..18: note = Synthetic Polypeptide
EEEEPQAPQESTPAPPKK

SEQ ID NO: 1038 - protein, 16 aa, synthetic construct
  REGION 1..16: note = Synthetic Polypeptide
SITILSTPEGTSAACK

SEQ ID NO: 1039 - protein, 19 aa, synthetic construct
  REGION 1..19: note = Synthetic Polypeptide
ETLASSDSFASTQPTHSWK

SEQ ID NO: 1040 - protein, 16 aa, synthetic construct
  REGION 1..16: note = Synthetic Polypeptide
VVVLMGSTSDLGHCEK

SEQ ID NO: 1041 - protein, 24 aa, synthetic construct
  REGION 1..24: note = Synthetic Polypeptide
VLLSNLSSTSHVPEVDPGSAELQK

SEQ ID NO: 1042 - protein, 11 aa, synthetic construct
  REGION 1..11: note = Synthetic Polypeptide
LFDSTTLEHQK

SEQ ID NO: 1043 - protein, 16 aa, synthetic construct
  REGION 1..16: note = Synthetic Polypeptide
TQLEGLQSTVTGHVER

SEQ ID NO: 1044 - protein, 21 aa, synthetic construct
  REGION 1..21: note = Synthetic Polypeptide
GSESGGSAVDSVAGEHSVSGR

SEQ ID NO: 1045 - protein, 15 aa, synthetic construct
  REGION 1..15: note = Synthetic Polypeptide
YEILQSVDDAAIVIK

SEQ ID NO: 1046 - protein, 19 aa, synthetic construct
  REGION 1..19: note = Synthetic Polypeptide
NDLSICGTLHSVDQYLNIK

SEQ ID NO: 1047 - protein, 13 aa, synthetic construct
  REGION 1..13: note = Synthetic Polypeptide
ILDSVGIEADDDR

SEQ ID NO: 1048 - protein, 16 aa, synthetic construct
  REGION 1..16: note = Synthetic Polypeptide
ILDSVGIEADDDRLNK

SEQ ID NO: 1049 - protein, 16 aa, synthetic construct
  REGION 1..16: note = Synthetic Polypeptide
IYVASVHQDLSDDDIK

SEQ ID NO: 1050 - protein, 12 aa, synthetic construct
  REGION 1..12: note = Synthetic Polypeptide
ELQSVKPQEAPK

SEQ ID NO: 1051 - protein, 18 aa, synthetic construct
  REGION 1..18: note = Synthetic Polypeptide
HYTEGAELVDSVLDVVRK

SEQ ID NO: 1052 - protein, 19 aa, synthetic construct
  REGION 1..19: note = Synthetic Polypeptide
LAEGSVTSVGSVNPAENFR

SEQ ID NO: 1053 - protein, 19 aa, synthetic construct
  REGION 1..19: note = Synthetic Polypeptide
GSPTSLGTWGSWIGPDHDK

SEQ ID NO: 1054 - protein, 14 aa, synthetic construct
  REGION 1..14: note = Synthetic Polypeptide
VLNSYWVGEDSTYK

SEQ ID NO: 1055 - protein, 11 aa, synthetic construct
  REGION 1..11: note = Synthetic Polypeptide
SLGTADVHFER

SEQ ID NO: 1056 - protein, 12 aa, synthetic construct
  REGION 1..12: note = Synthetic Polypeptide
MAGTAFDFENMK

SEQ ID NO: 1057 - protein, 13 aa, synthetic construct
  REGION 1..13: note = Synthetic Polypeptide
VLATAFDTTLGGR

SEQ ID NO: 1058 - protein, 19 aa, synthetic construct
  REGION 1..19: note = Synthetic Polypeptide
VELFLNSTAGASGGAYEHR

SEQ ID NO: 1059 - protein, 20 aa, synthetic construct
  REGION 1..20: note = Synthetic Polypeptide
APPPSGSAVSTAPQQKPIGK

SEQ ID NO: 1060 - protein, 17 aa, synthetic construct
  REGION 1..17: note = Synthetic Polypeptide
SQIFSTASDNQPTVTIK

SEQ ID NO: 1061 - protein, 14 aa, synthetic construct
  REGION 1..14: note = Synthetic Polypeptide
IYWGTATTGKPHVA

SEQ ID NO: 1062 - protein, 14 aa, synthetic construct
  REGION 1..14: note = Synthetic Polypeptide
MMLGTEGGEGFVVK

SEQ ID NO: 1063 - protein, 17 aa, synthetic construct
  REGION 1..17: note = Synthetic Polypeptide
FGAVWTGDNTAEWDHLK

SEQ ID NO: 1064 - protein, 15 aa, synthetic construct
  REGION 1..15: note = Synthetic Polypeptide
VSHVSTGGGASLELL

SEQ ID NO: 1065 - protein, 16 aa, synthetic construct
  REGION 1..16: note = Synthetic Polypeptide
VSHVSTGGGASLELLE

SEQ ID NO: 1066 - protein, 18 aa, synthetic construct
  REGION 1..18: note = Synthetic Polypeptide
VSHVSTGGGASLELLEGK

SEQ ID NO: 1067 - protein, 21 aa, synthetic construct
  REGION 1..21: note = Synthetic Polypeptide
ILISLATGHREEGGENLDQAR

SEQ ID NO: 1068 - protein, 19 aa, synthetic construct
  REGION 1..19: note = Synthetic Polypeptide
TLDQCIQTGVDNPGHPFIK

SEQ ID NO: 1069 - protein, 20 aa, synthetic construct
  REGION 1..20: note = Synthetic Polypeptide
SGFTLDDVIQTGVDNPGHPY

SEQ ID NO: 1070 - protein, 14 aa, synthetic construct
  REGION 1..14: note = Synthetic Polypeptide
DLTTGYDDSQPDKK

SEQ ID NO: 1071 - protein, 14 aa, synthetic construct
  REGION 1..14: note = Synthetic Polypeptide
FFFGTHETAFLGPK

SEQ ID NO: 1072 - protein, 14 aa, synthetic construct
  REGION 1..14: note = Synthetic Polypeptide
FPSLLTHNENMVAK

SEQ ID NO: 1073 - protein, 17 aa, synthetic construct
  REGION 1..17: note = Synthetic Polypeptide
YEDICPSTHNMDVPNIK

SEQ ID NO: 1074 - protein, 24 aa, synthetic construct
  REGION 1..24: note = Synthetic Polypeptide
DYALHWLVLGTHTSDEQNHLVVAR

SEQ ID NO: 1075 - protein, 10 aa, synthetic construct
  REGION 1..10: note = Synthetic Polypeptide
FGTINIVHPK

SEQ ID NO: 1076 - protein, 18 aa, synthetic construct
  REGION 1..18: note = Synthetic Polypeptide
SMVNTKPEKTEEDSEEVR

SEQ ID NO: 1077 - protein, 29 aa, synthetic construct
  REGION 1..29: note = Synthetic Polypeptide
VTLLTPAGATGSGGGTSGDSSKGEDKQDR

SEQ ID NO: 1078 - protein, 23 aa, synthetic construct
  REGION 1..23: note = Synthetic Polypeptide
PGETLTEILETPATSEQEAEHQR

SEQ ID NO: 1079 - protein, 18 aa, synthetic construct
  REGION 1..18: note = Synthetic Polypeptide
NSVQTPVENSTNSQHQVK

SEQ ID NO: 1080 - protein, 16 aa, synthetic construct
  REGION 1..16: note = Synthetic Polypeptide
  VARIANT 2..3: note = misc_feature - Xaa can be any naturally occurring amino acid
AXXITPVPGGVGPMTV

SEQ ID NO: 1081 - protein, 21 aa, synthetic construct
  REGION 1..21: note = Synthetic Polypeptide
STVLTPMFVETQASQGTLQTR

SEQ ID NO: 1082 - protein, 15 aa, synthetic construct
  REGION 1..15: note = Synthetic Polypeptide
TFTTQETITNAETAK

SEQ ID NO: 1083 - protein, 14 aa, synthetic construct
  REGION 1..14: note = Synthetic Polypeptide
SPVSTRPLPSASQK

SEQ ID NO: 1084 - protein, 19 aa, synthetic construct
  REGION 1..19: note = Synthetic Polypeptide
TNEQWQMSLGTSEDHQHFT

SEQ ID NO: 1085 - protein, 18 aa, synthetic construct
  REGION 1..18: note = Synthetic Polypeptide
  VARIANT 5: note = misc_feature - Xaa can be any naturally occurring amino acid
QEIIXQLDVTTSEYEKEK

SEQ ID NO: 1086 - protein, 24 aa, synthetic construct
  REGION 1..24: note = Synthetic Polypeptide
LLAFLLAELGTSGSIDGNNQLVIK

SEQ ID NO: 1087 - protein, 17 aa, synthetic construct
  REGION 1..17: note = Synthetic Polypeptide
  VARIANT 2: note = misc_feature - Xaa can be any naturally occurring amino acid
LXNMEIGTSLFDEEGAK

SEQ ID NO: 1088 - protein, 25 aa, synthetic construct
  REGION 1..25: note = Synthetic Polypeptide
AEKPAETPVATSPTATDSTSGDSSR

SEQ ID NO: 1089 - protein, 16 aa, synthetic construct
  REGION 1..16: note = Synthetic Polypeptide
LLETTDRPDGHQNNLR

SEQ ID NO: 1090 - protein, 19 aa, synthetic construct
  REGION 1..19: note = Synthetic Polypeptide
  VARIANT 8..9: note = misc_feature - Xaa can be any naturally occurring amino acid
AQTITSEXXSTTTTTHITK

SEQ ID NO: 1091 - protein, 16 aa, synthetic construct
  REGION 1..16: note = Synthetic Polypeptide
ADAVGMSTVPEVIVAR

SEQ ID NO: 1092 - protein, 16 aa, synthetic construct
  REGION 1..16: note = Synthetic Polypeptide
IHFPLATYAPVISAEK

SEQ ID NO: 1093 - protein, 16 aa, synthetic construct
  REGION 1..16: note = Synthetic Polypeptide
  VARIANT 3: note = misc_feature - Xaa can be any naturally occurring amino acid
  VARIANT 5..6: note = misc_feature - Xaa can be any naturally occurring amino acid
DTXVXXDTYNCDLHFK

SEQ ID NO: 1094 - protein, 14 aa, synthetic construct
  REGION 1..14: note = Synthetic Polypeptide
VVIGMDVAASEFFR

SEQ ID NO: 1095 - protein, 20 aa, synthetic construct
  REGION 1..20: note = Synthetic Polypeptide
  VARIANT 2..6: note = misc_feature - Xaa can be any naturally occurring amino acid
  VARIANT 10: note = misc_feature - Xaa can be any naturally occurring amino acid
GXXXXXIGLXVADLAESIMK

SEQ ID NO: 1096 - protein, 13 aa, synthetic construct
  REGION 1..13: note = Synthetic Polypeptide
ANPQVGVAFPHIK

SEQ ID NO: 1097 - protein, 23 aa, synthetic construct
  REGION 1..23: note = Synthetic Polypeptide
PQEAKPQEAAVAPEKPPASDETK

SEQ ID NO: 1098 - protein, 11 aa, synthetic construct
  REGION 1..11: note = Synthetic Polypeptide
HFSVEGQLEFR

SEQ ID NO: 1099 - protein, 14 aa, synthetic construct
  REGION 1..14: note = Synthetic Polypeptide
VATLGVEVHPLVFH

SEQ ID NO: 1100 - protein, 14 aa, synthetic construct
  REGION 1..14: note = Synthetic Polypeptide
HWPFQVINDGDKPK

SEQ ID NO: 1101 - protein, 18 aa, synthetic construct
  REGION 1..18: note = Synthetic Polypeptide
LPVPAFNVINGGSHAGNK

SEQ ID NO: 1102 - protein, 21 aa, synthetic construct
  REGION 1..21: note = Synthetic Polypeptide
EVANGIESLGVKPDLPPPPSK

SEQ ID NO: 1103 - protein, 19 aa, synthetic construct
  REGION 1..19: note = Synthetic Polypeptide
TYYDVLGVKPNATQEELKK

SEQ ID NO: 1104 - protein, 18 aa, synthetic construct
  REGION 1..18: note = Synthetic Polypeptide
ETVAVKPTENNEEEFTSK

SEQ ID NO: 1105 - protein, 13 aa, synthetic construct
  REGION 1..13: note = Synthetic Polypeptide
SLLVNPEGPTLMR

SEQ ID NO: 1106 - protein, 16 aa, synthetic construct
  REGION 1..16: note = Synthetic Polypeptide
NWMNSLGVNPHVNHLY

SEQ ID NO: 1107 - protein, 18 aa, synthetic construct
  REGION 1..18: note = Synthetic Polypeptide
HGLLVPNNTTDQELQHIR

SEQ ID NO: 1108 - protein, 18 aa, synthetic construct
  REGION 1..18: note = Synthetic Polypeptide
QELEFLEVQEEYIKDEQK

SEQ ID NO: 1109 - protein, 20 aa, synthetic construct
  REGION 1..20: note = Synthetic Polypeptide
LEGTLLKPNMVTPGHACTQK

SEQ ID NO: 1110 - protein, 11 aa, synthetic construct
  REGION 1..11: note = Synthetic Polypeptide
FVNVVPTFGKK

SEQ ID NO: 1111 - protein, 16 aa, synthetic construct
  REGION 1..16: note = Synthetic Polypeptide
EDLVFIFWAPESAPLK

SEQ ID NO: 1112 - protein, 19 aa, synthetic construct
  REGION 1..19: note = Synthetic Polypeptide
AIYIDASCLTWEGQQFQGK

SEQ ID NO: 1113 - protein, 21 aa, synthetic construct
  REGION 1..21: note = Synthetic Polypeptide
EQPQHPLHVTYAGAAVDELGK

SEQ ID NO: 1114 - protein, 18 aa, synthetic construct
  REGION 1..18: note = Synthetic Polypeptide
SPDGHLFQVEYAQEAVKK

SEQ ID NO: 1115 - protein, 8 aa, synthetic construct
  REGION 1..8: note = Synthetic Polypeptide
NYKPPAQK

SEQ ID NO: 1116 - protein, 11 aa, synthetic construct
  REGION 1..11: note = Synthetic Polypeptide
VYNYNHLMPTR

SEQ ID NO: 1117 - protein, 15 aa, synthetic construct
  REGION 1..15: note = Synthetic Polypeptide
LAEAELEYNPEHVSR

SEQ ID NO: 1118 - protein, 13 aa, synthetic construct
  REGION 1..13: note = Synthetic Polypeptide
MPYQYPALTPEQK

SEQ ID NO: 1119 - protein, 18 aa, synthetic construct
  REGION 1..18: note = Synthetic Polypeptide
TSSANNPNLMYQDECDRR

SEQ ID NO: 1120 - protein, 18 aa, synthetic construct
  REGION 1..18: note = Synthetic Polypeptide
VGINYQPPTVVPGGDLAK

SEQ ID NO: 1121 - protein, 15 aa, synthetic construct
  REGION 1..15: note = Synthetic Polypeptide
  VARIANT 6: note = misc_feature - Xaa can be any naturally occurring amino acid
YMACCXLYRGDVVPK

SEQ ID NO: 1122 - protein, 11 aa, synthetic construct
  REGION 1..11: note = Synthetic Polypeptide
SYCYVSKEELK

SEQ ID NO: 1123 - protein, 10 aa, synthetic construct
  REGION 1..10: note = Synthetic Polypeptide
EAAGIGILTV

SEQ ID NO: 1124 - protein, 10 aa, synthetic construct
  REGION 1..10: note = Synthetic Polypeptide
ELAGIGILTV

SEQ ID NO: 1125 - protein, 9 aa, synthetic construct
  REGION 1..9: note = Synthetic Polypeptide
YMDGTMSQV

SEQ ID NO: 1126 - protein, 140 aa, synthetic construct
  REGION 1..140: note = Synthetic Polypeptide
MHHHHHHHHHHKENQPENSQTPGGGSVPLAHSSSAFTIMDQVPFSVSVSQLQSSMHNALHIYMDGTMSQVQGSANDGVGAYGTVYKACDPHSGHFVALKSVGHGHSYTTAEELAGIGILTVILGVLGKPIPNPLLGLDST

SEQ ID NO: 1127 - protein, 115 aa, synthetic construct
  REGION 1..115: note = Synthetic Polypeptide
MHHHHHHHHHHKENQPENSQTPGGGSVPLAHSSSAFTIMDQVPFSVSVSQLVPLAHSSSAFTIMDQVPFSVSVSQLVPLAHSSSAFTIMDQVPFSVSVSQLGKPIPNPLLGLDST

SEQ ID NO: 1128 - RNA, 51 nt, synthetic construct
  misc_feature 1..51: note = Synthetic Polynucleotide
  modified_base 1: mod_base = OTHER; note = Modified with 7-methyl
  modified_base 1: mod_base = OTHER; note = Modified with inorganic triphosphate
  modified_base 2: mod_base = OTHER; note = Modified with 2'-O-methyl
  source 1..51: mol_type = other RNA
ggggaaataagagagaaaagaagagtaagaagaaatataagagccaccatg

SEQ ID NO: 1129 - RNA, 226 nt, synthetic construct
  misc_feature 1..226: note = Synthetic Polynucleotide
  modified_base 226: mod_base = OTHER; note = Modified with OH
  source 1..226: mol_type = other RNA
tgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 1130 - protein, 12 aa, synthetic construct
  REGION 1..12: note = Synthetic
HEYGAEALERAG

SEQ ID NO: 1131 - protein, 5 aa, synthetic construct
  REGION 1..5: note = Synthetic
KFERQ

SEQ ID NO: 1132 - skipped